SEQUENCE LISTING (126 sequences)

SEQ ID NO: 1 - DNA, 24 bp, synthetic construct
  source 1..24: mol_type = other DNA
gaagaatctgcttagggttaggcg

SEQ ID NO: 2 - DNA, 19 bp, synthetic construct
  source 1..19: mol_type = other DNA
gaattccacgatgctgatg

SEQ ID NO: 3 - DNA, 20 bp, synthetic construct
  source 1..20: mol_type = other DNA
cgagccccatgagtttgagg

SEQ ID NO: 4 - DNA, 21 bp, synthetic construct
  source 1..21: mol_type = other DNA
ccacagctgacattctttacc

SEQ ID NO: 5 - DNA, 19 bp, synthetic construct
  source 1..19: mol_type = other DNA
ctacaagcacaccacttcc

SEQ ID NO: 6 - DNA, 32 bp, synthetic construct
  source 1..32: mol_type = other DNA
caacttacttctgacaacgatcggaggaccga

SEQ ID NO: 7 - DNA, 32 bp, synthetic construct
  source 1..32: mol_type = other DNA
tagctccttcggtcctccgatcgttgtcagaa

SEQ ID NO: 8 - DNA, 26 bp, synthetic construct
  source 1..26: mol_type = other DNA
agaatagaccgagatagggttgagtg

SEQ ID NO: 9 - DNA, 26 bp, synthetic construct
  source 1..26: mol_type = other DNA
cactcaaccctatctcggtctattct

SEQ ID NO: 10 - DNA, 21 bp, synthetic construct
  source 1..21: mol_type = other DNA
gtactggaactggggggacag

SEQ ID NO: 11 - DNA, 19 bp, synthetic construct
  source 1..19: mol_type = other DNA
gtgttccgcattctgcaag

SEQ ID NO: 12 - DNA, 20 bp, synthetic construct
  source 1..20: mol_type = other DNA
cctctagagccgccatgacc

SEQ ID NO: 13 - DNA, 18 bp, synthetic construct
  source 1..18: mol_type = other DNA
ccctctagagccgccatg

SEQ ID NO: 14 - DNA, 23 bp, synthetic construct
  source 1..23: mol_type = other DNA
caagatccgccacaatatcgagg

SEQ ID NO: 15 - DNA, 31 bp, synthetic construct
  source 1..31: mol_type = other DNA
gtactcgagagaatgcacaatgaggcttatc

SEQ ID NO: 16 - DNA, 19 bp, synthetic construct
  source 1..19: mol_type = other DNA
caacacagtgctgtctggc

SEQ ID NO: 17 - DNA, 20 bp, synthetic construct
  source 1..20: mol_type = other DNA
caagatgagattggcatggc

SEQ ID NO: 18 - DNA, 30 bp, synthetic construct
  source 1..30: mol_type = other DNA
ccctctagagccgccatgaccctgcggtgc

SEQ ID NO: 19 - DNA, 52 bp, synthetic construct
  source 1..52: mol_type = other DNA
ctgtcgtaagtccgctacctggaatttatattcttccaagtaatcttttagt

SEQ ID NO: 20 - DNA, 28 bp, synthetic construct
  source 1..28: mol_type = other DNA
ccctctagagccgccatggaggagccgc

SEQ ID NO: 21 - DNA, 28 bp, synthetic construct
  source 1..28: mol_type = other DNA
ctgtcgtaagtccgcgtctgagtcaggc

SEQ ID NO: 22 - DNA, 20 bp, synthetic construct
  source 1..20: mol_type = other DNA
gaaagggtgtaacgcaactg

SEQ ID NO: 23 - DNA, 23 bp, synthetic construct
  source 1..23: mol_type = other DNA
gaaagggtgtaacgcaactgtcg

SEQ ID NO: 24 - DNA, 22 bp, synthetic construct
  source 1..22: mol_type = other DNA
cttctcattgggatctttgctc

SEQ ID NO: 25 - DNA, 19 bp, synthetic construct
  source 1..19: mol_type = other DNA
ccacaatatcgaggatggc

SEQ ID NO: 26 - DNA, 21 bp, synthetic construct
  source 1..21: mol_type = other DNA
cgagaaagggtgtaacgcaac

SEQ ID NO: 27 - DNA, 19 bp, synthetic construct
  source 1..19: mol_type = other DNA
gggtgtaacgcaactgtcg

SEQ ID NO: 28 - DNA, 24 bp, synthetic construct
  source 1..24: mol_type = other DNA
cagcaagcaggagtatgacgagtc

SEQ ID NO: 29 - DNA, 18 bp, synthetic construct
  source 1..18: mol_type = other DNA
catgccaatctcatcttg

SEQ ID NO: 30 - DNA, 29 bp, synthetic construct
  source 1..29: mol_type = other DNA
ttgtcaagaaagggtgtaacgcaactaag

SEQ ID NO: 31 - DNA, 20 bp, synthetic construct
  source 1..20: mol_type = other DNA
ctgttaatgaagccaaagag

SEQ ID NO: 32 - DNA, 19 bp, synthetic construct
  source 1..19: mol_type = other DNA
gcacatctgaacatagtag

SEQ ID NO: 33 - DNA, 20 bp, synthetic construct
  source 1..20: mol_type = other DNA
cagatgaagctcccagaatg

SEQ ID NO: 34 - DNA, 18 bp, synthetic construct
  source 1..18: mol_type = other DNA
gtcccagaatgcaagaag

SEQ ID NO: 35 - DNA, 20 bp, synthetic construct
  source 1..20: mol_type = other DNA
gctgcgaagtggaaaccatc

SEQ ID NO: 36 - DNA, 22 bp, synthetic construct
  source 1..22: mol_type = other DNA
cctccttctgcacacatttgaa

SEQ ID NO: 37 - DNA, 21 bp, synthetic construct
  source 1..21: mol_type = other DNA
gctcttcagttcgtgtgtgga

SEQ ID NO: 38 - DNA, 21 bp, synthetic construct
  source 1..21: mol_type = other DNA
gcctccttagatcacagctcc

SEQ ID NO: 39 - DNA, 19 bp, synthetic construct
  source 1..19: mol_type = other DNA
tgtccgtcagaacccatgc

SEQ ID NO: 40 - DNA, 21 bp, synthetic construct
  source 1..21: mol_type = other DNA
aaagtcgaagttccatcgctc

SEQ ID NO: 41 - DNA, 21 bp, synthetic construct
  source 1..21: mol_type = other DNA
agaggctcgctgtttctatgg

SEQ ID NO: 42 - DNA, 21 bp, synthetic construct
  source 1..21: mol_type = other DNA
gcaaggcccaatctgcttttt

SEQ ID NO: 43 - DNA, 25 bp, synthetic construct
  source 1..25: mol_type = other DNA
tggatctgttgtgatatccgctacc

SEQ ID NO: 44 - DNA, 23 bp, synthetic construct
  source 1..23: mol_type = other DNA
ctgcctgtgacccagaggaagag

SEQ ID NO: 45 - DNA, 19 bp, synthetic construct
  source 1..19: mol_type = other DNA
cttcaaggtgcacatggag

SEQ ID NO: 46 - DNA, 22 bp, synthetic construct
  source 1..22: mol_type = other DNA
ttgacactactcctgtttgccc

SEQ ID NO: 47 - DNA, 25 bp, synthetic construct
  source 1..25: mol_type = other DNA
aaaagttccaagtttcaaagctggg

SEQ ID NO: 48 - DNA, 21 bp, synthetic construct
  source 1..21: mol_type = other DNA
tgtcgactcctttgcttcgga

SEQ ID NO: 49 - DNA, 22 bp, synthetic construct
  source 1..22: mol_type = other DNA
aaatcgattgcacccacaagta

SEQ ID NO: 50 - DNA, 20 bp, synthetic construct
  source 1..20: mol_type = other DNA
agccgcacagaaagatcacc

SEQ ID NO: 51 - DNA, 23 bp, synthetic construct
  source 1..23: mol_type = other DNA
aagtgcaaaaatactaaggtggg

SEQ ID NO: 52 - DNA, 30 bp, synthetic construct
  source 1..30: mol_type = other DNA
agcgcgatcacatggtcctgctggagttcg

SEQ ID NO: 53 - DNA, 30 bp, synthetic construct
  source 1..30: mol_type = other DNA
ccgccgcatctaacacattgatcctagcag

SEQ ID NO: 54 - DNA, 30 bp, synthetic construct
  source 1..30: mol_type = other DNA
gcggacttacgacagttgcgttacaccctt

SEQ ID NO: 55 - DNA, 30 bp, synthetic construct
  source 1..30: mol_type = other DNA
ttgcggcgttagcggtagatcacgttatcg

SEQ ID NO: 56 - DNA, 1323 bp, synthetic construct
  source 1..1323: mol_type = other DNA
atgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaagcggcagcgagactcccgggacctcagagtccgccacaccagaaccccacccagtgttggagaagcttcggtccattaataactataaccccaaagattttgactggaatctgaaacatggccgggttttcatcattaagagctactctgaggacgatattcaccgttccattaagtataatatttggtgcagcacagagcatggtaacaagagactggatgctgcttatcgttccatgaacgggaaaggccccgtttacttacttttcagtgtcaacggcagtggacacttctgtggcgtggcagaaatgaaatctgctgtggactacaacacatgtgcaggtgtgtggtcccaggacaaatggaagggtcgttttgatgtcaggtggatttttgtgaaggacgttcccaatagccaactgcgacacattcgcctagagaacaacgagaataaaccagtgaccaactctagggacactcaggaagtgcctctggaaaaggctaagcaggtgttgaaaattatagccagctacaagcacaccacttccatttttgatgacttctcacactatgagaaacgccaagaggaagaagaaagtgttaaaaaggaacgtcaaggtcgtgggaaactcgag

SEQ ID NO: 57 - DNA, 1248 bp, synthetic construct
  source 1..1248: mol_type = other DNA
atgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaagcggcagcgagactcccgggacctcagagtccgccacaccagaaggccgggttttcatcattaagagctactctgaggacgatattcaccgttccattaagtataatatttggtgcagcacagagcatggtaacaagagactggatgctgcttatcgttccatgaacgggaaaggccccgtttacttacttttcagtgtcaacggcagtggacacttctgtggcgtggcagaaatgaaatctgctgtggactacaacacatgtgcaggtgtgtggtcccaggacaaatggaagggtcgttttgatgtcaggtggatttttgtgaaggacgttcccaatagccaactgcgacacattcgcctagagaacaacgagaataaaccagtgaccaactctagggacactcaggaagtgcctctggaaaaggctaagcaggtgttgaaaattatagccagctacaagcacaccacttccatttttgatgacttctcacactatgagaaacgccaagaggaagaagaaagtgttaaaaaggaacgtcaaggtcgtgggaaactcgag

SEQ ID NO: 58 - DNA, 4690 bp, synthetic construct
  source 1..4690: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaccagtgtgatggatatctgcagaattcgcccttggatccgaattcctgcagccccgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgagggggatccactagcatgaagggcgaattccagcacactggtaacccgtgtcggctccagatctggcctccgcgccgggttttggcgcctcccgcgggcgcccccctcctcacggcgagccgcgttgacattgattattgactaggcttttgcaaaaagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtaccgagctcggatccactagtccagtgtggtggaattctgcagatatccagcacagtggcggccgctcgagtctagaggatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggc

SEQ ID NO: 59 - DNA, 4104 bp, synthetic construct
  source 1..4104: mol_type = other DNA
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 60 - DNA, 7434 bp, synthetic construct
  source 1..7434: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtcaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaacgcatacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatggatccaaaagaaccgccgacggcagcgaattcgagcccaagaagaagaggaaagtctctggcagcgagacaccaggaacaagcgagtcagcaacaccagagagccaagagttctgtgactatggcaccaaagaggagtgcatgaaggctagcgacgctgatcgtccatgccgtaagctgcacttccgtcgcatcattaacaaacacaccgacgagagcctgggcgactgcagcttcctgaacacctgctttcacatggacacctgcaagtacgtgcactatgagatcgacgcgtgcatggatagcgaagctccgggcagcaaagatcacaccccgagccaggaactggccctgacccagagcgtgggtggtgacagcagcgcggatcgtctgttcccaccacagtggatctgctgcgacattcgttacctggatgtgagcatcctgggcaagtttgctgttgtgatggccgacccgccgtgggatattcacatggagctgccgtatggtaccctgaccgacgatgaaatgcgtcgcctgaacatcccggtgctgcaggacgatggcttcctgtttctgtgggtgacgggtcgtgctatggagctgggtcgtgaatgcctgaacctgtggggttacgagcgtgtggacgaaatcatttgggtgaaaaccaaccagctgcagcgtatcattcgcaccggccgtaccggtcactggctgaaccacggcaaggagcactgcctggtgggcgtgaaaggcaacccgcagggctttaaccagggtctggactgcgatgtgatcgtggctgaagtgcgcagcaccagccacaagccggacgagatctacggcatgattgaacgcctgagcccgggtacccgtaaaattgagctgttcggccgtccgcacaacgtgcagccgaactggatcaccctgggcaaccagctggacggtattcacctgctggacccagatgtggtggctcgctttaaacagcgttatccggatggcatcattagcaaaccgaagaatctgtaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 61 - DNA, 2908 bp, synthetic construct
  source 1..2908: mol_type = other DNA
attgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcagctagcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatttagactaccccaaaaacgaaggggactaaaacgggtcttcgagaagaccttttttttgaattctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttag

SEQ ID NO: 62 - DNA, 678 bp, synthetic construct
  source 1..678: mol_type = other DNA
atggatagcactgagaacgtcatcaagcccttcatgcgcttcaaggtgcacatggagggctccgtgaacggccacgagttcgagatcgagggcgagggcgagggcaagccctacgagggcacccagaccgccaagctgcaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggcaccttcatctaccacgtgaagttcatcggcgtgaacttcccctccgacggccccgtaatgcagaagaagactctgggctgggagccctccaccgagcgcctgtacccccgcgacggcgtgctgaagggcgagatccacaaggcgctgaagctgaagggcggcggccactacctggtggagttcaagtcaatctacatggccaagaagcccgtgaagctgcccggctactactacgtggactccaagctggacatcacctcccacaacgaggactacaccgtggtggagcagtacgagcgcgccgaggcccgccaccacctgttccagtag

SEQ ID NO: 63 - DNA, 717 bp, synthetic construct
  source 1..717: mol_type = other DNA
atggtgagcaagggcgaggagctgttcaccggggtggtgcccatcctggtcgagctggatggcgatgtaaatggccacaagttcagcgtgtccggcgagggcgagggcgatgccacctacggcaagctcaccctgaagttcatctgcaccaccggcaagctgcccgtgccctggcccaccctcgtcaccaccctcacctacggcgtgcagtgcttcagccgctaccccgatcacatgaagcagcacgatttcttcaagtccgccatgcccgaaggctacgtccaggagcgcaccatcttcttcaaggatgatggcaattaccgtacccgcgccgaggtgaagttcgagggcgataccctggtgaatcgcatcgagctgaagggcatcgatttcaaggaggatggcaatatcctggggcacaagctggagtacaattacaatagccacaatgtctatatcatggccgataagcagaagaatggcatcaaggtgaatttcaagatccgccacaatatcgaggatggcagcgtgcagctcgccgatcactaccagcagaatacccccatcggcgatggccccgtgctgctgcccgataatcactacctgagcacccagtccgccctgagcaaagatcccaatgagaagcgcgatcacatggtcctgctggagttcgtcaccgccgccgggatcactctcggcatggatgagctgtacaag

SEQ ID NO: 64 - DNA, 81 bp, synthetic construct
  source 1..81: mol_type = other DNA
gcggacttacgacagttgcgttacaccctttctcgacaaaacctaacttgcgcagaaaacatgccaatctcatcttggctt

SEQ ID NO: 65 - DNA, 135 bp, synthetic construct
  source 1..135: mol_type = other DNA
ttgcttagccatggcttcccgccggaggtggaggagcaggatgatggcacgctgcccatgtcttgtgcccaggagagcgggatggaccgtcaccctgcagcctgtgcttctgctaggatcaatgtgttagatgcg

SEQ ID NO: 66 - DNA, 12 bp, synthetic construct
  source 1..12: mol_type = other DNA
gacttacgacag

SEQ ID NO: 67 - protein, 195 aa, synthetic construct
PHPVLEKLRSINNYNPKDFDWNLKHGRVFIIKSYSEDDIHRSIKYNIWCSTEHGNKRLDAAYRSMNGKGPVYLLFSVNGSGHFCGVAEMKSAVDYNTCAGVWSQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 68 - protein, 195 aa, synthetic construct
PHPVLEKLRSINNYNPKDFDWNLKHGRVFIIKSYSEDDIHRSIKYNIACSTEHGNKRLDAAYRSMNGKGPVYLLFSVNGSGHFCGVAEMKSAVDYNTCAGVASQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 69 - protein, 170 aa, synthetic construct
GRVFIIKSYSEDDIHRSIKYNIWCSTEHGNKRLDAAYRSMNGKGPVYLLFSVNGSGHFCGVAEMKSAVDYNTCAGVWSQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 70 - protein, 170 aa, synthetic construct
GRVFIIKSYSEDDIHRSIKYNIACSTEHGNKRLDAAYRSMNGKGPVYLLFSVNGSGHFCGVAEMKSAVDYNTCAGVASQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 71 - protein, 195 aa, synthetic construct
PHPVLEKLRSINNYNPKDFDWNLKHGRVFIIKSYSENDIHRSIKYNIWCSTEHGNKRLDAAYRSMNGKGPVYLLFSVNGSGHFCGVAEMKSAVDYNTCAGVWSQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 72 - protein, 195 aa, synthetic construct
HPVLEKLKAAHSYNPKEFEWNLKSGRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 73 - protein, 171 aa, synthetic construct
GRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 74 - protein, 195 aa, synthetic construct
HPVLEKLKAAHSYNPKEFEWNLKSGRVFIIKSYSEDNIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 75 - protein, 195 aa, synthetic construct
VHPVLEKLKAINNYNPKDFDWNLKNGRVFIIKSYSEDDIHRSIKYSIWCSTEHGNKRLDAAYRSLNGKGPLYLLFSVNGSGHFCGVAEMKSVVDYNAYAGVWSQDKWKGKFEVKWIFVKDVPNNQLRHIRLENNDNKPVTNSRDTQEVPLEKAKQVLKIIATFKHTTSIFDDFAHYEKRQEEEEAMRRERNRNKQ

SEQ ID NO: 76 - protein, 165 aa, synthetic construct
SKLKYVLQDARFFLIKSNNHENVSLAKAKGVWSTLPVNEKKLNLAFRSARSVILIFSVRESGKFQGFARLSSESHHGGSPIHWVLPAGMSAKMLGGVFKIDWICRRELPFTKSAHLTNPWNEHKPVKIGRDGQEIELECGTQLCLLFPPDESIDLYQVIHKMRHK

SEQ ID NO: 77 - protein, 144 aa, synthetic construct
PVRYFIMKSSNLRNLEISQQKGIWSTTPSNERKLNRAFWESSIVYLVFSVQGSGHFQGFSRMSSEIGREKSQDWGSAGLGGVFKVEWIRKESLPFQFAHHLLNPWNDNKKVQISRDGQELEPLVGEQLLQLWERLPLGEKNTTD

SEQ ID NO: 78 - protein, 229 aa, synthetic construct [sequence omitted]

SEQ ID NO: 79 - protein, 198 aa, synthetic construct [sequence omitted]

SEQ ID NO: 80 - protein, 199 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 81 - protein, 385 aa, synthetic construct
QLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 82 - protein, 15 aa, synthetic construct
SGSETPGTSESATPE

SEQ ID NO: 83 - protein, 16 aa, synthetic construct
SGSETPGTSESATPES

SEQ ID NO: 84 - protein, 15 aa, synthetic construct
GGGGSGGGGSGGGGS

SEQ ID NO: 85 - protein, 50 aa, synthetic construct
GGGGSGGGGSGGGGSGGGGSGGGGSGGGGSGGGGSGGGGSGGGGSGGGGS

SEQ ID NO: 86 - protein, 17 aa, synthetic construct
AEAAAKEAAAKEAAAKA

SEQ ID NO: 87 - DNA, 1368 bp, synthetic construct
  source 1..1368: mol_type = other DNA
atgagcagcgaaaccggtccggtggcggttgacccgaccctgcgtcgtcgtattgagccgcacgagttcgaagtgttctttgatccgcgtgagctgcgtaaggaaacctgcctgctgtacgaaattaactggggtggccgtcacagcatctggcgtcacaccagccagaacaccaacaagcacgttgaggtgaacttcatcgaaaaatttaccaccgagcgttacttctgcccgaacacccgttgcagcattacctggtttctgagctggagcccgtgcggtgaatgcagccgtgcgatcaccgagttcctgagccgttatccgcacgttaccctgtttatctacattgcgcgtctgtatcaccacgcggacccgcgtaaccgtcaaggtctgcgtgatctgatcagcagcggcgtgaccatccagattatgaccgagcaagaaagcggttactgctggcgtaacttcgttaactatagcccgagcaacgaagcgcattggccgcgttacccgcacctgtgggtgcgtctgtacgttctggagctgtattgcatcattctgggcctgccgccgtgcctgaacattctgcgtcgtaagcagccgcaactgaccttctttaccatcgcgctgcagagctgccactaccaacgtctgccgccgcacattctgtgggcgaccggtctgaagagcggcagcgaaaccccgggtaccagcgaaagcgcgaccccggagccgcacccggtgctggagaaactgcgtagcatcaacaactataacccgaaggacttcgattggaacctgaaacacggtcgtgtttttatcattaagagctacagcgaagacgatatccaccgtagcattaaatataacatctggtgcagcaccgagcacggcaacaagcgtctggacgcggcgtaccgtagcatgaacggtaaaggcccggtgtatctgctgttcagcgttaacggtagcggccacttttgcggtgtggcggaaatgaaaagcgcggttgattacaacacctgcgcgggtgtgtggagccaggacaagtggaaaggccgtttcgatgttcgttggatttttgtgaaggacgttccgaacagccaactgcgtcacatccgtctggagaacaacgaaaacaaaccggtgaccaacagccgtgatacccaggaagtgccgctggaaaaggcgaaacaagttctgaagatcattgcgagctacaaacacaccaccagcatcttcgacgattttagccactatgagaagcgtcaggaagaggaagagagcgtgaagaaggagcgtcaaggtcgtggcaaactggagtacccgtatgacgttccggattatgcgtaaattggaagtggataa

SEQ ID NO: 88 - DNA, 1293 bp, synthetic construct
  source 1..1293: mol_type = other DNA
atgagcagcgaaaccggtccggtggcggttgacccgaccctgcgtcgtcgtattgagccgcacgagttcgaagtgttctttgatccgcgtgagctgcgtaaggaaacctgcctgctgtacgaaattaactggggtggccgtcacagcatctggcgtcacaccagccagaacaccaacaagcacgttgaggtgaacttcatcgaaaaatttaccaccgagcgttacttctgcccgaacacccgttgcagcattacctggtttctgagctggagcccgtgcggtgaatgcagccgtgcgatcaccgagttcctgagccgttatccgcacgttaccctgtttatctacattgcgcgtctgtatcaccacgcggacccgcgtaaccgtcaaggtctgcgtgatctgatcagcagcggcgtgaccatccagattatgaccgagcaagaaagcggttactgctggcgtaacttcgttaactatagcccgagcaacgaagcgcattggccgcgttacccgcacctgtgggtgcgtctgtacgttctggagctgtattgcatcattctgggcctgccgccgtgcctgaacattctgcgtcgtaagcagccgcaactgaccttctttaccatcgcgctgcagagctgccactaccaacgtctgccgccgcacattctgtgggcgaccggtctgaagagcggcagcgaaaccccgggtaccagcgaaagcgcgaccccggagggtcgtgtttttatcattaagagctacagcgaagacgatatccaccgtagcattaaatataacatctggtgcagcaccgagcacggcaacaagcgtctggacgcggcgtaccgtagcatgaacggtaaaggcccggtgtatctgctgttcagcgttaacggtagcggccacttttgcggtgtggcggaaatgaaaagcgcggttgattacaacacctgcgcgggtgtgtggagccaggacaagtggaaaggccgtttcgatgttcgttggatttttgtgaaggacgttccgaacagccaactgcgtcacatccgtctggagaacaacgaaaacaaaccggtgaccaacagccgtgatacccaggaagtgccgctggaaaaggcgaaacaagttctgaagatcattgcgagctacaaacacaccaccagcatcttcgacgattttagccactatgagaagcgtcaggaagaggaagagagcgtgaagaaggagcgtcaaggtcgtggcaaactggagtacccgtatgacgttccggattatgcgtaaattggaagtggataa

SEQ ID NO: 89 - protein, 7 aa, synthetic construct
PKKKRKV

SEQ ID NO: 90 - protein, 9 aa, synthetic construct
LPPLERLTL

SEQ ID NO: 91 - protein, 27 aa, synthetic construct
MDPVVVLGLCLSCLLLLSLWKQSYGGG

SEQ ID NO: 92 - protein, 21 aa, synthetic construct
METDTLLLWVLLLWVPGSTGD

SEQ ID NO: 93 - protein, 10 aa, synthetic construct
EQKLISEEDL

SEQ ID NO: 94 - protein, 14 aa, synthetic construct
GKPIPNPLLGLDST

SEQ ID NO: 95 - protein, 9 aa, synthetic construct
IPNPLLGLD

SEQ ID NO: 96 - protein, 8 aa, synthetic construct
DYKDDDDK

SEQ ID NO: 97 - protein, 22 aa, synthetic construct
DYKDHDGDYKDHDIDYKDDDDK

SEQ ID NO: 98 - protein, 158 aa, synthetic construct
ISLIAALAVDHVIGMETVMPWNLPADLAWFKRNTLNKPVIMGRHTWESIGRPLPGRKNIILSSQPSTDDRVTWVKSVDEAIAACGDVPEIMVIGGGRVYEQFLPKAQKLYLTHIDAEVEGDTHFPDYEPDDWESVFSEFHDADAQNSHSYCFEILERR

SEQ ID NO: 99 - RNA, 12 nt, synthetic construct
  source 1..12: mol_type = other RNA
gacttatgacag

SEQ ID NO: 100 - RNA, 12 nt, synthetic construct
  source 1..12: mol_type = other RNA
gacttacgacag

SEQ ID NO: 101 - DNA, 15 bp, synthetic construct
  source 1..15: mol_type = other DNA
ggacttacgacagtt

SEQ ID NO: 102 - RNA, 12 nt, synthetic construct
  source 1..12: mol_type = other RNA
ggcttacgacag

SEQ ID NO: 103 - RNA, 12 nt, synthetic construct
  source 1..12: mol_type = other RNA
gacttacgagag

SEQ ID NO: 104 - protein, 6 aa, synthetic construct
HHHHHH

SEQ ID NO: 105 - DNA, 12604 bp, synthetic construct
  source 1..12604: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgctagcgtttaaacgggccctctagagccgccatggtgagcaagggcgaggagctgttcaccggggtggtgcccatcctggtcgagctggatggcgatgtaaatggccacaagttcagcgtgtccggcgagggcgagggcgatgccacctacggcaagctcaccctgaagttcatctgcaccaccggcaagctgcccgtgccctggcccaccctcgtcaccaccctcacctacggcgtgcagtgcttcagccgctaccccgatcacatgaagcagcacgatttcttcaagtccgccatgcccgaaggctacgtccaggagcgcaccatcttcttcaaggatgatggcaattaccgtacccgcgccgaggtgaagttcgagggcgataccctggtgaatcgcatcgagctgaagggcatcgatttcaaggaggatggcaatatcctggggcacaagctggagtacaattacaatagccacaatgtctatatcatggccgataagcagaagaatggcatcaaggtgaatttcaagatccgccacaatatcgaggatggcagcgtgcagctcgccgatcactaccagcagaatacccccatcggcgatggccccgtgctgctgcccgataatcactacctgagcacccagtccgccctgagcaaagatcccaatgagaagcgcgatcacatggtcctgctggagttcgtcaccgccgccgggatcactctcggcatggatgagctgtacaaggcggacttacgacagttgcgttacaccctttctcgacaaaacctaacttgcgcagaaaacatgccaatctcatcttggcttatcagtctgattgcggcgttagcggtagatcacgttatcggcatggaaaccgtcatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggttattggcggcggtcgcgtttatgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgataagcctcattgtgcattctctcgagtacccctacgacgtgcccgactacgcctgagggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaccagtgtgatggatatctgcagaattcgcccttggatccgaattcctgcagccccgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgagggggatccactagcatgaagggcgaattccagcacactggtaacccgtgtcggctccagatctggcctccgcgccgggttttggcgcctcccgcgggcgcccccctcctcacggcgagccgcgttgacattgattattgactaggcttttgcaaaaagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtaccgagctcggatccactagtccagtgtggtggaattctgcagatatccagcacagtggggtttagtgaaccgtcagatccgctagagatccgcggccgctaatacgactcactatagggagagccgccaccatgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaagcggcagcgagactcccgggacctcagagtccgccacaccagaaccccacccagtgttggagaagcttcggtccattaataactataaccccaaagattttgactggaatctgaaacatggccgggttttcatcattaagagctactctgaggacgatattcaccgttccattaagtataatatttggtgcagcacagagcatggtaacaagagactggatgctgcttatcgttccatgaacgggaaaggccccgtttacttacttttcagtgtcaacggcagtggacacttctgtggcgtggcagaaatgaaatctgctgtggactacaacacatgtgcaggtgtgtggtcccaggacaaatggaagggtcgttttgatgtcaggtggatttttgtgaaggacgttcccaatagccaactgcgacacattcgcctagagaacaacgagaataaaccagtgaccaactctagggacactcaggaagtgcctctggaaaaggctaagcaggtgttgaaaattatagccagctacaagcacaccacttccatttttgatgacttctcacactatgagaaacgccaagaggaagaagaaagtgttaaaaaggaacgtcaaggtcgtgggaaactcgagtacccctacgacgtgcccgactacgcctgagtttaaaatcgatggtacactcgaggttaacgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcatcccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggttccggcgcaacaaacttctctctgctgaaacaagccggagatgtcgaagagaatcctggaccgatggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcgccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtgtaaagtagatgccgaccgaacaagagctgatttcgagaacgcctcagccagcaactcgcgcgagcctagcaaggcaaatgcgagagaacggccttacgcttggtggcacagttctcgtccacagttcgctaagctcgctcggctgggtcgcgggagggccggtcgcagtgattcaggcccttctggattgtgttggtccccagggcacgattgtcatgcccacgcactcgggtgatctgactgatcccgcagattggagatcgccgcccgtgcctgccgattgggtgcagatccgtcgagttaacaaaagaaaaggggggactggaagggctaattcactcccaacgaagacaagatatcataacttcgtatagcatacattatacgaagttatcggctagctggtccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 106 - DNA, 9483 bp, synthetic construct
  source 1..9483: mol_type = other DNA
agggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgagggggatccactagcatgaagggcgaattccagcacactggtaacccgtgtcggctccagatctggcctccgcgccgggttttggcgcctcccgcgggcgcccccctcctcacggcgagccgcgttgacattgattattgactaggcttttgcaaaaagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtaccgagctcggatccactagtccagtgtggtggaattctgcagatatccagcacagtggggtttagtgaaccgtcagatccgctagagatccgcggccgctaatacgactcactatagggagagccgccaccatgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaagcggcagcgagactcccgggacctcagagtccgccacaccagaaccccacccagtgttggagaagcttcggtccattaataactataaccccaaagattttgactggaatctgaaacatggccgggttttcatcattaagagctactctgaggacgatattcaccgttccattaagtataatatttggtgcagcacagagcatggtaacaagagactggatgctgcttatcgttccatgaacgggaaaggccccgtttacttacttttcagtgtcaacggcagtggacacttctgtggcgtggcagaaatgaaatctgctgtggactacaacacatgtgcaggtgtgtggtcccaggacaaatggaagggtcgttttgatgtcaggtggatttttgtgaaggacgttcccaatagccaactgcgacacattcgcctagagaacaacgagaataaaccagtgaccaactctagggacactcaggaagtgcctctggaaaaggctaagcaggtgttgaaaattatagccagctacaagcacaccacttccatttttgatgacttctcacactatgagaaacgccaagaggaagaagaaagtgttaaaaaggaacgtcaaggtcgtgggaaactcgagtacccctacgacgtgcccgactacgcctgagtttaaaatcgatggtacactcgaggttaacgaattctaccttacccagagtgcaggtgtgtggagatccctcctgccttgacattgagcagccttagagggtgggggaggctcaggggtcaggtctctgttcctgcttattggggagttcctggcctggcccttctatgtctccccaggtaccccagtttttctgggttcacccagagtgcagatgcttgaggaggtgggaagggactatttgggggtgtctggctcaggtgccatgcctcactggggctggttggcacctgcatttcctgggagtggggctgtctcagggtagctgggcacggtgttcccttgagtgggggtgtagtgagtgttcctagctgccacgcctttgccttcacctatgggatcgtggctgtcagttaattaaccttccgcgggagctcacggggagagccccccgccaaagcccccagggatgtaattgcatccctcttccgctagggggcagcagcgagccgcccggggctccgctccggtccggcgctccccccgcatccccgagccggagccggcagcgtgcggggacagcccggcacggggaaggtggcacgcgatcgctttcctctgaacgcttctcgctgctctttgagcctgcagacacctggggggatacggggaaaaagctttaggctgaaagagagatttagaatgacagaatcatagaatggcctgggttgcaaaggagcacagtgctcacccagctccaaccccctgctatgtgcagggtcgccaaccagcagcccaggctgcccagagccacatccagcctggccttgaatgcctgcagggatggggcatccacagcctccttgggcaacctgttcagtgcgtcacggatccaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagctgtagctagcacaaccatggatagcactgagaacgtcatcaagcccttcatgcgcttcaaggtgcacatggagggctccgtgaacggccacgagttcgagatcgagggcgagggcgagggcaagccctacgagggcacccagaccgccaagctgcaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggcaccttcatctaccacgtgaagttcatcggcgtgaacttcccctccgacggccccgtaatgcagaagaagactctgggctgggagccctccaccgagcgcctgtacccccgcgacggcgtgctgaagggcgagatccacaaggcgctgaagctgaagggcggcggccactacctggtggagttcaagtcaatctacatggccaagaagcccgtgaagctgcccggctactactacgtggactccaagctggacatcacctcccacaacgaggactacaccgtggtggagcagtacgagcgcgccgaggcccgccaccacctgttccagtagggctagctggtccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 107 - DNA, 9483 bp, synthetic construct; identical to SEQ ID NO: 106
  source 1..9483: mol_type = other DNA

SEQ ID NO: 108 - DNA, 81 bp, synthetic construct; identical to SEQ ID NO: 64
  source 1..81: mol_type = other DNA

SEQ ID NO: 109 - DNA, 81 bp, synthetic construct
  source 1..81: mol_type = other DNA
gcggcgttacgacagttgcgttacaccctttctcgacaaaacctaacttgcgcagaaaacatgccaatctcatcttggctt

SEQ ID NO: 110 - DNA, 81 bp, synthetic construct
  source 1..81: mol_type = other DNA
gcggacttacgtcagttgcgttacaccctttctcgacaaaacctaacttgcgcagaaaacatgccaatctcatcttggctt

SEQ ID NO: 111 - DNA, 81 bp, synthetic construct
  source 1..81: mol_type = other DNA
gcggagttacgacagttgcgttacaccctttctcgtcaaaacctaacttgcgcagaaaacatgccaatctcatcttggctt

SEQ ID NO: 112 - DNA, 81 bp, synthetic construct
  source 1..81: mol_type = other DNA
gcggagttacgacagttgcgttacaccctttctcgacaaagcctaacttgcgcagaaaacatgccaatctcatcttggctt

SEQ ID NO: 113 - DNA, 81 bp, synthetic construct
  source 1..81: mol_type = other DNA
gcggagttacgacagttgcgttacaccctttctcgacaaaacctagcttgcgcagaaaacatgccaatctcatcttggctt

SEQ ID NO: 114 - DNA, 81 bp, synthetic construct
  source 1..81: mol_type = other DNA
gcggagttacgacagttgcgttacaccctttctcgacaaaacctaacttgcgcagaaagcatgccaatctcatcttggctt

SEQ ID NO: 115 - DNA, 39 bp, synthetic construct
  source 1..39: mol_type = other DNA
gcggacttacgacagttgcgtccaatctcatcttggctt

SEQ ID NO: 116 - DNA, 81 bp, synthetic construct
  source 1..81: mol_type = other DNA
gcggccttacgtcagttgcgttacaccctttctcggcaaagcctagcttgcgcagaaagcatgccaatctcatcttggctt

SEQ ID NO: 117 - protein, 100 aa, synthetic construct
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 118 - protein, 173 aa, Homo sapiens
TSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLR

SEQ ID NO: 119 - protein, 181 aa, synthetic construct
MDSLLMNRREFLYQFKNVRWAKGRRETYLCYVVKRRDSATSFSLDFGYLRNKNGCHVELLFLRYISDWDLDPGRCYRVTWFISWSPCYDCARHVADFLRGNPNLSLRIFTARLYFCEAGRREPEGLRRLHRAGVQIAIMTFKDYFYCWNTFVENHGRTFKAWEGLHENSVRLSRQLRRILL

SEQ ID NO: 120 - protein, 215 aa, synthetic construct
RRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 121 - protein, 1226 aa, Homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLRSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 122 - protein, 394 aa, Homo sapiens
LPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 123 - protein, 52 aa, synthetic construct
AEAAAKEAAAKEAAAKEAAAKEAAAKEAAAKEAAAKEAAAKEAAAKEAAAKA

SEQ ID NO: 124 - protein, 102 aa, synthetic construct
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 125 - protein, 393 aa, Homo sapiens [sequence omitted]

SEQ ID NO: 126 - protein, 198 aa, Homo sapiens
MTLRCLEPSGNGGEGTRSQWGTAGSAEEPSPQAARLAKALRELGQTGWYWGSMTVNEAKEKLKEAPEGTFLIRDSSHSDYLLTISVKTSAGPTNLRIEYQDGKFRLDSIICVKSKLKQFDSVVHLIDYYVQMCKDKRTGPEAPRNGTVHLYLTKPLYTSAPSLQHLCRLTINKCTGAIWGLPLPTRLKDYLEEYKFQV

(19) United States
(12) Patent Application Publication
(10) Pub. No.: US 2024/0207446 A1
(43) Pub. Date: Jun. 27, 2024

(54) M6A-COUPLED EFFECTOR PROTEIN EXPRESSION SYSTEM AND METHODS OF MAKING AND USING SAME

(71) Applicant: Duke University, Durham, NC (US)

(72) Inventor: Kathryn Meyer, Durham, NC (US)

(73) Assignee: Duke University, Durham, NC (US)

(21) Appl. No.: 18/486,146

(22) Filed: Oct. 12, 2023

Related U.S. Application Data
(60) Provisional application No. 63/531,948, filed on Aug. 10, 2023.
(60) Provisional application No. 63/415,395, filed on Oct. 12, 2022.

(51) Int. Cl. A61K 48/00; A61P 35/02; C12N 9/06; C12N 9/22; C12N 9/78; C12N 15/11; C12N 15/62

(52) U.S. Cl. CPC A61K 48/005; A61P 35/02; C12N 9/003; C12N 9/22; C12N 9/78; C12N 15/111; C12N 15/62; C12N 2310/20; C12Y 305/04004; C12Y 305/04005

(57) ABSTRACT

The present disclosure provides systems and methods for m⁶A-dependent delivery and m⁶A-dependent delivery targeted of a polypeptide to a cell. In certain embodiments, compositions, systems, and methods are provided that provide for m⁶A-dependent delivery of effector proteins, for example, effector proteins, such as a tumor suppression proteins and m6a regulation systems, mediated by CRISPRi in embodiments.

### CROSS REFERENCE TO RELATED APPLICATIONS

This application claims priority to, and the benefit of, U.S. Provisional Application No. 63/415,395, filed on Oct. 12, 2022, and U.S. Provisional Application No. 63/531,948, filed on Aug. 10, 2023, the entire contents of both of which are incorporated by reference as if fully set forth herein.

### STATEMENT REGARDING FEDERALLY FUNDED RESEARCH

This invention was made with government support under Grant Nos. DP1DA046584 and R0IMH118366 awarded by the National Institutes of Health/National Institute on Drug Abuse and National Institutes of Health/National Institute of Mental Health, respectively. The government has certain rights in the invention.

### REFERENCE TO A SEQUENCE LISTING SUBMITTED AS A XML FILE VIA EFS-WEB

The official copy of the sequence listing named “1412057 (DU7940US) Sequence Listing.xml”, created on Jan. 16, 2024, and having a size of 172 KB is submitted electronically via Patent Center in .xml format and is hereby incorporated by reference in its entirety.

### BACKGROUND

The N⁶-methyladenosine (m⁶A) modification is found in thousands of cellular mRNAs and is a critical regulator of gene expression and cellular physiology. In pathological instances, m⁶A modifications may be dysregulated, contributing to several human diseases. For example, m⁶A dysregulation can lead to hypermethylation of oncogenic mRNAs and, in turn, leads to increased translation and cancer progression. The m⁶A methyltransferase machinery therefore has emerged as a promising therapeutic target.

Ongoing study of m⁶A modifications enabled by new tools provides clues as to strategies for overcoming hypermethylation. Current strategies for overcoming hypermethylation are focused on developing drugs that inhibit methyltransferase such as, for example, m(6)A methyltransferase (METTL3). However, as they can impact the methylation of all mRNAs, these approaches can have unwanted effects. Thus, targeted approaches for decreasing m⁶A hypermethylation and expression of upregulated oncogenes caused by m⁶A dysregulation are necessary avoid to globally affecting m⁶A modifications in unwanted areas, such as non-pathological tissue or cells.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Provided herein is a N⁶-methyladenosine (m⁶A)-coupled effector protein expression system and methods of introducing same into a cell, tissue, and/or animal model to achieve m⁶A-dependent protein expression.

In some embodiments, the m⁶A-coupled effector protein expression system comprises (a) a nucleic acid sequence encoding a fusion protein, wherein the fusion protein comprises an m⁶A binding domain of a YT521-B homology (YTH) domain-containing protein fused to a catalytic domain of a cytidine deaminase, and (b) a nucleic acid sequence encoding an effector protein (e.g., a protein that modulates expression of one or more proteins in the cell) and dihydrofolate reductase (DHFR). In some embodiments, the m⁶A-coupled effector protein expression system comprises (a) a nucleic acid sequence encoding a fusion protein, wherein the fusion protein comprises an m⁶A binding domain of a YT521-B homology (YTH) domain-containing protein fused to a catalytic domain of a cytidine deaminase, and (b) a nucleic acid sequence encoding an effector protein (e.g., a protein that modulates expression of one or more proteins in the cell), a M⁶A sensing domain, and dihydrofolate reductase (DHFR).

In some embodiments, the expression system is a vector system wherein a first plasmid comprises the nucleic acid sequence encoding the fusion protein comprising an N⁶-methyladenosine (m⁶A) binding domain of a YT521-B homology (YTH) domain-containing protein fused to a catalytic domain of a cytidine deaminase, and a second plasmid comprises the nucleic acid sequence encoding an effector protein (e.g., a protein that modulates expression of one or more proteins in the cell or otherwise targets a component of an expression system to a cell or within a cell) and dihydrofolate reductase (DHFR).

In some embodiments, the catalytic domain of the cytosine deaminase is the catalytic domain of apolipoprotein B mRNA editing enzyme (APOBEC-1). In some embodiments, the effector protein is a tumor suppressor protein, for example, METTL3. In some embodiments, the effector protein is an RNA-guided endonuclease. In some embodiments, the RNA-guided endonuclease is a dead RNA-guided endonuclease, for example, dead Cas9 (dCas9). In some embodiments, the effector protein comprises dCas9 linked or fused to a transcriptional regulator, for example, a transcriptional repressor (e.g., KRAB).

In further embodiments, the expression system can comprise: a first DNA construct comprising a nucleic acid sequence encoding a fusion protein, wherein the fusion protein comprises (i) a catalytically-dead RNA-targeting CRISPR-Cas system enzyme fused to (ii) a catalytic domain of a cytidine deaminase fused to (iii) an N6-methyladenosine (m6A) binding domain of a YT521-B homology (YTH) domain-containing protein; a second DNA construct comprising a nucleic acid sequence encoding: an effector protein; a m6A sensor sequence; and a polypeptide encoding dihydrofolate reductase (DHFR); and a guide RNA configured to bind to the nucleic acid of the second DNA construct. In embodiments, the cytidine deaminase can be APOBEC-1. In embodiments, the effector protein can be a tumor suppressor protein. In some embodiments, the effector protein can be a p53 or a SOCS2. In some embodiments, the dead RNA-guided endonuclease can be a dead type VI dCas13. In some embodiments, the fusion protein can further comprise a nuclear localization sequence (NLS).

In embodiments, described herein is an expression system comprising: (a) a first DNA construct comprising a polynucleotide encoding a fusion protein, wherein the fusion protein comprises an N⁶-methyladenosine (m⁶A) binding domain of a YT521-B homology (YTH) domain-containing protein fused to a catalytic domain of a cytidine deaminase or a catalytic domain of an adenosine deaminase; and a second DNA construct comprising a polynucleotide encoding a heterologous polypeptide, the polynucleotide encoding a heterologous polypeptide comprising: polynucleotide encoding an effector protein; polynucleotide encoding a m⁶A sensor sequence; and a polynucleotide encoding a dihydrofolate reductase (DHFR).

In embodiments, the m⁶A binding domain comprises a sequence having at least 90% or greater sequence identity to SEQ ID Nos: 66 or 108-116. In embodiments, the m⁶A binding domain is fused to the catalytic domain via a peptide linker. In embodiments, the catalytic domain comprises a polypeptide having at least 95% identity to SEQ ID NO 78 or a catalytic fragment thereof, SEQ ID NO: 79 or a catalytic fragment thereof, SEQ ID NO: 80 or a catalytic fragment thereof; or SEQ ID NO: 81. In embodiments, a vector comprises the first DNA construct, a second DNA construct, or both. In embodiments, the nucleic acid sequence encoding a fusion protein, the nucleic acid sequence encoding a heterologous polypeptide and a polypeptide encoding dihydrofolate reductase (DHFR), or both, are operably linked to a first promoter. In embodiments, the system further comprises a nucleic acid sequence encoding a selectable marker operably linked to a second promoter. In embodiments, the first promoter is a constitutive or an inducible promoter. In embodiments, the first promoter is a constitutive or an inducible promoter. In embodiments, the cytidine deaminase is APOBEC-1. In embodiments, the effector protein is a tumor suppressor protein or a catalytically dead RNA-guided endonuclease. In embodiments, the tumor suppressor protein is suppressor of cytokine signaling 2 (SOC2) or p53 or one of the proteins listed in Table 1. In embodiments, the catalytically dead RNA-guided endonuclease is a dCas9 or a dCas13.

In embodiments, described herein is a polynucleotide comprising a nucleic acid sequence encoding an effector protein polypeptide, a m⁶A sensor sequence, and a polypeptide encoding dihydrofolate reductase (DHFR). Also described herein are vectors and host cells comprising one or more components of expression systems as described herein, as well as non-human transgenic animals comprising one or more components of expression vectors as described herein. Described herein additionally are kits comprising any one or more components of expression systems as described herein.

Further described herein are methods. In embodiments, described herein are methods of increasing expression of a tumor suppressor protein in one or more cells, comprising introducing the expression system of claim 1 into the one or more cells, for example, hepatocellular carcinoma (HCC) cells. In embodiments, the tumor suppression protein is SOCS2 or p53 or one of the proteins listed in Table 1.

Described herein are methods of reducing m⁶A effector regulator expression in a sample or a subject. In embodiments, described herein is a method of reducing M6A effector regulator expression, comprising: introducing an expression system into a subject having or suspected of having a cancer, wherein the expression system comprises: a first DNA construct comprising a polynucleotide encoding a fusion protein, wherein the fusion protein comprises an N⁶-methyladenosine (m⁶A) binding domain of a YT521-B homology (YTH) domain-containing protein fused to a catalytic domain of a cytidine deaminase or a catalytic domain of an adenosine deaminase; and a second DNA construct comprising a polynucleotide encoding a heterologous polypeptide, the polynucleotide encoding a heterologous polypeptide comprising: a polynucleotide encoding a catalytically-dead RNA-guided endonuclease; a polynucleotide encoding a m⁶A sensor sequence; and a polynucleotide encoding a dihydrofolate reductase (DHFR); an sgRNA configured to bind to an m⁶A regulator. In embodiments, the sgRNA is configured to bind to a m⁶A regulator listed in Table 2. In embodiments, the cancer comprises at least one of acute myeloid leukemia (AML), glioblastoma (GBM), lung cancer, endometrial cancer, cervical cancer, ovarian cancer, breast cancer, colorectal cancer (CRC), a hepatocellular carcinoma (HCC), pancreatic cancer, gastric cancer, prostate cancer, or renal cell carcinoma. In embodiments, the cancer is a cancer listed in Table 1 or Table 2. In embodiments, the catalytically-dead RNA-guided endonuclease is a dCas9 or dCas13.

Described herein are methods of reducing m⁶A hypermethylation in a subject or sample. In embodiments, methods comprising: introducing an expression system into a subject having or suspected of having a cancer, wherein the expression system comprises: a first DNA construct comprising a polynucleotide encoding a fusion protein, wherein the fusion protein comprises an N⁶-methyladenosine (m⁶A) binding domain of a YT521-B homology (YTH) domain-containing protein fused to a catalytic domain of a cytidine deaminase or a catalytic domain of an adenosine deaminase; and a second DNA construct comprising a polynucleotide encoding a heterologous polypeptide, the polynucleotide encoding a heterologous polypeptide comprising: a polynucleotide encoding a catalytically-dead RNA-guided endonuclease; a polynucleotide encoding a m⁶A sensor sequence; and a polynucleotide encoding a dihydrofolate reductase (DHFR); an sgRNA configured to bind to an m⁶A regulator. The sgRNA is configured to bind to a m⁶A regulator listed in Table 2. The cancer comprises at least one of acute myeloid leukemia (AML), glioblastoma (GBM), lung cancer, endometrial cancer, cervical cancer, ovarian cancer, breast cancer, colorectal cancer (CRC), a hepatocellular carcinoma (HCC), pancreatic cancer, gastric cancer, prostate cancer, or renal cell carcinoma. The cancer is a cancer listed in Table 1 or Table 2. The catalytically-dead RNA-guided endonuclease is a dCas9 or dCas13.

In embodiments, described herein are methods of inhibiting cancer cells. In an embodiment, a method of inhibiting a cancer cell, the method comprising: introducing the expression system as described herein into the cancer cell, wherein the cancer cell comprises m⁶A RNA hypermethylation, and wherein the second DNA construct comprising a polynucleotide encoding an effector protein, the effector protein comprising a tumor suppressor protein.

The cancer cell can comprise an acute myeloid leukemia (AML) cell, a glioblastoma (GBM) cell, a lung cancer cell, an endometrial cancer, a cervical cancer cell, an ovarian cancer cell, a breast cancer cell, a colorectal cancer (CRC) cell, a hepatocellular carcinoma (HCC) cell, a pancreatic cancer cell, a gastric cancer cell, a prostate cancer cell, or a renal cell carcinoma cell. In an embodiment, the lung cancer cell is a non-small cell lung carcinoma cell. In an embodiment, the cancer cell is a hepatocellular carcinoma cell. In an embodiment, the tumor suppressor protein comprises at least one of the tumor suppressor proteins listed in Table 1. In an embodiment, expression of the tumor suppressor protein upregulates downstream signaling targets. In an embodiment, the tumor suppressor protein comprises p53. In an embodiment, expression of p53 upregulates at least one of CDKN1A or GADD45A. In an embodiment, the tumor suppressor protein comprises suppressor of cytokine signaling 2 (SOCS2). In an embodiment, the expression system is introduced into the cancer cell by transfection, viral infection, or electroporation. In an embodiment of methods as described herein, inhibiting the cancer cell comprises decreasing at least one of cell proliferation, cell migration, or metastasis.

Described herein are methods of treating a subject having a cancer. In embodiments, methods of treating a subject having a cancer characterized by m⁶A RNA hypermethylation, the methods comprise inhibiting a cancer cell according to the methods as described above. In embodiments, the cancer comprises at least one of acute myeloid leukemia (AML), glioblastoma (GBM), lung cancer, endometrial cancer, cervical cancer, ovarian cancer, breast cancer, colorectal cancer (CRC), a hepatocellular carcinoma (HCC), pancreatic cancer, gastric cancer, prostate cancer, or renal cell carcinoma. In embodiments, the cancer comprises hepatocellular carcinoma. In embodiments, expression of the tumor suppressor protein results in decreasing at least one of cell proliferation, cell migration, or metastasis of the cancer. In embodiments, the expression system is introduced into the subject by viral infection or electroporation.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 is a schematic of the m⁶A sensor system according to certain embodiments of this disclosure. The m⁶A reporter mRNA is shown with the m⁶A sensor sequence expanded. When this sequence is unmethylated, APO1-YTH does not bind to the sensor sequence and no editing takes place. As a result, GFP-DHFR is produced and rapidly degraded (left panel). Methylation of either adenosine (red) in the sensor sequence results in recruitment of APO1-YTH and C-to-U editing of either or both convertible stop codons. Translation leads to GFP production and cell fluorescence (right panel).

FIGS. 2A-2M show that GEMS depends on m⁶A recognition. FIG. 2A. HEK293T cells were transfected with the GEMS reporter mRNA alone or the reporter mRNA together with APO1-YTH and imaged 24 h later. Cells expressing the reporter mRNA with APO1-YTH exhibit robust EGFP fluorescence, whereas cells expressing the reporter mRNA alone are dark. Scale bar: 100 μm. FIG. 2B. RT-PCR and Sanger sequencing of the m⁶A sensor sequence from cells in (FIG. 2A) shows C-to-U editing (C-to-T in cDNA) of the convertible stop codon cytidines (marked with an asterisk) only in the presence of APO1-YTH. Quantification of % C2U at the indicated cytidines is shown below the sequencing traces. ***p<0.001. n=3 biological replicates. FIG. 2C. Western blot from cells in (FIG. 2A) indicates EGFP production from the GEMS reporter mRNA only in cells expressing APO1-YTH. FIG. 2D. HEK293T cells were transfected with the GEMS system containing APO1-YTH or m⁶A binding-deficient APO1-YTHmut and imaged after 24 h. Cells expressing APO1-YTH contain EGFP fluorescence whereas cells expressing APO1-YTHmut are dark. Scale bar: 100 μm. FIG. 2E. Western blot from cells in (FIG. 2D) indicates loss of EGFP production from the GEMS reporter mRNA when cells co-express APO1-YTHmut. FIG. 2F. RT-PCR and Sanger sequencing from cells in (FIG. 2D) shows C-to-U editing of the m⁶A sensor sequence only in cells co-expressing APO1-YTH. Quantification of % C2U at the indicated cytidines is shown below the sequencing traces. ***p<0.001. n=2 biological replicates. FIGS. 2G-2L show that GEMS mRNA methylation mirrors endogenous mRNA methylation. FIG. 2G. HEK293T cells were co-transfected with the m⁶A reporter mRNA and APO1-YTH or APO1-YTHmut, then subjected to flow cytometry. Robust EGFP fluorescence is detected only in cells expressing the reporter mRNA and APO1-YTH. FIG. 2H. HEK293T cells were transfected with the GEMS system, and flow cytometry was used to sort cells into three populations based on EGFP fluorescence intensity. RT-qPCR-based m⁶A quantification of the sensor sequence shows an increase in m⁶A with increasing EGFP fluorescence. **p<0.01, n=2 biological replicates. FIG. 2I. RNA was isolated from sorted cell populations in (FIG. 2H) and analyzed by RT-PCR/Sanger sequencing. C-to-U editing of the m⁶A sensor sequence is increased in cells with higher levels of EGFP fluorescence. FIG. 2J. HEK293T cells were transfected with GEMS containing the full length m⁶A sensor sequence or a version with RAC motifs mutated (GEMS ARAC). EGFP fluorescence is abolished in cells expressing GEMS ARAC. Scale bar: 100 μm. FIG. 2K. Top schematic shows the m6A reporter mRNA with a portion of the m6A sensor sequence expanded. This sequence is based off of a sequence within the human ACTB 3′UTR (bottom schematic), which contains two m6A sites at positions A1216 and A1222 that have been shown to be methylated in several m6A mapping studies. FIG. 2L. RNA was isolated from HEK293T cells expressing the GEMS system for 24 h, followed by RT-PCR and Sanger sequencing. Sequencing traces show C-to-U editing of site A1222 in endogenous ACTB and the second consensus A in the m⁶A sensor sequence. N.s.=no significant difference, n=3 biological replicates. FIG. 2M. Relative m⁶A quantification using an RT-qPCR m⁶A detection approach shows similar levels of methylation of the m⁶A sensor sequence (GEMS GAC) and endogenous ACTB site A1222 on which the sensor sequence is based. M⁶A is not detected at non-consensus adenosines in the m⁶A sensor sequence (UAC and CAG). Dotted line at 0.5 represents the minimum cutoff value indicating the presence of m⁶A. Schematic below shows the presence of the indicated adenosines within the m⁶A sensor sequence. ***p<0.001. n=3 biological replicates.

FIGS. 3A-3T show that GEMS is METTL3-dependent and responds to changes in METTL-3 levels. FIG. 3A. Mass spectrometry analysis of purified mRNA from METTL3 degron cells shows a decrease in m⁶A in auxin-treated cells. ***p<0.001, n=2 biological replicates. FIG. 3B. The GEMS system was transfected into HEK293T cells containing an auxin-inducible degron tag fused to endogenous METTL3. Addition of auxin leads to reduced EGFP fluorescence. Scale bar: 100 μm. FIG. 3C. Western blot confirms loss of METTL3 and EGFP following auxin treatment of cells in (FIG. 3B). FIG. 3D. Quantification of EGFP/EGFP-DHFR ratio from western blot samples in (FIG. 3C) shows a decrease in EGFP/EGFP-DHFR in response to auxin treatment. ***p<0.001, n=5 biological replicates. FIG. 3E. Sanger sequencing traces show C-to-U editing of the m⁶A sensor sequence in RNA extracts from cells in (FIG. 3B). Editing is reduced in auxin-treated cells. ***p<0.001, n=3 biological replicates FIG. 3F. RT-qPCR quantification of m⁶A sensor sequence methylation decreased m⁶A after auxin-mediated METTL3 depletion. ***p<0.001, n=3 biological replicates. FIG. 3G. RT-qPCR quantification of m⁶A reporter mRNA expression was performed on RNA samples extracted from METTL3 degron cells with and without auxin treatment. N.s.=no significant difference, n=2 biological replicates. FIG. 3H. Densitometry analysis of western blot data was used to quantify total reporter mRNA protein production (EGFP+EGFP-DHFR) relative to cyclophilin A in METTL3 degron cells expressing the GEMS system. n.s.=no significant difference, n=3 biological replicates. FIG. 3I. HEK293T cells were transfected with GEMS for 24 hours and then treated with cycloheximide (CHX) and collected after 0, 20, 40, 60, 90, and 120 minutes of treatment. RNA was extracted at each time point and m⁶A reporter mRNA abundance was measured by RT-qPCR. N.s.=no significant difference, n=3 biological replicates. FIG. 3J. The GEMS system was transfected into HEK293T cells in the presence or absence of METTL3 overexpression. EGFP fluorescence is increased in METTL3-overexpressing cells. Scale bar: 100 μm. FIG. 3K. Western blot analysis shows an increase in EGFP protein expression in cells overexpressing METTL3. FIG. 3L. Quantification of EGFP/EGFP-DHFR ratio from western blot data indicates increased EGFP/EGFP-DHFR in METTL3-overexpressing cells. *p<0.05, n=3 biological replicates. FIG. 3M. Sanger sequencing analysis of RNA extracts from cells in (FIG. 3J) shows increased C-to-U editing of the m⁶A sensor sequence in response to METTL3 overexpression. **p<0.01, n=3 biological replicates. FIG. 3N. RNA was extracted from cells transfected with the GEMS system with or without simultaneous METTL3 overexpression and subjected to RT-qPCR to measure the expression of the m⁶A reporter mRNA. N.s.=no significant difference, n=3 biological replicates. FIG. 3O. RNA was extracted from HEK293T cells transfected with GEMS with or without simultaneous overexpression of METTL3, followed by RT-qPCR-based m⁶A quantification of the m⁶A sensor sequence. ***p<0.001, n=3 biological replicates. FIG. 3P Correlation between the change in m⁶A level in the reporter mRNA and EGFP protein production (EGFP/EGFP-DHFR) in response to METTL3 overexpression. Line represents best-fit and shaded area represents confidence interval around the fit. R=0.971; n=4 biological replicates. FIG. 3Q. Schematic showing the main components of the GEMS plasmid with the addition of DsRed under the control of a separate promoter. FIG. 3R. HEK293T cells infected with Cas9 and either METTL3 sgRNA or AAVS1 sgRNA (control) were transfected with the GEMS system and subjected to flow cytometry based on EGFP and DsRed fluorescence. The proportion of cells in the indicated flow-sorted populations that contain METTL3 indels is shown. FIG. 3S. RNA samples were prepared from cell populations sorted in (i) and subjected to RT-PCR/Sanger sequencing of the m⁶A sensor sequence. C-to-U editing is only detected in the EGFP+ population. FIG. 3T. RNA was isolated from sorted cell populations and analyzed by RT-qPCR for METTL3 expression. The DsRed+/EGFP− population shows significantly lower METTL3 expression. ***p<0.001, n=2 biological replicates.

FIGS. 4A-4G show that GEMS detects differences in methylation across cell types. FIG. 4A. The GEMS system containing an internal m⁶A-independent DsRed reporter was transfected into HEK293T, HeLa, and Huh-7 cells followed by fluorescence microscopy 24 h later. M⁶A-coupled EGFP fluorescence is reduced in Huh-7 cells compared to HEK293T and HeLa cells. Scale bar: 100 μm. FIG. 4B. Western blot analysis of cells in (FIG. 4A) shows decreased EGFP expression in Huh-7 cells compared to HEK293T and HeLa cells. FIG. 4C. Quantification of EGFP/EGFP-DHFR ratio relative to DsRed expression in HEK293T, HeLa, and Huh-7 cells. N.s.=no significant difference, 915 ***p<0.001; n=3 biological replicates. FIG. 4D. Sanger sequencing shows C-to-U editing of the m⁶A sensor sequence in RNA samples from cells in (FIG. 4A). Huh-7 cells have reduced C-to-U editing compared to HEK293T and HeLa cells. Quantification of % C-to-U is shown on the right. ***p<0.001; n=3 biological replicates. FIG. 4E. The GEMS system expressing APO1-YTH or APO1-YTHmut was transfected into the indicated cell types followed by fluorescence microscopy 24 h later. In all cell types, EGFP fluorescence was detected in the presence of APO1-YTH but not APO1-YTHmut, indicating m⁶A-dependent activity of the GEMS system. Scale bar: 10 μm. FIG. 4F. Cell lysates were prepared from cells transfected as in (a) and analyzed by western blot. All cell types show decreased EGFP protein production in the presence of APO1-YTHmut. FIG. 4G. RT-PCR/Sanger sequencing analysis of the m⁶A sensor sequence shows C-to-U editing in all cell types tested which is absent in the presence of APO1-YTHmut. Quantification of editing is shown below. N.s.=not statistically significant. N=3 biological replicates. FIG. 4H. RT-qPCR-based m⁶A quantification shows reduced m⁶A in the m⁶A sensor sequence in Huh-7 cells compared to HEK293T and HeLa cells. Dotted line indicates minimum m⁶A detection threshold. N.s.=no significant difference, **p<0.01, n=3 biological replicates.

FIG. 4I. Bioanalyzer traces are shown for purified mRNA samples from HEK293T, HeLa, and Huh-7 cells that were subsequently analyzed by mass spectrometry to quantify cellular m⁶A. The traces confirm removal of rRNA in each sample. FIG. 4J. Mass spectrometry was used to quantify m⁶A in purified mRNA from HEK29T, HeLa, and Huh-7 cells.

FIGS. 5A-5F show that GEMS senses changes in m⁶A caused by small molecule inhibition of METTL3. FIG. 5A. EGFP fluorescence from the GEMS system is reduced in HEK293T cells treated with the METTL3 inhibitor STM2457. GEMS-expressing cells were treated with 30 μM STM2457 for 24 h. Scale bar: 100 μm. FIG. 5B. Quantitative microscopy was performed on HEK293T cells expressing the GEMS system and treated with 30 μM STM2457. Treatment with STM2457 shows a significant reduction in EGFP fluorescence intensity. ***p<0.001; n>400 cells per condition. EGFP signal in each cell was normalized to DsRed. FIG. 5C. Western blot shows decreased EGFP protein in STM2457-treated cells. FIG. 5D. Densitometry analysis indicates reduced EGFP/EGFP-DHFR ratio in cells treated with 30 μM STM2457 for 24 h. ***p<0.001; n=2 biological replicates. FIG. 5E. HEK293T cells were treated with 10 or 30 μM of STM2457 for 24 hours followed by transfection with the GEMS system containing DsRed as an m⁶A-uncoupled internal control. Increasing amounts of STM2457 lead to increased depletion of m⁶A-coupled EGFP fluorescence. Scale bar: 100 μm. FIG. 5F. Western blot from cells in (FIG. 5E) shows decreased production of EGFP protein with increasing doses of STM2457. FIG. 5G. Sanger sequencing traces (top) and quantification (bottom) of the m⁶A sensor sequence from cells in (FIG. 5E) indicates decreased editing with increasing amounts of STM2457. N=3 biological replicates. FIG. 5H. Mass spectrometry analysis of purified mRNA from cells in (FIG. 5E) indicates depletion of m⁶A following STM2457 treatment. ***p<0.001, n=2 biological replicates. FIG. 5I. RNA was extracted from cells in FIG. 5E that were treated with 30 μM STM2457 or DMSO and subjected to RT-qPCR to measure abundance of the m⁶A reporter mRNA. N.s.=no significant difference; n=2 biological replicates. FIG. 5J. Sanger sequencing of the m⁶A sensor sequence in RNA samples from cells in FIG. 5A shows reduced C-to-U editing in cells treated with STM2457. Quantification of % C-to-U is shown on the right. ***p<0.001; n=3 biological replicates. FIG. 5K. RT-qPCR-based m⁶A detection was used to quantify relative m⁶A levels of endogenous ACTB A1222 and the m⁶A sensor sequence. STM2457 treatment leads to similar reductions in m⁶A in endogenous ACTB and the m⁶A sensor sequence. ***p<0.001; n=3 biological replicates. FIG. 5L. HEK293T cells were treated as in (FIG. 5E) but transfected with a version of GEMS containing EGFP fused to a PEST destabilization domain. The EGFP signal shows greater depletion at lower doses of STM2457 compared to (FIG. 5E), indicating improved sensitivity of GEMS-EGFP-PEST as a readout for changes in m⁶A compared to GEMS-EGFP. FIG. 5M. Western blot from cells in (FIG. 5I) shows decreased production of EGFP protein with increasing doses of STM2457. FIG. 5N. Sanger sequencing traces of the m⁶A sensor sequence from cells in (FIG. 5I) indicates decreased editing with increasing amounts of STM2457. N=3 biological replicates. FIG. 5O. Quantification of EGFP/EGFP-DHFR ratio following STM2457 treatment of HEK293T cells expressing GEMS with EGFP or EGFP-PEST. The EGFP-PEST version shows an improved response at low doses of STM2457 compared to EGFP. ***p<0.001, **p<0.01, n=3 biological replicates. FIG. 5P is a cartoon depicting an example of an alternative FP that could be utilized in the GEMS system in place of EGFP. This schematic shows primary neurons that are infected with a lentivirus expressing a photoconvertible FP such as Dendra2, which emits green fluorescence that is converted to red fluorescence upon exposure to UV light. New Dendra2 protein can the subsequently be identified by green fluorescence.

FIG. 6 is a schematic of the m⁶A feedback system according to certain embodiments of this disclosure. METTL3 transcription leads to methylation of the sensor sequence and translation of dCas9-KRAB. Constitutive expression of METTL3 sgRNA targets dCas9-KRAB to the METTL3 locus to inhibit transcription. This results in decreased methylation of the sensor sequence and dCas9-KRAB depletion, allowing METTL3 transcription to resume.

FIG. 7 shows that dCas9-KRAB can be expressed in place of GFP in the m⁶A reporter mRNA. HEK293T cells were transfected with the m⁶A sensor system using a plasmid in which GFP was replaced with the coding sequence for dCas9-KRAB. Western blot shows expression of dCas9-KRAB and APO1-YTH. Cyclophilin A is shown as a loading control.

FIGS. 8A-8F show that dCas13-tethered APO1-YTH enables targeted m⁶A sensor sequence editing. FIG. 8A. Schematic showing the main components of the GEMS system with dCas13-APO1-YTH (dCas13-GEMS). Location of regions in the m⁶A reporter mRNA targeted by the indicated gRNAs is shown. FIG. 8B. HEK293T cells were co-transfected with dCas13-GEMS and the indicated gRNA. Only gRNAs targeting within the m⁶A sensor sequence enable GEMS activity (EGFP fluorescence). gRNA CTL=scrambled gRNA control. Scale bar: 100 μm. FIG. 8C1. RNA from cells in (FIG. 8B) was subjected to RT-PCR/Sanger sequencing targeting the m⁶A sensor sequence and known m⁶A sites in four cellular mRNAs (ACTB A1222, HERC2 A14782, NIPA1 A6089, and SMUG1 A1303). In cells expressing dCas13-GEMS and a gRNA targeting the sensor sequence, C-to-U editing is only detected in the m⁶A sensor sequence and not in cellular mRNAs. In contrast, cells expressing the APO1-YTH version of GEMS have editing of both the sensor sequence and cellular mRNAs. Asterisks denote m⁶A sites. FIG. 8C2. Top row shows RT-PCR/Sanger sequencing-based quantification of C-to-U editing of the m6A sensor sequence after expression of the GEMS system in HEK293T cells. This version of the GEMS system contains the APO1-YTH protein. Bottom row shows the same, but for cells expressing a version of the GEMS system with dCas13-APO1-YTH and a m6A reporter mRNA-targeting gRNA. FIG. 8D. Quantification of editing at the 2 convertible stop codons within the m⁶A sensor sequence from I. n.s.=no significant difference. ***p<0.001. n=2 biological replicates. FIG. 8E. RNA was extracted from cells in (b) and RT-qPCR-based m⁶A quantification was used to measure m⁶A in the m⁶A sensor sequence. n.s.=no significant difference, n=3 biological replicates. FIG. 8F. HEK293T cells were treated with STM2457 for 16 hours and then co-transfected with dCas13-GEMS and the indicated gRNAs. EGFP fluorescence activated by dCas13-GEMS is decreased in response to STM2457 treatment. Scale bar: 100 μm.

FIGS. 9A-9R show that m⁶A-coupled effector protein delivery counteracts the effects of m⁶A hypermethylation in cancer cells. FIG. 9A. Schematic showing m⁶A-coupled expression of a tumor suppressor protein to counteract the effects of m⁶A hypermethylation in cancer cells. FIG. 9B. Left: schematic shows the GEMS system Middle: Schematic showing the results of previously published studies (paper above) which found that hypermethylation of the SOCS2 mRNA leads to its degradation and reduced SOCS2 protein expression in liver cancer cells. SOCS2 is an inhibitor of the JAK/STAT pathway and acts as a tumor suppressor in hepatocellular carcinoma Right: Schematic of the GEMS system in which EGFP has been replaced with SOCS2. FIG. 9C. Huh-7 cells treated with increasing amounts of STM2457 show a dose-dependent increase in SOCS2 mRNA expression as measured by RT-qPCR. These data are consistent with m⁶A-mediated inhibition of SOCS2 abundance. ***p<0.001, **p<0.01. n=3 biological replicates. FIG. 9D. GEMS was used to deliver either EGFP (GEMS-EGFP) or SOCS2 (GEMS-SOCS2) into Huh-7 cells. Western blot indicates robust expression of SOCS2 in cells expressing GEMS-SOCS2. FIG. 9E. RT-qPCR shows SOCS2 coding sequence expression in Huh-7 cells transfected with GEMS-SOCS2. FIG. 9F. Sanger sequencing of the m⁶A sensor sequence from cells in (FIG. 9D) indicates similar C-to-U editing rates of the GEMS-EGFP and GEMS-SOCS2 mRNAs. n.s.=no significant difference; n=3 biological replicates FIG. 9G. Quantification of EGFP/EGFP-DHFR ratio and SOCS2/SOCS2-DHFR ratio from western blot data from cells expressing GEMS-EGFP or GEMS-SOCS2 indicates similar ratios. FIG. 9H. Western blot analysis of downstream SOCS2 targets shows a decrease in STAT5 and JAK2 phosphorylation in Huh-7 cells expressing GEMS-SOCS2. FIG. 9I. RT-qPCR shows reduced expression of SOCS2 target mRNAs IGF1 and CyclinD1 in Huh-7 cells expressing GEMS-SOCS2. ***p<0.001, n=3 biological replicates. FIG. 9J. Cell growth assays show reduced growth of Huh-7 cells transfected with 952 GEMS-SOCS2 compared to non-transfected cells (Control). Growth curves for both were normalized to cells expressing GEMS-EGFp. n=3 biological replicates. FIG. 9K. Huh-7 cell migration is diminished following expression of GEMS-SOCS2 compared to GEMS-EGFP. FIG. 9L. Western blot shows elevated p53 levels in Huh-7 cells expressing GEMS-p53 compared to GEMS-EGFP. Top panel shows brightfield images of cells migration; bottom panel shows quantification of the total number of cells migrated. ***p<0.001; n=3 biological replicates. FIGS. 9M-90 shows that GEMS achieves m⁶A-coupled p53 expression in cancer cells. FIG. 9M. Huh-7 cells expressing GEMS-EGFP or GEMS-p53 were subjected to RT-PCR and Sanger sequencing of the m⁶A sensor sequence. Similar C-to-U editing of target cytidines is achieved with GEMS-EGFP and GEMS-p53. FIG. 9N. RT-qPCR shows TP53 coding sequence expression in Huh-7 cells transfected with GEMS-p53. FIG. 9O. RT-qPCR shows reduced expression of p53 target mRNAs CDKN1A and GADD45A in Huh-7 cells expressing GEMS-p53. ***p<0.001, n=3 biological replicates. FIG. 9P. Cell growth is reduced in Huh-7 cells transfected with GEMS-p53 compared to non-transfected cells (Control). Growth curves for both were normalized to cells expressing GEMS-EGFP. ***p<0.001; n=3 biological replicates. FIG. 9Q. Brightfield images (top) and quantification (bottom) of Huh-7 cell migration measured 24 h after transfection with GEMS-EGFP or GEMS-p53. ***p<0.001; n=3 biological replicates. FIG. 9R. Comparison of the effects of GEMS-p53 delivery into Huh-7 cells (which express mutant p53) and HepG2 cells (which express wild type p53). The number of cells following GEMS-p53 transfection relative to GEMS-EGFP transfection for each cell type across 5 days is shown. ***p<0.001, n=3 biological replicates

FIGS. 10A-10D show that GEMS enables tunable protein expression with m⁶A levels. FIG. 10A. Huh-7 and HepG2 cells were transfected with either GEMS-p53 or GEMS-SOCS2. Western blot shows increased production of SOCS2 and p53 proteins in HepG2 cells. FIG. 10B. Quantification of p53/p53-DHFR and SOCS2/SOCS2-DHFR ratios in Huh-7 and HepG2 cells shows increased ratios in HepG2 cells compared to Huh-7 cells. *p<0.05, n=2 biological replicates. FIG. 10C. Top: Sanger sequencing traces of the m⁶A sensor sequence from cells in (FIG. 10A) indicates increased editing of convertible stop codons in HepG2 cells compared to Huh-7 cells. Bottom: quantification of C-to-U editing. ***p<0.001; n=3 biological replicates. FIG. 10D. RT-qPCR-based m⁶A quantification shows increased m⁶A in the m⁶A sensor sequence in HepG2 cells compared to Huh-7 cells. Dotted line indicates minimum m⁶A detection threshold. **p<0.01, *p<0.05; n=3 biological replicates.

FIG. 11: Neurons can be isolated from transgenic mice expressing the APOBEC1-YTH enzyme and then the m6A reporter mRNA could be introduced with viral infection or other means to examine m6A dynamics. This could also be done using mice that express the GEMS system.

FIG. 12: the GEMS system is compatible with HTS, so it could be used for HTS studies such as those seeking to identify cellular proteins/pathways that control m6A abundance or drugs/small molecules that inhibit METTL3 or m6A demethylases.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

Unless defined otherwise, all technical and scientific terms used herein have the same meaning as commonly understood by one of ordinary skill in the art to which this disclosure belongs. All patents, patent applications and publications referred to throughout the disclosure herein are incorporated by reference in their entirety.

Articles “a” and “an” are used herein to refer to one or to more than one (i.e. at least one) of the grammatical object of the article. By way of example, “an element” means at least one element and can include more than one element.

The use of any and all examples or exemplary language (e.g., “such as”) provided herein, is intended merely to better illustrate the invention and does not pose a limitation on the scope of the invention unless otherwise claimed.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

“About” is used to provide flexibility to a numerical range endpoint by providing that a given value may be “slightly above” or “slightly below” the endpoint without affecting the desired result.

The use herein of the terms “including,” “comprising,” or “having” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

As used throughout, the term “nucleic acid” or “nucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar properties as the reference nucleic acid and are metabolized in a manner similar to naturally occurring nucleotides. A nucleic acid sequence can comprise combinations of deoxyribonucleic acids and ribonucleic acids. Such deoxyribonucleic acids and ribonucleic acids include both naturally occurring molecules and synthetic analogues. The polynucleotides of the invention also encompass all forms of sequences including, but not limited to, single-stranded forms, double-stranded forms, hairpins, stem-and-loop structures, and the like.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

As used throughout the term “mRNA” or “mRNA transcript” refers to a single-stranded RNA having at least one open reading frame that can be translated by a cell to express a protein, The cell can be an in vitro cell or an in vivo cell.

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bond”.

“Contacting” as used herein, e.g., as in “contacting a cell” refers to contacting a cell directly or indirectly in vitro, ex vivo, or in vivo (i.e., within a subject as defined herein). Contacting a cell may include addition of a compound (e.g., a genetically encoded m⁶A-coupled effector protein delivery system) to a cell, or administration to a subject. Contacting encompasses administration to a solution, cell, tissue, mammal, subject, patient, or human. Further, contacting a cell includes adding an agent to a cell culture.

As used herein, the terms “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like, as well as animal models, such as transgenic animals, and the like. The methods and compositions disclosed herein can be used on a sample either in vitro (for example, on isolated cells or tissues) or in vivo in a subject (i.e., living organism, such as a patient or animal model). In embodiments of methods as described herein, the sample comprises a plurality of cells.

As used throughout, a catalytic domain of a cytidine deaminase is a polypeptide comprising a cytidine deaminase, for example, Apolipoprotein B mRNA Editing Enzyme Catalytic Subunit (APOBEC1), activation induced cytidine deaminase (AICDA), Apolipoprotein B mRNA editing enzyme, catalytic polypeptide-like 3A (APOBEC3A), or a catalytic fragment of any thereof, that catalyzes deamination of cytidine (“C”) to uridine (“U”) in RNA molecules. As used throughout, a catalytic domain of an adenosine deaminase, is a polypeptide comprising an adenosine deaminase, for example, double-stranded RNA-specific adenosine deaminase (ADAR1), or a catalytic fragment thereof, that catalyzes deamination of adenosine (“A”) to inosine (“I”) in RNA molecules. In some embodiments, the catalytic domain retains at least about 75%, 80%, 90%, 95%, or 99% of the enzymatic activity of the wildtype deaminase from which the domain is derived.

As used throughout, the term “Cas9 polypeptide” means a Cas9 protein or a fragment thereof present in any bacterial species that encodes a Type II CRISPR/Cas9 system. See, for example, Makarova et al. Nature Reviews, Microbiology, 9: 467-477 (2011), including supplemental information, hereby incorporated by reference in its entirety. For example, the Cas9 protein or a fragment thereof can be from Streptococcus pyogenes. Full-length Cas9 is an endonuclease comprising a recognition domain and two nuclease domains (HNH and RuvC, respectively) that creates double-stranded breaks in DNA sequences. In the amino acid sequence of Cas9, HNH is linearly continuous, whereas RuvC is separated into three regions, one left of the recognition domain, and the other two right of the recognition domain flanking the HNH domain. Cas9 from Streptococcus pyogenes is targeted to a genomic site in a cell by interacting with a guide RNA that hybridizes to a 20-nucleotide DNA sequence that immediately precedes an NGG motif recognized by Cas9. This results in a double-strand break in the genomic DNA of the cell.

As used throughout, a dCas9 polypeptide is a deactivated or nuclease-dead Cas9 (dCas9) that has been modified to inactivate Cas9 nuclease activity. Modifications include, but are not limited to, altering one or more amino acids to inactivate the nuclease activity or the nuclease domain. For example, and not to be limiting, D10A and H840A mutations can be made in Cas9 from Streptococcus pyogenes to inactivate Cas9 nuclease activity. Other modifications include removing all or a portion of the nuclease domain of Cas9, such that the sequences exhibiting nuclease activity are absent from Cas9. Accordingly, a dCas9 may include polypeptide sequences modified to inactivate nuclease activity or removal of a polypeptide sequence or sequences to inactivate nuclease activity. The dCas9 retains the ability to bind to DNA even though the nuclease activity has been inactivated. Accordingly, dCas9 includes the polypeptide sequence or sequences required for DNA binding but includes modified nuclease sequences or lacks nuclease sequences responsible for nuclease activity. It is understood that similar modifications can be made to inactivate nuclease activity in other site-directed nucleases, for example in Cpf1 or C2c2.

In some examples, the dCas9 protein is a full-length Cas9 sequence from S. pyogenes lacking the polypeptide sequence of the RuvC nuclease domain and/or the HNH nuclease domain and retaining the DNA binding function. In other examples, the dCas9 protein sequences have at least 30%, 40%, 50%, 60%, 70%, 80%, 90%, 95%, 98% or 99% identity to Cas9 polypeptide sequences lacking the RuvC nuclease domain and/or the HNH nuclease domain and retains DNA binding function. In other examples, the dCas9 protein sequence is encoded by a polynucleotide that has at least 30%, 40%, 50%, 60%, 70%, 80%, 90%, 95%, 98% or 99% to SEQ ID NO: 59.

As used throughout, the term “Cas13 polypeptide” means a Cas13 protein or a fragment thereof present in any bacterial species that encodes a Type VI CRISPR/Cas13 system. Exemplary Cas13 polypeptides include dPspCas13b, dLwaCas13a, and dRfxCas13d. Additional Cas13 polypeptides are described, for example, in Abudayyeh et al., Science. 2016 August 5; 353(6299): aaf5573. doi:10.1126/science.aaf5573, including supplemental information, hereby incorporated by reference in its entirety; Cox et al., Science 358, 1019-1027 (2017) including supplemental information, hereby incorporated by reference in its entirety; and Tang et al., Front. Cell Dev. Biol., 27 Jul. 2021 Sec. Epigenomics and Epigenetics Volume 9-2021; doi: 10.3389/fcell.2021.677587. For example, the Cas13 protein or a fragment thereof with ssRNA targeting activity can be from Leptotrichia wadei, Leptotrichia shahii, Prevotella sp. P5-125 (PspCas13b), or Ruminococcus flavefaciens. Generally, Cas13 enzymes have two higher eukaryotes and prokaryotes nucleotide-binding (HEPN) endoRNase domains that mediate precise RNA cleavage with a preference for targets with protospacer flanking sites (PFSs) observed biochemically and in bacteria.

As used throughout, a dCas13 polypeptide is a deactivated or nuclease-dead Cas13 (dCas13) that has been modified to inactivate Cas13 nuclease activity. Modifications include, but are not limited to, altering one or more amino acids to inactivate the nuclease activity or the nuclease domain. For example, and not to be limiting, H133A and H1058A mutations can be made in Cas13 HEPN domains from Prevotella sp. P5-125 (PspCas13b) to inactivate Cas13 nuclease activity (see, for example, Cox et al., Science 358, 1019-1027 (2017) including supplemental information, hereby incorporated by reference in its entirety, and International Patent Publication WO 2019/005884, also incorporated by reference in its entirety). Other modifications include removing all or a portion of the nuclease domain of Cas13 (for example, A984-1090 H133A of Cas13b is from Prevotella sp. P5-125; see, for example, Programmable m(6)A modification of cellular RNAs with a Cas13-directed methyltransferase. Wilson C, Chen P J, Miao Z, Liu D R. Nat Biotechnol. 2020 Jun. 29. pii: 10.1038/s41587-020-0572-6. doi: 10.1038/s41587-020-0572-6. 10.1038/s41587-020-0572-6 PubMed 32601430), such that the sequences exhibiting nuclease activity are absent from Cas13. Exemplary dCas13 polypeptide mutations include R474A/R1046A in dCas13 from L. wadei and mutations R239R/H244A/ and R858A/H863A from Ruminococcus flavefaciens strain XPD3002. Accordingly, a dCas13 may include polypeptide sequences modified to inactivate nuclease activity or removal of a polypeptide sequence or sequences to inactivate nuclease activity. The dCas13 retains the ability to target ssRNA even though the nuclease activity has been inactivated. Accordingly, dCas13 includes the polypeptide sequence or sequences required for ssRNA targeting but includes modified nuclease sequences or lacks nuclease sequences responsible for nuclease activity.

In some examples, the dCas13 protein is a full-length Cas13 sequence from L. wadei, L. shahii, Prevotella sp. P5-125 (PspCas13b), or R. flavefaciens having one or more mutations in one or more HEPN domains and retaining the ssRNA targeting function. In other examples, the dCas13 protein sequences have at least 30%, 40%, 50%, 60%, 70%, 80%, 90%, 95%, 98% or 99% identity to Cas13 polypeptide sequences with HEPN mutations and retains RNA binding function. In other examples, the dCas13 protein sequence is encoded by a dCas13 polynucleotide coding fragment that has at least 30%, 40%, 50%, 60%, 70%, 80%, 90%, 95%, 98% or 99% to the corresponding dCas13 polynucleotide coding fragment present in SEQ ID NO: 60.

### I. Introduction

N⁶-methyladenosine (m⁶A) is the most abundant internal mRNA modification and influences several steps of the RNA life cycle, including splicing, stability, and translation 1, 2. The majority of m⁶A sites in cells are deposited co-transcriptionally by a single methyltransferase, METTL3, which interacts with additional accessory proteins to target RNAs for methylation. In mammals, m⁶A occurs in a unique consensus sequence which at its core consists of RAC (R=G or A), and It is enricled in proximal 3′UTRs and in the vicinity of the stop codon^{3, 4}. m⁶A carries out its diverse RNA regulatory functions by recruiting m⁶A binding proteins, which mediate the ability of m⁶A to impact the expression of thousands of cellular mRNAs.

Consistent with the broad roles for m⁶A in gene expression control, m⁶A has emerged as an important regulator of cellular function. m⁶A is necessary for several physiological processes, including stem cell maintenance, development, innate immunity, and learning and memory⁵⁻⁷. Additionally, dynamic regulation of m⁶A provides a mechanism for cells to fine-tune gene expression in response to changing cellular conditions. For instance, some forms of cellular stress can lead to hyper- or hypomethylated states which impact the expression of stress response genes 8-10 and synaptic activity alters mRNA methylation in the brain to control the expression of synaptic plasticity genes^{5, 11-13} In addition, abnormal regulation of m⁶A levels in cells contributes to a variety of human diseases, including cardiovascular disease, the response to viral infection, and several cancers¹⁴⁻¹⁶ METTL3 and other methyltransferase complex proteins are often upregulated in cancer, leading to elevated levels of m⁶A that promote the expression of genes that support cancer cell proliferation and migration. Thus, detecting changes in m⁶A levels across cell types or under certain cellular conditions is important for understanding how m⁶A contributes to cellular function in both healthy and disease states.

Much of the progress that has been made in understanding m⁶A regulation in cells has been through the development of new tools that have enabled m⁶A detection. Strategies for detecting global changes in cellular m⁶A levels have primarily used three approaches: m⁶A antibodies, thin-layer chromatography, or mass spectrometry. However, these methods suffer from several limitations, including high cost, the need for large amounts of RNA, and multiple sample processing steps. Moreover, antibody-based methods suffer from non-specificity, mass spectrometry requires specialized equipment, and TLC depends on radioactivity. More recently, alternatives to antibody-based global m⁶A mapping have been developed¹⁷⁻²¹, but these methods often require substantial amounts of input RNA. Importantly, all current strategies involve isolation of RNA from cells and therefore do not enable real-time monitoring of m⁶A methylation in living cells. These limitations have been a major barrier for understanding how cellular m⁶A is dynamically regulated. In addition, no method exists for providing a specific readout of cellular m⁶A methylation in a manner compatible with high-throughput screening (HTS). This has substantially limited drug discovery efforts aimed at identifying inhibitors of METTL3, and it has prevented other high-throughput studies designed to identify factors that regulate m⁶A in cells.

Based on the aforementioned deficiencies, there existed a great need to develop a simple, low-cost method for detecting adenosine methylation in living cells which is also compatible with HTS. As described at least in International Patent Application PCT/US2022/079709 (which is incorporated by reference as if fully set forth herein), progress has been made in this area. Genetically Encoded m⁶A Sensor technology (also referred to herein as “GEMS”) is described at least in International Patent Application PCT/US2022/079709, which can couple protein expression, such as a fluorescent signal, with cellular mRNA methylation. Sensors and methods as described therein can detect changes in m⁶A levels caused by pharmacological inhibition of the m⁶A methyltransferase, giving it potential utility for drug discovery efforts.

However, prior methods for studying m⁶A required RNA isolation and did not provide a real-time readout of mRNA methylation in living cells, leading to the development of technology such as the Genetically Encoded m⁶A Sensor technology (also referred to herein as “GEMS”). Other aspects of GEMS system components are additional described for example, at least in U.S. Pat. No. 11,680,109, which is incorporated by reference as if fully set forth herein.

Some of these prior approaches to date, however, may risk editing of off-target endogenous RNAs when fusion proteins comprising N⁶-methyladenosine (m⁶A) binding domain of a YT521-B homology (YTH) domains are utilized. Furthermore, while drug discovery efforts have been made aimed at METTL3 inhibition, targeted delivery of therapeutics (such as tumor suppressors and cell cycle proteins, for example), an area of research which can see improvements.

Described herein are constructs, expression systems, methods, kits, animals, and cells relating to programmable sensors and methods which can be programmed for targeted delivery of cells to achieve m⁶A-dependent delivery of custom protein payloads in cells. Thus, constructs, expression systems, and methods as described herein can provide a versatile platform based on m⁶A sensing, allowing for (at least): (1) a simple readout for m⁶A methylation; (2) a system for m⁶A-coupled protein expression; and (3) a system for targeted m⁶A-coupled protein expression. Furthermore, the GEMS systems as described can be modified for effector protein expression (e.g., expression of proteins related to tumor suppression or cell cycle regulation, such as p53 or suppressor of tumor signaling 2 (SOCS2)) or an RNA-guided endonuclease that has been modified to remove cleavage activity (e.g., a “dead” CAS protein). Systems as described herein additionally can be employed in transgenic or knock-in animals or cells derived from animal models as described herein.

Disclosed herein are compositions, systems, and methods related to overcoming the aforementioned limitations.

Disclosed herein are genetically encoded sensors for m⁶A which can provide a fluorescent readout when m⁶A is deposited on mRNA. The sensor may be used for detecting mRNA methylation in a variety of cell types (without intending to be limiting in immortalized or primary tumor cells in vitro, for example), and for responding to small molecule inhibition of the m⁶A methyltransferase, METTL3, as discussed. In addition, as disclosed herein, the m⁶A sensor platform can be utilized to express effector proteins of interest instead of a reporter protein (i.e., eGFP), such as anti-tumor therapeutics or tumor suppression proteins. For example, sensors as described herein can achieve m⁶A-coupled delivery of anti-tumor therapeutics (for example, tumor suppressor proteins to slow the growth of cancer cells through the expression of p53 or other tumor suppressor proteins) in cancer cells that have elevated m⁶A levels.

Additionally, components of the compositions, systems, and methods as described herein can be targeted to prevent off-target effects (such as unwanted editing of off-target RNAs in physiologically normal or otherwise healthy cells) utilizing catalytically-dead CRISPR-associated (Cas) enzymes, for example, of RNA-targeting (also referred to herein as “RNA-guided”) type III (i.e., Csm/Csr), type VI (i.e. Cas13), or type II (i.e., Cas9) CRISPR-Cas systems. Altogether, the system provides a simple, highly versatile approach that can be used for sensing m⁶A in living cells and coupling mRNA methylation to effector protein expression.

### II. Expression Systems

Provided herein is an expression system comprising: (a) a first DNA construct comprising a nucleic acid sequence encoding a fusion protein, wherein the fusion protein comprises an N⁶-methyladenosine (m⁶A) binding domain of a YT521-B homology (YTH) domain-containing protein fused to a catalytic domain of a cytidine deaminase or a catalytic domain of an adenosine deaminase (e.g., APOBEC1); and (b) a second DNA construct comprising (i) a nucleic acid sequence encoding an effector polypeptide; (ii) a m⁶A sensor sequence; and (iii) a polypeptide encoding dihydrofolate reductase (DHFR). The nucleic acid sequence encoding an effector; (ii) a m⁶A sensor sequence; and (iii) a polypeptide encoding dihydrofolate reductase (DHFR) is also referred to as the mRNA reporter sequence or effector sequence. Also provided is a nucleic acid sequence comprising a nucleic acid sequence encoding an effector protein, a m⁶A sensor sequence, and, a polypeptide encoding dihydrofolate reductase (DHFR).

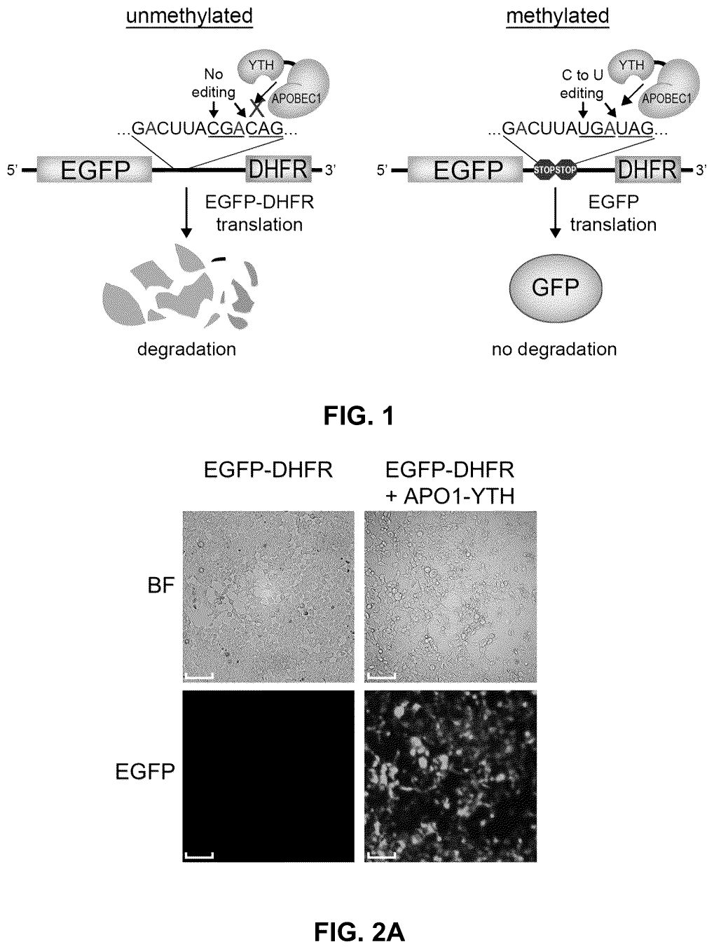

The m⁶A methylation sensor system previously discovered by the inventors, as described in PCT/US2022/079709, U.S. Pat. No. 11,680,109, and Meyer, K. D., “DART-seq: an antibody-free method for global m(6)A detection,” Nat Methods. 2019 December, 16(12):1275-1280 (published online Sep. 23, 2019); doi: 10.1038/s41592-019-0570-0, the entire contents of all of which (including sequence information and any supplemental information) are incorporated by reference in their entirety as fully set forth herein includes at least two components: 1) expression of APO1-YTH, and 2) expression of a protein in the presence of m⁶A (FIG. 1; the reporter protein eGFP is shown, which can be interchanged for an effector protein as described herein). The mRNA of the effector protein comprises the coding sequence for an effector protein (for example, a dCas or a tumor suppression protein), followed by a short m⁶A “sensor sequence” (for example, 5′GACUUACGACAG3′), which contains two m⁶A consensus motifs (GAC) and two tandem “convertible” stop codon sequences that are in-frame with EGFP (FIG. 1). The m⁶A sensor sequence can be modified from a similar sequence in the human ACTB mRNA 3′UTR, which contains two methylated GAC sequences that have been reported in many different cell types. When unedited, the convertible stop codons encode arginine and glutamine (CGA and CAG, respectively). However, C-to-U editing produces two stop codons (UGA and UAG) (FIG. 1). Downstream of the m⁶A sensor sequence and in-frame with EGFP is the coding sequence for a destabilization domain modified from the Escherichia coli dihydrofolate reductase gene (DHFR). This DHFR destabilization domain induces rapid, proteasome-mediated degradation of proteins to which it is tethered. Thus, when the GFP-DHFR m⁶A reporter mRNA is introduced into cells together with APO1-YTH, if the reporter mRNA is not methylated, there will be no editing of the m⁶A sensor sequence by APO1-YTH and the full-length GFP-DHFR protein will be translated. The result is rapid degradation of GFP-DHFR and no fluorescence (FIG. 1, left panel). However, if either of the GAC sequences within the m⁶A sensor sequence is methylated, APO1-YTH will bind to the m⁶A and deaminate one or both cytidine residues within the two convertible stop codons of the sensor sequence. The result is translation of GFP followed by translation termination before the ribosome encounters the DHFR sequence. The GFP protein will not be degraded since it will not be fused to DHFR, resulting in GFP fluorescence (FIG. 1, right panel). Thus, this system provides a simple fluorescent readout for the presence of m⁶A (i.e., no m⁶A=no GFP fluorescence; m⁶A=GFP fluorescence). Again, although eGFP is shown in FIG. 1, it would be understood that the eGFP polypeptide can be interchanged for an effector protein polypeptide.

Although the m⁶A sensor system uses m⁶A-coupled GFP expression as a readout, any gene of interest can be cloned in place of GFP to achieve m⁶A-dependent protein expression. Such an m⁶A-coupled effector protein delivery system has several potential applications (e.g., in cancer therapy). Additional aspects of expression systems are provided in Sections I and II above.

The recombinant nucleic acids provided herein can be included in expression cassettes for expression in a host cell or an organism of interest. The cassette will include 5′ and 3′ regulatory sequences operably linked to a recombinant nucleic acid provided herein that allows for expression of the modified polypeptide. The cassette may additionally contain at least one additional gene or genetic element to be co-transformed into the organism. Where additional genes or elements are included, the components are operably linked. Alternatively, the additional gene(s) or element(s) can be provided on multiple expression cassettes. Such an expression cassette is provided with a plurality of restriction sites and/or recombination sites for insertion of the polynucleotides to be under the transcriptional regulation of the regulatory regions. The expression cassette may additionally contain a selectable marker gene. The expression cassette will include in the 5′ to 3′ direction of transcription: a transcriptional and translational initiation region (i.e., a promoter), a polynucleotide of the invention, and a transcriptional and translational termination region (i.e., termination region) functional in the cell or organism of interest. The promoters of the invention are capable of directing or driving expression of a coding sequence in a host cell. The regulatory regions (i.e., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention.

Additional regulatory signals include, but are not limited to, transcriptional initiation start sites, operators, activators, enhancers, other regulatory elements, ribosomal binding sites, an initiation codon, termination signals, and the like. See Sambrook et al. (1992) Molecular Cloning: A Laboratory Manual, ed. Maniatis et al. (Cold Spring Harbor Laboratory Press, Cold Spring Harbor, N.Y.) (hereinafter “Sambrook 11”); Davis et al., eds. (1980) Advanced Bacterial Genetics (Cold Spring Harbor Laboratory Press), Cold Spring Harbor, N.Y., and the references cited therein.

Further provided is a vector comprising a nucleic acid or expression cassette set forth herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene (See generally, Sambrook et al. Molecular Cloning: A Laboratory Manual, 4^{th} ed., Cold Spring Harbor Laboratory Press, Cold Spring Harbor, 2012). The vector, for example, can be a plasmid.

The expression vectors described herein can also include the nucleic acids as described herein under the control of an inducible promoter such as the tetracycline inducible promoter or a glucocorticoid inducible promoter. The nucleic acids of the present invention can also be under the control of a tissue-specific promoter to promote expression of the nucleic acid in specific cells, tissues or organs. Any regulatable promoter, such as a metallothionein promoter, a heat-shock promoter, and other regulatable promoters, of which many examples are well known in the art are also contemplated. Furthermore, a Cre-loxP inducible system can also be used, as well as a Flp recombinase inducible promoter system, both of which are known in the art.

Provided herein is a m⁶A-coupled effector protein expression system and methods of introducing same into a cell, tissue, and/or animal model to achieve m⁶A-dependent protein expression. In some embodiments, the m⁶A-coupled effector protein expression system comprises (a) a nucleic acid sequence encoding a fusion protein, wherein the fusion protein comprises an N⁶-methyladenosine (m⁶A) binding domain of a YT521-B homology (YTH) domain-containing protein fused to a catalytic domain of a cytidine deaminase, and (b) a nucleic acid sequence encoding an effector protein and dihydrofolate reductase (DHFR). In some embodiments, the catalytic domain of the cytosine deaminase is the catalytic domain of apolipoprotein B mRNA editing enzyme (APOBEC-1). Also provided is a vector comprising any of the nucleic acid sequences described herein.

In some embodiments, the effector protein is a tumor suppressor protein, for example, METTL3. In some embodiments, the effector protein is an RNA-guided endonuclease. In some embodiments, the RNA-guided endonuclease is a dead RNA-guided endonuclease, for example, dead Cas9 (dCas9). In some embodiments, the effector protein comprises dCas9 linked or fused to a transcriptional regulator, for example, a transcriptional repressor (e.g., KRAB). In some embodiments, the effector protein comprises dCas9 linked or fused to a transcriptional activator. In any of the methods described herein, one or more guide RNAs can be introduced into the cell to guide the dCas9 to a specific site in the genome of the cell.

Also provided is a DNA construct comprising a promoter operably linked to a recombinant nucleic acid described herein. A nucleic acid is “operably linked” when it is placed into a functional relationship with another nucleic acid sequence. Numerous promoters can be used in the constructs described herein. A promoter is a region or a sequence located upstream and/or downstream from the start of transcription that is involved in recognition and binding of RNA polymerase and other proteins to initiate transcription. The promoter can be a eukaryotic or a prokaryotic promoter. In some embodiments the promoter is an inducible promoter. In some embodiments, the promoter is a constitutive promoter.

Any of the nucleic acid sequences provided herein can be included in expression cassettes for expression in a host cell or an organism of interest. The cassette will include 5′ and 3′ regulatory sequences operably linked to a recombinant nucleic acid provided herein that allows for expression of the modified polypeptide. A nucleic acid is “operably linked” when it is placed into a functional relationship with another nucleic acid sequence. Numerous promoters can be used in the constructs described herein. A promoter is a region or a sequence located upstream and/or downstream from the start of transcription that is involved in recognition and binding of RNA polymerase and other proteins to initiate transcription. The promoter can be a eukaryotic or a prokaryotic promoter. In some embodiments the promoter is an inducible promoter. In some embodiments, the promoter is a constitutive promoter.

In some embodiments, the nucleic acid sequence encoding a fusion protein comprising an N⁶-methyladenosine (m⁶A) binding domain of a YT521-B homology (YTH) domain-containing protein fused to a catalytic domain of a cytidine deaminase or a catalytic domain of an adenosine deaminase is operably linked to an inducible promoter, e.g., a tetracycline inducible promoter; and the nucleic acid construct encoding the mRNA reporter sequence is operably linked to a constitutive promoter (e.g., a CMV promoter)”

A “constitutive” promoter is a promoter that is active under most environmental and developmental conditions. Examples of constitutive promoters include, but are not limited to, a CMV promoter, a U6 promoter, a PGK promoter, a EF-1α promoter and a SV40 promoter.

An “inducible” promoter is a promoter that is active under environmental or developmental regulation, for example, regulated by the presence or absence of a drug. Examples of inducible promoters include, but are not limited to, the pL promoter (induced by an increase in temperature), the pBAD promoter, (induced by the addition of arabinose to the growth medium). the tetracycline-controlled transcriptional activation system (Tet-On/Tet-Off, Bujard and Gossen, PNAS, 89(12):5547-5551 (1992)), the Lac switch inducible system (Wyborski et al., Environ Mol Mutagen, 28(4):447-58 (1996)), the ecdysone-inducible gene expression system (No et al., PNAS, 93(8):3346-3351 (1996)), the cumate gene-switch system (Mullick et al., BMC Biotechnology, 6:43 (2006)), and the tamoxifen-inducible gene expression (Zhang et al., Nucleic Acids Research, 24:543-548 (1996)). Furthermore, a Cre-loxP inducible system can also be used, as well as a Flp recombinase inducible promoter system, both of which are known in the art.

In some embodiments, the promoter is a cell-specific or tissue-specific promoter. When using a cell- or tissue-specific promoter, expression occurs primarily, but not exclusively, in a particular cell or tissue. For example, expression can occur in at least 90%, 95%, or 99% of the targeted cell or tissue. It will be understood, however, that tissue-specific promoters may have a detectable amount of background or base activity in those tissues where they are mostly silent.

Examples of tissue-specific promoters include, but are not limited to, liver-specific promoters (e.g., APOA2, SERPINA1, CYP3A4, MIR122), pancreatic-specific promoters (e.g., insulin, insulin receptor substrate 2, pancreatic and duodenal homeobox 1, Aristaless-like homeobox 3, and pancreatic polypeptide), cardiac-specific promoters (e.g., myosin, heavy chain 6, myosin, light chain 2, troponin I type 3, natriuretic peptide precursor A, solute carrier family 8), central nervous system promoters (e.g., glial fibrillary acidic protein, internexin neuronal intermediate filament protein, Nestin, myelin-associated oligodendrocyte basic protein, myelin basic protein, tyrosin hydroxylase, and Forkhead box A2), skin-specific promoters (e.g., Filaggrin, Keratin 14 and transglutaminase 3), pluripotent and embryonic germ layer promoters (e.g., POU class 5 homeobox 1, Nanog homeobox, Nestin, and MicroRNA 122).

The cassette may additionally contain at least one additional gene or genetic element to be co-transformed into the organism (i.e., a cell, plurality of cells, tissue, or animal). Where additional genes or elements are included, the components are operably linked. Alternatively, the additional gene(s) or element(s) can be provided on multiple expression cassettes. Such an expression cassette is provided with a plurality of restriction sites and/or recombination sites for insertion of the polynucleotides to be under the transcriptional regulation of the regulatory regions. The expression cassette may additionally contain a selectable marker gene. The expression cassette will include in the 5′ to 3′ direction of transcription: a transcriptional and translational initiation region (i.e., a promoter), a polynucleotide of the invention, and a transcriptional and translational termination region (i.e., termination region) functional in the cell or organism of interest. The promoters of the invention are capable of directing or driving expression of a coding sequence in a host cell. The regulatory regions (i.e., promoters, transcriptional regulatory regions, and translational termination regions) may be endogenous or heterologous to the host cell or to each other. As used herein the term “heterologous” refers to a nucleotide sequence or polypeptide not normally found in a given cell in nature. As such, a heterologous nucleotide sequence or heterologous polypeptide may be: (a) foreign to its host cell (i.e., is exogenous to the cell); (b) naturally found in the host cell (i.e., endogenous) but present at an unnatural quantity in the cell (i.e., greater or lesser quantity than naturally found in the host cell); or (c) be naturally found in the host cell but positioned outside of its natural locus.

Additional regulatory signals include, but are not limited to, transcriptional initiation start sites, operators, activators, enhancers, other regulatory elements, ribosomal binding sites, an initiation codon, termination signals, and the like. See Sambrook et al. (1992) Molecular Cloning: A Laboratory Manual, ed. Maniatis et al. (Cold Spring Harbor Laboratory Press, Cold Spring Harbor, N.Y.) (hereinafter “Sambrook 11”); Davis et al., eds. (1980) Advanced Bacterial Genetics (Cold Spring Harbor Laboratory Press), Cold Spring Harbor, N.Y., and the references cited therein.

In preparing the expression cassette, the various DNA fragments may be manipulated, to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Further provided is a vector comprising a nucleic acid or expression cassette set forth herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene (See generally, Sambrook et al. Molecular Cloning: A Laboratory Manual, 4^{th} ed., Cold Spring Harbor Laboratory Press, Cold Spring Harbor, 2012). The vector, for example, can be a plasmid.

In some embodiments, a vector comprises the first DNA construct. In some embodiments, a vector comprises the second DNA construct. In some embodiments, a vector comprises the first and second DNA construct. In some embodiments, the vector is a plasmid. In some embodiments, a vector comprises the first DNA construct, the second DNA construct and a nucleic acid encoding a selectable marker. In some embodiments, the first DNA construct and the second DNA construct are operably linked to a first promoter, and the nucleic acid sequence encoding a selectable marker is operably linked to a second promoter (i.e., a promoter that is different from the first promoter). In some embodiments, the selectable marker is a fluorescent protein, that is different from the effector protein or the fluorescent protein encoded by second DNA construct, for example, dsRed. An exemplary dual-promoter construct that can be modified to express effector proteins as described herein, for example, but exchanging the nucleic acid sequence encoding a fluorescent report for an effector protein comprises: (1) a nucleic acid sequence encoding an effector protein, a m⁶A reporter sequence and DHFR; (2) a nucleic acid sequence encoding a fusion protein (APOBEC1-YTH); and (3) a nucleic acid sequence encoding dsRed (provided herein as SEQ ID NO: 107). In certain embodiments, the first DNA construct and second DNA construct do not contain nucleic acid sequences encoding a fluorescent protein.

There are numerous E. coli expression vectors known to one of ordinary skill in the art, which are useful for the expression of any of the nucleic acid sequences described herein (e.g., any of the fusion proteins described herein). Other microbial hosts suitable for use include bacilli, such as Bacillus subtilis, and other enterobacteriaceae, such as Salmonella, Senatia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. Additionally, yeast expression can be used.

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

As used throughout, a “fusion protein” is a protein comprising two different polypeptide sequences, i.e. a binding domain and a catalytic domain, that are joined or linked to form a single polypeptide. The two amino acid sequences are encoded by separate nucleic acid sequences that have been joined so that they are transcribed and translated to produce a single polypeptide. In some embodiments, the fusion protein comprises, in the following order, a m⁶A binding domain, and a catalytic domain of a cytidine deaminase or an adenosine deaminase.

As used throughout, “m⁶A” refers to posttranscriptional methylation of an adenosine residue in the RNA of prokaryotes and eukaryotes (e.g., mammals, insects, plants and yeast).

As used throughout an “m⁶A sensor sequence” is a sequence comprising one or more m⁶A methylation consensus motifs (GAC). The m⁶A sensor sequence can also comprise at least one sequence that can be converted to a stop codon when the m⁶A sensor sequence is methylated in the cell. In the constructs described herein, the m⁶A sensor sequence is in-frame with the nucleic acid encoding the heterologous protein, e.g. a reporter protein. The m⁶A sensor sequence is flanked by the nucleic acid sequence encoding the heterologous protein (e.g., reporter protein) and the nucleic acid sequence encoding a destabilization domain, e.g., DHFR. When the construct is methylated in the cell, a C to U modification generates a stop codon in the m⁶A sensor sequence. The stop codon prevents expression of the destabilization domain, thus preventing degradation of the heterologous protein. Exemplary m6A sensor sequences include, but are not limited to, a nucleic acid sequence comprising, consisting of, or consisting essentially of, SEQ ID NOs: 66 and 108-116. Nucleic acid sequences having at least 90, 91%, 92%, 93%, 94%, 95%, 96%, 97%, 98%, 99%, or 100% identity with a nucleic acid sequence comprising, consisting essentially of, or consisting of SEQ ID NOs: 66 and 108-116 are also provided. One of skill in the art would understand that these sequences are merely exemplary because any m⁶A sensor sequence comprising at least one m⁶A methylation consensus motif (GAC) (e.g., one, two, three, four etc.) can be used as a sensor sequence.

As used throughout, a m⁶A binding domain of a YT521-B homology (YTH) domain-containing protein is a polypeptide fragment of a YTH domain-containing protein that binds to m⁶A-containing sequence (e.g., a RNA, such as a mRNA or a m⁶A sensor sequence). The m⁶A binding domain derived from a YT521-B homology (YTH) domain-containing protein can be of any size as long as it retains binding activity and is not the full-length YTH domain-containing protein. In some embodiments, the binding domain retains at least about 75%, 80%, 90%, 95%, or 99% of the binding activity of the wildtype YTH domain-containing protein from which the binding domain is derived.

In some embodiments, the DNA construct encodes a m⁶A binding domain comprising a polypeptide having at least 95% identity, for example, at least about 95%, 96%, 97%, 98% or 99% identity, to SEQ ID NO: 67 (amino acid sequence of YTHDF2-YTH, a m⁶A binding domain of YTHDF2), SEQ ID NO: 68 (amino acid sequence of YTHDF2-YTH_W432A_W486A, a mutated m⁶A binding domain of YTHDF2), SEQ ID NO: 69 (amino acid sequence of YTHDF2-YTHmut, an amino acid sequence that includes the YTH domain of YTHDF2, and does not include the m6A-binding domain), SEQ ID NO: 70 (amino acid sequence of YTHDF2-YTHmut, an amino acid sequence comprising SEQ ID NO: 69, with a W432A mutation and a W486a mutation), SEQ ID NO: 71 (amino acid sequence of YTHDF2-YTH D422N, a mutated m⁶A binding domain of YTHDF2), SEQ ID NO: 72 (amino acid sequence of a m⁶A binding domain of YTHDF1), SEQ ID NO: 73 (amino acid sequence of YTHDF1mut, an amino acid sequence that includes the YTH domain of YTHDF2, and does not include the m⁶A-binding domain), SEQ ID NO: 74 (amino acid sequence of YTHDF1 D401N, a mutated m⁶A binding domain of YTHDF1), SEQ ID NO: 75 (amino acid sequence of a m⁶A binding domain of YTHDF3); SEQ ID NO: 76 (amino acid sequence of a m⁶A binding domain of YTHDC1) or SEQ ID NO: 77 (amino acid sequence of a m⁶A binding domain of YTHDC2).

As used throughout, a catalytic domain of a cytidine deaminase is a polypeptide comprising a cytidine deaminase, for example, Apolipoprotein B MRNA Editing Enzyme Catalytic Subunit (APOBEC1 or APO1), activation induced cytidine deaminase (AICDA) or Apolipoprotein B mRNA editing enzyme, catalytic polypeptide-like 3A (APOBEC3A), or a catalytic fragment thereof, that catalyzes deamination of cytidine (“C”) to uridine (“U”) in RNA molecules. As used throughout, a catalytic domain of an adenosine deaminase, is a polypeptide comprising an adenosine deaminase, for example, double-stranded RNA-specific adenosine deaminase (ADAR1), or a catalytic fragment thereof, that catalyzes deamination of adenosine (“A”) to inosine (“I”) in RNA molecules. In some embodiments, the catalytic domain retains at least about 75%, 80%, 90%, 95%, or 99% of the enzymatic activity of the wildtype deaminase from which the domain is derived.

In some embodiments, the catalytic domain comprises a polypeptide having at least 95% identity, for example, at least about 95%, 96%, 97%, 98% or 99% identity, to SEQ ID NO: 78 (amino acid sequence of rAPOBEC1) or its catalytic domain (SEQ ID NO: 120), SEQ ID NO: 13 (amino acid sequence of hAICDA) or its catalytic domain (SEQ ID NO: 79); SEQ ID NO: 80 (amino acid sequence of hAPOBEC3A) or its catalytic domain (SEQ ID NO: 128); SEQ ID NO: 81 (amino acid sequence of ADAR2) or its catalytic domain (SEQ ID NO: 119); or SEQ ID NO: 121 (amino acid sequence of ADAR1) or its catalytic domain (SEQ ID NO: 122).

The catalytic domain can also comprise a polypeptide having at least 95% identity to SEQ ID NO: 119 (amino acid sequence of catalytic domain of ADAR2), as set forth in U.S. Patent Application Publication No. 20190010478.

In some embodiments, the DNA construct encodes a m⁶A binding domain fused to the catalytic domain via a peptide linker. The peptide linker can be about 2 to about 150 amino acids in length. For example, the linker can be a linker of from about 5 to about 20 amino acids in length, from about 5 to about 25 amino acids in length, from about 10 to about 30 amino acids in length, 5 to about 35 amino acids in length, from about 5 to about 40 amino acids in length, from about 5 to about 45 amino acids in length, from about 5 to about 50 amino acids in length, from about 5 to about 55 amino acids in length, from about 5 to about 60 amino acids in length, from about 5 to about 65 amino acids in length, from about 5 to about 70 amino acids in length, from about 5 to about 75 amino acids in length, from about 5 to about 80 amino acids in length, from about 5 to about 85 amino acids in length, from about 5 to about 90 amino acids in length, from about 5 to about 95 amino acids in length, from about 5 to about 100 amino acids in length, from about 5 to about 105 amino acids in length, from about 5 to about 110 amino acids in length, from about 5 to about 115 amino acids in length, from about 5 to about 120 amino acids in length, from about 5 to about 125 amino acids in length, from about 5 to about 130 amino acids in length, from about 5 to about 135 amino acids in length, from about 5 to about 140 amino acids in length, from about 5 to about 145 amino acids in length, or from about 5 to about 150 amino acids in length.

Exemplary peptide linkers include, but are not limited to, peptide linkers comprising SEQ ID NO: 82 (SGSETPGTSESATPE), SEQ ID NO: 83 (SGSETPGTSESATPES), SEQ ID NO: 84 ((GGGGS)₃), SEQ ID NO: 85 ((GGGGS)₁₀), SEQ ID NO: 117 ((GGGGS)₂₀), SEQ ID NO: 86 (A(EAAAK)₃A), SEQ ID NO: 123 (A(EAAAK)₁₀A), or SEQ ID NO: 124 (A(EAAAK)₂MA).

In some embodiments, the fusion protein further comprises a localization element. In some embodiments, the localization element is fused to the N-terminus or the C-terminus of the fusion protein. As used herein, a localization element targets or localizes the fusion protein to one or more subcellular compartments. Subcellular compartments include but are not limited to, the nucleus, the endoplasmic reticulum, the mitochondria, chromatin, the cellular membrane, and RNA granules (for example, P-bodies, stress granules and transport granules). In some embodiments, the fusion protein can be targeted to the nuclear lamina, nuclear speckles nuclear paraspeckles in the nucleus of a cell. In some embodiments, the protein can be targeted to the outer mitochondrial membrane or the inner mitochondrial membrane.

Exemplary localization elements include, but are not limited to, a peptide comprising a nuclear localization signal, for example, SEQ ID NO: 89 (PKKKRKV), a peptide comprising a nuclear export signal, for example, SEQ ID NO: 90 (LPPLERLTL), a peptide comprising an endoplasmic reticulum targeting sequence, for example, SEQ ID NO: 91 (MDPVVVLGLCLSCLLLLSLWKQSYGGG), or SEQ ID NO: 92 (METDTLLLWVLLLWVPGSTGD), a peptide comprising a Myc tag, for example, SEQ ID NO: 93 (EQKLISEEDL), a peptide comprising a V5 tag, for example, SEQ ID NO:94 (GKPIPNPLLGLDST) or SEQ ID NO: 95 (IPNPLLGLD), a peptide comprising a FLAG tag, for example, SEQ ID NO: 96 (DYKDDDDK), a peptide comprising a 3×FLAG tag, for example, SEQ ID NO: 97 (DYKDHDGDYKDHDIDYKDDDDK) and a peptide comprising a DHFR destabilization domain, for example, SEQ ID NO: 98 (ISLIAALAVDHVIGMETVMPWNLPADLAWFKRNTLNKPVIMGRHTWESIGRPLPGRKNI ILSSQPSTDDRVTWVKSVDEAIAACGDVPEIMVIGGGRVYEQFLPKAQKLYLTHIDAEVE GDTHFPDYEPDDWESVFSEFHDADAQNSHSYCFEILERR). HA tags and NLS tags can also be utilized as known in the art.

Exemplary targeting effector proteins, such as catalytically-inactive RNA-guided endonucleases are provided above in the definitions above (for example, dCas9 and dCas13).

Exemplary effector proteins being tumor suppression proteins include p53 and SOCS2. In some embodiments, p53 comprises a polypeptide (or a polynucleotide encoding a polypeptide) having at least 95% identity, for example, at least about 95%, 96%, 97%, 98% or 99% identity, to SEQ ID NO: 125. In some embodiments, human SOCS2 comprises a polypeptide (or a polynucleotide encoding a polypeptide) having at least 95% identity, for example, at least about 95%, 96%, 97%, 98% or 99% identity, to SEQ ID NO: 126. Other tumor suppression proteins may be utilized, for example, those that affect the cell cycle or other proteins that are upstream or downstream of the JAK/STAT signaling pathway.

### III. Polypeptides

Provided herein are polypeptides that relate to methyladenosine (m⁶A) sensors and systems for detecting m⁶A modifications, in addition to effector protein expression systems and systems for targeting sensing and/or effector expression. Polypeptides as described herein can comprise more than one coding sequence for a protein of interest that are translationally fused so as to create a fusion protein. Provided herein are polypeptides encoded by any of the polynucleotides as described herein.

Modifications to any of the polypeptides or proteins provided herein are made by known methods. By way of example, modifications are made by site specific mutagenesis of nucleotides in a nucleic acid encoding the polypeptide, thereby producing a DNA encoding the modification, and thereafter expressing the DNA in recombinant cell culture to produce the encoded polypeptide. Techniques for making substitution mutations at predetermined sites in DNA having a known sequence are well known. For example, M13 primer mutagenesis and PCR-based mutagenesis methods can be used to make one or more substitution mutations. Any of the nucleic acid sequences provided herein can be codon-optimized to alter, for example, maximize expression, in a host cell or organism.

The amino acids in the polypeptides described herein can be any of the 20 naturally occurring amino acids, D-stereoisomers of the naturally occurring amino acids, unnatural amino acids, and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-587 (2013); Xie et al. “Adding amino acids to the genetic repertoire,” 9(6): 548-54 (2005)); and all references cited therein. B and γ amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

Also contemplated are conservative amino acid substitutions. By way of example, conservative amino acid substitutions can be made in one or more of the amino acid residues, for example, in one or more lysine residues of any of the polypeptides provided herein. One of skill in the art would know that a conservative substitution is the replacement of one amino acid residue with another that is biologically and/or chemically similar. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

By way of example, when an arginine to serine is mentioned, also contemplated is a conservative substitution for the serine (e.g., threonine). Nonconservative substitutions, for example, substituting a lysine with an asparagine, are also contemplated.

### IV. Polynucleotides

Recombinant nucleic acids encoding any of the polypeptides described herein are also provided.

As used throughout, the term “nucleic acid” or “nucleotide” refers to deoxyribonucleic acids (DNA) or ribonucleic acids (RNA) and polymers thereof in either single- or double-stranded form. It is understood that when an RNA is described, its corresponding cDNA is also described, wherein uridine is represented as thymidine. Unless specifically limited, the term encompasses nucleic acids containing known analogues of natural nucleotides that have similar properties as the reference nucleic acid and are metabolized in a manner similar to naturally occurring nucleotides. A nucleic acid sequence (i.e., a polynucleotide) can comprise combinations of deoxyribonucleic acids and ribonucleic acids. Such deoxyribonucleic acids and ribonucleic acids include both naturally occurring molecules and synthetic analogues. The polynucleotides of the invention also encompass all forms of sequences including, but not limited to, single-stranded forms, double-stranded forms, hairpins, stem-and-loop structures, and the like.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues. See Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994).

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning, and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an Iectation (E) of 10, M=1, N=−2, and a comparison of both strands. For amino acid sequences, the BLASTP program uses as defaults a word size (W) of 3, Iexpectation (E) of 10, and the BLOSUM62 scoring matrix. See Henikoff & Henikoff, Proc. Natl. Acad. Sci. USA 89:10915 (1989).

The BLAST algorithm also performs a statistical analysis of the similarity between two sequences. See, e.g., Karlin & Altshcul, Proc. Nat'l. Acad. Sci. USA 90:5873-5787 (1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

A target-specific guide RNA (gRNA) can comprise a nucleotide sequence that is complementary to a polynucleotide or RNA target sequence as described herein (for example one encoding a GEMS as described herein), and thereby mediates binding of the Cas-gRNA complex by hybridization at the target site. A target-specific guide RNA (gRNA) can comprise a nucleotide sequence that is complementary to a polynucleotide or RNA target sequence as described herein (for example METLL3, or other methylation target or therapeutic target in the cell, for example, a regulator of the cell cycle or protein involved in the JAK/STAT signaling pathway), and thereby mediates binding of the Cas-gRNA complex by hybridization at the target site. In certain embodiments, the gRNA is 5-50 nucleotides, 10-30 nucleotides, 15-25 nucleotides, 18-22 nucleotides, or 19-21 nucleotides in length, or any length between the stated ranges, including, for example, 10, 11, 12, 13, 14, 15, 16, 17, 18, 19, 20, 21, 22, 23, 24, 25, 26, 27, 28, 29, 30, 31, 32, 33, 34, or 35 nucleotides in length.

### V. Constructs, Vectors, Host Cells, and Animal Models

### A. Constructs

Provided herein are DNA constructs comprising aspects of expression systems as described herein, for example, components as described in Section I and II above.

The recombinant nucleic acids provided herein can be included in expression cassettes for expression in a host cell or an organism of interest. The cassette will include 5′ and 3′ regulatory sequences operably linked to a recombinant nucleic acid provided herein that allows for expression of the modified polypeptide. The cassette may additionally contain at least one additional gene or genetic element to be co-transformed into the cell or organism. Where additional genes or elements are included, the components are operably linked. Alternatively, the additional gene(s) or element(s) can be provided on multiple expression cassettes. Such an expression cassette is provided with a plurality of restriction sites and/or recombination sites for insertion of the polynucleotides to be under the transcriptional regulation of the regulatory regions. The expression cassette may additionally contain a selectable marker gene. The expression cassette will include in the 5′ to 3′ direction of transcription: a transcriptional and translational initiation region (i.e., a promoter), a polynucleotide of the invention, and a transcriptional and translational termination region (i.e., termination region) functional in the cell or organism of interest. The promoters of the invention are capable of directing or driving expression of a coding sequence in a host cell. The regulatory regions (i.e., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention.

Additional regulatory signals include, but are not limited to, transcriptional initiation start sites, operators, activators, enhancers, other regulatory elements, ribosomal binding sites, an initiation codon, termination signals, and the like. See Sambrook et al. (1992) Molecular Cloning: A Laboratory Manual, ed. Maniatis et al. (Cold Spring Harbor Laboratory Press, Cold Spring Harbor, N.Y.); Davis et al., eds. (1980) Advanced Bacterial Genetics (Cold Spring Harbor Laboratory Press), Cold Spring Harbor, N.Y., and the references cited therein.

The expression cassette can also comprise a selectable marker gene for the selection of transformed cells. Marker genes include genes conferring antibiotic resistance, such as those conferring hygromycin resistance, ampicillin resistance, gentamicin resistance, neomycin resistance, to name a few. Additional selectable markers are known and any can be used.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be used.

### B. Vectors

Further provided is a vector comprising a nucleic acid or expression cassette set forth herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene. See generally, Sambrook et al. Molecular Cloning: A Laboratory Manual, 4^{th} ed., Cold Spring Harbor Laboratory Press, Cold Spring Harbor, 2012. The vector, for example, can be a plasmid.

There are numerous E. coli expression vectors known to one of ordinary skill in the art, which are useful for the expression of a nucleic acid. Other microbial hosts suitable for use include bacilli, such as Bacillus subtilis, and other enterobacteriaceae, such as Salmonella, Senatia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. Additionally, yeast expression can be used. Provided herein is a nucleic acid encoding a polypeptide of the present invention, wherein the nucleic acid can be expressed by a yeast cell. More specifically, the nucleic acid can be expressed by Pichia pastoris or S. cerevisiae.

Mammalian cells also permit the expression of proteins in an environment that favors important post-translational modifications such as folding and cysteine pairing, addition of complex carbohydrate structures, and secretion of active protein. Vectors useful for the expression of active proteins in mammalian cells are known in the art and can contain genes conferring hygromycin resistance, geneticin or G418 resistance, or other genes or phenotypes suitable for use as selectable markers, or methotrexate resistance for gene amplification. A number of suitable host cell lines capable of secreting intact human proteins have been developed in the art, and include CHO cells, HeLa cells, HEK-293 cells, HEK-293T cells, U2OS cells, or any other primary or transformed cell line. Other suitable host cell lines include COS-7 cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc.

The expression vectors described herein can also include the nucleic acids as described herein under the control of an inducible promoter such as the tetracycline inducible promoter or a glucocorticoid inducible promoter. The nucleic acids of the present invention can also be under the control of a tissue-specific promoter to promote expression of the nucleic acid in specific cells, tissues or organs. Any regulatable promoter, such as a metallothionein promoter, a heat-shock promoter, and other regulatable promoters, of which many examples are well known in the art are also contemplated. Furthermore, a Cre-loxP inducible system can also be used, as well as a Flp recombinase inducible promoter system, both of which are known in the art.

Insect cells also permit the expression of the polypeptides. Recombinant proteins produced in insect cells with baculovirus vectors undergo post-translational modifications similar to that of wild-type mammalian proteins.

Also provided herein is a vector comprising the polynucleotides as described herein. The vector may be a DNA vector or a RNA vector. In some embodiments, the vector is a non-viral vector (e.g., a plasmid or naked DNA) or a viral vector. In some embodiments, the vector is a viral vector. Examples of viral vectors include, but are not limited to, an adeno-associated virus (AAV) vector, a retroviral vector, a lentiviral vector, a herpes simplex viral vector, or an adenoviral vector. It is understood that any of the viral vectors described herein can be packaged into viral particles or virions for administration to the subject.

In some aspects, the disclosure provides a virus comprising the nucleic acid comprising a nucleotide sequence encoding a polypeptide as described herein or the viral vector as described herein. The virus may be a AAV, a lentivirus, or a retrovirus.

Non-viral vectors can also be used to deliver the polynucleotides described herein. Accordingly, in some embodiments, the vector is a non-viral vector. For example, non-viral systems, such as naked DNA formulated as a microparticle, may be used. In some embodiments, delivery may include using virus-like particles (VLPs), cationic liposomes, nanoparticles, cell-derived nanovesicles, direct nucleic acid injection, hydrodynamic injection, use of nucleic acid condensing peptides and non-peptides. In one approach, virus-like particles (VLP's) are used to deliver the polypeptide(s). The VLP comprises an engineered version of a viral vector, where nucleic acids are packaged into VLPs through alternative mechanisms (e.g., mRNA recruitment, protein fusions, protein-protein binding). See Itaka and Kataoka, 2009, “Recent development of nonviral gene delivery systems with virus-like structures and mechanisms,” Eur J Pharma and Biopharma 71:475-483; and Keeler et al., 2017, “Gene Therapy 2017: Progress and Future Directions” Clin. Transl. Sci. (2017) 10, 242-248, incorporated by reference.

### C. Host Cells and Animal Models

Aspects of this disclosure include host cells and transgenic animals comprising the nucleic acid sequences or constructs described herein as well as methods of making such cells and transgenic animals.

a. Host Cells

A host cell comprising a nucleic acid or a vector or an expression as described herein is provided. The host cell can be an in vitro, ex vivo, or in vivo host cell. Populations of any of the host cells described herein are also provided. A cell culture comprising one or more host cells described herein is also provided. Methods for the culture and production of many cells, including cells of bacterial (for example E. coli and other bacterial strains), animal (especially mammalian), and archebacterial origin are available in the art. See e.g., Sambrook, Ausubel, and Berger (all supra), as well as Freshney (1994) Culture of Animal Cells, a Manual of Basic Technique, 3rd Ed., Wiley-Liss, New York and the references cited therein; Doyle and Griffiths (1997) Mammalian Cell Culture: Essential Techniques John Wiley and Sons, NY; Humason (1979) Animal Tissue Techniques, 4th Ed. W.H. Freeman and Company; and Ricciardelli, et al., (1989) In vitro Cell Dev. Biol. 25:1016-1024.

The host cell can be a prokaryotic cell, including, for example, a bacterial cell. Alternatively, the cell can be a eukaryotic cell, for example, a mammalian cell. In some embodiments, the cell can be an HEK293T cell, a Chinese hamster ovary (CHO) cell, a COS-7 cell, a HELA cell, an avian cell, a myeloma cell, a Pichia cell, an insect cell or a plant cell. A number of other suitable host cell lines have been developed and include myeloma cell lines, fibroblast cell lines, and a variety of tumor cell lines such as melanoma cell lines. The vectors containing the nucleic acid segments of interest can be transferred or introduced into the host cell by well-known methods, which vary depending on the type of cellular host. Host cells can be derived from any of the animals models discussed in (b) below.

In some embodiments, the provided cells express the protein stably or transiently by introducing an expression system (or any component thereof) into the cell. Stable expression of the protein in a cell refers to integration of any of the nucleic acids, DNA constructs, or vectors described herein into the genome of the cell, thereby allowing the cell to express the protein. Transient expression refers to expression of the protein directly from any of the nucleic acids, DNA constructs, and/or vectors following introduction into the cell (i.e., the gene encoding the protein is not integrated into the genome of the cell).

As used herein, the phrase “introducing” in the context of introducing a nucleic acid into a cell refers to the translocation of the nucleic acid sequence from outside a cell to inside the cell. In some cases, introducing refers to translocation of the nucleic acid from outside the cell to inside the nucleus of the cell. Various methods of such translocation are contemplated, including but not limited to, electroporation, nanoparticle delivery, viral delivery, contact with nanowires or nanotubes, receptor mediated internalization, translocation via cell penetrating peptides, liposome mediated translocation, DEAE dextran, lipofectamine, calcium phosphate or any method now known or identified in the future for introduction of nucleic acids into prokaryotic or eukaryotic cellular hosts. A targeted nuclease system (e.g., an RNA-guided nuclease, a transcription activator-like effector nuclease (TALEN), a zinc finger nuclease (ZFN), or a megaTAL (MT) (Li et al. Signal Transduction and Targeted Therapy 5, Article No. 1 (2020)) can also be used to introduce a nucleic acid, for example, a nucleic acid encoding a fusion protein and/or mRNA transcript (e.g, mRNA reporter mRNA) described herein, into a host cell.

In some embodiments, the provided cells express the protein constitutively or inducibly. Constitutive expression refers to ongoing, continuous expression of a gene (i.e., of a protein), whereas inducible expression refers to gene (protein) expression that is responsive to a stimulus. Inducible expression is generally regulated via an inducible promoter, a description of which is included above.

The CRISPR/Cas9 system, an RNA-guided nuclease system that employs a Cas9 endonuclease, can be used to edit the genome of a host cell or organism. Other RNA-guided CAS effector proteins can be used as well, for example, Cas13. The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

Any of the components encoded by the nucleic acid constructs described herein, for example, fusion proteins or a m⁶A/effector protein fusion protein, can be purified or isolated from a host cell or population of host cells. For example, a recombinant nucleic acid encoding any of the fusion proteins described herein can be introduced into a host cell under conditions that allow expression of the fusion protein. In some embodiments, the recombinant nucleic acid is codon-optimized for expression. After expression in the host cell, the fusion protein can be isolated or purified. Similarly, any of the nucleic acids encoding a m⁶A reporter mRNA described herein can be introduced into a host cell under conditions that allow transcription of the m⁶A reporter mRNA. After expression in the host cell, the m⁶A reporter mRNA can be isolated or purified.

b. Animal Models

Also provided is a non-human transgenic animal comprising a mammalian host cell that comprises any of the nucleic acid sequences or constructs described herein. Methods for making transgenic animals, include, but are not limited to, oocyte pronuclear DNA microinjection, intracytoplasmic sperm injection, embryonic stem cell manipulation, somatic nuclear transfer, recombinase systems (for example, Cre-LoxP systems, Flp-FRT systems and others), zinc finger nucleases (ZNFs), transcriptional activator-like effector nucleases (TALENs) and clustered regularly interspaced short palindromic repeat/CRISPR-associated protein 9 (CRISPR/Cas9). See, for example, Volobueva et al. Braz. J. Med. Biol. Res. 52(5): e8108 (2019)).

The term “transgenic animal” as used herein means an animal into which a genetic modification has been introduced by a genetic engineering procedure and in particular an animal into which has been introduced an exogenous nucleic acid, and may loosely also encompass “knock in” animals. That is the animal comprises a nucleic acid sequence which is not normally present in the animal. Such animals can be created by a one-for-one substitution of DNA sequence information in a predetermined genetic locus or the insertion of sequence information not found within the locus.

A transgenic animal may be developed, for example, from embryonic cells into which the genetic modification (e.g. exogenous nucleic acid sequence) has been directly introduced or from the progeny of such cells. The exogenous nucleic acid is introduced artificially into the animal (e.g. into a founder animal). Animals that are produced by transfer of an exogenous nucleic acid through breeding of the animal comprising the nucleic acid (into whom the nucleic acid was artificially introduced), which are progeny animals, are also included. Representative examples of non-human mammals include, but are not limited to non-human primates, mice, rats, rabbits, pigs, goats, sheep, horses, zebrafish and cows. A cell or a population of cells from any of the non-human transgenic animals provided herein is also provided.

The exogenous nucleic acid may be integrated into the genome of the animal or it may be present in an non-integrated form, e.g. as an autonomously-replicating unit, for example, an artificial chromosome which does not integrate into the genome, but which is maintained and inherited substantially stably in the animal. In some embodiments, the exogenous nucleic acid is under the control of a cell-specific or tissue-specific promoter. For example, transgenic animals that express a fusion protein and a mRNA reporter sequence in specific cells or tissues can be produced by introducing one or more nucleic acids into fertilized eggs, embryonic stem cells or the germline of the animal, wherein the one or more nucleic acids are under the control of a specific promoter which allows expression of the nucleic acid fusion protein and mRNA reporter sequence in specific types of cells or tissues. As used herein, a protein or mRNA is expressed predominantly in a given tissue, cell type, cell lineage or cell, when 90% or greater of the observed expression occurs in the given tissue cell type, cell lineage or cell.

In some embodiments, the exogenous nucleic acid in the animal is under the control of a constitutive or an inducible promoter, as described above. Inducible systems can also be used to allow expression of the fusion and/or mRNA reporter sequence at designated times during development, expanding the temporal specificity of fusion protein and/or mRNA reporter expression in the transgenic animal.

Included are both progenitor and progeny animals. Progeny animals include animals which are descended from the progenitor as a result of sexual reproduction or cloning and which have inherited genetic material from the progenitor. Thus, the progeny animals comprise the genetic modification introduced into the parent. A transgenic animal may be developed, for example, from embryonic cells into which the genetic modification (e.g. exogenous nucleic acid sequence) has been directly introduced or from the progeny of such cells. The exogenous nucleic acid is introduced artificially into the animal (e.g. into a founder animal). Animals that are produced by transfer of an exogenous nucleic acid through breeding of the animal comprising the nucleic acid (into whom the nucleic acid was artificially introduced), which are progeny animals, are also included.

Although the present disclosure is described primarily in a mouse, one of ordinary skill in the art would understand that other non-human mammals, for example, rodent, rabbit, bovine, ovine, canine, feline, equine, porcine, camelid, non-human primate, and other mammals, can also be engineered to express aspects of the present disclosure in a similar fashion, and these transgenic animals can also be used for applications as disclosed herein. A cell or a population of cells from any of the non-human transgenic animals provided herein is also provided.

### A. Pharmaceutical Compositions

Also provided herein are pharmaceutical compositions of the nucleic acids, the vectors, the viruses, or the cells described herein. The pharmaceutical compositions described herein are for delivery to subjects in need thereof by any suitable route or a combination of different routes. The pharmaceutical compositions can be delivered to a subject, so as to allow expression of the polypeptide in cells of the subject and produce an effective amount of the polypeptide that treats a condition in the subject. In some embodiments, the pharmaceutical composition comprising the nucleic acid, the vector, the virus, or the cell as described herein further comprises a pharmaceutically acceptable excipient or carrier.

The terms “pharmaceutically acceptable carrier” and “pharmaceutically acceptable excipient” are used interchangeably and refer to a substance or compound that aids or facilitates preparation, storage, administration, delivery, effectiveness, absorption by a subject, or any other feature of the composition for its intended use or purpose. Such pharmaceutically acceptable carrier is not biologically or otherwise undesirable and can be included in the compositions of the present invention without causing a significant adverse toxicological effect on the subject or interacting in a deleterious manner with the other components of the pharmaceutical composition.

In some approaches, sterile injectable solutions can be prepared with the vectors in the required amount and an excipient suitable for injection into a human patient. In some embodiments, the pharmaceutically and/or physiologically acceptable excipient is particularly suitable for administration to the cardiac muscle. For example, a suitable carrier may be buffered saline or other buffers, e.g., HEPES, to maintain pH at appropriate physiological levels, stabilizing agents, adjuvants, diluents, or surfactants. In some embodiments, the pharmaceutically acceptable excipient comprises a non-ionic detergent, such as, for example, Pluronic F-681. For injection, the excipient will typically be a liquid. Exemplary pharmaceutically acceptable excipients include sterile, pyrogen-free water and sterile, pyrogen-free, phosphate buffered saline. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. The preparation of pharmaceutically acceptable carriers, excipients and formulations is described in, e.g., Remington: The Science and Practice of Pharmacy, 22nd edition, Loyd V. Allen et al, editors, Pharmaceutical Press (2012). See also Bennicelli et al., “Reversal of blindness in animal models of leber congenital amaurosis using optimized AAV2-mediated gene transfer,” Mol Ther. (2008); 16(3):458-65. A variety of known carriers are also provided in U.S. Pat. Nos. 7,629,322, and 6,764,845, incorporated herein by reference.

### VI. Methods

Provided herein are methods for inducing m⁶A methylation-dependent expression of a heterologous polypeptide (comprising an effector protein) in one or more cells, a biological samples (for example a group of cells or tissue biopsy from a mammalian subject), or a subject having or suspected of having a cancer as described herein (or otherwise derived from a cancer in the case of cells in vitro), comprising introducing or administering any of the expression systems described herein into one or more cells, the sample, or the subject. As set forth above, when any of the expression systems described herein is introduced into a cell, sample, or subject, if m⁶A methylation occurs in the cell, the effector protein expressed by the expression system, i.e., a mRNA comprising a heterologous protein, a m⁶A sensor sequence and a destabilization domain (e.g., DHFR), will be methylated (at the m⁶A sensor sequence). Upon methylation, C to U editing results in a stop codon in the m⁶A sensor sequence that inhibits expression of DHFR, thus allowing the heterologous protein to be expressed without degradation.

In embodiments, the cell can be an in vitro, ex vivo or in vivo cell. The cell may be a mammalian cell or a rodent cell.

Provided herein are also methods for administering or introducing any of the expression systems described herein to a cell, sample, or subject as described herein. The administering or introducing to one or more cells, a sample, or a subject, can be by mechanisms known in the art to introduce exogenous nucleic acids into cells, for example, lipofection, nucleofection, or electroporation. Alternatively, the skilled artisan would understand that aspects of expression systems as described herein can be cloned into viral expression vectors and packaged into an adenoviral (AAV) or lentiviral (LV) vector, and subsequently used to transduce the exogenous genetic material into the cell, sample, or subject.

Also provided is a virus (e.g., an AAV, a lentivirus, or a retrovirus) comprising any of the nucleic acids or vectors described in this disclosure.

Also provided is a cell comprising any of the nucleic acids, vectors, or viruses described in this disclosure.

Provided herein is also a pharmaceutical composition comprising any of the nucleic acids, vectors, viruses, or cells described herein, and a pharmaceutically acceptable excipient.

### A. Tumor Suppression

One aspect provided in this disclosure is a method of inhibiting a cancer cell, the method comprising introducing into the cancer cell the expression system as provided in this disclosure. In some embodiments, inhibiting the cancer cell by methods as described herein results in decreasing at least one of cell proliferation, cell migration, or metastasis.

In some embodiments of this method, the cancer cell can comprise m⁶A RNA hypermethylation. In some embodiments, the cancer cell comprises an acute myeloid leukemia (AML) cell, a glioblastoma (GBM) cell, a lung cancer cell, an endometrial cancer, a cervical cancer cell, an ovarian cancer cell, a breast cancer cell, a colorectal cancer (CRC) cell, a hepatocellular carcinoma (HCC) cell, a pancreatic cancer cell, a gastric cancer cell, a prostate cancer cell, or a renal cell carcinoma cell. In certain embodiments, the lung cancer cell is a non-small cell lung carcinoma cell. In certain embodiments, the cancer cell is a hepatocellular carcinoma cell.

In some embodiments, the second DNA construct comprises a polynucleotide encoding an effector protein, wherein the effector protein comprises a tumor suppressor protein. In some embodiments, expression of the tumor suppressor protein upregulates downstream signaling targets. The tumor suppressor protein may comprise at least one of the tumor suppressor proteins listed in Table 1. In certain embodiments, the tumor suppressor protein can comprise p53. In some embodiments, expression of p53 upregulates at least one of CDKN1A or GADD45A. In certain embodiments, the tumor suppressor protein comprises suppressor of cytokine signaling 2 (SOCS2).

The expression system may be introduced into the cancer cell by viral infection (in particular, adenoviral, lentiviral, or AAV infection).

In another aspect of this disclosure, provided herein is a method of treating a subject having a cancer characterized by m⁶A RNA hypermethylation, the method comprising introducing into a cancer cell in the subject the expression system as provided in this disclosure. In some embodiments, the method comprises inhibiting a cancer cell of the subject's cancer in the subject. In some embodiments, expression of the tumor suppressor protein results in decreasing at least one of cell proliferation, cell migration, or metastasis of the cancer.

In some embodiments, the cancer can comprise acute myeloid leukemia (AML), glioblastoma (GBM), lung cancer, endometrial cancer, cervical cancer, ovarian cancer, breast cancer, colorectal cancer (CRC), a hepatocellular carcinoma (HCC), pancreatic cancer, gastric cancer, prostate cancer, and/or renal cell carcinoma. In certain embodiments, the cancer comprises hepatocellular carcinoma.

In some embodiments, the expression system can be introduced into the subject by viral infection (adenoviral, lentiviral, AAV).

While SOCS2 and p53 are provided as examples, the skilled artisan would recognize that other tumor suppression proteins can be expressed depending on the type of cancer by cloning a coding sequence of any of the gene products from Table 1 into the expression system as the effector protein.

Effects of expression of tumor suppression proteins according to the present disclosure include: inhibition of mitogenic signaling pathways; inhibition of cell cycle progression; inhibition of “pro-growth” programs of metabolism and angiogenesis; inhibition of invasion and metastasis; stabilization of the genome; DNA repair factors; and induction of apoptosis.

Additional examples are provided in Table 1 below:

TABLE 1
Embodiments of Tumor Suppression Proteins and Cancer
Types According to the Present Disclosure:

| Gene | Type of Cancer |
|---|---|
| APC | Colon/rectum carcinoma |
| BRCA1 | Breast and ovarian carcinomas |
| BRCA2 | Breast carcinoma |
| CDH1 | Autosomal dominant familial gastric |
| | carcinoma |
| CDKN2A | Melanomas, Leukemias, and Carcinomas |
| DPC4 | Pancreatic carcinoma |
| INK4 | Melanoma, lung carcinoma, brain tumors, |
| | leukemias, lymphomas |
| MADR2 | Colon/rectum carcinoma |
| NF1 | Neurofibrosarcoma |
| NF2 | Meningioma |
| p53 (i.e., TP53) | Brain tumors; breast, colon/rectum, |
| | esophageal, liver, and lung carcinomas; |
| | sarcomas; leukemias and lymphomas |
| PARP-1 | Breast carcinoma |
| PTC | Basal cell carcinoma |
| PTEN | Brain tumors; melanoma; prostate, |
| | endometrial, kidney, and lung carcinomas |
| Rb | Retinoblastoma; sarcomas; bladder, breast, |
| | and lung carcinomas |
| SOCS2 | hepatocellular carcinoma |
| VHL | Renal cell carcinoma |
| WT1 | Wilms' tumor |

### B. m⁶A Regulator Targeting

Described herein are methods of reducing m⁶A effector regulator expression in a sample or a subject. In particular, CRISPRi can be utilized to knock-down expression of m⁶A “writers”, which are proteins that are responsible for m⁶A dysregulation (in particular hypermethylation) observed in cancer cells.

In embodiments, described herein is a method of reducing m⁶A effector regulator expression, comprising: introducing an expression system into a subject having or suspected of having a cancer, wherein the expression system comprises: a first DNA construct comprising a polynucleotide encoding a fusion protein, wherein the fusion protein comprises an N⁶-methyladenosine (m⁶A) binding domain of a YT521-B homology (YTH) domain-containing protein fused to a catalytic domain of a cytidine deaminase or a catalytic domain of an adenosine deaminase; and a second DNA construct comprising a polynucleotide encoding a heterologous polypeptide, the polynucleotide encoding a heterologous polypeptide comprising: a polynucleotide encoding a catalytically-dead RNA-guided endonuclease; a polynucleotide encoding a m⁶A sensor sequence; and a polynucleotide encoding a dihydrofolate reductase (DHFR); an sgRNA configured to bind to an m⁶A regulator. In embodiments, the sgRNA is configured to bind to a m⁶A regulator listed in Table 2. In embodiments, the cancer comprises at least one of acute myeloid leukemia (AML), glioblastoma (GBM), lung cancer, endometrial cancer, cervical cancer, ovarian cancer, breast cancer, colorectal cancer (CRC), a hepatocellular carcinoma (HCC), pancreatic cancer, gastric cancer, prostate cancer, or renal cell carcinoma. In embodiments, the cancer is a cancer listed in Table 1 or Table 2. In embodiments, the catalytically-dead RNA-guided endonuclease is a dCas9 or dCas13.

Described herein are methods of reducing m⁶A hypermethylation in a subject or sample. In embodiments, methods comprising: introducing an expression system into a subject having or suspected of having a cancer, wherein the expression system comprises: a first DNA construct comprising a polynucleotide encoding a fusion protein, wherein the fusion protein comprises an N⁶-methyladenosine (m⁶A) binding domain of a YT521-B homology (YTH) domain-containing protein fused to a catalytic domain of a cytidine deaminase or a catalytic domain of an adenosine deaminase; and a second DNA construct comprising a polynucleotide encoding a heterologous polypeptide, the polynucleotide encoding a heterologous polypeptide comprising: a polynucleotide encoding a catalytically-dead RNA-guided endonuclease; a polynucleotide encoding a m⁶A sensor sequence; and a polynucleotide encoding a dihydrofolate reductase (DHFR); an sgRNA configured to bind to an m⁶A regulator. The sgRNA is configured to bind to a m⁶A regulator listed in Table 2. The cancer comprises at least one of acute myeloid leukemia (AML), glioblastoma (GBM), lung cancer, endometrial cancer, cervical cancer, ovarian cancer, breast cancer, colorectal cancer (CRC), a hepatocellular carcinoma (HCC), pancreatic cancer, gastric cancer, prostate cancer, or renal cell carcinoma. The cancer is a cancer listed in Table 1 or Table 2. The catalytically-dead RNA-guided endonuclease is a dCas9 or dCas13.

A subject can be a subject having or suspected of having a cancer as described herein. Introducing to the subject can comprise viral infection or electroporation. Modulating m⁶A levels by affecting an m⁶A regulator can decreasing at least one of cell proliferation, cell migration, or metastasis.

While METTL3 is provided as an example, the skilled artisan would recognize that other tumor suppression proteins can be expressed depending on the type of cancer by cloning a coding sequence of any of the gene products in Table 2 below into the expression system as the effector protein in order to reduce hypermethylation (or the effects thereof). It would be recognized that m⁶A “washers” may be also be expressed as tumor suppression proteins, while m⁶A “writers” can be targeted by the catalytically-dead RNA-guided endonuclease to block protein expression of the writers.

Additional aspects of CRISPi generally, can be found, for example in Carroll & Giacca, CRISPR activation and interference as investigative tools in the cardiovascular system, Int. J. of Biochem. &Cell Bio., Volume 155, February 2023, 106348, the contents of which regarding CRISPRi are incorporated by reference as if fully set forth herein.

TABLE 2
Embodiments of M6A Regulators and Cancer Types According to the Present Disclosure:

| Pathway | m6A Regulator | Cancer | Function |
|---|---|---|---|
| C-myc pathway | METTL3 | Lung cancer | promote growth and migration |
| | METTL3 | Bladder cancer | promote cell proliferation, invasion and survival |
| | METTL3 | Oral squamous | promote growth, invasion, migration and progression |
| | | cell carcinoma, | |
| | | Colorectal | |
| | | cancer, Prostate | |
| | | carcinoma | |
| | METTL3 | Gastric cancer | promote proliferation and metastasis |
| | METTL3 | Acute myeloid | inhibit diferentiation and increase proliferation |
| | | leukemia | |
| | FTO | Colorectal cancer | inhibit apoptosis and improve cell proliferation, migration, and |
| | | | invasion |
| | IGF2BP2 | Thyroid cancer | promote proliferation, invasion, migration and anti-apoptosis |
| | YTHDF2 | Glioblastoma | support glioblastoma stem cells viability |
| PI3K/AKT/mTOR pathway | YTHDF1 | Gastric cancer | |
| | FTO | Endometrial | promote invasion and metastasis |
| | | cancer | |
| | YTHDF1 | Colorectal | promote tumorigenicity and cell cycle |
| | | carcinoma | |
| | METTL3 | Hepatocellular | accelerate development |
| | | carcinoma | |
| | | | Hepatocellular carcinoma |
| | METTL3 | Colorectal | promote the stemness and chemoresistance |
| | | carcinoma | |
| | METTL3 | Nasopharyngeal | promote cisplatin resistance |
| | | carcinoma (NPC) | |
| | METTL14 | Breast cancer | promote stemness and progression |
| p53 pathway | METTL3 | Colorectal | promote multidrug resistance |
| | YTHDF1 and | Melanoma | promote the development |
| | HNRNPA2B1 | | |
| | METTL14 | Pancreatic cancer | promote the growth and metastasis |
| | METTL3 | Breast cancer | promote the proliferation |
| EMT signaling pathway | METTL14 | Colorectal | mediate EMT process |
| | | carcinoma | |
| | METTL3 | Gastric cancer | accelerate the EMT |
| | METTL3 | Hepatocellular | promote the EMT |
| | | carcinoma | |
| | YTHDF3 | Hepatocellular | facilitate migration, invasion, and EMT |
| | | carcinoma | |
| | METTL3 | Lung cancer, | accelerate the EMT and promote the development |
| | | Ovarian cancer, | |
| | | Colorectal | |
| | | carcinoma | |
| MAPK signaling pathway. | METTL3 | Colorectal | promote metastasis |
| | | carcinoma | |
| P38/ERK | METTL3 | Colorectal | suppress proliferation, migration and invasion |
| | | carcinoma | |
| ERK1/2 and STAT3 | HNRNPA2B1 | Breast cancer | promote the tumorigenicity, and decrease apoptosis |
| pathways | | | |
| BCL-2 | METTL3 | Breast cancer | accelerate proliferation, decrease the apoptosis |

(Adapted from Pan J, Huang T, Deng Z, Zou C. Roles and therapeutic implications of m6A modification in cancer immunotherapy. Front Immunol. 2023 Mar. 7; 14:1132601. doi: 10.3389/fimmu.2023.1132601. PMID: 36960074; PMCID: PMC10028070). Additional m6a regulators can be found, for example, in Gu. et al. RNA m6A Modification in Cancers: Molecular Mechanisms and Potential Clinical Applications, Cell, The Innovation 1, 100066, Nov. 25, 2020, as well as Chen, X Y., Zhang, J. & Zhu, J S. The role of m6A RNA methylation in human cancer. Mol Cancer 18, 103 (2019). Doi: 10.1186/s12943-019-1033-z; Chang, G., et al., RNa m6A Modification in Cancers: Molecular Mechanisms and Potential Clinical Applications. The Innovation, Vol. 1, Issue 3, Article 100066, Nov. 25, 2020. doi: 10.1016/j.xinn.2020.100066; and Chen, X. Y., et al. The role of m6A RNA methylation in human cancer. Molecular Cancer, Vol. 18, Article 103 (2019). doi: 10.1186/s12943-019-1033-z, molecular-cancer.biomedcentral.com/articles/10.1186/s12943-019-1033-z the contents of all of which are incorporated by reference regarding m⁶A regulators and cancers and effectors of regulators the m⁶A regulators.

Also provided are methods of treating a disease or disorder in a subject in need thereof, wherein the method comprises administering any of the expressions systems described herein to the subject. In some methods, the subject has cancer. In some methods, the subject is diagnosed with a disease or disorder (e.g., cancer).

As used herein, the term “administering” “administration”, or “administer” means delivering the pharmaceutical composition as described herein to a target cell or a subject. Administration refers to the act of introducing, injecting or otherwise physically delivering a substance as it exists outside the body (e.g., one or more nucleic acids, vectors, viruses, cells, or pharmaceutical compositions described herein) into a subject. The compositions described herein can be delivered to subjects in need thereof by any suitable route or a combination of different routes. Any suitable route of administration or combination of different routes can be used, including systemic administration (e.g., intravenous, intravascular, or intra-arterial injection), local injection into the heart muscle, local injection into the CNS (e.g., intracranial injection, intracerebral injection, intracerebroventricular, or injection into the Cerebrospinal fluid (CSF) via the cerebral ventricular system, cisterna magna, or intrathecal space), or local injection at other bodily sites (e.g. intraocular, intramuscular, subcutaneous, intradermal, or transdermal injection). In some embodiments, the compositions described herein are administered into the coronary arteries. In some embodiments, the compositions described herein are administered into the coronary sinus.

As used herein the terms “treatment”, “treat”, or “treating” refers to a clinical intervention made in response to a disease, disorder or physiological condition manifested by a patient or to which a patient may be susceptible. The aim of treatment includes the reduction, alleviation, slowing, or stopping the progression or worsening of a disease, disorder, or condition including reducing or preventing one or more of the effects or symptoms of the disease, disorder, or condition and/or the remission of the disease, disorder or condition, for example, a cardiac condition, in the subject. Thus, in the disclosed methods, treatment can refer to a 10%, 20%, 30%, 40%, 50%, 60%, 70%, 80%, 90%, or 100% reduction in the severity of a cardiac condition. For example, a method for treating a cardiac condition is considered to be a treatment if there is a 10% reduction in one or more symptoms of a cardiac condition in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease or symptoms of the disease.

Administration can be performed by injection, by use of an osmotic pump, by electroporation, or by other means. In some approaches, administration of the compositions of the present disclosure can be performed before, after, or simultaneously with surgical treatment.

Dosage values may depend on the nature of the product and the severity of the condition. It is to be understood that for any particular subject, specific dosage regimens can be adjusted over time and in course of the treatment according to the individual need and the professional judgment of the person administering or supervising the administration of the compositions. Accordingly, dosage ranges set forth herein are exemplary only and are not intended to limit the scope or practice of the claimed composition.

A therapeutically effective amount of such a composition may vary according to factors such as the disease state, age, sex, weight of the individual, and whether it is used concomitantly with other therapeutic agents. Dosage regimens may be adjusted to provide the optimum response. A suitable dose can also depend on the particular viral vector used, or the ability of the viral vector to elicit a desired response in the individual. A therapeutically effective amount is also one in which any toxic or detrimental effects of the viral vector are outweighed by the therapeutically beneficial effects. Other factors determining a dose can include, e.g., other medical disorders concurrently or previously affecting the subject, the general health of the subject, the genetic disposition of the subject, diet, time of administration, and any other additional therapeutics that are administered to the subject. It should also be understood that a specific dosage and treatment regimen for any particular subject also depends upon the judgment of the treating medical practitioner.

The effective amount of the compositions described herein can be determined by one of ordinary skill in the art. One of skill in the art will appreciate that an effective amount of a composition, for example, comprising an AAV or a lentivirus, can be empirically determined. An effective amount of any of the compositions described herein will vary and can be determined by one of skill in the art through experimentation and/or clinical trials. For example, quantification of genome copies (GC), vector genomes (VG), virus particles (VP), or infectious viral titer may be used as a measure of the dose contained in a formulation or suspension. Any method known in the art can be used to determine the GC, VG, VP or infectious viral titer of the virus compositions of the invention, including as measured by qPCR, digital droplet PCR (ddPCR), UV spectrophotometry, ELISA, next-generation sequencing, or fluorimetry as described in, e.g., in Dobkin et al., “Accurate Quantification and Characterization of Adeno-Associated Viral Vectors.” Front Microbiol 10: 1570-1583 (2019); Lock et al., “Absolute determination of single-stranded and self-complementary adeno-associated viral vector genome titers by droplet digital PCR.” Hum Gene Ther Methods 25: 115-125 (2014); Sommer, et al., “Quantification of adeno-associated virus particles and empty capsids by optical density measurement.” Mol Ther 7: 122-128 (2003); Grimm, et al. “Titration of AAV-2 particles via a novel capsid ELISA: packaging of genomes can limit production of recombinant AAV-2.” Gene Ther 6: 1322-1330 (1999); Maynard et al., “Fast-Seq: A Simple Method for Rapid and Inexpensive Validation of Packaged Single-Stranded Adeno-Associated Viral Genomes in Academic Settings.” Hum Gene Ther 30(6): 195-205 (2019); Piedra, et al., “Development of a rapid, robust, and universal picogreen-based method to titer adeno-associated vectors.” Hum Gene Ther Methods 26: 35-42 (2015); which are incorporated herein by reference. For intravenous injection, an exemplary human dosage range in vector particles (vp) may be between 5×10e13-10×10e14 vp per kilogram bodyweight (vp/kg) in a volume of 1-100,000 μl. In one embodiment, an exemplary human dose for intramuscular (cardiac muscle injection) or intracoronary delivery may be 1×10e14-5×10e14 vp per injection into the heart in a volume of 1-1000 μl.

In one approach, the composition is administered in a single dosage selected from those above listed. In another embodiment, the method involves administering the compositions in two or more dosages (e.g., split dosages). In another embodiment, multiple injections are made at different locations. In another embodiment, a second administration of the composition is performed at a later time point. Such time point may be weeks, months or years following the first administration. In some embodiments, multiple treatments may be required in any given subject over a lifetime.

### EXAMPLES

As mentioned above, a targeted m⁶A-coupled effector protein delivery system can be used in cancer therapy. For example, METTL3 is elevated in many cancers, and hypermethylation of oncogenic mRNAs leads to increased translation and cancer progression (Vu et al. “The N(6)-methyladenosine (m(6)A)-forming enzyme METTL3 controls myeloid differentiation of normal hematopoietic and leukemia cells,” Nat Med. 2017; 23(11):1369-76). Current strategies for overcoming this have focused on developing drugs that inhibit METTL3. However, this approach can have unwanted effects since it can impact the methylation of all mRNAs. Thus, using the m⁶A sensor system to express a tumor suppressor protein or to deliver CRISPR systems targeting upregulated oncogenes offers a more targeted approach.

Additionally, the m⁶A sensor system can be used to develop an m⁶A-coupled effector protein expression system. To demonstrate the utility and versatility of this technology, m⁶A sensor systems can be engineered and utilized to deliver a tumor suppressor protein to counteract the effects of hypermethylation in cancer cells, and (in embodiments) to express METTL3-targeting CRISPRi tools to maintain cellular m⁶A levels through a METTL3 feedback mechanism or express other tumor suppression proteins (such as cycle proteins like p53 for example). The utility of the system, to influence physiological outcomes can also be studied.

### Example 1: Materials and Methods

Cell culture. All cell types used in this study were cultured at 37° C. and 5% CO2 using the recommended cell type-specific growth medium. HEK293T cells (ATCC, CRL-3216), HeLa cells (ATCC, CRM-CCL-2), and NIH/3T3 cells (ATCC, CRL-1658) were cultured in Dulbecco's Modified 430 Eagle's Medium (DMEM, Corning). A549 cells (ATCC, CCL-185) and CHO-K1 cells (ATCC, CCL61) were cultured in Ham's F-12K (Kaighn's) Medium (Gibco). Huh-7 cells (obtained through the Duke University Cell Culture Facility) were cultured in Dulbecco's Modified Eagle's Medium (DMEM, Corning) with the addition of 12.5 mL of 1M HEPES (Fisher Scientific). HepG2 cells (ATCC, HB-8065) were cultured in Gibco Minimum Essential Media (MEM, Gibco) with the addition of 1% Sodium Pyruvate (Fisher Scientific) and 1% NEAA (Fisher Scientific). METTL3 degron cells were cultured as for HEK293T cells. All cell lines were cultured with the addition of 10% fetal bovine serum (Avantor) and 10 units/mL Penicillin/10 μg/mL Streptomycin (Gibco) to their respective growth media. HEK293T cells were tested for mycoplasma infection by the Duke University Cell Culture Facility and were confirmed to be mycoplasma-free.

Plasmids and cloning. The sequence for the EGFP-DHFR reporter mRNA was synthesized using custom gene synthesis (IDT gblock). All RAC consensus motifs within the EGFP sequence were mutated to avoid m⁶A methylation and potential editing of the EGFP coding sequence. Synonymous/codon-445 optimized mutations were used when possible. The EGFP and DHFR coding sequences are separated by a linker region which contains the first 81 nt of the human ACTB 3′UTR with some modifications (Table 2). The “m⁶A sensor sequence” consists of 5′-GCGGACUUACGACAG-3′ and contains the m⁶A sites at positions 1216 and 1222 of ACTB, with mutations of some nearby residues to enable C-to-U editing sites that produce in-frame stop codons (Table 3). The DHFR sequence contains the E. coli DHFR gene as previously described²⁵. This EGFP-DHFR gblock sequence was cloned into the pCMV-APOBEC1-YTH plasmid²⁰ at Not1/XhoI sites. The resulting plasmid, pCMV-EGFP-DHFR, was used for experiments involving expression of EGFP-DHFR alone or co-transfected with APOBEC1-YTH or APOBEC1-YTHmut. For all other experiments, the pGEMS plasmid was used, which contains CMV-EGFP-DHFR and EF1a-APOBEC1-YTH. pGEMSmut is the same plasmid but contains EF1a-APOBEC1-YTHmut. To generate pGEMS and pGEMSmut, we first generated iDuet101A-APOBEC1-YTH and iDuet101A-APOBEC1-YTHmut by cloning APOBEC1-YTH/YTHmut from pCMV-APOBEC1-YTH/YTHmut into iDuet101A (a gift from Linzhao Cheng, Addgene plasmid #17629) using XbaI/ClaI sites. EGFP-DHFR was then cloned into iDuet101A-APOBEC1-YTH and -YTHmut at NruI and SanDI sites to generate pGEMS and pGEMSmut, both of which contain the EGFP-DHFR sequence under control of the CMV promoter and APOBEC1-YTH or APOBEC1-YTHmut under control of the EF1a promoter. The puromycin-P2A-rtTA from TLCV2 (a gift from Adam Karpf, Addgene #87360) was also inserted into pGEMS and pGEMSmut using Gibson assembly.

The hPGK-DsRed-Express2 construct was cloned out of LVDP-CArG-RE-GPR (a gift from Stelios Andreadis, Addgene plasmid #89762) and subcloned into pGEMS and pGEMSmut by Gibson assembly to generate pGEMS-II and pGEMSmut-II. For experiments using GEMS-EGFP-PEST, the PEST destabilization domain was subcloned from the pCAG-GFP-PEST plasmid (a gift from Debra Silver) and inserted at the c-terminus of EGFP to produce pGEMS-II-PEST. To generate pGEMS-SOCS2 and pGEMS-p53, human SOCS2 and p53 CDSs were amplified from a cDNA library prepared from HeLa cells and subcloned into pGEMS-II in place of EGFP.

The GEMS-dCas13 system (dCas13-NLS-APO1-YTH) was adapted from pCMV-dCas13-M3nls (a gift from David Liu, plasmid #155366), by subcloning dCas13-NLS upstream of APO1-YTH in the pGEMS-II-PEST plasmid. dCas13 gRNA sequences (listed in Table 3) were subcloned into the pC016 plasmid (a gift from Feng Zhang, plasmid #91906).

Plasmid Transfection. Transfections were performed using Fugene HD (Promega) according to the manufacturer's instructions. For METTL3 inhibition experiments, cells were treated with 10 μM or 30 μM of STM2457 (MedChemExpress) for 16 hours prior to transfection. Cells were treated with 0.1% DMSO (VWR Life Science) as a control. For experiments using METTL3 degron cells, 0.1 mg/mL of auxin or equivalent volume of H2O (control) was added to the cells for 24 hours prior to plasmid transfection.

Microscopy. All images were obtained using a Leica DMi8 inverted fluorescence microscope. Images were processed using the Leica LAS X software. 4-5 fields of view were obtained per sample, and representative images were selected for each experiment.

Quantitative Microscopy. HEK293T cells were plated in 10 cm cell culture-treated dishes at a density of 2.2×10⁶ cells per plate and allowed to grow overnight. Cells were then treated with 30 μM of STM2457 or DMSO control for 16 hours. Cells were then transfected with the GEMS-PEST-DsRed plasmid and allowed to grow for an additional 16 hours. Prior to imaging, media was replaced with 1×PBS with 1 μg/mL Hoechst nuclear fluorescent stain (ThermoFisher Scientific) for 30 minutes. Cells were imaged using a Leica DMi8 fluorescence microscope. Images were analyzed by ImageJ image analysis software. First, fluorescence channels were separated and RGB fluorescence channels were converted to grayscale (16-bit). Binary image thresholds were set in relation to DsRed fluorescence signal and background noise was subtracted. Individual cells were selected, and pixel intensities were generated of each individual cell within the field of view. Similar binary image thresholding, cell selection, and intensity datasets were generated for the EGFP image channel. Data were analyzed by dividing EGFP signal intensity for each cell by average DsRed signal intensity. Data were plotted in JMP software (IMP 17.0, SAS Institute Inc.) by normalized EGFP signal intensity for each treatment. Boxplots represent interquartile range of the data and whiskers represent minimum and maximum data points excluding outliers. Statistical significance was calculated using a two-way t-test assuming unequal variance.

Western blotting. Cells were dissociated in culture plates using TrypLE (Gibco) and collected by centrifugation (6,000 rpm, 10 minutes, 4° C.). Cell pellets were resuspended in 150 μL chilled standard RIPA buffer (1% Triton X-100, 0.1% Sodium Deoxycholate, 0.1% SDS) prepared with the addition of Complete Mini protease inhibitor (Sigma Aldrich) immediately before use. Cells were resuspended and incubated on ice in RIPA buffer for 30 minutes. Cell lysates were cleared by centrifugation (13,000 rpm, 10 minutes, 4° C.) and mixed in a 1:1 ratio with NuPage LDS sample buffer (Invitrogen) with 5% Beta-Mercaptoethanol (Sigma). Samples were separated by gel electrophoresis on NuPage 4-12% Bis-Tris SDS-PAGE gels (Invitrogen), then transferred onto PVDF membranes (Amersham) using semi-dry transfer (Trans-Blot Turbo, Biorad). Membranes were blocked with 4% milk powder in 0.1% PBST and incubated with the appropriate primary antibody overnight at 4° C. Membranes were then washed with 0.1% PBST and incubated for 1 hour at room temperature in secondary antibody. Blots were washed and incubated in chemiluminescent ECL reagent solution (Amersham) then imaged in a ChemiDoc MP imaging system (BioRad) under chemiluminescent and colorimetric light. In western blot images, cyclophilin A is shown as a loading control and all western blots are representative of a minimum of 3 independent biological replicates. The following antibodies were used in this study: GFP tag Polyclonal antibody (Proteintech, 50430-2-AP). Anti-HA rabbit monoclonal antibody (Cell Signaling, 3724). Anti-Cyclophilin A antibody (Cell Signaling, 2175S). Anti-DsRed-Express2 Monoclonal Antibody (Fisher Scientific, CF180014). Anti-METTL3 antibody (abcam, ab195352). Anti-SOCS2 antibody (abcam, ab109245). Anti-p53 (7F5) Rabbit monoclonal antibody (Cell Signaling, 2527S). Recombinant Anti-JAK2 Antibody (abcam, ab108596). Recombinant Anti-STAT5 (phosphorylated Y694) (abcam, ab32364). Anti-STAT5 antibody (Cell Signaling, 25656S). Secondary antibodies used in this study: Goat Anti-Rabbit IgG HRP (Abcam, ab6721), Goat anti-mouse IgG HRP (Fisher Scientific, 62-6520).

For densitometry analysis, western blot images were quantified using ImageJ software 58. EGFP band intensity was normalized to either EGFP-DHFR or (EGFP+EGFP-DHFR) as indicated in each experiment. Similar densitometry analysis was used to measure total protein production (EGFP+EGFP-DHFR), normalized to the Cyclophilin A loading control. At least two replicates were used for each western blot quantification analysis. Bars are plotted as mean intensity ratio and error bars represent standard deviation.

Sanger sequencing and RT-qPCR. Cells were dissociated in culture using TrypLE (Gibco) treatment for 2 minutes and collected by centrifugation (6,000 rpm, 10 minutes, 4° C.). RNA was extracted using the RNA easy Plus Mini kit (Qiagen) according to the manufacturer's protocol. Extracted RNA was purified for genomic and plasmid DNA contamination by incubation with 1 μL DNase I at 37° C. for 30 minutes, and purified RNA was precipitated in 2.5 volumes of isopropanol overnight. RNA quantification was performed using the Qubit 4.0 fluorometer following the Qubit RNA broad range assay kit (Invitrogen). 500 ng of RNA was used for reverse transcription with the iScript reverse transcription supermix (Biorad) following the manufacturer's protocol. PCR was then used to 550 amplify the region of interest in the m⁶A sensor mRNA sequence (see Table 2). PCR products were column purified before Sanger sequencing using QiaQuick spin columns (Qiagen) and 10 μL reactions were submitted for standard amplicon sequencing (Azenta Life Sciences). C-to-U editing percentage was calculated using the EditR web server 59. To measure gene expression using gene-specific oligos, qPCR was performed using iTaq universal SYBR green Supermix (Biorad). 20 μL reactions were set up with 1 μL of cDNA for each sample, in 3 technical replicates. qPCR was performed on a Biorad CFX Duet real-time PCR instrument, and results were analyzed by normalizing threshold cycle of each target gene to 18S rRNA according to established methods⁶⁰. At least 2 biological replicates were used for each sample, and results are plotted as mean relative fold expression comparing treatment to the control group as indicated. Error bars represent standard deviation, and statistical significance was calculated using a two-way t-test assuming unequal variance.

m⁶A detection using RT-qPCR. RNA was extracted and treated with DNase I as described above, and a relative quantification of m⁶A by RT-qPCR was adapted from²⁸. 4 reverse transcription reactions were set up using 150 ng of RNA for each: 2 reactions using BstI polymerase (NEB), and 2 reactions using Superscript reverse transcriptase enzyme (Fisher Scientific). For each reverse transcriptase, one reaction included a primer adjacent (+) to the site being tested (reverse compliment reverse oligo immediately downstream of the site), and the other reaction included a primer that is non-adjacent (−) to the site. The BstI reaction consisted of 10 U BstI polymerase, 50 mM dNTPs, 500 nM oligos (adjacent (+) or non-adjacent (−)), in 1× ThermoPol Buffer. Reactions were incubated in a thermal cycler with the following cycling settings: 3 minutes at 25° C., 30 minutes at 50° C., and 3 minutes at 85° C. Superscript III (SSIII) reactions consisted of 200 U Superscript III, 1M DTT, 25 mM MgCl2+, 10 mM dNTPs, 500 nM oligos (adjacent or non-adjacent), 2 μL 10× FS Buffer, and water up to 20 μL. SSIII thermal cycler settings were set according to manufacturer's protocol. All 4 reactions were used as a template in a qPCR reaction, in 3 technical replicates, using primers that flank the m⁶A site being tested. Threshold cycle values were obtained and relative m⁶A was calculated using the formula: 2−(CT Bst(−)−Ct SSIII(−)/Ct Bst(+)−Ct SSIII(+)). At least 2 biological replicates were used for each sample. Error bars represent standard deviation, and statistical significance was calculated using a two-way t-test assuming unequal variance.

Flow cytometry. After 24 hours, the culture media was replaced with media containing 2 μM puromycin for an additional 72 hours to select against the non-infected cells. Cells were then cultured in puromycin-free media for 48 hours, followed by 589 transfection with the GEMS-EGFP system. After 24 hours, cells were dissociated by TrypLE (Gibco) treatment for 10 minutes at 37° C. and 5% CO2. Trypsinized cells were resuspended in 5 mL of growth media containing 1% FBS and passed through a 4 μm cell filter to further separate the culture into single cells. Flow cytometry analysis was performed on a Sony MA900 cell sorter. Cell suspensions were first sorted by size and forward scatter to gate on live cells (BSC-A vs. FSC-A) and to eliminate doublets (FSC-H vs. FSC-A). 2 lasers were used to sort EGFP-positive cells (laser excitation 488 nm) and DsRed-positive cells (laser excitation 561 nm). Cells were sorted in a 4-way channel and collected in 5 mL conical tubes containing 1 mL of 1×PBS. Thresholds for EGFP and DsRed negative fluorescence were pre-calibrated using non-transfected HEK293T cells. Collection stopped when 500,000 cells were collected in the target populations (DsRed+/EGFP− and DsRed+/EGFP+). Sorted cells were collected by centrifugation (6,000 rpm, 10 minutes, 4° C.), and genomic DNA was extracted and analyzed by sequencing using METTL3 locus-specific primers. Indels at the METTL3 locus were identified by aligning the obtained sequences with the genomic sequence of endogenous METTL3 (GRCh38; chr14:21503198-21503835). Results were reported as the percentage of cells containing METTL3 indels out of the total number of cells obtained in each sorted cell population.

For APO1-YTH vs. APO1-YTHmut analysis, HEK293T cells were co-transfected with EGFP-DHFR and either the APO1-YTH or APO1-YTHmut plasmid. Cells were collected 24 hours later and samples were prepared for flow cytometry analysis as described above. Samples were analyzed using a Sony MA900 cell sorter and 1 million cells were recorded to measure EGFP fluorescence (last excitation 488 nm). FCS files were analyzed using Floreada.io software and plotted on a density plot as the frequency of events vs. EGFP fluorescence for each sample.

Mass spectrometry analysis. Total RNA was extracted as described above, and mRNA was purified using two rounds of oligo(dT) purification with Dynabeads oligo-(dT) mRNA purification kit (Invitrogen), followed by 2 rounds of rRNA depletion using NEBNext rRNA Depletion Kit (V2.0, NEB), and an additional two rounds of oligo(dT) purification with Dynabeads oligo-(dT) mRNA purification kit (Invitrogen). All mRNA purification steps were performed following the manufacturer's instructions. Purified mRNA quality was checked using a Bioanalyzer high sensitivity RNA analysis 6000 pico kit (Agilent). For mass spectrometry analysis, 100-200 ng of purified mRNA was incubated with 2 U of Nuclease P1 (Sigma) with 2.5 mM ZnCl and 25 mM NaCl at 37° C. for 2 hours. mRNA samples were treated with 5 U of antarctic phosphatase (NEB) for 2 h at 37° C. Samples were then processed using the Xevo TQ-S mass spectrometry system. All nucleosides were quantified by retention time and ion mass transitions of 268.2 to 133.2 (A) and 282.2 to 150.1 (m⁶A). Data were plotted as a percentage of m⁶A relative to A. At least 2 biological replicates were performed for each sample.

Cell growth assays. Huh-7 and HepG2 cells were plated in 6-well culture plates and transiently transfected with the indicated plasmids. 12 hours after transfection, cells were dissociated using TrypLE (Gibco) treatment for 2 minutes at 37° C. and 5% CO2. Trypsinized cells were resuspended in 5 mL of cell type-specific growth media, and an aliquot was used to count the number of cells in the culture using a hemocytometer. 10,000 cells for each sample were plated in one well of a 6-well culture plate for a total of 6 wells per condition. A hemocytometer was used to count the number of cells in each well every 24 hours for 5 days. Counts were performed in 3 technical replicates, and the average number of cells was used to calculate the ratio as indicated in each experiment.

Migration assays. Huh-7 cells were plated in 6-well culture plates and transiently transfected with the indicated plasmids. 12 hours after transfection, cells were dissociated using TrypLE (Gibco) treatment for 2 minutes at 37° C. and 5% CO2. 500 cells from each sample were plated on the top of a 6.5 mm transwell membrane with 8 μm pores (Corning). Culture medium inside the transwell chamber was formulated without the addition of FBS, while the culture medium at the bottom of the well included 10% FBS as a chemoattractant. 24 hours after plating the cells in the transwells, transwells were washed twice with 1×PBS, and the non-migrated cells were cleared using a cotton swab on the top of the transwell membrane. The membrane was fixed with methanol for 30 minutes, then washed with 1×PBS. Membranes were then stained with 5% Crystal Violet (VWR) for 30 minutes, then washed 3 times with 1×PBS. Transwell membranes were placed on a microscope glass slide and imaged under a brightfield 20× objective. At least 4 images were obtained for each condition and representative images were selected.

### Example 2: m⁶A Sensor Design and Validation

A system for sensing m⁶A in cellular mRNAs was envisioned. That system has three main features: 1) it is genetically encoded to enable m⁶A sensing in living cells, 2) it is versatile and capable of being used in a variety of cell and tissue types, and 3) it provides a simple readout compatible with high-throughput studies. To achieve these goals, a system was designed that uses a reporter mRNA which produces a fluorescent protein (EGFP) only when the mRNA is methylated. This simple system was referred to as GEMS (genetically encoded m⁶A sensor), and, therefore couples cellular fluorescence with m⁶A methylation.

To achieve m⁶A-dependent production of EGFP in the GEMS system, the DART-seq was used. DART-seq is a method that previously developed for m⁶A detection²⁰. DART-seq identifies m⁶A residues in cells by using a fusion protein consisting of the YTH domain, which directly binds to m⁶A sites, tethered to the cytidine deaminase APOBEC1. When the APOBEC1-YTH fusion protein is expressed in cells, it binds to m⁶A and catalyzes C-to-U editing of nearby cytidine residues (FIG. 1). This property of the APOBEC1-YTH fusion protein (hereafter APO1-YTH) could be harnessed to develop a system in which m⁶A-dependent C-to-U editing produces a stop codon that alters the expression of EGFP and provides a readout for m⁶A.

The GEMS system contains two components: APO1-YTH and an m⁶A reporter mRNA (FIG. 1). The reporter mRNA contains the coding sequence for EGFP followed by an m⁶A “sensor sequence”, which contains two m⁶A consensus motifs (GAC) and two tandem convertible codons in-frame with EGFP. When unedited, these codons encode arginine and glutamine (CGA and CAG, respectively). However, C-to-U editing produces two stop codons (UGA and UAG). The surrounding m⁶A sensor sequence is modified from a similar sequence in the human ACTB mRNA 3′UTR which contains two methylated GAC sequences that have been reported in many different cell types^{3, 4, 20, 22-24}. Downstream of the m⁶A sensor sequence and in-frame with EGFP is the coding sequence for a destabilization domain modified from the Escherichia coli dihydrofolate reductase gene (ecDHFR). This destabilization domain was previously engineered to induce rapid, proteasome-mediated degradation of proteins to which it is tethered²⁵. Thus, when the EGFP-DHFR m⁶A reporter mRNA is introduced into cells together with APO1-YTH, if the reporter mRNA is not methylated, there will be no editing of the m⁶A sensor sequence by APO1-YTH and the full-length EGFP-DHFR protein will be translated. The result will be rapid degradation of EGFP-DHFR and no fluorescence (FIG. 1). However, if either of the GAC sequences within the m⁶A sensor sequence is methylated, APO1-YTH will bind to the m⁶A and deaminate one or both cytidine residues within the two convertible stop codons of the sensor sequence. The result will be translation of EGFP followed by translation termination before the ribosome encounters the DHFR sequence. The EGFP protein will not be degraded since it will not be fused to DHFR, resulting in EGFP fluorescence (FIG. 1). Thus, this system provides a simple fluorescent readout for the presence of m⁶A (no m⁶A=no EGFP fluorescence; m⁶A=EGFP fluorescence).

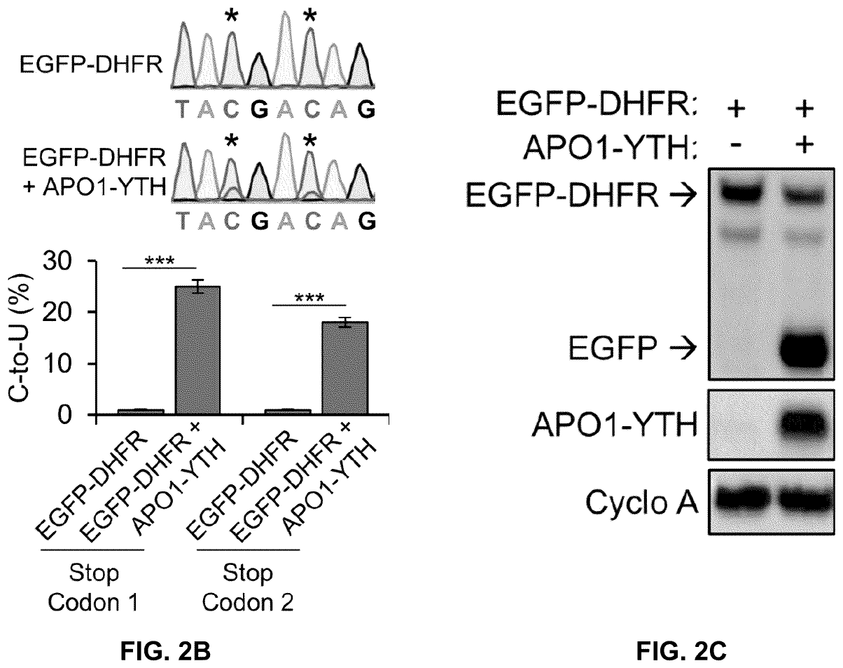

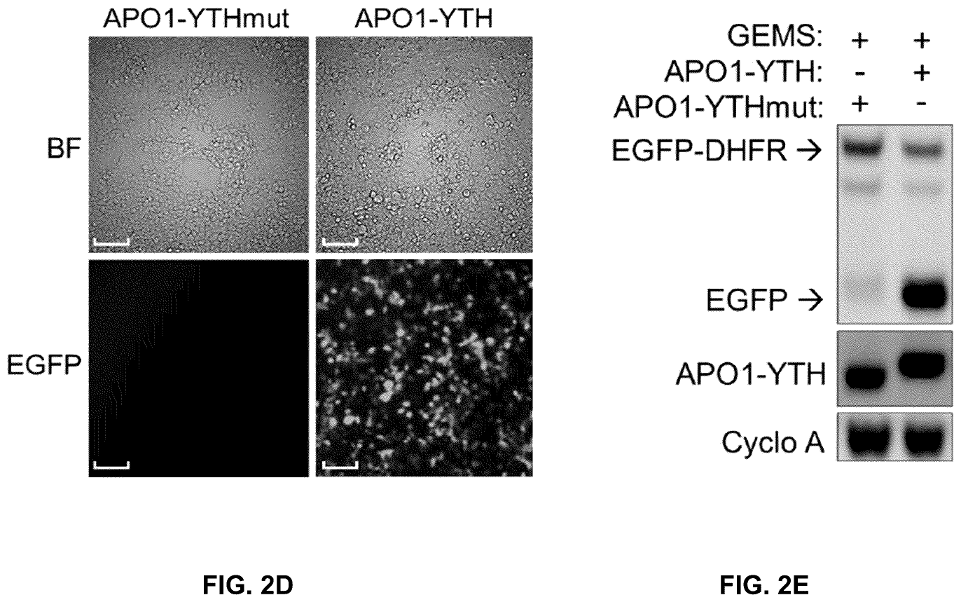

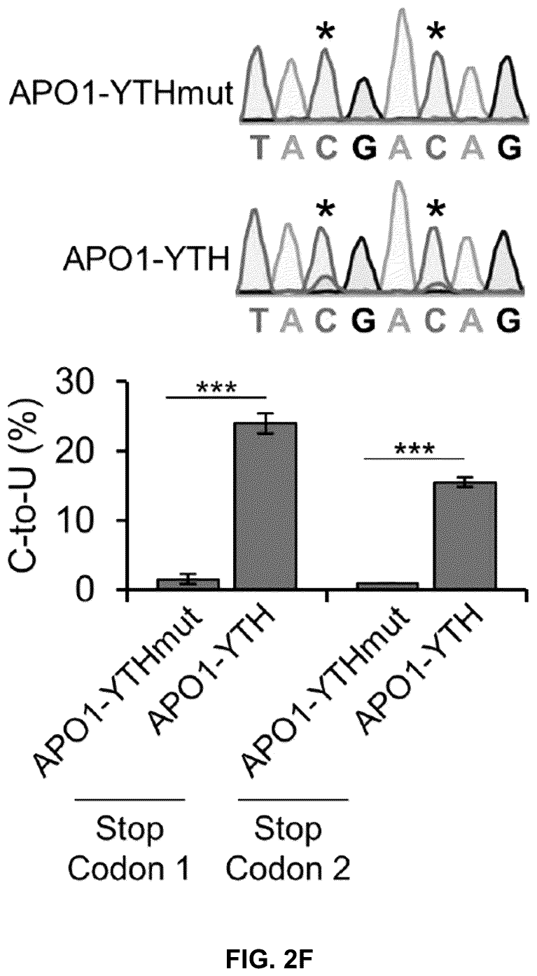

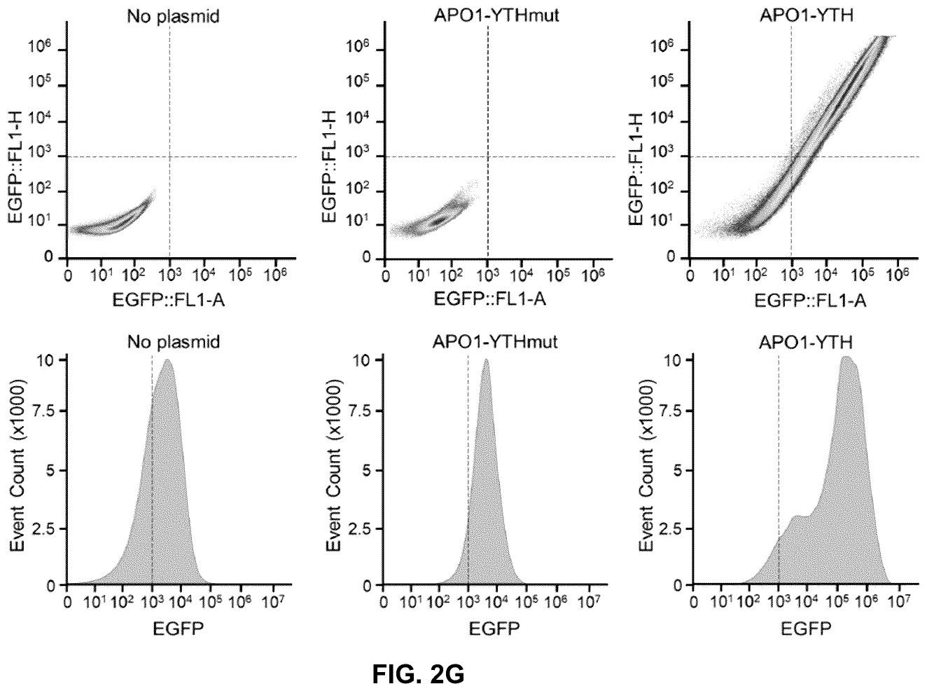

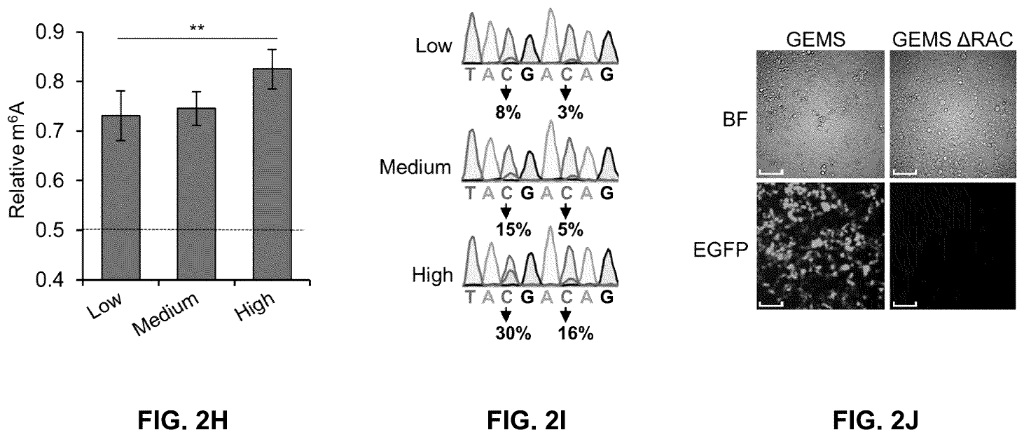

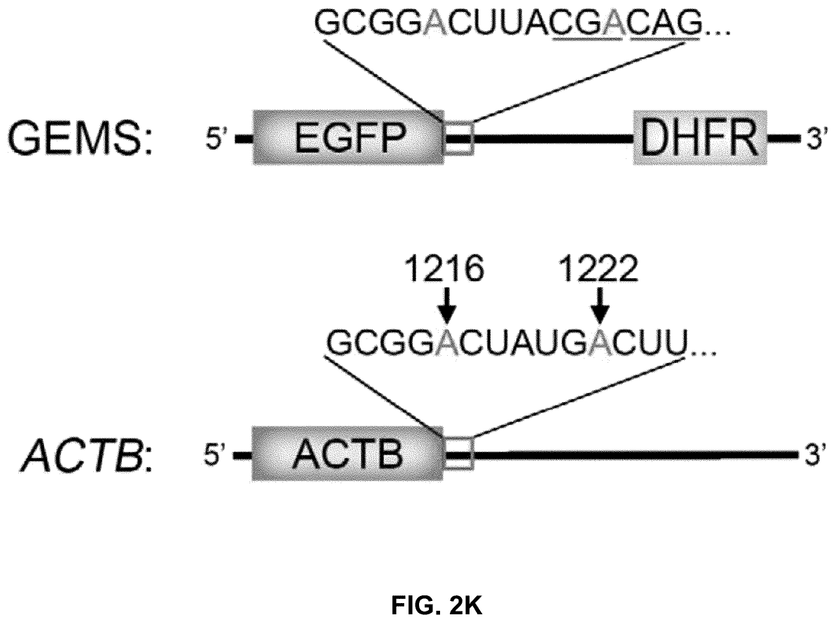

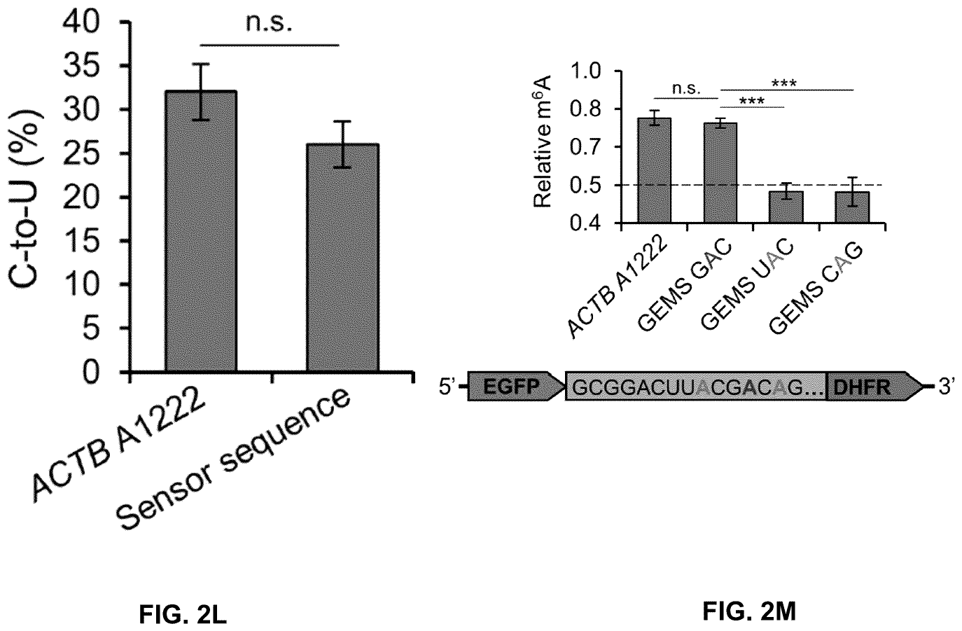

To determine whether the GEMS system can sense cellular mRNA methylation, the system was transfected into HEK293T cells and assessed cellular fluorescence 24 hours later. Cells expressing APO1-YTH together with the m⁶A reporter mRNA exhibit robust EGFP fluorescence, whereas cells only expressing the m⁶A reporter mRNA are dark (FIG. 2A). In addition, Sanger sequencing of the reporter mRNA indicates C-to-U editing of the convertible stop codon sequences only in cells expressing APO1-YTH (FIG. 2B). This is further confirmed by western blot, which shows that cells expressing the reporter mRNA together with APO1-YTH produce both EGFP and EGFP-DHFR, whereas cells lacking APO1-YTH only produce EGFP-DHFR (FIG. 2C).

As an additional control to demonstrate that EGFP production and cellular fluorescence are due to recognition of m⁶A by APO1-YTH, cells were transfected with the m⁶A reporter mRNA and APO1-YTHmut, a mutant version of the APO1-YTH fusion protein which lacks the full m⁶A binding region of the YTH domain and exhibits greatly reduced m⁶A-binding activity²⁰. This resulted in loss of EGFP fluorescence and EGFP protein production as well as decreased editing of the m⁶A sensor sequence (FIGS. 2D-2G), indicating that EGFP fluorescence and m⁶A sensor sequence editing are due to m⁶A recognition by APO1-YTH. Consistent with this, when the cells expressing the GEMS system were subjected to flow cytometry and sorted cells based on EGFP fluorescence, methylation and C-to-U editing of the m⁶A sensor sequence were found to be increased in cells with higher EGFP fluorescence (FIGS. 2H-2I). Additionally, when the RAC motifs within the m⁶A sensor sequence were mutated to preclude methylation, GEMS activity was abolished (FIG. 2J), further demonstrating that GEMS activity depends on m⁶A modification of the reporter mRNA. FIG. 2K. Top schematic shows the m6A reporter mRNA with a portion of the m6A sensor sequence expanded. This sequence is based off of a sequence within the human ACTB 3′UTR (bottom schematic), which contains two m6A sites at positions A1216 and A1222 that have been shown to be methylated in several m6A mapping studies.

Both the EGFP and EGFP-DHFR protein products are detected in cells expressing the GEMS system (FIGS. 2C, 2E). This likely reflects the presence of both methylated and unmethylated copies of the reporter mRNA, which is consistent with the known sub-stoichiometric abundance of m6A in cellular mRNAs^{17, 19, 22, 26, 27}. Therefore, the level of methylation in the m⁶A sensor sequence was analyzed to determine if it is comparable to that of endogenous mRNAs. Previously, the proportion of APO1-YTH-mediated C-to-U editing (% C2U) near m6A sites is correlated with m6A stoichiometry^{20, 26}. The stoichiometry of m6A in cellular mRNAs is generally low, with most sites estimated to be at levels of 40% or less^{17, 19, 22, 26, 27} Therefore, if the m⁶A sensor is a faithful representation of cellular mRNA methylation, one may expect C-to-U editing levels of the m⁶A sensor sequence to be comparable to endogenous mRNA. Indeed, when comparing the editing rate of the m⁶A sensor sequence to the editing seen in the region of the endogenous ACTB 3′UTR on which the sensor sequence is modeled, highly similar values were observed (FIG. 2L). Additionally, an orthogonal, RT-qPCR-based method for m⁶A detection and quantification²⁸ was used to target the m⁶A sensor sequence as well as endogenous ACTB. This validated that m⁶A consensus adenosines within the sensor sequence are methylated in cells at a similar level as the corresponding region in endogenous ACTB, whereas non-consensus adenosines are unmethylated (FIG. 2M).

Altogether, these data demonstrate that the GEMS system produces robust EGFP fluorescence that depends both on the m6A-binding ability of APO1-YTH and on methylation and C-to-U editing of the m6A sensor sequence. Furthermore, methylation of the GEMS reporter mRNA mirrors the m6A level seen in a similar region of the ACTB mRNA, indicating that GEMS is an accurate representation of endogenous cellular mRNA methylation.

### Example 3: GEMS Responds to Changes in METTL3 Expression Levels

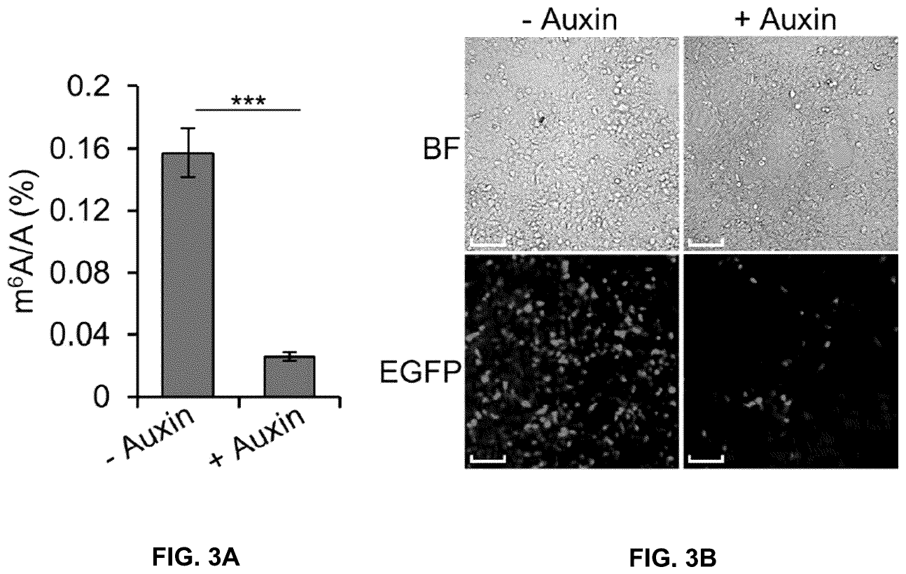

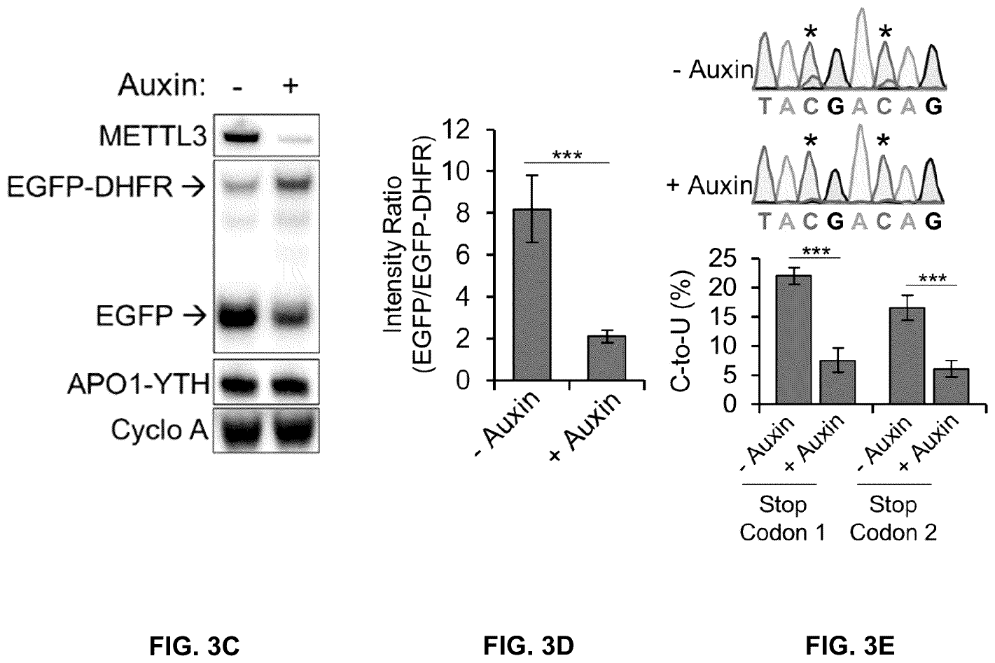

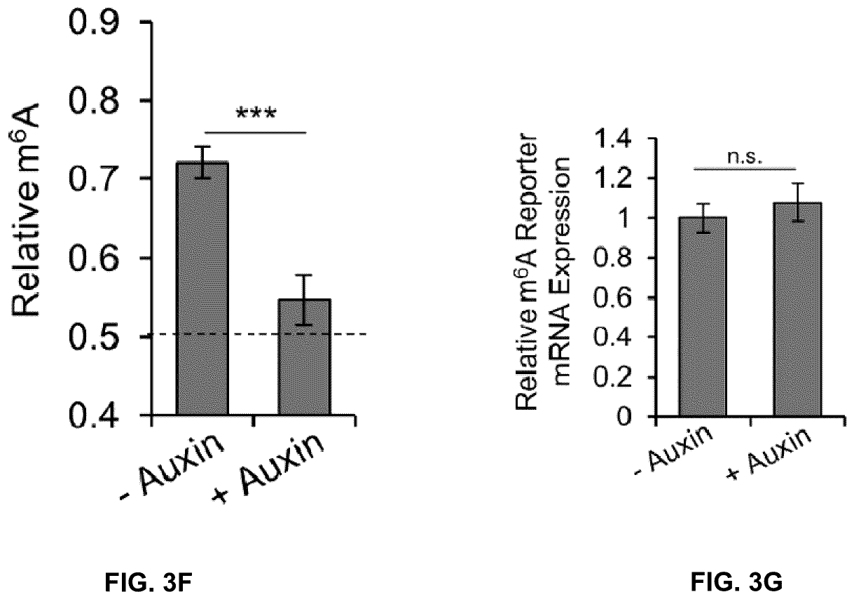

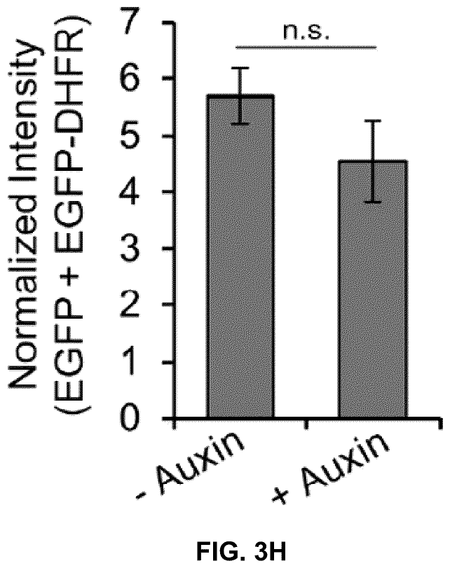

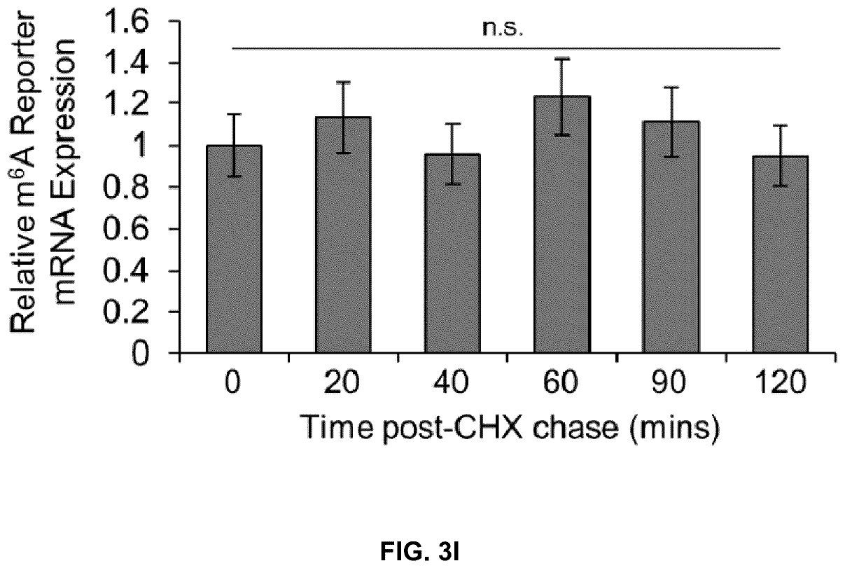

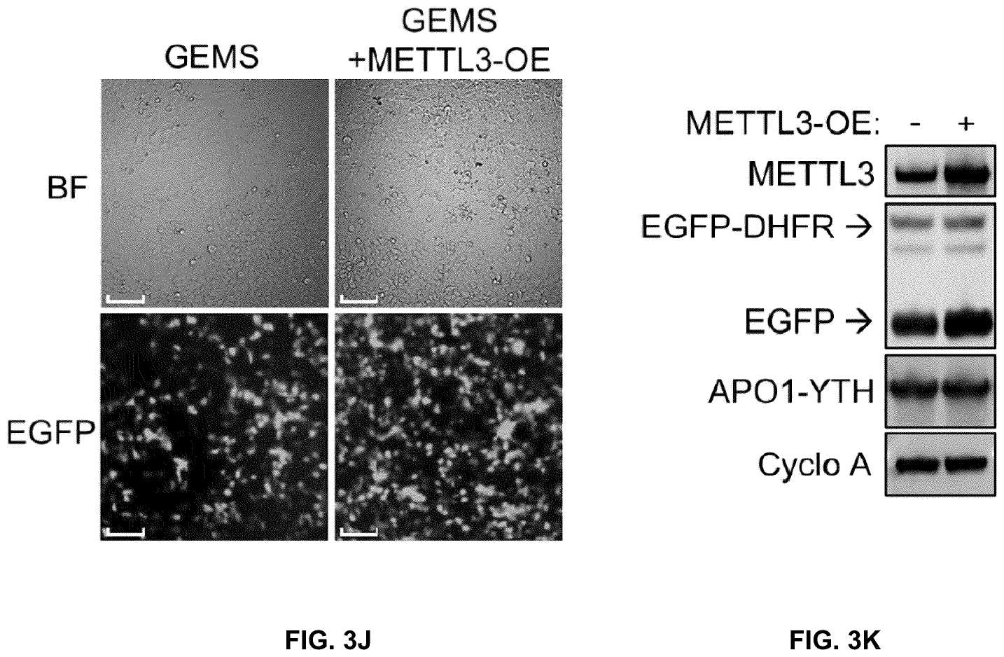

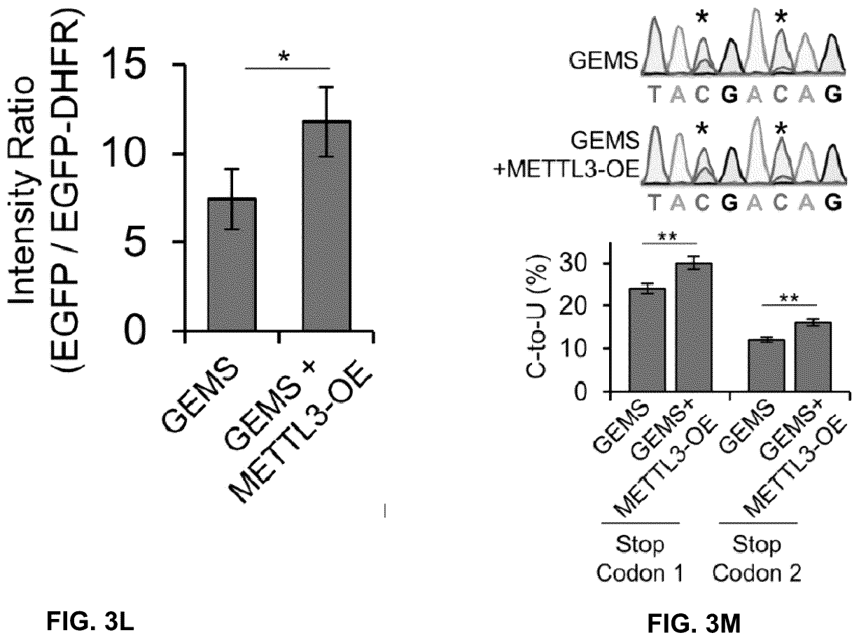

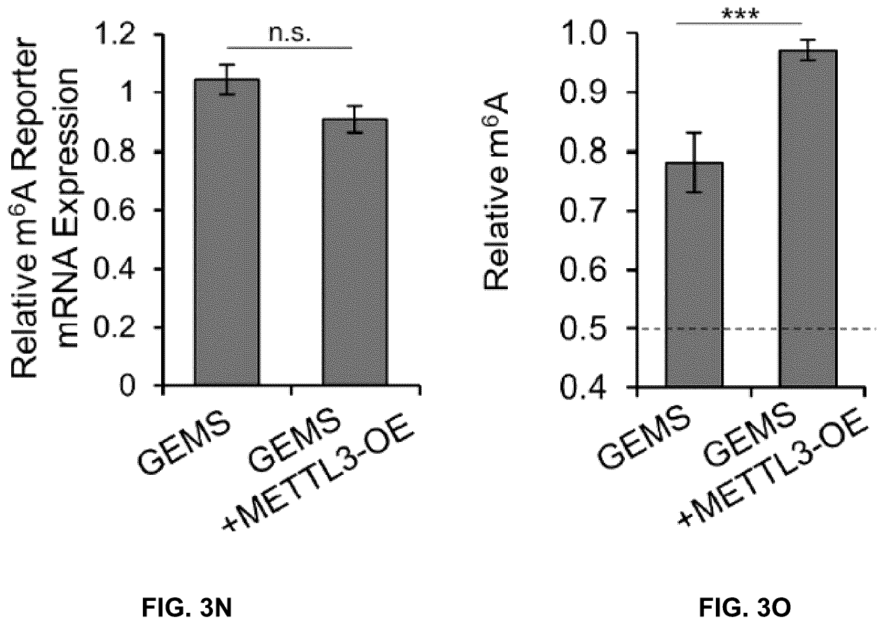

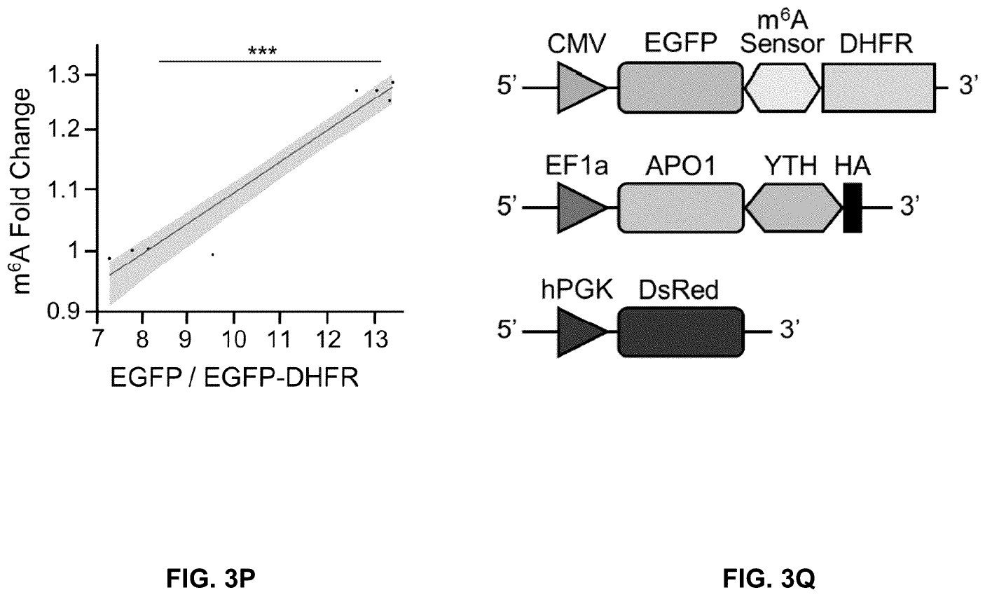

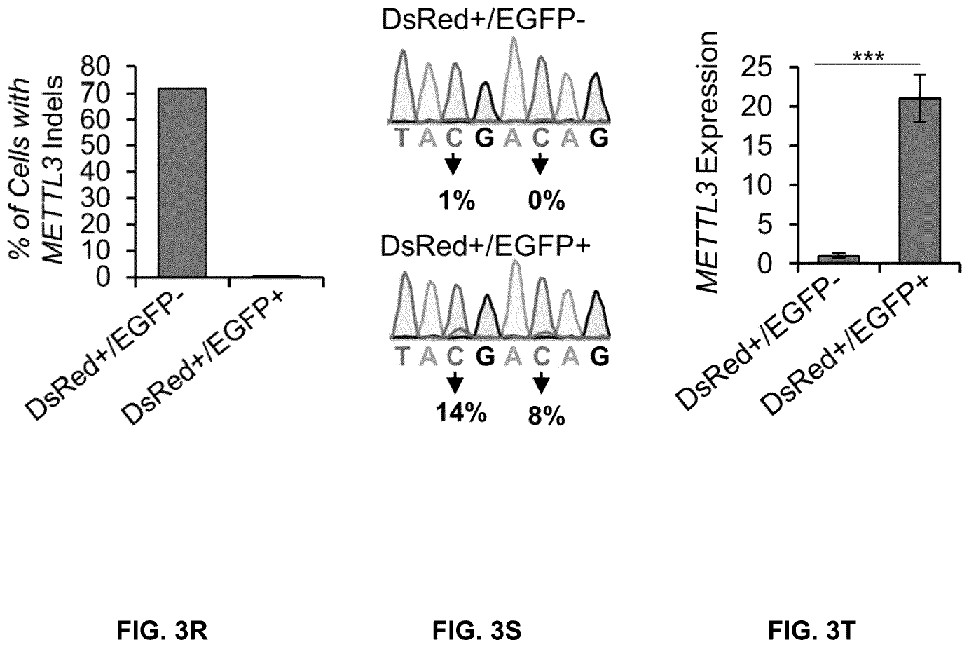

This example discusses that the m⁶A sensor is METTL3-dependent. The GEMS system was expressed in HEK293T cells that contain an auxin-inducible degradation tag at the endogenous METTL3 locus and which exhibit decreased levels of m⁶A in the presence of auxin (FIG. 3A). Substantially reduced EGFP fluorescence was observed in auxin-treated cells compared to DMSO-treated cells (FIG. 3B). This was accompanied by a reduced EGFP:EGFP-DHFR ratio as assessed by western blot and decreased C-to-U editing of the m⁶A sensor sequence (FIGS. 3C-3E). Importantly, it was confirmed that these effects are due to loss of m⁶A, as mass spectrometry- and RT-qPCR-based m⁶A quantification showed reduced methylation of cellular mRNAs and the m⁶A sensor sequence, respectively, in auxin-treated cells (FIG. 3F, FIG. 3A). Also, m⁶A reporter mRNA abundance was quantified in METTL3-depleted cells after 24 hours; similar levels of abundance was found as in cells without METTL3 depletion, indicating that methylation of the reporter mRNA does not alter its stability (FIG. 3G). Furthermore, loss of m⁶A does not alter total protein production from the reporter mRNA (FIG. 3H). Lastly, the premature termination codons introduced by C-to-U editing of the reporter mRNA do not trigger nonsense-mediated decay (NMD), as treatment of cells with cycloheximide to limit NMD has no effect on reporter mRNA levels (FIG. 3I). Consistent with this, the reporter mRNA lacks introns and is therefore not expected to be susceptible to exon junction complex-dependent NMD.

To determine whether the GEMS system can also detect elevated methylation caused by increased levels of METTL3, GEMS was introduced into HEK293T cells together with exogenous expression of METTL3. This led to increased EGFP fluorescence, a higher EGFP:EGFP-DHFR ratio, and increased C-to-U editing of the sensor sequence (FIGS. 3J-3M). Overexpression of METTL3 also led to an increase in methylation of the sensor sequence without affecting reporter mRNA stability (FIGS. 3N-3O)—as little as a 1.29-fold increase in reporter mRNA methylation was sufficient to produce a significant increase in EGFP (FIG. 3P). Thus, the GEMS system is sensitive to both increased and decreased cellular m⁶A methylation caused by changes in METTL3 expression.

Since GEMS uses EGFP fluorescence as a readout for m⁶A, factors that inhibit general transcription, translation, or fluorescent protein (FP) production could potentially lead to a false readout and limit the utility of GEMS for some applications. To address this, the GEMS system was modified to include DsRed under the control of a separate promoter to control for transcription and general FP production (FIG. 3Q). Then, tests were performed to see whether selective reduction of EGFP (m⁶A-coupled) compared to DsRed (m⁶A-uncoupled) fluorescent signal could be used to detect genetic disruption of METTL3.

HEK293T cells were infected with a Cas9-expressing lentivirus and sgRNAs targeting either METTL3 or the AAVS1 safe harbor gene locus followed by transfection with the GEMS system and flow cytometry to isolate cells based on red/green fluorescence. Cells were then subjected to targeted sequencing of the METTL3 locus to determine whether CRISPR-induced indels are enriched in DsRed+/EGFP− cells, which would be expected if selective reduction of EGFP fluorescence reflects METTL3 disruption. Indeed, METTL3 indels are substantially higher in DsRed+/EGFP− cells compared to DsRed+/EGFP+ cells (FIG. 3R). This is accompanied by a decrease in C-to-U editing of the m⁶A sensor sequence, with nearly undetectable editing in the DsRed+/EGFP− pool of cells (FIG. 3S). Consistent with this, METTL3 expression is significantly decreased in the DsRed+/EGFP− pool of cells (FIG. 3T). Altogether, these data show that GEMS can be used to detect genetic perturbation of METTL3 and demonstrate its sensitivity to both increased and decreased METTL3 expression through m⁶A-coupled FP production.

### Example 4: GEMS Detects Differential Methylation Across Cell Types

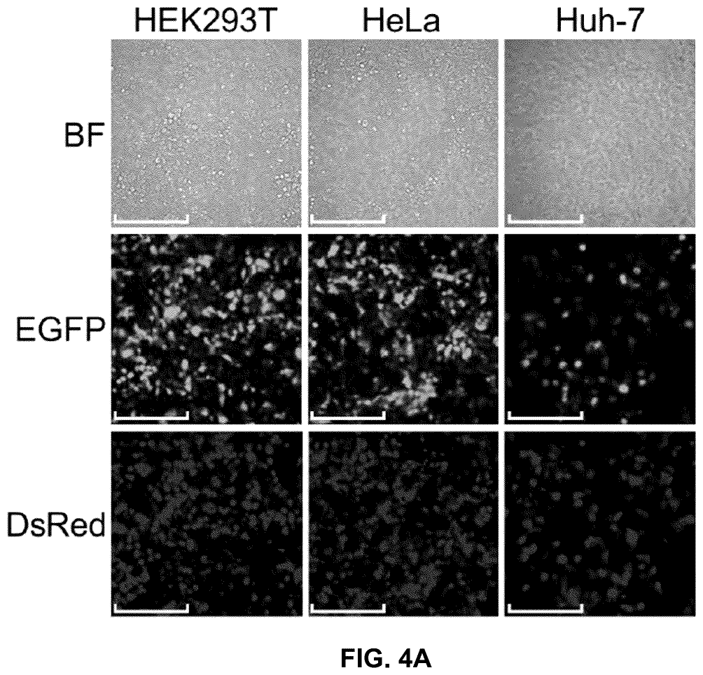

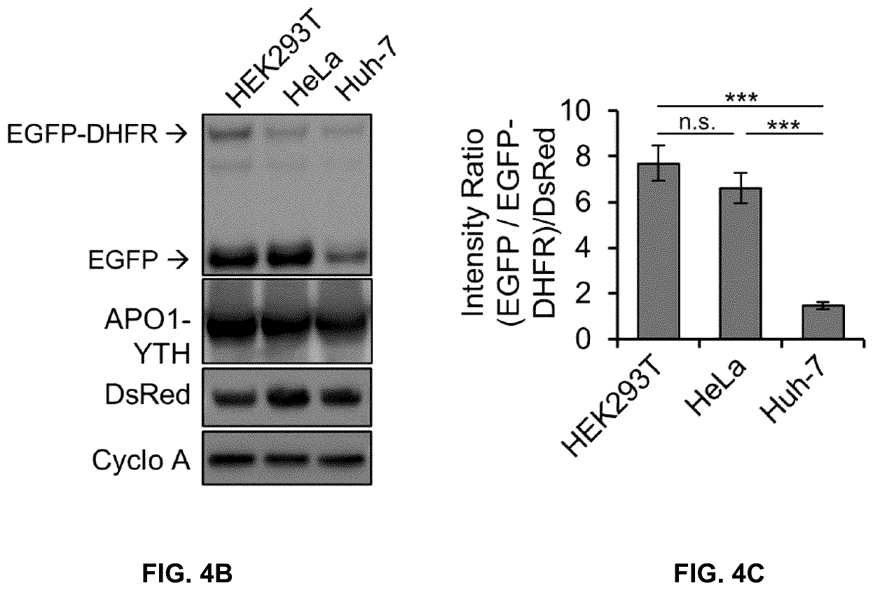

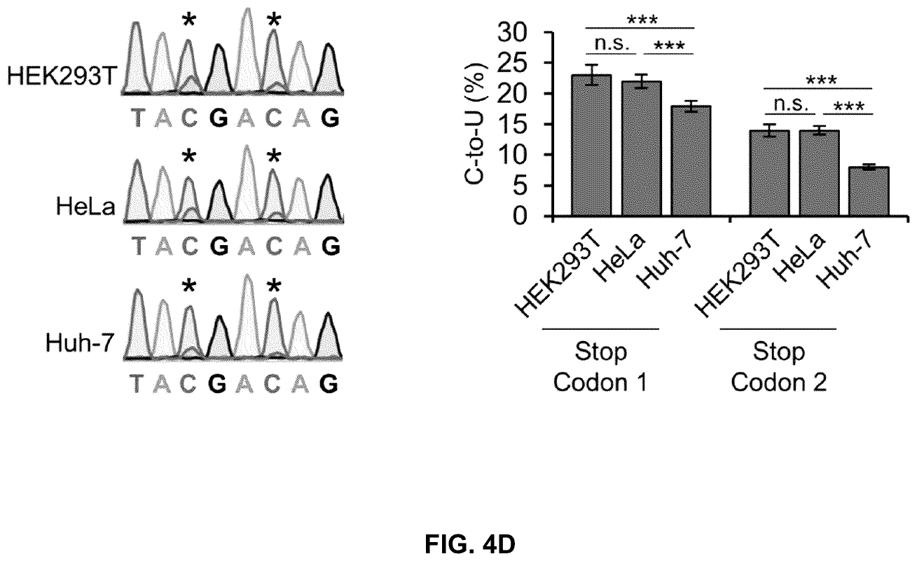

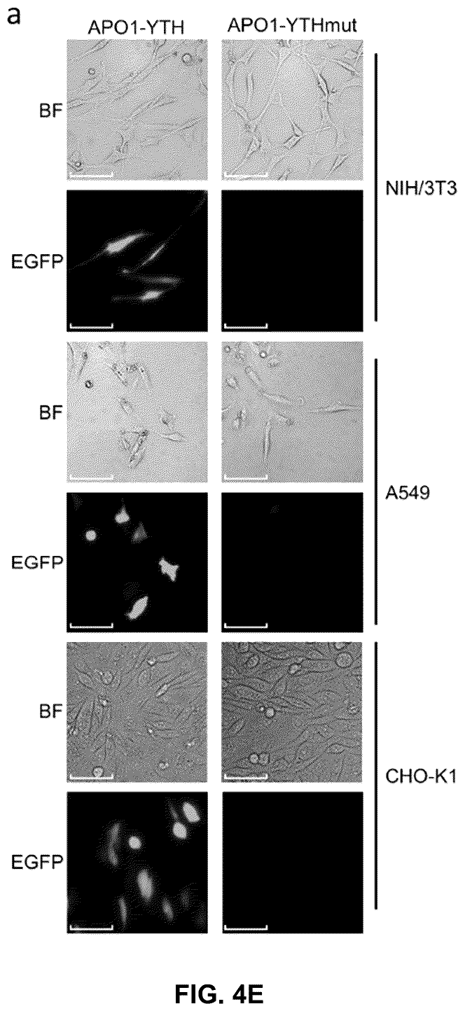

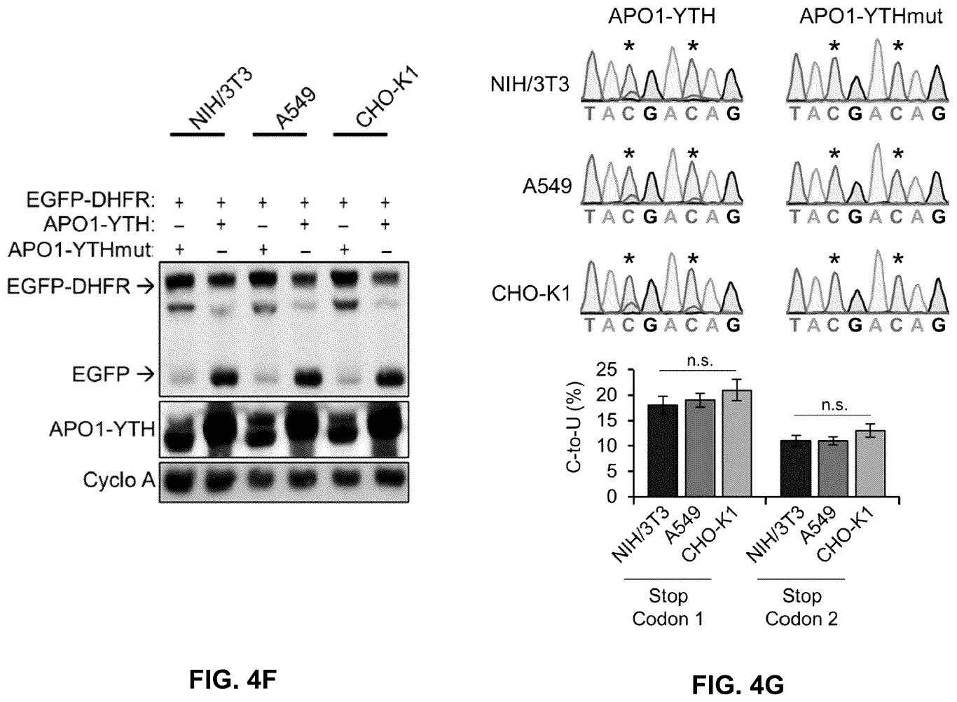

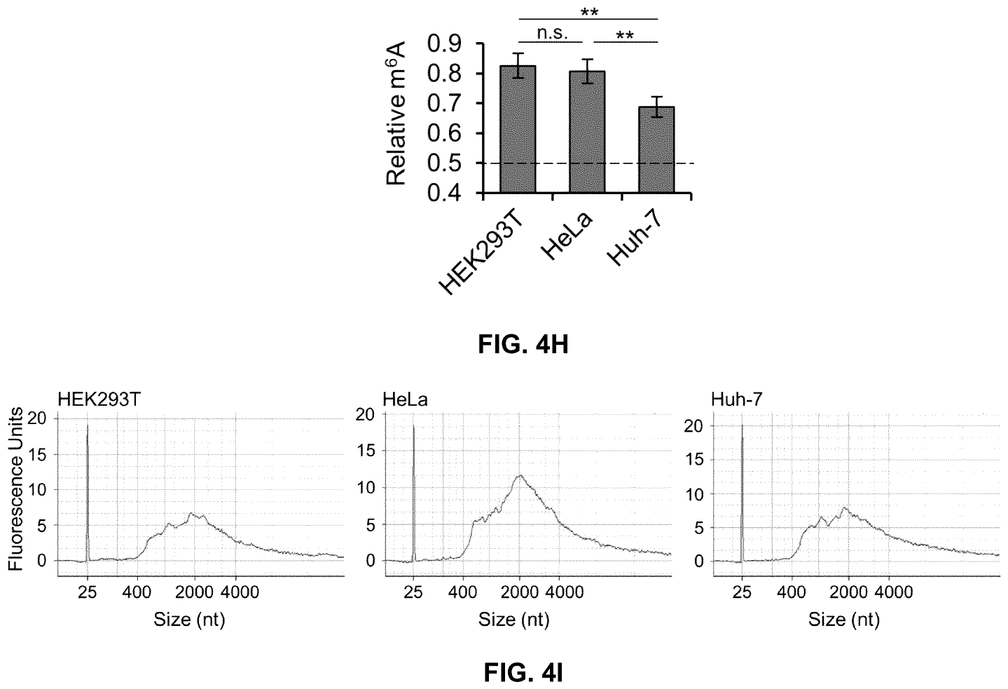

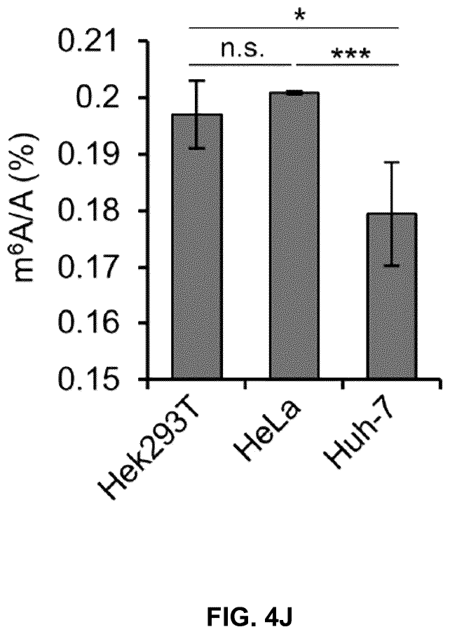

This example discusses the utility of GEMS for sensing m⁶A across diverse cell types by expressing the system in a variety of mouse and human cell lines. For each cell type, EGFP protein production and fluorescence were observed, as well as editing of the m⁶A sensor sequence, indicating that the GEMS system is active (FIGS. 4A-4D, FIGS. 4E-4G). It was confirmed that this is due to m⁶A recognition, since cells were dark and had greatly reduced C-to-U editing of the sensor sequence when APO1-YTH was replaced with APO1-YTHmut (FIGS. 4E-4G). Thus, GEMS can be used in a variety of different cell types to detect m⁶A.

Interestingly, some cell types were observed to have higher levels of EGFP fluorescence and sensor sequence editing than others. This could reflect different levels of m⁶A and perhaps GEMS can be used to report differential mRNA methylation across distinct cell types. To test this, the system was expressed in three commonly used human cell lines (HEK293T, HeLa, and Huh-7) with DsRed as an internal control. It was found that EGFP fluorescence, EGFP:EGFP-DHFR ratio, and m⁶A sensor sequence editing are highly similar in HEK293T and HeLa cells but substantially reduced in Huh-7 cells (FIGS. 4A-4D). This is not caused by differences in expression of APO1-YTH, as similar levels were observed across the three cell types (FIG. 4B). Furthermore, quantification of sensor sequence methylation in each cell type revealed similar m⁶A levels in HEK293T and HeLa cells but reduced levels in Huh-7 cells (FIG. 4H), indicating that EGFP fluorescence can be used to detect cell type-dependent differences in methylation of the GEMS reporter mRNA.

GEMS uses a single mRNA to sense m⁶A. To validate that GEMS activity reflects mRNA methylation levels globally, mRNA was purified from HEK293T, HeLa, and Huh-7 cells and performed mass spectrometry to quantify m⁶A levels (FIG. 4J). Consistent with the relative GEMS activity across the three cell lines, mRNA from HEK293T and HeLa cells have similar levels of m⁶A, whereas the amount of m⁶A in Huh-7 cellular mRNA is reduced (FIG. 4E, FIG. 4I). Thus, these data demonstrate that the GEMS system can be used to sense differences in m⁶A methylation of mRNAs across different cell types.

### Example 5: GEMS Detects Pharmacological Inhibition of METTL3

The m⁶A methyltransferase machinery has recently emerged as a promising therapeutic target for the potential treatment of cancer and other diseases²⁹⁻³². However, efforts to identify METTL3 inhibitors have been hampered by the lack of methods that provide a simple readout for m⁶A methyltransferase activity in living cells on a scale that is compatible with HTS. Since GEMS couples m⁶A methylation with cellular fluorescence, it has potential utility as a HTS-compatible technology for determining the effects of drugs or small molecules on m⁶A levels in cells.

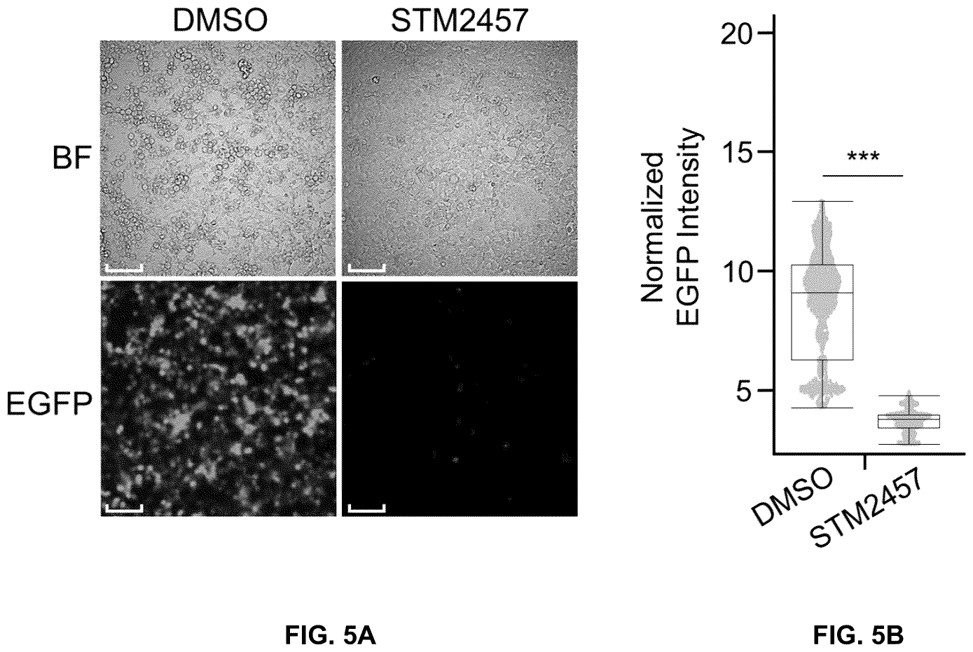

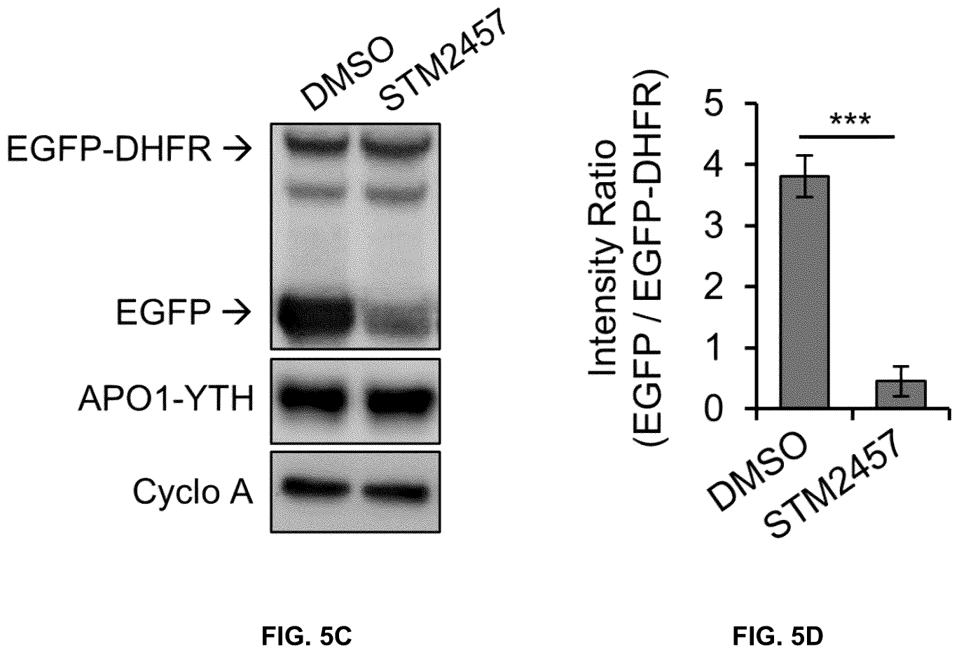

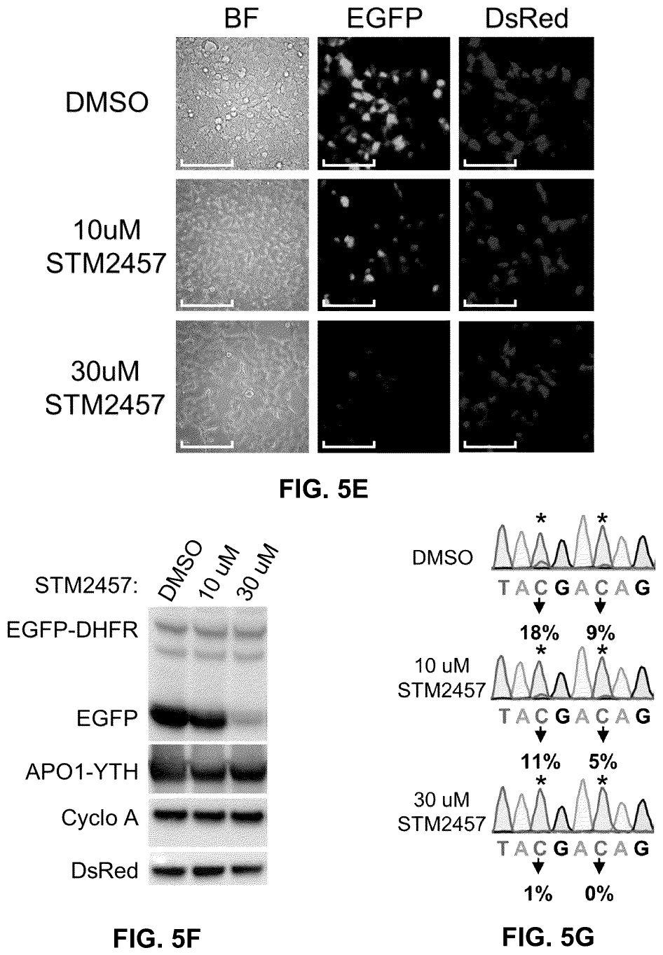

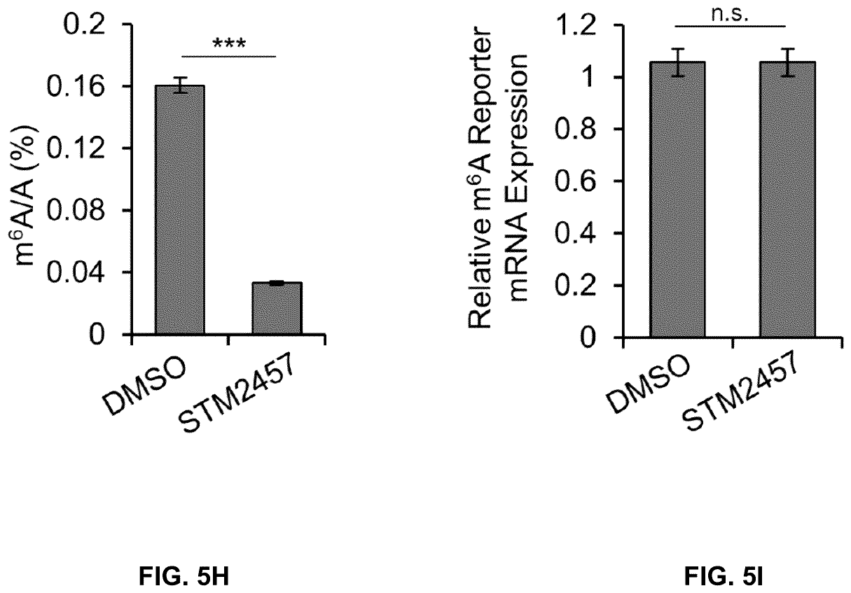

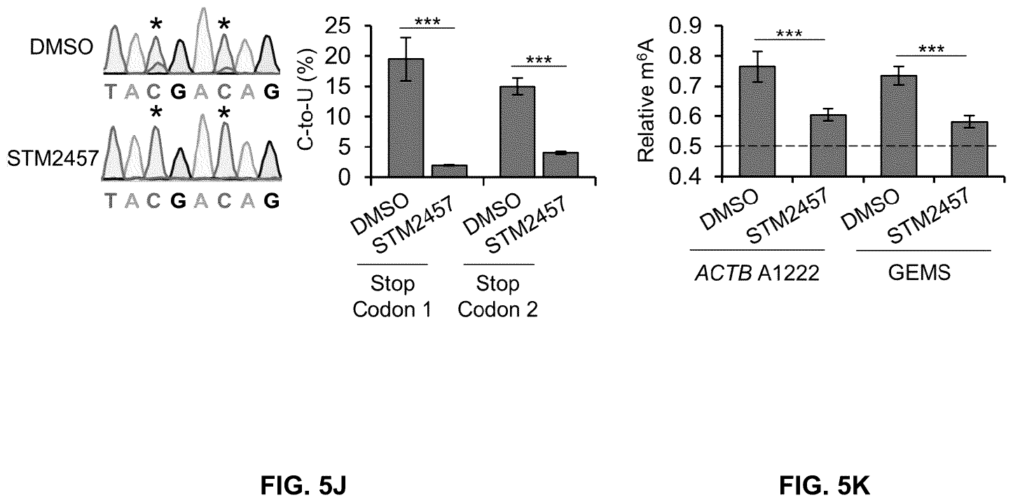

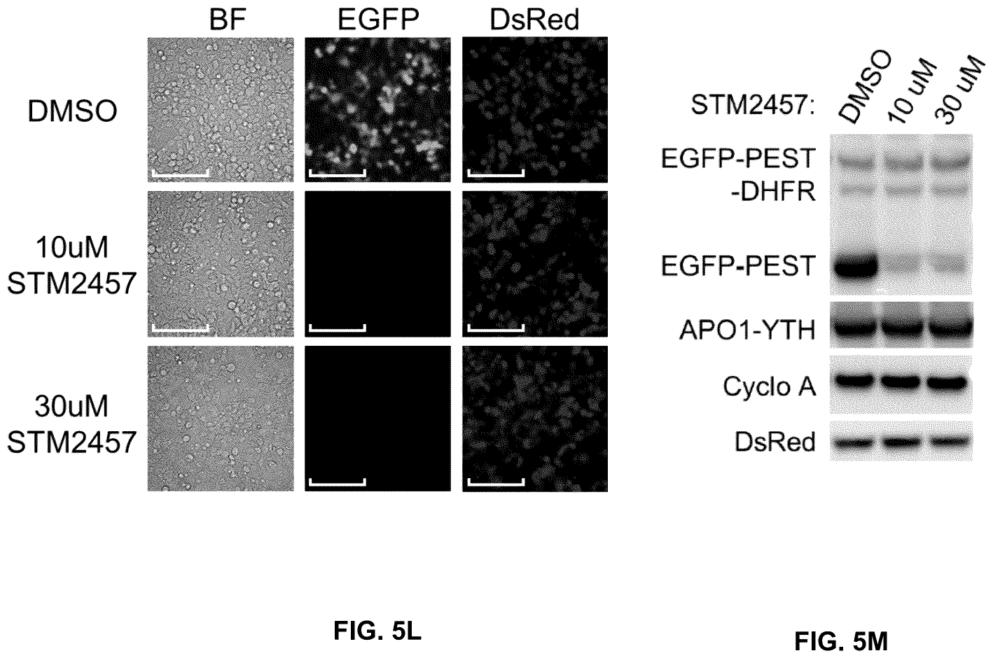

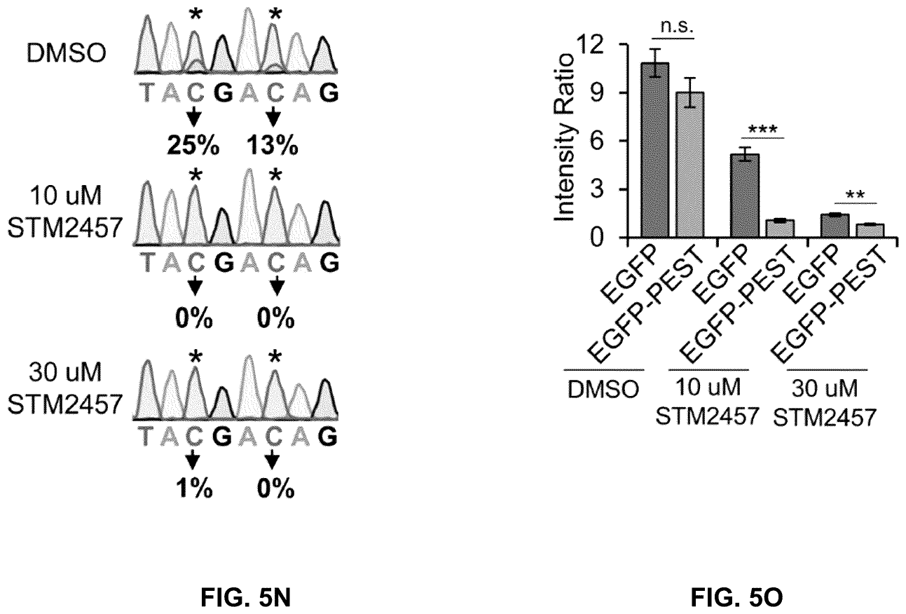

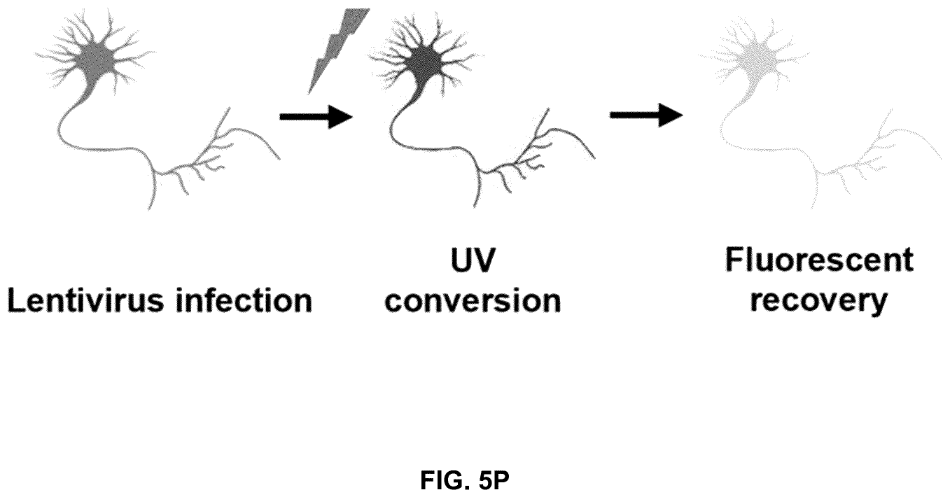

To explore whether the GEMS system can detect pharmacological inhibition of METTL3, HEK293T cells expressing GEMS were subjected to STM2457, a small molecule inhibitor of METTL3²⁹, and performed quantitative microscopy. A significant decrease in EGFP fluorescence was observed following STM2457 treatment (FIGS. 5A-5D), an effect that is exacerbated with increasing doses of STM2457 (FIGS. 5E-5I). This is accompanied by reduced C-to-U editing of the sensor sequence and reduced m⁶A in the sensor sequence (FIG. 5J, FIG. 5K).

The ability of the GEMS system to report m⁶A reduction depends in part on the half-life of EGFP: if cellular mRNA methylation decreases, this can potentially be difficult to detect due to the presence of pre-existing EGFP protein. It may be that an improved GEMS system could be developed by tagging EGFP with a destabilizing domain to reduce its half-life in cells. A PEST degradation sequence was therefore added to the EGFP coding sequence in the GEMS reporter mRNA; this modified system was tested for its ability to respond to METTL3 inhibition with STM2457. Indeed, the EGFP-PEST reporter enabled improved detection of m⁶A depletion compared to the original EGFP version (FIGS. 5L-5O). Altogether, these data show that the GEMS system effectively detects m⁶A depletion mediated by pharmacological inhibition of METTL3 and demonstrate that the use of more short-lived FPs in the GEMS system can improve the detection of m⁶A dynamics in cells. FIG. 5P is a cartoon depicting an example of an alternative FP that could be utilized in the GEMS system in place of EGFP. This schematic shows primary neurons that are infected with a lentivirus expressing a photoconvertible FP such as Dendra2, which emits green fluorescence that is converted to red fluorescence upon exposure to UV light. New Dendra2 protein can the subsequently be identified by green fluorescence.

### Example 6: m⁶A-Coupled CRISPRi Targeting

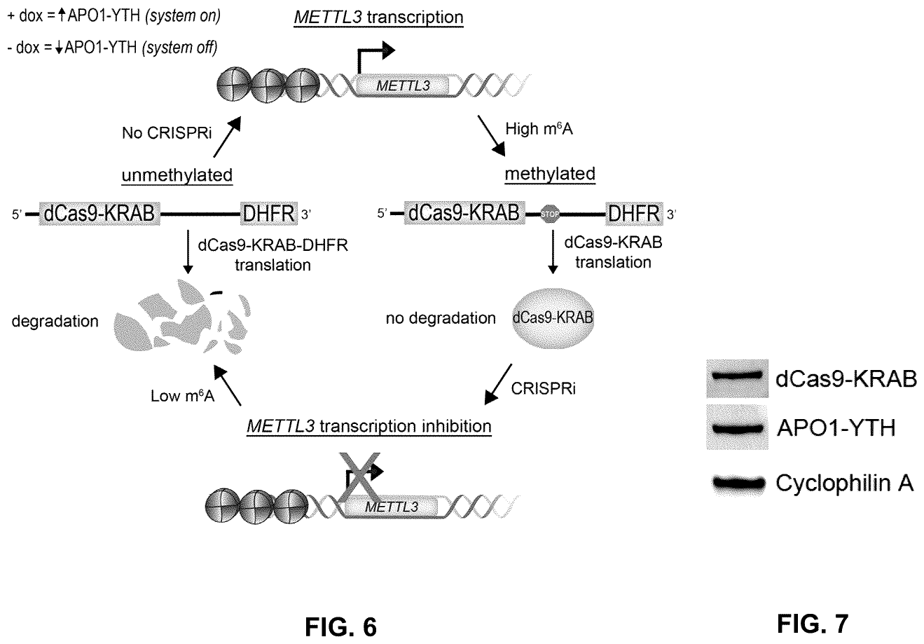

Because the APO1-YTH protein edits cellular methylated mRNAs in addition to the GEMS reporter mRNA, it could potentially lead to unwanted effects in cells. Therefore, an alternative approach was developed to target APO1-YTH specifically to the reporter mRNA and reduce editing of endogenous cellular transcripts. An additional application of the m⁶A-coupled effector protein delivery system is driving expression of CRISPR tools that target METTL3. This can provide an m⁶A-dependent feedback mechanism which reduces METTL3 expression when m⁶A levels become too high and could therefore serve as a way to maintain m⁶A homeostasis in cells. This can be tested by developing a system that expresses m⁶A-coupled CRISPRi tools to inhibit METTL3 transcription (FIG. 6). Embodiments employing dCas9 and dCas13 are described below, but other catalytically-inactive RNA-guided endonucleases may be employed.

dCas9

In the present embodiment, the GFP sequence of the m⁶A sensor system can be replaced with dCas9-KRAB, which is a fusion protein consisting of inactive Cas9 tethered to the Kruppel-associated box (KRAB) transcriptional repressor (Alerasool et al., An efficient KRAB domain for CRISPRi applications in human cells. Nat Methods. 2020; 17(11):1093-6). Then, a U6-METTL3 sgRNA cassette can be introduced into this plasmid. The result will be constitutive expression of the METTL3 sgRNA but only m⁶A-dependent dCas9-KRAB expression in the presence of doxycyclin, which induces APO1-YTH (FIG. 6). The efficacy of METTL3 sgRNA targeting with CRISPRi can be tested separately in HEK293T cells before choosing which sgRNA sequence to use.

Then, a lentivirus expressing this “m⁶A feedback system” can be packaged and infect HEK293T cells. RNA and protein can be isolated at various timepoints over the course of 72 hours (this can be expanded to longer times as needed). Sensor sequence methylation can be measured using SELECT. Sensor sequence editing can be evaluated with Sanger sequencing. Western blot can be used to assess METTL3, APO1-YTH, and dCas9-KRAB/dCas9-KRAB-DHFR protein levels. Global m⁶A levels in cellular mRNA can also be measured using UPLC-MS/MS. Collectively, these readouts can provide important quantitative metrics of how the m⁶A feedback system responds to gain/loss of m⁶A and how effective the feedback system is at maintaining m⁶A levels as the cell cycles between high and low levels of METTL3. As an additional approach, the cycling of m⁶A levels can be assessed using the m⁶A sensor system. Cells infected with the m⁶A feedback system can be transfected with the GFP-encoding m⁶A reporter mRNA. Live-cell imaging will be used to monitor GFP fluorescence over the course of 72 hours (or longer, as needed).

To determine whether a new protein of interest can be produced in place of GFP in the m⁶A sensor system, dCas9-KRAB was cloned in place of GFP. Robust dCas9-KRAB expression was detected using western blot (FIG. 7), demonstrating that other polypeptides can be expressed from the m⁶A reporter mRNA in place of GFP.

The dCas9-KRAB effector protein delivery system can be expressed in METTL3 degron cells to show that auxin treatment (which leads to METTL3 degradation) reduces dCas9-KRAB expression. The system can also be used to show that STM2457, a METTL3 inhibitor, reduces dCas9-KRAB expression when the system is expressed in wildtype cells. Finally, experiments can be done to show that METTL3 overexpression increases dCas9-KRAB expression. [0218] gRNAs that target other genes of interest (e.g., oncogenes) can be evaluated using this system. It is understood that any gene in the genome of a cell can be targeted by the dCas9-KRAB effector protein, as long as one or more gRNA guide the dCas9-KRAB to the gene of interest.

Any polypeptide can be expressed in a cell by using the m⁶A-coupled effector protein expression system. Other proteins of interest include, but are not limited to SOCS2 and other tumor suppressors, which can be expressed in cancer cells to determine if expression of a tumor suppressor can reduce cancer cell proliferation, migration, and colony formation.

dCas13

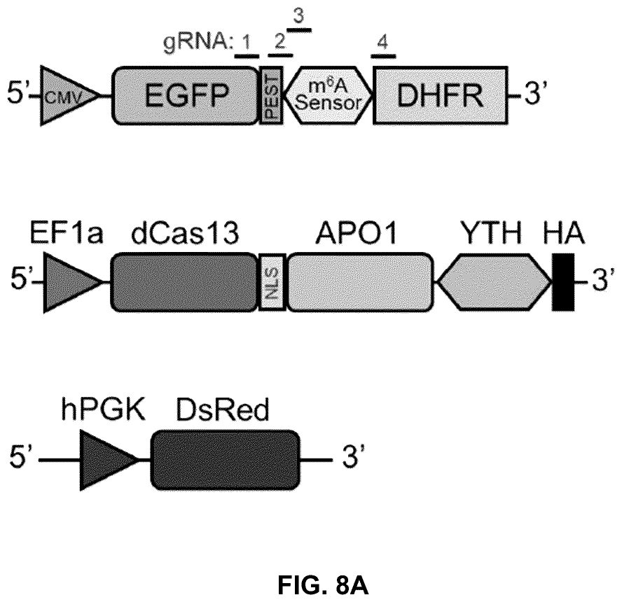

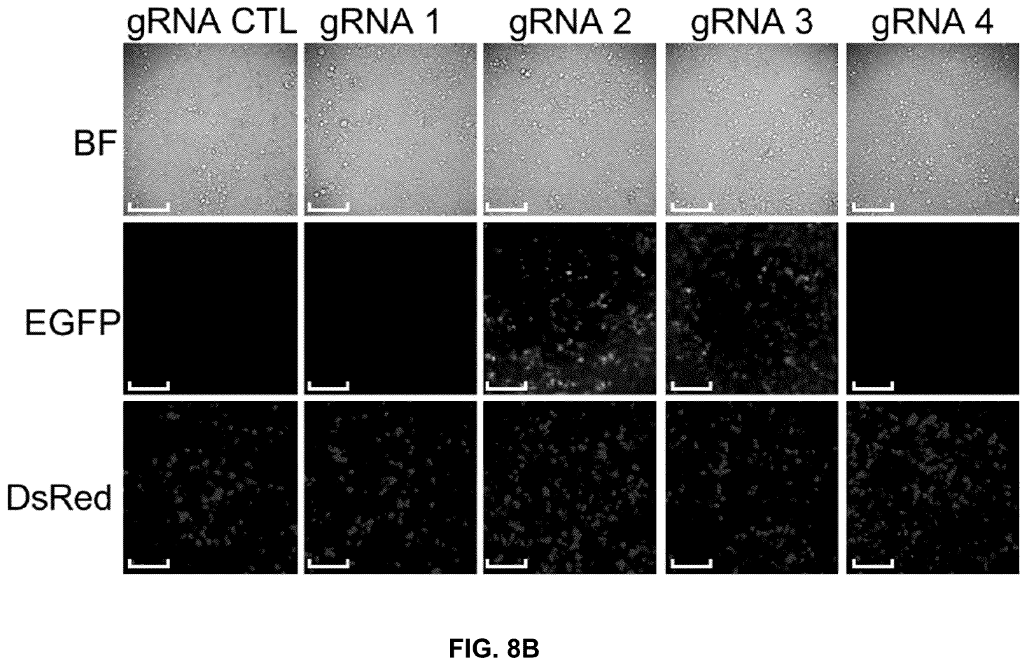

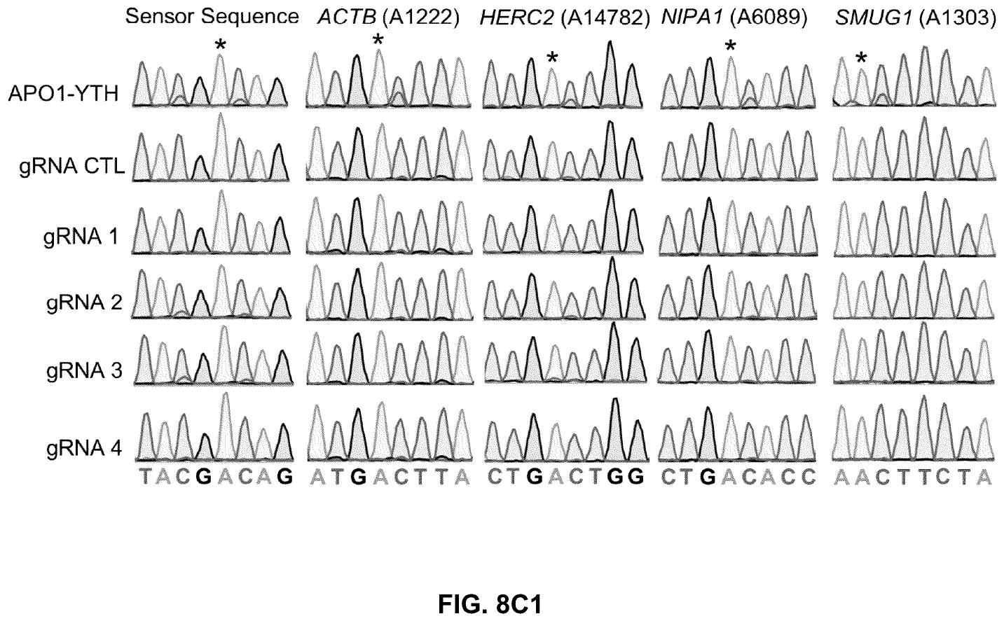

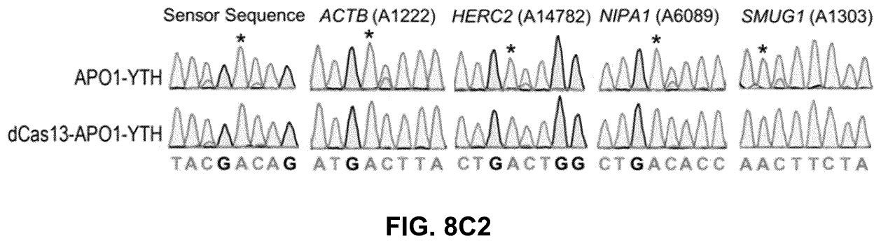

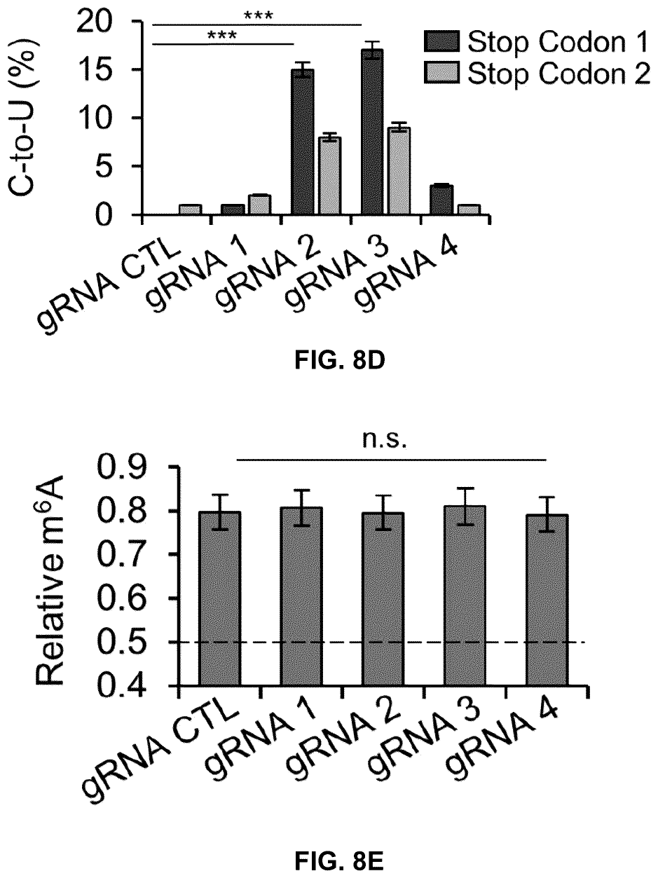

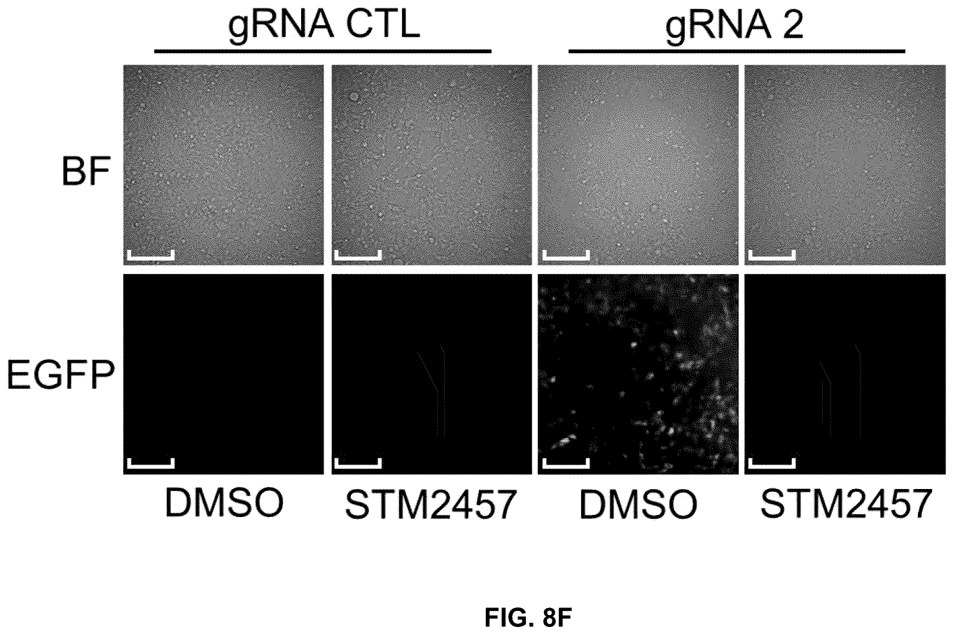

Previous studies have shown that dCas13 can be tethered to the m⁶A methyltransferase machinery and coupled with guide RNA (gRNA)-mediated targeting to achieve methylation of cellular mRNAs of interest³³. A similar approach of fusing dCas13 to APO1-YTH might enable targeted m⁶A recognition and C-to-U editing of the sensor sequence in the GEMS reporter mRNA. Thus, APO1-YTH in the GEMS system was replaced with dCas13-APO1-YTH and co-expressed this in cells together with a gRNA targeting the m⁶A sensor sequence (FIG. 8A). This led to EGFP expression and m⁶A sensor sequence editing which was strongest for gRNAs binding closest to the sensor sequence adenosines (FIGS. 8B-8D). In contrast, cells co-expressing a non-targeting gRNA were dark and had no sensor sequence editing despite unchanged sensor sequence methylation (FIGS. 8B-8E). Additionally, treatment with STM2457 abolished GEMS activity, indicating that dCas13-APO1-YTH retains its requirement for recognizing m⁶A-modification of the reporter mRNA (FIG. 8F). Importantly, replacing APO1-YTH with dCas13-tethered APO1-YTH did not cause C-to-U editing of methylated cellular mRNAs (FIGS. 8C1-8C2). Altogether, these data show that dCas13-fused APO1-YTH can be used in the GEMS system together with a reporter mRNA-targeting gRNA to achieve m⁶A-dependent sensor sequence editing and GEMS activation while reducing editing of endogenous cellular mRNAs.

### Example 7: m⁶A-Coupled Effector Protein Delivery Slows Cancer Cell Growth

The m⁶A-coupled payload delivery system can be used to influence cellular function. METTL3 expression is elevated in several cancers, and m⁶A hypermethylation has been shown to promote cancer cell proliferation and tumorigenesis⁴. Although pharmacological inhibition of METTL3 is a promising strategy for counteracting the effects of hypermethylation of transcripts associated with cancer progression, such approaches may have unwanted consequences because they also influence methylation of other RNAs in the cell. Since the GEMS system can couple protein expression with m⁶A methylation, it could replace EGFP with effector proteins of interest to overcome the oncogenic effects of mRNA hypermethylation in cancer cells.

A protein expression system described herein can be tested by infecting Huh-7 cells with a lentivirus expressing the system (or other means of introducing exogenous polynucleotides into a cell, for example, lipofection, nucleofection, or electroporation). The GFP-expressing sensor system can be used as a control, with both systems including APO1-YTH under an inducible promoter. Cells can be treated with doxycycline, and cell proliferation and colony formation will be tested over the course of 72 hours using established protocols (Chen et al.). METTL3 inhibition with STM2457 will be used in wildtype cells as a control to confirm METTL3-dependent effects on proliferation and colony formation.

The m⁶A feedback system can be tested using a similar approach with Huh-7 cells as well as MOLM-13 cells, an AML cell line with high levels of METTL3 that exhibits reduced proliferation and colony formation in response to STM2457. Thus, in both cell types, it is expected that expression of the m⁶A feedback system can lead to high levels of m⁶A sensor sequence methylation and editing. Effector protein or other therapeutic delivery can lead to METTL3 transcription inhibition, reduced cell proliferation and colony formation as the cell cycles from high to low m⁶A.

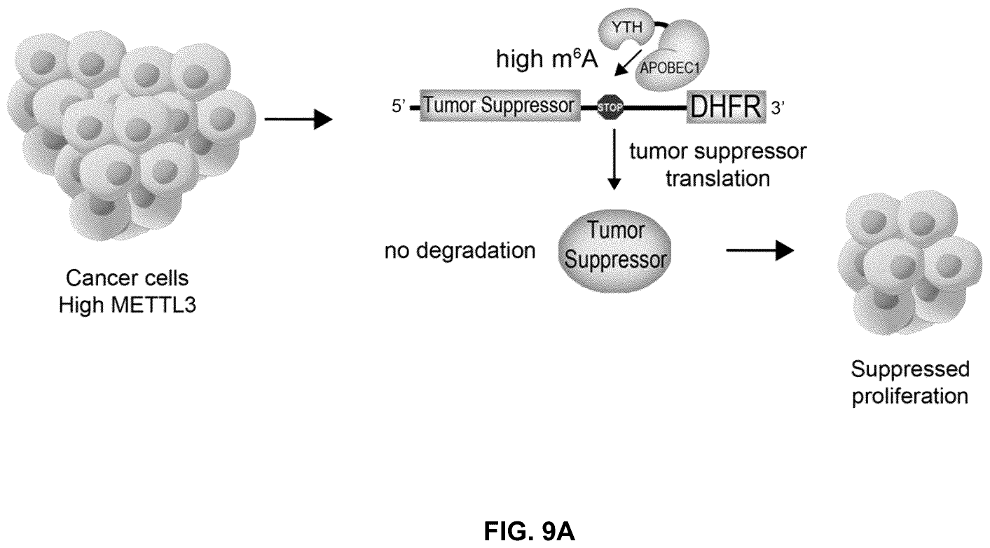

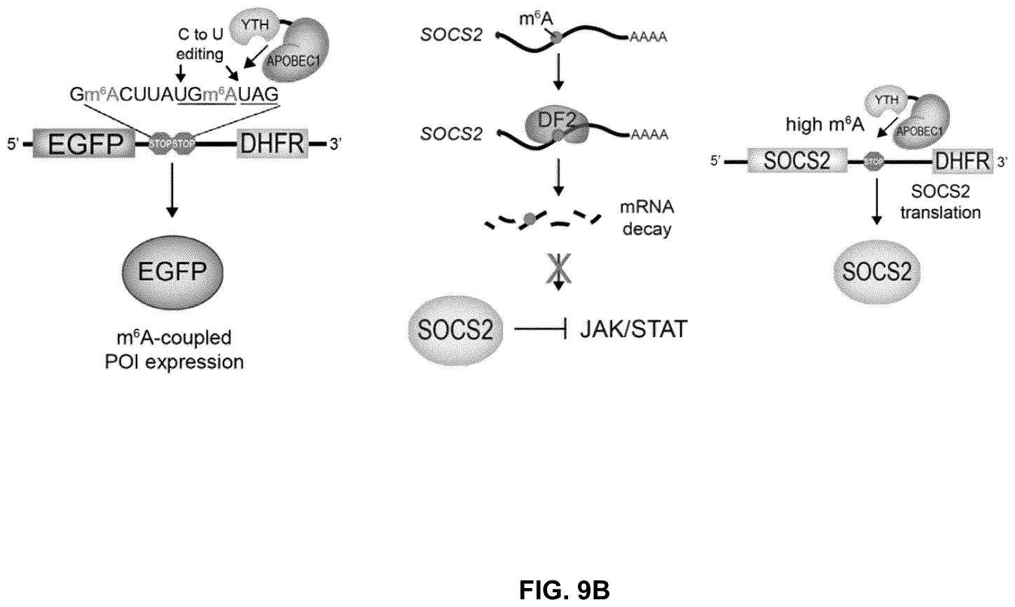

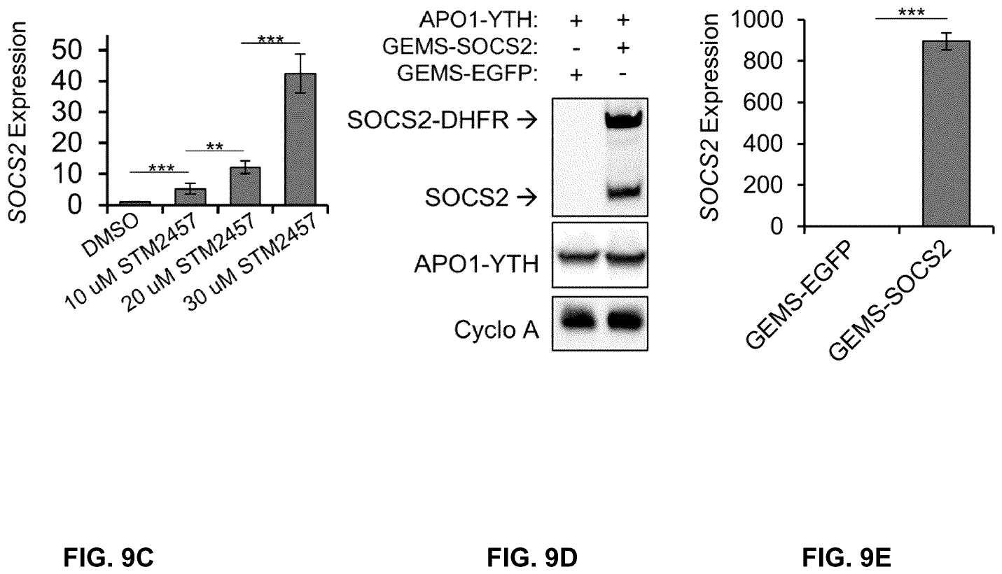

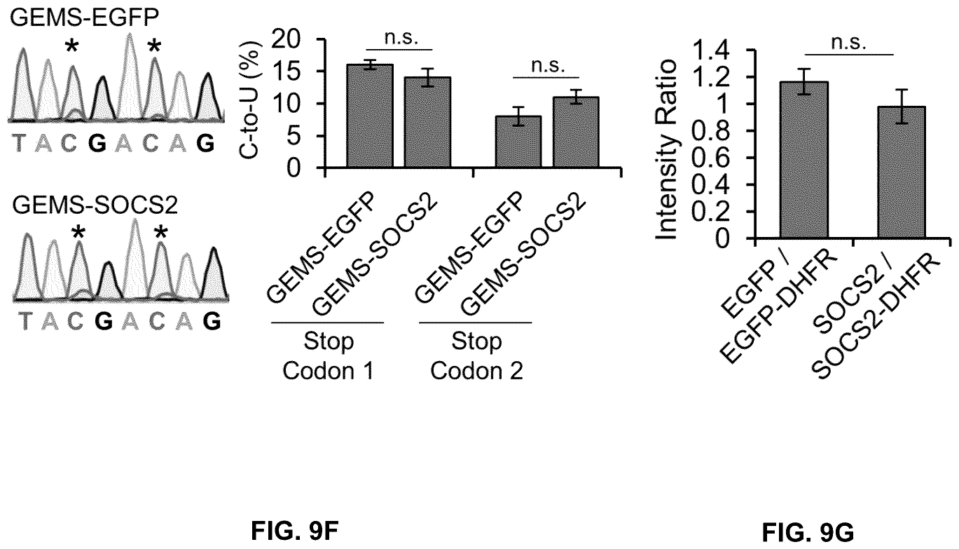

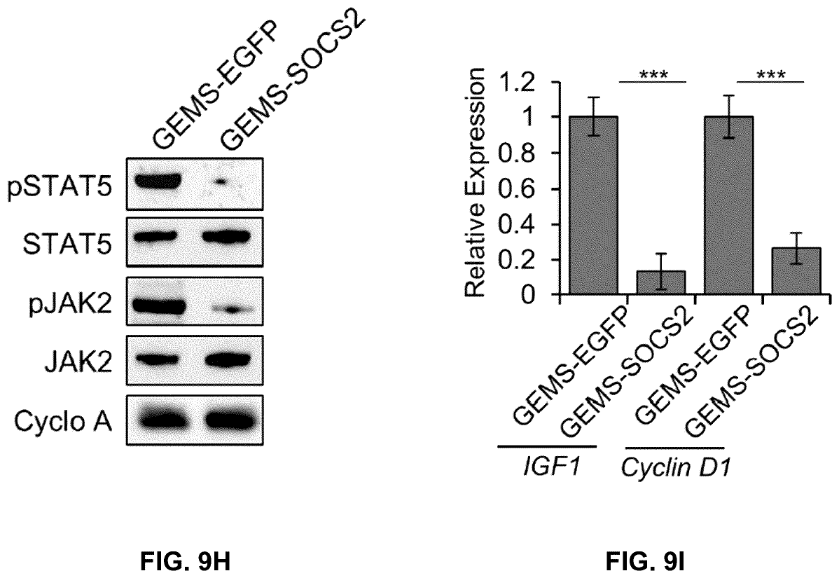

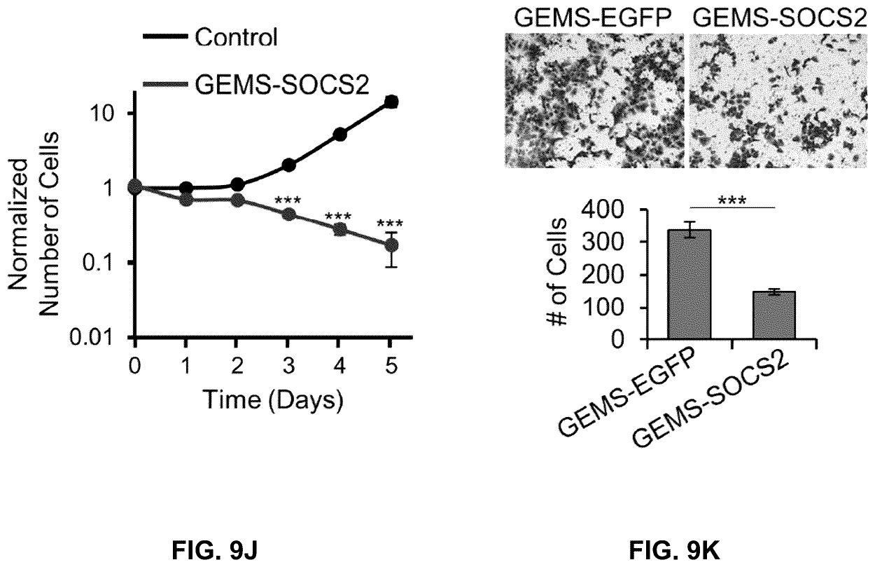

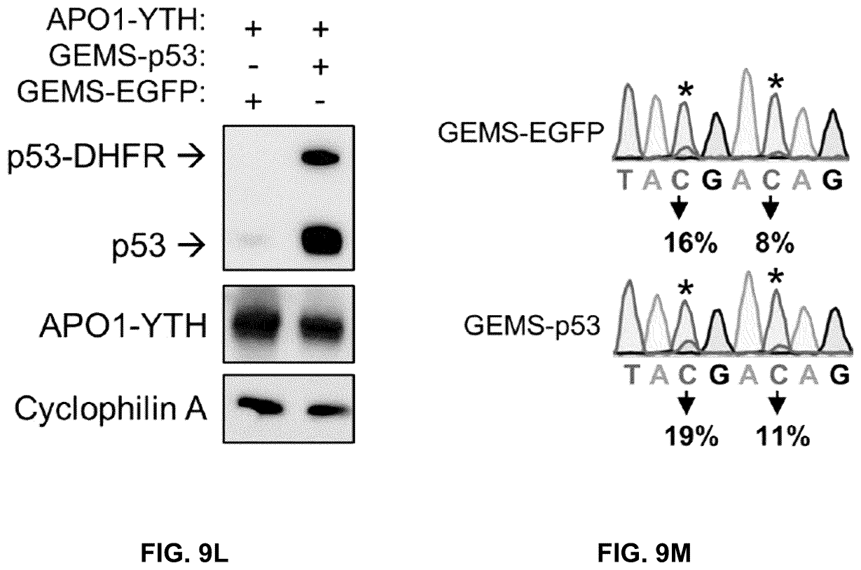

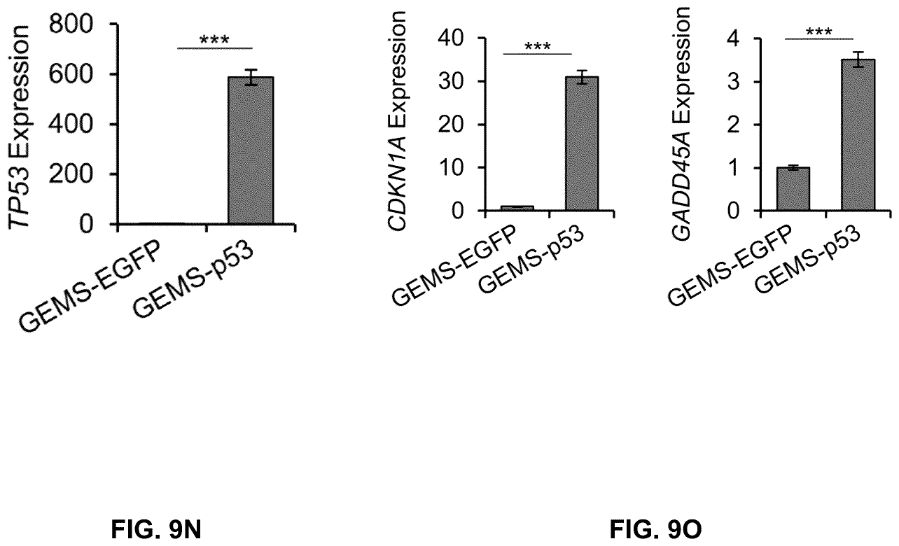

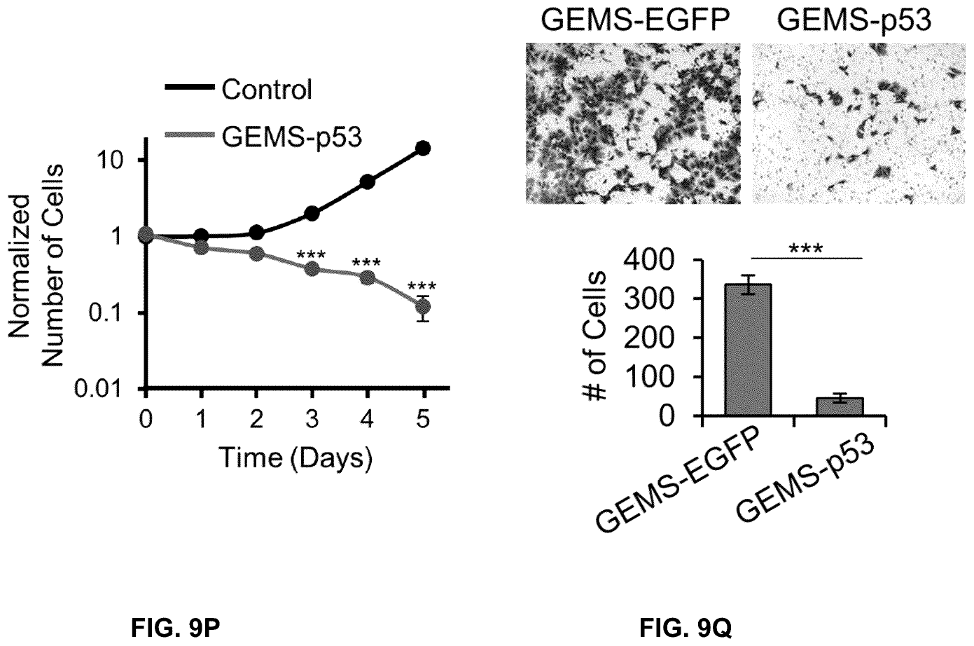

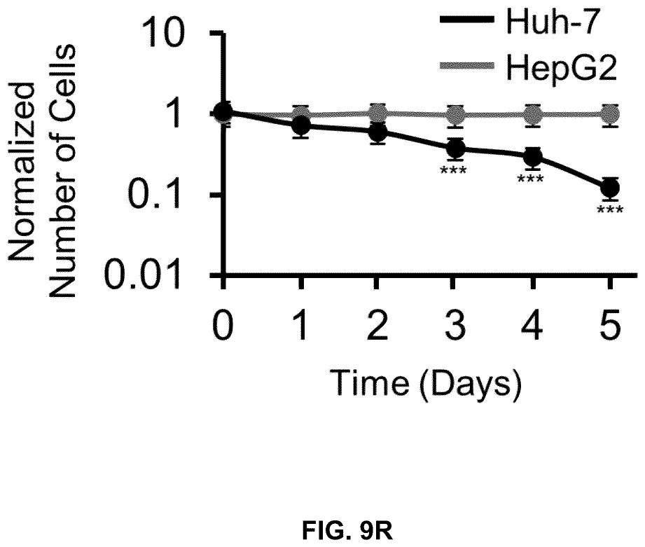

Huh-7 cells are a hepatocyte-derived carcinoma cell line frequently used to model hepatocellular carcinoma (HCC). Previous studies have shown that METTL3 and other methyltransferase complex components are upregulated in HCC and associated with increased disease severity and cancer progression^{38,39}. One mechanism for this is through hypermethylation of the SOCS2 mRNA, which acts as a tumor suppressor in HCC^{40, 41} Elevated m⁶A methylation of SOCS2 promotes its degradation and reduces SOCS2 protein levels to accelerate cancer cell growth^{36, 42}. This system was chosen because METTL3-induced hypermethylation of SOCS2 mRNA leads to m⁶A-dependent transcript degradation and a reduction in SOCS2 protein, which in turn promotes HCC cell proliferation, migration, and colony formation (Chen et al. RNA N6-methyladenosine methyltransferase-like 3 promotes liver cancer progression through YTHDF2-dependent posttranscriptional silencing of SOCS2,” Hepatology. 2018; 67(6):2254-70.) One could use the GEMS system to couple cellular m⁶A levels with SOCS2 protein expression and effectively rescue the loss of SOCS2 protein that is caused by hypermethylation of the SOCS2 mRNA (FIGS. 9A-9B).

To test this, the EGFP sequence in the GEMS reporter mRNA was replaced with the coding sequence for SOCS2. Expression of GEMS-SOCS2 in Huh-7 cells led to robust expression of SOCS2 protein and m⁶A sensor sequence editing (FIGS. 9C-9F). Importantly, expression of the GEMS-SOCS2 system in Huh-7 cells led to similar levels of sensor sequence editing and stop codon usage as GEMS-EGFP, indicating that the EGFP coding sequence can be effectively replaced with other sequences without compromising the ability of the system to sense m⁶A (FIGS. 9F-9G).

To confirm activity of the GEMS-delivered SOCS2 protein, the JAK-STAT signaling pathway, which is inhibited by SOCS family proteins⁴³⁻⁴⁵, was examined. Reduced levels of phosphorylated JAK2 and STAT5 were observed; both JAK2 and STAT5 are known targets of SOCS2-mediated inhibition 45 (FIG. 9H). Additionally, significant downregulation of IGF1 and CyclinD1 genes was observed in GEMS-SOCS2-expressing cells, consistent with the role of SOCS2 in negatively regulating IGF1 and CyclinD1 transcription⁴⁶⁻⁴⁸ (FIG. 9I).

Previous studies have shown that m⁶A-mediated SOCS2 depletion promotes cancer cell proliferation and migration^{40, 41} To determine whether GEMS-SOCS2 expression can reverse these effects, Huh-7 cells were transfected with GEMS-SOCS2 and measured cell growth over the course of 5 days. Expression of GEMS-SOCS2 significantly reduced Huh-7 cell growth, indicating that SOCS2 delivery with the GEMS system can counteract the effects of m⁶A hypermethylation on cancer cell proliferation (FIG. 9J). Additionally, Huh-7 cells transfected with GEMS-SOCS2 exhibit reduced migration capacity compared to cells expressing GEMS-EGFP (FIG. 9K). Thus, m⁶A-coupled delivery of SOCS2 overcomes the effects of SOCS2 hypermethylation and slows cancer cell growth and migration.

In other embodiments, The SOCS2 coding sequence can be cloned in place of GFP in the m⁶A sensor system described above using a lentiviral backbone. Huh-7 cells can then be infected with the system. APO1-YTH expression can be induced with doxycycline treatment. RNA and protein will be collected at various timepoints over the course of 72 hours. Sensor sequence methylation and editing can be measured with SELECT and Sanger sequencing, respectively, and SOCS2/SOCS2-DIFR and APO1-YTH levels can be assessed by Western blot. These studies can establish the timing and amount of SOCS2 protein expression that can be achieved by the system. To confirm that SOCS2 expression is m6A-dependent, the experiments can be repeated in cells treated with STM2457 to inhibit METTL3. Expression of the GFP-encoding m⁶A sensor system can be used in parallel as a control.

Next, the GEMS system was analyzed as a possible general strategy to deliver tumor suppressors to inhibit cancer cell growth. The tumor suppressor protein p53 regulates transcriptional programs involved in cell cycle arrest, apoptosis, and DNA repair and plays a critical role in the prevention of cancer progression^{49, 50}. Consistent with this, the TP53 gene is mutated in nearly half of human cancers^{51, 52} Huh-7 cells express mutated p53 (Y220C) which is stable but has impaired DNA binding and transcriptional activity^{53, 54}. Therefore, the wild type TP53 coding sequence was cloned into the GEMS system and introduced it into Huh-7 cells. This led to robust p53 expression and upregulation of downstream p53 transcription targets, including CDKN1A and GADD45A (FIGS. 9L-90). Expression of GEMS-p53 also slowed Huh-7 cell growth and migration (FIGS. 9P-9Q). Additionally, when the effect of GEMS-p53 expression on the growth of HepG2 cells (which do not harbor TP53 mutations⁵⁵), was tested, it was found that the growth reduction was specific to Huh-7 cells (FIG. 9R). Overall, these data demonstrate that the GEMS system can be used to couple m⁶A methylation with the expression of tumor suppressor proteins to slow cancer cell growth and migration.

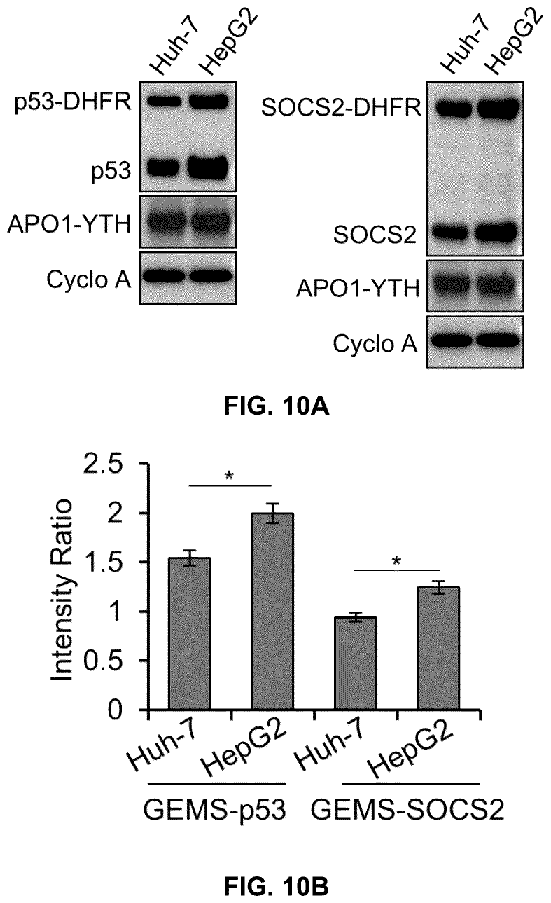

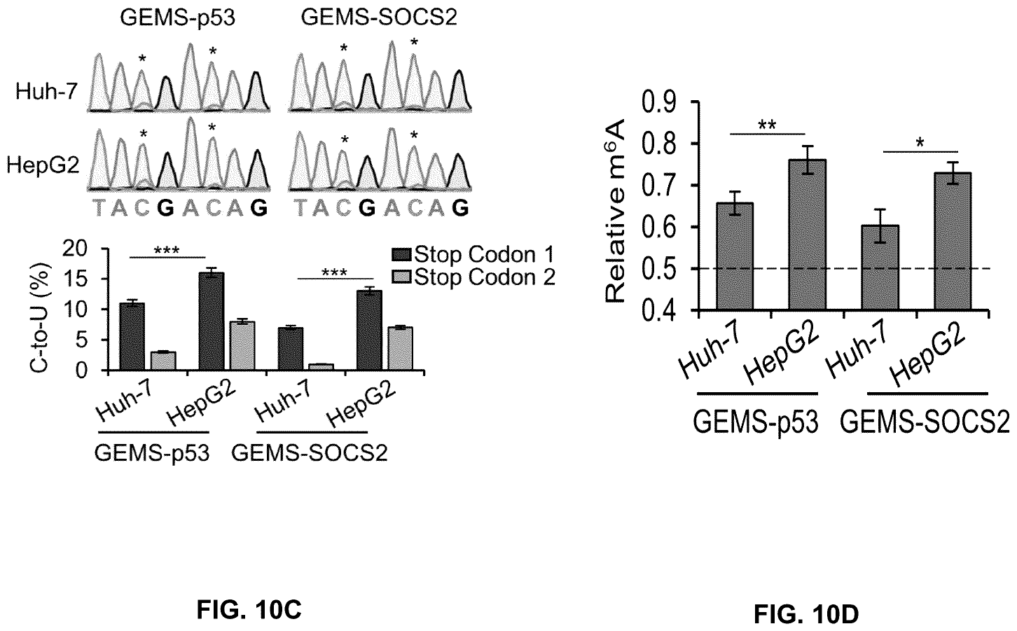

A major advantage of the GEMS platform is that it enables protein output to be tuned to m⁶A levels. Indeed, the amounts of SOCS2 and p53 protein delivered by the GEMS system were compared in HepG2 and Huh-7 cells—elevated levels of both proteins were found in HepG2 cells, which have higher m6A⁴¹ (FIGS. 10A-10D). In theory, the system can be programmed to deliver any effector protein of interest in an m⁶A-dependent manner, making it an attractive strategy for tuning gene expression in a highly targeted manner in response to mRNA methylation levels.

### Discussion

Disclosed herein is a genetically encoded m⁶A sensor system which provides a fluorescent readout in cells when m⁶A is deposited on mRNA. This disclosure offers a simple, low-cost method for cellular m⁶A sensing which can be implemented in virtually any cell or tissue type and easily carried out by a standard molecular biology lab. The ability of GEMS to sense changes in m⁶A methylation in living cells makes it an attractive system for monitoring m⁶A dynamics in a variety of cell types and conditions. Indeed, as disclosed herein, GEMS can be used as a readout for m⁶A in a variety of mouse and human cell lines and that relative differences in EGFP reporter fluorescence can be used to identify differences in methylation levels between cell types. GEMS may also have wide utility for studies of m⁶A dynamics in cells. Since the sensitivity of GEMS for reporting changes in mRNA methylation depends in part on the half-life of EGFP, using a reporter protein with a short half-life will improve the sensitivity of GEMS for sensing dynamic regulation of m⁶A. Consistent with this, as discussed above, adding a PEST sequence to EGFP substantially reduces sensor protein longevity and improves the ability to detect changes in m⁶A caused by pharmacological inhibition of METTL3. Depending on the application, photoconvertible proteins or other reporter proteins could also be substituted for EGFP to further improve detection of m⁶A dynamics.

Also, GEMS may be utilized for in vivo monitoring of m⁶A. This could be achieved either through the generation of transgenic animals expressing the two main components of the GEMS system or by introducing GEMS into a desired tissue of interest using viral-mediated or other delivery methods. Such studies might be useful for monitoring the in vivo effects of m⁶A methylation inhibitors, for examining how certain conditions or stresses alter m⁶A, or for understanding tissue-specific differences in methylation.

Due to its simple design and ability to sense m⁶A in living cells, the GEMS system may be useful for a variety of HTS-based approaches. For instance, the factors that control m⁶A methylation in cells are still not completely understood, so GEMS may be useful for global knockout screens designed to identify cellular proteins that influence m⁶A. Additionally, GEMS will be highly enabling for drug discovery efforts, as it provides a simple method for screening drug or small molecule libraries to identify novel inhibitors of METTL3. Other methyltransferase complex proteins such as METTL14 and WTAP have also been implicated in human disease and are upregulated in several cancers^{56, 57} so such screens have the potential to uncover inhibitors of these proteins as well.

Although GEMS opens up several new avenues for both low- and high-throughput studies of m⁶A, there are some important considerations when using the system. For instance, factors that influence proteasomal degradation could impact EGFP-DHFR stability and therefore cellular fluorescence. Additionally, changes in transcription or translation rates could influence FP production, although the use of m⁶A-uncoupled internal reporters such as DsRed can help mitigate this. Lastly, since GEMS requires APO1-YTH expression, factors that influence the fusion protein's activity or m⁶A recognition could impact the system. APO1-YTH also edits cellular methylated RNAs in addition to the GEMS reporter mRNA, which could influence other processes in the cell. Importantly, as discussed above, tethering APO1-YTH to dCas13 enables targeted editing of the GEMS reporter mRNA and reduces editing of cellular mRNAs. Additional refining of the GEMS system based on this approach may further improve its functionality by limiting unwanted effects of APO1-YTH-mediated editing of endogenous methylated RNAs.

In addition to its utility as an m⁶A-coupled fluorescent reporter, the GEMS system can be programmed to deliver protein payloads of interest in an m⁶A-dependent manner. As discussed above, GEMS may be used to express SOCS2 and p53 in liver cancer cells, leading to slowed cell growth and reduced migration capacity. Thus, the GEMS system can be used both to rescue the expression of proteins whose production is decreased by mRNA hypermethylation, as in the case of SOCS2, or as a general strategy for tumor suppressor protein expression in cells with elevated m⁶A, as with p53. In theory, any protein of interest can be expressed using the GEMS system, opening up numerous possibilities for m⁶A-coupled effector protein expression as a means of achieving desired cellular outcomes or counteracting the effects of high or low levels of m⁶A. For instance, GEMS could be used to deliver CRISPR/Cas9 tools targeting METTL3 itself, which could be used to activate or inhibit METTL3 expression in response to changing levels of m⁶A and therefore maintain m⁶A homeostasis. Given the numerous associations between m⁶A dysregulation and human disease, the GEMS system has potential utility as a novel therapeutic strategy.

### Example 8: System-Expressing Cells from Transgenic/Knock-In Animals

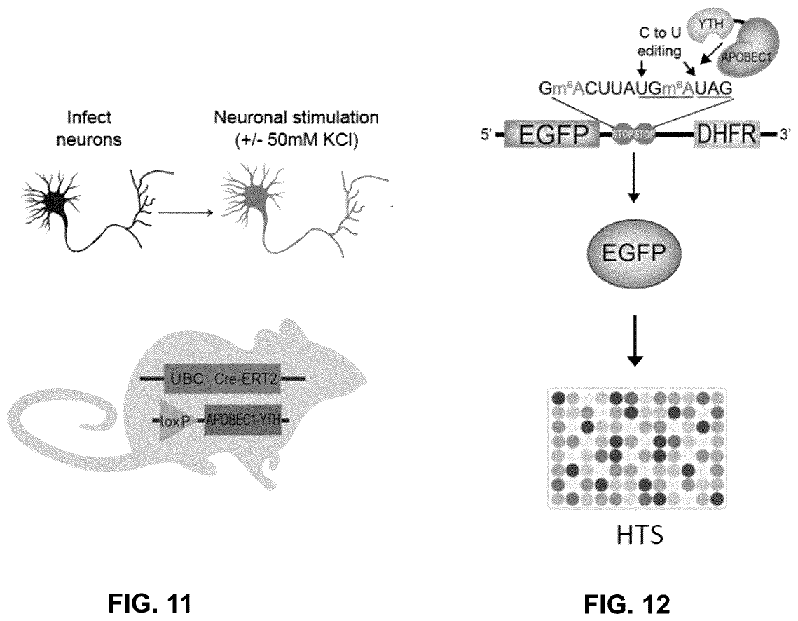

FIG. 11 shows an embodiment of host cells as described herein and methods of use. Briefly, primary neurons can be isolated from transgenic mice expressing the APOBEC1-YTH enzyme. The cells can be cultured and then the m6A reporter mRNA could be introduced with viral infection or other means to examine m6A dynamics. Alternatively, mice that express the GEMS system can be created, and neurons (or other cells) isolated and cultured utilizing methods known in the art.

### Example 9: GEMS Utilization for HTS Applications

FIG. 12: the GEMS system is compatible with HTS, so it could be used for HTS studies such as those seeking to identify cellular proteins/pathways that control m⁶A abundance or drugs/small molecules that inhibit METTL3 or m6A demethylases. As shown in FIG. 12, the expression system could be introduced to cell of choice, plated, and cultured m⁶A abundance or drugs/small molecules that inhibit METTL3 or m6A demethylases could then be studied using reporters, such as fluorescent reporters and measured using plate readers as known in the art. Below is a list of references relevant to this disclosure.
1. Zaccara, S., Ries, R. J., and Jaffrey, S. R. (2019). Reading, writing and erasing mRNA methylation. Nat. Rev. Mol. Cell Biol. 20. 10.1038/s41580-019-0168-5.
2. Shi, H., Wei, J., and He, C. (2019). Where, When, and How: Context-Dependent Functions of RNA Methylation Writers, Readers, and Erasers. Mol. Cell 74. 10.1016/j.molcel.2019.04.025.
3. Meyer, K. D., Saletore, Y., Zumbo, P., Elemento, O., Mason, C. E., and Jaffrey, S. R. (2012). Comprehensive analysis of mRNA methylation reveals enrichment in 3′ UTRs and near stop codons. Cell. 10.1016/j.cell.2012.05.003.
4. Dominissini, D., Moshitch-Moshkovitz, S., Schwartz, S., Salmon-Divon, M., Ungar, L., Osenberg, S., Cesarkas, K., Jacob-Hirsch, J., Amariglio, N., Kupiec, M., et al. (2012). Topology of the human and mouse m6A RNA methylomes revealed by m6A-seq. Nature. 10.1038/naturel 1112.
5. Flamand, M. N., and Meyer, K. D. (2019). The epitranscriptome and synaptic plasticity. Curr. Opin. Neurobiol. 10.1016/j.conb.2019.04.007.
6. Roundtree, I. A., Evans, M. E., Pan, T., and He, C. (2017). Dynamic RNA Modifications in Gene Expression Regulation. Cell 169. 10.1016/j.cell.2017.05.045.
7. Meyer, K. D., and Jaffrey, S. R. (2017). Rethinking m6A readers, writers, and erasers. Annu. Rev. Cell Dev. Biol. 33. 10.1146/annurev-cellbio-100616-060758.
8. Meyer, K. D., Patil, D. P., Zhou, J., Zinoviev, A., Skabkin, M. A., Elemento, O., Pestova, T. V., Qian, S. B., and Jaffrey, S. R. (2015). 5′ UTR m6A Promotes Cap-Independent Translation. Cell. 10.1016/j.cell.2015.10.012.
9. Ries, R. J., Zaccara, S., Klein, P., Olarerin-George, A., Namkoong, S., Pickering, B. F., Patil, D. P., Kwak, H., Lee, J. H., and Jaffrey, S. R. (2019). m6A enhances the phase separation potential of mRNA. Nature 571. 10.1038/s41586-019-1374-1.
10. Zhou, J., Wan, J., Shu, X. E., Mao, Y., Liu, X. M., Yuan, X., Zhang, X., Hess, M. E., Brüning, J. C., and Qian, S. B. (2018). N6-Methyladenosine Guides mRNA Alternative Translation during Integrated Stress Response. Mol. Cell 69. 10.1016/j.molcel.2018.01.019.
11. Engel, M., Eggert, C., Kaplick, P. M., Eder, M., Röh, S., Tietze, L., Namendorf, C., Arloth, J., Weber, P., Rex-Haffner, M., et al. (2018). The Role of m6A/m-RNA Methylation in Stress Response Regulation. Neuron 99. 10.1016/j.neuron.2018.07.009.
12. Widagdo, J., Zhao, Q. Y., Kempen, M. J., Tan, M. C., Ratnu, V. S., Wei, W., Leighton, L., Spadaro, P. A., Edson, J., Anggono, V., et al. (2016). Experience-dependent accumulation of N6-methyladenosine in the prefrontal cortex is associated with memory processes in mice. J. Neurosci. 36. 10.1523/JNEUROSCI.4053-15.2016.
13. Zhang, Z., Wang, M., Xie, D., Huang, Z., Zhang, L., Yang, Y., Ma, D., Li, W., Zhou, Q., Yang, Y. G., et al. (2018). METTL3-mediated N 6-methyladenosine mRNA modification enhances long-term memory consolidation. Cell Res. 28. 10.1038/s41422-018-0092-9.
14. Dai, D., Wang, H., Zhu, L., Jin, H., and Wang, X. (2018). N6-methyladenosine links RNA metabolism to cancer progression review-article. Cell Death Dis. 9. 10.1038/s41419-017-0129-x.
15. Jiang, X., Liu, B., Nie, Z., Duan, L., Xiong, Q., Jin, Z., Yang, C., and Chen, Y. (2021). The role of m6A modification in the biological functions and diseases. Signal Transduct. Target. Ther. 6. 10.1038/s41392-020-00450-x.
16. Williams, G. D., Gokhale, N. S., and Homer, S. M. (2019). Regulation of Viral Infection by the RNA Modification N6-Methyladenosine. Annu. Rev. Virol. 6. 10.1146/annurev-virology-092818-015559.
17. Garcia-Campos, M. A., Edelheit, S., Toth, U., Safra, M., Shachar, R., Viukov, S., Winkler, R., Nir, R., Lasman, L., Brandis, A., et al. (2019). Deciphering the “m6A Code” via Antibody-Independent Quantitative Profiling. Cell 178. 10.1016/j.cell.2019.06.013.
18. Zhang, Z., Chen, L. Q., Zhao, Y. L., Yang, C. G., Roundtree, I. A., Zhang, Z., Ren, J., Xie, W., He, C., and Luo, G. Z. (2019). Single-base mapping of m6A by an antibody-independent method. Sci. Adv. 5. 10.1126/sciadv.aax0250.
19. Liu, C., Sun, H., Yi, Y., Shen, W., Li, K., Xiao, Y., Li, F., Li, Y., Hou, Y., and Lu, B. (2022). Absolute quantification of single-base m6A methylation in the mammalian transcriptome using GLORI. Nat. Biotechnol., 1-12.
20. Meyer, K. D. (2019). DART-seq: an antibody-free method for global m6A detection. Nat. Methods. 10.1038/s41592-019-0570-0.
21. Hu, L., Liu, S., Peng, Y., Ge, R., Su, R., Senevirathne, C., Harada, B. T., Dai, Q., Wei, J., Zhang, L., et al. (2022). m6A RNA modifications are measured at single-base resolution across the mammalian transcriptome. Nat. Biotechnol. 40. 10.1038/s41587-022-01243-z.
22. Liu, N., Parisien, M., Dai, Q., Zheng, G., He, C., and Pan, T. (2013). Probing N6-methyladenosine RNA modification status at single nucleotide resolution in mRNA and long noncoding RNA. RNA 19. 10.1261/rna.041178.113.
23. Linder, B., Grozhik, A. V., Olarerin-George, A. O., Meydan, C., Mason, C. E., and Jaffrey, S. R. (2015). Single-nucleotide-resolution mapping of m6A and m6Am throughout the transcriptome. Nat. Methods. 10.1038/nmeth.3453.
24. Ke, S., Alemu, E. A., Mertens, C., Gantman, E. C., Fak, J. J., Mele, A., Haripa, B., Zucker-Scharff, I., Moore, M. J., Park, C. Y., et al. (2015). A majority of m6A residues are in the last exons, allowing the potential for 3′ UTR regulation. Genes Dev. 29.
25. Iwamoto, M., Bjorklund, T., Lundberg, C., Kirik, D., and Wandless, T. J. (2010). A general chemical method to regulate protein stability in the mammalian central nervous system. Chem. Biol. 10.1016/j.chembiol.2010.07.009.
26. Tegowski, M., Flamand, M. N., and Meyer, K. D. (2022). scDART-seq reveals distinct m6A signatures and mRNA methylation heterogeneity in single cells. Mol. Cell 82. 10.1016/j.molcel.2021.12.038.
27. Xiao, Y.-L., Liu, S., Ge, R., Wu, Y., He, C., Chen, M., and Tang, W. (2023). Transcriptome-wide profiling and quantification of N6-methyladenosine by enzyme-assisted adenosine deamination. Nat. Biotechnol., 1-11.
28. Castellanos-Rubio, A., Santin, I., Olazagoitia-Garmendia, A., Romero-Garmendia, I., Jauregi-Miguel, A., Legarda, M., and Bilbao, J. R. (2019). A novel RT-QPCR-based assay for the relative quantification of residue specific m6A RNA methylation. Sci. Rep. 9. 10.1038/s41598-019-40018-6.
29. Yankova, E., Blackaby, W., Albertella, M., Rak, J., De Braekeleer, E., Tsagkogeorga, G., Pilka, E. S., Aspris, D., Leggate, D., Hendrick, A. G., et al. (2021). Small-molecule inhibition of METTL3 as a strategy against myeloid leukaemia. Nature. 10.1038/s41586-021-03536-w.
30. Cui, Q., Shi, H., Ye, P., Li, L., Qu, Q., Sun, G., Sun, G., Lu, Z., Huang, Y., Yang, C. G., et al. (2017). m6A RNA Methylation Regulates the Self-Renewal and Tumorigenesis of Glioblastoma Stem Cells. Cell Rep. 18. 10.1016/j.celrep.2017.02.059.
31. Tzelepis, K., De Braekeleer, E., Yankova, E., Rak, J., Aspris, D., Domingues, A. F., Fosbeary, R., Hendrick, A., Leggate, D., Ofir-Rosenfeld, Y., et al. (2019). Pharmacological Inhibition of the RNA m6a Writer METTL3 As a Novel Therapeutic Strategy for Acute Myeloid Leukemia. Blood 134. 10.1182/blood-2019-127962.
32. Huang, H., Weng, H., and Chen, J. (2020). m6A Modification in Coding and Non-coding RNAs: Roles and Therapeutic Implications in Cancer. Cancer Cell 37. 10.1016/j.ccell.2020.02.004.
33. Wilson, C., Chen, P. J., Miao, Z., and Liu, D. R. (2020). Programmable m6A modification of cellular RNAs with a Cas13-directed methyltransferase. Nat. Biotechnol. 10.1038/s41587-020-0572-6.
34. Lan, Q., Liu, P. Y., Haase, J., Bell, J. L., Hüttelmaier, S., and Liu, T. (2019). The critical role of RNA m6A methylation in cancer. Cancer Res. 79, 1285-1292.
35. Liu, J., Eckert, M. A., Harada, B. T., Liu, S.-M., Lu, Z., Yu, K., Tienda, S. M., Chryplewicz, A., Zhu, A. C., and Yang, Y. (2018). m6A mRNA methylation regulates AKT activity to promote the proliferation and tumorigenicity of endometrial cancer. Nat. Cell Biol. 20, 1074-1083.
36. Xu, J., Chen, Q., Tian, K., Liang, R., Chen, T., Gong, A., Mathy, N. W., Yu, T., and Chen, X. (2020). m6A methyltransferase METTL3 maintains colon cancer tumorigenicity by suppressing SOCS2 to promote cell proliferation. Oncol. Rep. 44, 973-986.
37. Pinello, N., Sun, S., and Wong, J. J.-L. (2018). Aberrant expression of enzymes regulating m6A mRNA methylation: implication in cancer. Cancer Biol. Med. 15, 323.
38. Lin, Y., Wei, X., Jian, Z., and Zhang, X. (2020). METTL3 expression is associated with glycolysis metabolism and sensitivity to glycolytic stress in hepatocellular carcinoma. Cancer Med. 9. 10.1002/cam4.2918.
39. Liu, X., Qin, J., Gao, T., Li, C., Chen, X., Zeng, K., Xu, M., He, B., Pan, B., Xu, X., et al. (2020). Analysis of METTL3 and METTL14 in hepatocellular carcinoma. Aging (Albany. NY). 12. 10.18632/aging.103959.
40. Liu, J., Liu, Z., Li, W., and Zhang, S. (2021). SOCS2 is a potential prognostic marker that suppresses the viability of hepatocellular carcinoma cells. Oncol. Lett. 21. 10.3892/OL.2021.12660.
41. Cui, M., Sun, J., Hou, J., Fang, T., Wang, X., Ge, C., Zhao, F., Chen, T., Xie, H., Cui, Y., et al. (2016). The suppressor of cytokine signaling 2 (SOCS2) inhibits tumor metastasis in hepatocellular carcinoma. Tumor Biol. 37. 10.1007/s13277-016-5215-7.
42. Chen, M., Wei, L., Law, C. T., Tsang, F. H. C., Shen, J., Cheng, C. L. H., Tsang, L. H., Ho, D. W. H., Chiu, D. K. C., Lee, J. M. F., et al. (2018). RNA N6-methyladenosine methyltransferase-like 3 promotes liver cancer progression through YTHDF2-dependent posttranscriptional silencing of SOCS2. Hepatology 67. 10.1002/hep.29683.
43. O'Shea, J. J., and Murray, P. J. (2008). Cytokine Signaling Modules in Inflammatory Responses. Immunity 28. 10.1016/j.immuni.2008.03.002.
44. O'Sullivan, L. A., Liongue, C., Lewis, R. S., Stephenson, S. E. M., and Ward, A. C. (2007). Cytokine receptor signaling through the Jak-Stat-Socs pathway in disease. Mol. Immunol. 801 44. 10.1016/j.molimm.2006.11.025.
45. Durham, G. A., Williams, J. J. L., Nasim, M. T., and Palmer, T. M. (2019). Targeting SOCS Proteins to Control JAK-STAT Signalling in Disease. Trends Pharmacol. Sci. 40. 10.1016/j.tips.2019.03.001.
46. Zhou, Y., Zhang, Z., Wang, N., Chen, J., Zhang, X., Guo, M., John Zhong, L., and Wang, Q. (2018). Suppressor of cytokine signalling-2 limits IGF1R-mediated regulation of epithelial-mesenchymal transition in lung adenocarcinoma article. Cell Death Dis. 9. 10.1038/s41419-018-0457-5.
47. Dobie, R., MacRae, V. E., Huesa, C., van′t Hof, R., Ahmed, S. F., and Farquharson, C. (2014). Direct stimulation of bone mass by increased GH signalling in the osteoblasts of Socs2−/− mice. J. Endocrinol. 223. 10.1530/JOE-14-0292.
48. Zhang, Y., Ma, Y., Wu, G., Xie, M., Luo, C., Huang, X., Tian, F., Chen, J., and Li, X. (2021). SENP1 promotes MCL pathogenesis through regulating JAK-STAT5 pathway and SOCS2 expression. Cell Death Discov. 7. 10.1038/s41420-021-00578-x.
49. Hu, J., Cao, J., Topatana, W., Juengpanich, S., Li, S., Zhang, B., Shen, J., Cai, L., Cai, X., and Chen, M. (2021). Targeting mutant p53 for cancer therapy: direct and indirect strategies. J. Hematol. Oncol. 14. 10.1186/s13045-021-01169-0.
50. Zhang, C., Liu, J., Xu, D., Zhang, T., Hu, W., and Feng, Z. (2020). Gain-of-function mutant p53 in cancer progression and therapy. J. Mol. Cell Biol. 12. 10.1093/jmcb/mjaa040.
51. Ozaki, T., and Nakagawara, A. (2011). Role of p53 in cell death and human cancers. Cancers (Basel). 3, 994-1013.
52. Hollstein, M., Sidransky, D., Vogelstein, B., and Harris, C. C. (1991). p53 mutations in human cancers. Science (80-.). 253, 49-53.
53. Bauer, M. R., Jones, R. N., Tareque, R. K., Springett, B., Dingler, F. A., Verduci, L., Patel, K. J., Fersht, A. R., Joerger, A. C., and Spencer, J. (2019). A structure-guided molecular chaperone approach for restoring the transcriptional activity of the p53 cancer mutant Y220C. Future Med. Chem. 11, 2491-2504.
54. Liu, X., Wilcken, R., Joerger, A. C., Chuckowree, I. S., Amin, J., Spencer, J., and Fersht, A. R. (2013). Small molecule induced reactivation of mutant p53 in cancer cells. Nucleic Acids Res. 41, 6034-6044.
55. Courtois, S., Verhaegh, G., North, S., Luciani, M.-G., Lassus, P., Hibner, U., Oren, M., and Hainaut, P. (2002). ΔN-p53, a natural isoform of p53 lacking the first transactivation domain, counteracts growth suppression by wild-type p53. Oncogene 21, 6722-6728. 10.1038/sj.onc.1205874.
56. Chen, X. Y., Zhang, J., and Zhu, J. S. (2019). The role of m6A RNA methylation in human cancer. Mol. Cancer 18. 10.1186/s12943-019-1033-z.
57. Guan, Q., Lin, H., Miao, L., Guo, H., Chen, Y., Zhuo, Z., and He, J. (2022). Functions, mechanisms, and therapeutic implications of METTL14 in human cancer. J. Hematol. Oncol. 15. 10.1186/s13045-022-01231-5.
58. Abràmoff, M. D., Magalhães, P. J., and Ram, S. J. (2004). Image processing with imageJ. Biophotonics Int. 11. 10.1201/9781420005615.ax4.
59. Kluesner, M. G., Nedveck, D. A., Lahr, W. S., Garbe, J. R., Abrahante, J. E., Webber, B. R., and Moriarity, B. S. (2018). EditR: a method to quantify base editing from Sanger sequencing. Cris. J. 1, 239-250.
60. Kuchipudi, S. V., Tellabati, M., Nelli, R. K., White, G. A., Perez, B. B., Sebastian, S., Slomka, M. J., Brookes, S. M., Brown, I. H., Dunham, S. P., et al. (2012). 18S rRNA is a reliable normalisation gene for real time PCR based on influenza virus infected cells. Virol. J. 9. 10.1186/1743-422X-9-230.

Additional aspects of sequences relating to polynucleotides and polypeptides as described herein and in Table 3 below can be found, for example, in PCT/US2022/079709, U.S. Pat. No. 11,680,109, and Meyer, K. D., “DART-seq: an antibody-free method for global m(6)A detection,” Nat Methods. 2019 December, 16(12):1275-1280 (published online Sep. 23, 2019); doi: 10.1038/s41592-019-0570-0, the entire contents of all of which (including sequence information and any supplemental information) are incorporated by reference in their entirety as fully set forth herein.

TABLE 3
INFORMAL SEQUENCE LISTING

| | Oligonucleotide, | | |
|---|---|---|---|
| SEQ | polypeptide, | | |
| ID | plasmid, or | | Description and |
| NO | sequence name | Sequence (5′ to 3′) | Notes |
| 1 | FM36_CMV5′UpseqFW | GAAGAATCTGCTTAGGGTTAGGCG | sequencing oligo at |
| | | | CMV promoter |
| | | | upstream of eGFP |
| 2 | FM37_DHFR3′seqFW | GAATTCCACGATGCTGATG | sequencing oligo at |
| | | | DHFR domain |
| | | | downstream of |
| | | | EGFP |
| 3 | FM38_APOBEC5′seqFW | CGAGCCCCATGAGTTTGAGG | sequencing oligo at |
| | | | 5′ end of APO1 |
| 4 | FM39_APOBEC3′seqFW | CCACAGCTGACATTCTTTACC | sequencing oligo at |
| | | | 3′ end of APO1 |
| 5 | FM40_YTH3′seqFW | CTACAAGCACACCACTTCC | sequencing oligo at |
| | | | 3′ end of YTH |
| | | | domain |
| | | | downstream of |
| | | | APO1 |
| 6 | FM65_TLCV2tempR2 | CAACTTACTTCTGACAACGATCGGAGG | sequencing oligo in |
| | | ACCGA | the middle of AMP |
| | | | resistance cassette |
| 7 | FM66_TLCV2tempF1 | TAGCTCCTTCGGTCCTCCGATCGTTGT | sequencing oligo in |
| | | CAGAA | the middle of AMP |
| | | | resistance cassette |
| 8 | FM87_ODCmid1 | AGAATAGACCGAGATAGGGTTGAGTG | sequencing oligo in |
| | | | the middle of F1 |
| | | | origin of |
| | | | replication cassette |
| 9 | FM88_ODCmid2 | CACTCAACCCTATCTCGGTCTATTCT | sequencing oligo in |
| | | | the middle of F1 |
| | | | origin of |
| | | | replication cassette |
| 10 | FM149_dsREDN | GTACTGGAACTGGGGGGACAG | sequencing oligo at |
| | | | 5′ end of dsRED- |
| | | | Express2 |
| 11 | FM150_hPGK_F | GTGTTCCGCATTCTGCAAG | sequencing oligo in |
| | | | hPGK promoter |
| | | | upstream of |
| | | | dsRED-Express2 |
| 12 | FM255_SOCS2ampFW | CCTCTAGAGCCGCCATGACC | sanger GEMS- |
| | | | SOCS2 (use with |
| | | | FM2-actm6aRV) |
| 13 | FM256_P53ampFW | CCCTCTAGAGCCGCCATG | sanger GEMS-p53 |
| | | | (use with FM2- |
| | | | actm6aRV) |
| 14 | FM1-actm6aFW | CAAGATCCGCCACAATATCGAGG | sanger GEMS- |
| | | | EGFP (use with |
| | | | FM2-actm6aRV) |
| 15 | FM2-actm6aRV | GTACTCGAGAGAATGCACAATGAGGCT | Used for amplicon |
| | | TATC | generation for |
| | | | sanger in GEMS- |
| | | | EGFP/SOCS2/p53 |
| 16 | ActinFwd | CAACACAGTGCTGTCTGGC | sanger ACTB |
| | | | A1222; Tegowski |
| | | | and Meyer (2022) |
| 17 | ActinRev | CAAGATGAGATTGGCATGGC | sanger ACTB |
| | | | A1222; Tegowski |
| | | | and Meyer (2022) |
| 18 | FM186_595FragFW | CCCTCTAGAGCCGCCATGACCCTGCGG | amplify SOCS2 |
| | | TGC | from cDNA for |
| | | | GEMS-SOCS2 |
| | | | cloning |
| 19 | FM187_595FragRV | CTGTCGTAAGTCCGCTACCTGGAATTT | amplify SOCS2 |
| | | ATATTCTTCCAAGTAATCTTTTAGT | from cDNA for |
| | | | GEMS-SOCS2 |
| | | | cloning |
| 20 | FM202_600FragFW | CCCTCTAGAGCCGCCATGGAGGAGCCG | amplify p53 from |
| | | C | cDNA for GEMS- |
| | | | SOCS2 cloning |
| 21 | FM203_600FragRV | CTGTCGTAAGTCCGCGTCTGAGTCAGG | amplify p53 from |
| | | C | cDNA for GEMS- |
| | | | SOCS2 cloning |
| 22 | FM59_repadj2RV | GAAAGGGTGTAACGCAACTG | m⁶A sensor |
| | | | sequence adjacent |
| | | | RAC site 2 for |
| | | | SELECT |
| 23 | FM60_repadj1RV | GAAAGGGTGTAACGCAACTGTCG | m6A sensor |
| | | | sequence adjacent |
| | | | RAC site 1 for |
| | | | SELECT |
| 24 | FM61_repnonadjRV | CTTCTCATTGGGATCTTTGCTC | nonadjacent for |
| | | | both m⁶A sensor |
| | | | sequence RAC |
| | | | sites for SELECT |
| 25 | FM62_qPCRFW | CCACAATATCGAGGATGGC | qPCR for both |
| | | | m⁶A sensor |
| | | | sequence RAC |
| | | | sites for SELECT |
| 26 | FM126_adjRep1625 | CGAGAAAGGGTGTAACGCAAC | m6A sensor |
| | | | sequence adjacent |
| | | | non-RAC site for |
| | | | SELECT |
| 27 | FM127_adjRep1620 | GGGTGTAACGCAACTGTCG | m⁶A sensor |
| | | | sequence adjacent |
| | | | non-RAC site for |
| | | | SELECT |
| 28 | ACTBqPCRFwd | CAGCAAGCAGGAGTATGACGAGTC | qPCR for ACTB |
| | | | A1222 for |
| | | | SELECT |
| 29 | ACTB_nonAdjRev | CATGCCAATCTCATCTTG | nonadjacent for |
| | | | ACTB A1222 for |
| | | | SELECT |
| 30 | ActinA1222_AdjRev | TTGTCAAGAAAGGGTGTAACGCAACTA | ACTB A1222 |
| | | AG | sequence adjacent |
| | | | RAC site for |
| | | | SELECT; |
| | | | Tegowski and |
| | | | Meyer (2022) |
| | | | (reference 26) |
| 31 | FM201_SOCS2FW | CTGTTAATGAAGCCAAAGAG | qPCR of SOCS2 |
| | | | expression |
| 32 | FM201_SOCS2RV | GCACATCTGAACATAGTAG | qPCR of SOCS2 |
| | | | expression |
| 33 | FM214_p53FW | CAGATGAAGCTCCCAGAATG | qPCR of p53 |
| | | | expression |
| 34 | FM215_p53RV | GTCCCAGAATGCAAGAAG | qPCR of p53 |
| | | | expression |
| 35 | FM233_CCND1FW | GCTGCGAAGTGGAAACCATC | qPCR of CyclinD1 |
| | | | expression |
| 36 | FM234_CCNDIRV | CCTCCTTCTGCACACATTTGAA | qPCR of CyclinD1 |
| | | | expression |
| 37 | FM239_IGF1FW | GCTCTTCAGTTCGTGTGTGGA | qPCR of IGF1 |
| | | | expression |
| 38 | FM240_IGF1RV | GCCTCCTTAGATCACAGCTCC | qPCR of IGF1 |
| | | | expression |
| 39 | FM259_P21FW | TGTCCGTCAGAACCCATGC | qPCR of p21 |
| | | | expression |
| 40 | FM260_P21RV | AAAGTCGAAGTTCCATCGCTC | qPCR of p21 |
| | | | expression |
| 41 | FM263_RRM2AFW | AGAGGCTCGCTGTTTCTATGG | qPCR of RRM2A |
| | | | expression |
| 42 | FM263_RRM2ARV | GCAAGGCCCAATCTGCTTTTT | qPCR of RRM2A |
| | | | expression |
| 43 | AK_22_M3F | tggatctgttgtgatatccgctacc | qPCR of METTL3 |
| | | | expression |
| 44 | AK_23_M3R | ctgcctgtgacccagaggaagag | qPCR of METTL3 |
| | | | expression |
| 45 | FM220_dsREDNfw | CTTCAAGGTGCACATGGAG | qPCR of dsRed- |
| | | | Express2 |
| | | | expression (use |
| | | | with |
| | | | FM149_dsREDN) |
| 46 | HZ_SMUG1_F | TTGACACTACTCCTGTTTGCCC | sanger sequencing |
| | | | of SMUG1 m⁶A |
| | | | site |
| 47 | HZ_SMUG1_R | AAAAGTTCCAAGTTTCAAAGCTGGG | sanger sequencing |
| | | | of SMUG1 m⁶A |
| | | | site |
| 48 | HZ_HERC2_F | TGTCGACTCCTTTGCTTCGGA | sanger sequencing |
| | | | of HERC2 m⁶A |
| | | | site |
| 49 | HZ_HERC2_R | AAATCGATTGCACCCACAAGTA | sanger sequencing |
| | | | of HERC2 m⁶A |
| | | | site |
| 50 | HZ_NIPA1_F | AGCCGCACAGAAAGATCACC | sanger sequencing |
| | | | of NIPA1 m⁶A site |
| 51 | HZ_NIPA1_R | AAGTGCAAAAATACTAAGGTGGG | sanger sequencing |
| | | | of NIPA1 m⁶A site |
| 52 | dCas13_GEMS-gRNA1 | AGCGCGATCACATGGTCCTGCTGGAGT | Guide RNA co- |
| | | TCG | transfected with |
| | | | GEMS-dCas13 |
| | | | targeted system. |
| | | | Targeting as |
| | | | marked in |
| | | | FIGS. 8A-8F: end |
| | | | of EGFP |
| 53 | dCas13_GEMS-gRNA2 | CCGCCGCATCTAACACATTGATCCTAG | Guide RNA co- |
| | | CAG | transfected with |
| | | | GEMS-dCas13 |
| | | | targeted system. |
| 54 | dCas13_GEMS-gRNA3 | GCGGACTTACGACAGTTGCGTTACACC | Guide RNA co- |
| | | CTT | transfected with |
| | | | GEMS-dCas13 |
| | | | targeted system. r |
| 55 | dCas13_GEMS-gRNA4 | TTGCGGCGTTAGCGGTAGATCACGTTA | Guide RNA co- |
| | | TCG | transfected with |
| | | | GEMS-dCas13 |
| | | | targeted system. |
| | | | Targeting as |
| | | | marked in |
| | | | FIGS. 8A-8F: start |
| | | | of DHFR |
| 56 | pCMV-APOBEC1-YTH | atgagctcagagactggcccagtggct | APOBEC1-YTH- |
| | | gtggaccccacattgagacggcggatc | HA to clone in |
| | | gagccccatgagtttgaggtattcttc | GEMS: |
| | | gatccgagagagctccgcaaggagacc | Addgene #131636, |
| | | tgcctgctttacgaaattaattggggg | the sequence of |
| | | ggccggcactccatttggcgacataca | which is accessible |
| | | tcacagaacactaacaagcacgtcgaa | at addgene.org/ |
| | | gtcaacttcatcgagaagttcacgaca | browse/sequence/ |
| | | gaaagatatttctgtccgaacacaagg | 260783/ and |
| | | tgcagcattacctggtttctcagctgg | incorporated |
| | | agcccatgcggcgaatgtagtagggcc | by reference |
| | | atcactgaattcctgtcaaggtatccc | |
| | | cacgtcactctgtttatttacatcgca | |
| | | aggctgtaccaccacgctgacccccgc | |
| | | aatcgacaaggcctgcgggatttgatc | |
| | | tcttcaggtgtgactatccaaattatg | |
| | | actgagcaggagtcaggatactgctgg | |
| | | agaaactttgtgaattatagcccgagt | |
| | | aatgaagcccactggcctaggtatccc | |
| | | catctgtgggtacgactgtacgttctt | |
| | | gaactgtactgcatcatactgggcctg | |
| | | cctccttgtctcaacattctgagaagg | |
| | | aagcagccacagctgacattctttacc | |
| | | atcgctcttcagtcttgtcattaccag | |
| | | cgactgcccccacacattctctgggcc | |
| | | accgggttgaaaagcggcagcgagact | |
| | | cccgggacctcagagtccgccacacca | |
| | | gaaccccacccagtgttggagaagctt | |
| | | cggtccattaataactataaccccaaa | |
| | | gattttgactggaatctgaaacatggc | |
| | | cgggttttcatcattaagagctactct | |
| | | gaggacgatattcaccgttccattaag | |
| | | tataatatttggtgcagcacagagcat | |
| | | ggtaacaagagactggatgctgcttat | |
| | | cgttccatgaacgggaaaggccccgtt | |
| | | tacttacttttcagtgtcaacggcagt | |
| | | ggacacttctgtggcgtggcagaaatg | |
| | | aaatctgctgtggactacaacacatgt | |
| | | gcaggtgtgtggtcccaggacaaatgg | |
| | | aagggtcgttttgatgtcaggtggatt | |
| | | tttgtgaaggacgttcccaatagccaa | |
| | | ctgcgacacattcgcctagagaacaac | |
| | | gagaataaaccagtgaccaactctagg | |
| | | gacactcaggaagtgcctctggaaaag | |
| | | gctaagcaggtgttgaaaattatagcc | |
| | | agctacaagcacaccacttccattttt | |
| | | gatgacttctcacactatgagaaacgc | |
| | | caagaggaagaagaaagtgttaaaaag | |
| | | gaacgtcaaggtcgtgggaaactcgag | |
| 57 | pCMV-APOBEC1-YTHmut | ATGAGCTCAGAGACTGGCCCAGTGGCT | APOBEC1- |
| | | GTGGACCCCACATTGAGACGGCGGATC | YTHmutant-HA to |
| | | GAGCCCCATGAGTTTGAGGTATTCTTC | clone in |
| | | GATCCGAGAGAGCTCCGCAAGGAGACC | GEMSmut: |
| | | TGCCTGCTTTACGAAATTAATTGGGGG | Addgene #131637, |
| | | GGCCGGCACTCCATTTGGCGACATACA | the sequence of |
| | | TCACAGAACACTAACAAGCACGTCGAA | which is accessible |
| | | GTCAACTTCATCGAGAAGTTCACGACA | at addgene.org/ |
| | | GAAAGATATTTCTGTCCGAACACAAGG | 131637/ and |
| | | TGCAGCATTACCTGGTTTCTCAGCTGG | incorporated |
| | | AGCCCATGCGGCGAATGTAGTAGGGCC | by reference |
| | | ATCACTGAATTCCTGTCAAGGTATCCC | |
| | | CACGTCACTCTGTTTATTTACATCGCA | |
| | | AGGCTGTACCACCACGCTGACCCCCGC | |
| | | AATCGACAAGGCCTGCGGGATTTGATC | |
| | | TCTTCAGGTGTGACTATCCAAATTATG | |
| | | ACTGAGCAGGAGTCAGGATACTGCTGG | |
| | | AGAAACTTTGTGAATTATAGCCCGAGT | |
| | | AATGAAGCCCACTGGCCTAGGTATCCC | |
| | | CATCTGTGGGTACGACTGTACGTTCTT | |
| | | GAACTGTACTGCATCATACTGGGCCTG | |
| | | CCTCCTTGTCTCAACATTCTGAGAAGG | |
| | | AAGCAGCCACAGCTGACATTCTTTACC | |
| | | ATCGCTCTTCAGTCTTGTCATTACCAG | |
| | | CGACTGCCCCCACACATTCTCTGGGCC | |
| | | ACCGGGTTGAAAAGCGGCAGCGAGACT | |
| | | CCCGGGACCTCAGAGTCCGCCACACCA | |
| | | GAAGGCCGGGTTTTCATCATTAAGAGC | |
| | | TACTCTGAGGACGATATTCACCGTTCC | |
| | | ATTAAGTATAATATTTGGTGCAGCACA | |
| | | GAGCATGGTAACAAGAGACTGGATGCT | |
| | | GCTTATCGTTCCATGAACGGGAAAGGC | |
| | | CCCGTTTACTTACTTTTCAGTGTCAAC | |
| | | GGCAGTGGACACTTCTGTGGCGTGGCA | |
| | | GAAATGAAATCTGCTGTGGACTACAAC | |
| | | ACATGTGCAGGTGTGTGGTCCCAGGAC | |
| | | AAATGGAAGGGTCGTTTTGATGTCAGG | |
| | | TGGATTTTTGTGAAGGACGTTCCCAAT | |
| | | AGCCAACTGCGACACATTCGCCTAGAG | |
| | | AACAACGAGAATAAACCAGTGACCAAC | |
| | | TCTAGGGACACTCAGGAAGTGCCTCTG | |
| | | GAAAAGGCTAAGCAGGTGTTGAAAATT | |
| | | ATAGCCAGCTACAAGCACACCACTTCC | |
| | | ATTTTTGATGACTTCTCACACTATGAG | |
| | | AAACGCCAAGAGGAAGAAGAAAGTGTT | |
| | | AAAAAGGAACGTCAAGGTCGTGGGAAA | |
| | | CTCGAG | |
| 58 | iDuet101a | GTCGACGGATCGGGAGATCTCCCGATC | GEMS backbone |
| | | CCCTATGGTGCACTCTCAGTACAATCT | plasmid: |
| | | GCTCTGATGCCGCATAGTTAAGCCAGT | Addgene #17629, |
| | | ATCTGCTCCCTGCTTGTGTGTTGGAGG | the sequence of |
| | | TCGCTGAGTAGTGCGCGAGCAAAATTT | which is accessible |
| | | AAGCTACAACAAGGCAAGGCTTGACCG | at addgene.org/ |
| | | ACAATTGCATGAAGAATCTGCTTAGGG | 17629/ and |
| | | TTAGGCGTTTTGCGCTGCTTCGCGATG | incorporated |
| | | TACGGGCCAGATATACGCGTTGACATT | by reference |
| | | GATTATTGACTAGTTATTAATAGTAAT | |
| | | CAATTACGGGGTCATTAGTTCATAGCC | |
| | | CATATATGGAGTTCCGCGTTACATAAC | |
| | | TTACGGTAAATGGCCCGCCTGGCTGAC | |
| | | CGCCCAACGACCCCCGCCCATTGACGT | |
| | | CAATAATGACGTATGTTCCCATAGTAA | |
| | | CGCCAATAGGGACTTTCCATTGACGTC | |
| | | AATGGGTGGAGTATTTACGGTAAACTG | |
| | | CCCACTTGGCAGTACATCAAGTGTATC | |
| | | ATATGCCAAGTACGCCCCCTATTGACG | |
| | | TCAATGACGGTAAATGGCCCGCCTGGC | |
| | | ATTATGCCCAGTACATGACCTTATGGG | |
| | | ACTTTCCTACTTGGCAGTACATCTACG | |
| | | TATTAGTCATCGCTATTACCATGGTGA | |
| | | TGCGGTTTTGGCAGTACATCAATGGGC | |
| | | GTGGATAGCGGTTTGACTCACGGGGAT | |
| | | TTCCAAGTCTCCACCCCATTGACGTCA | |
| | | ATGGGAGTTTGTTTTGGCACCAAAATC | |
| | | AACGGGACTTTCCAAAATGTCGTAACA | |
| | | ACTCCGCCCCATTGACGCAAATGGGCG | |
| | | GTAGGCGTGTACGGTGGGAGGTCTATA | |
| | | TAAGCAGCGCGTTTTGCCTGTACTGGG | |
| | | TCTCTCTGGTTAGACCAGATCTGAGCC | |
| | | TGGGAGCTCTCTGGCTAACTAGGGAAC | |
| | | CCACTGCTTAAGCCTCAATAAAGCTTG | |
| | | CCTTGAGTGCTTCAAGTAGTGTGTGCC | |
| | | CGTCTGTTGTGTGACTCTGGTAACTAG | |
| | | AGATCCCTCAGACCCTTTTAGTCAGTG | |
| | | TGGAAAATCTCTAGCAGTGGCGCCCGA | |
| | | ACAGGGACTTGAAAGCGAAAGGGAAAC | |
| | | CAGAGGAGCTCTCTCGACGCAGGACTC | |
| | | GGCTTGCTGAAGCGCGCACGGCAAGAG | |
| | | GCGAGGGGCGGCGACTGGTGAGTACGC | |
| | | CAAAAATTTTGACTAGCGGAGGCTAGA | |
| | | AGGAGAGAGATGGGTGCGAGAGCGTCA | |
| | | GTATTAAGCGGGGGAGAATTAGATCGC | |
| | | GATGGGAAAAAATTCGGTTAAGGCCAG | |
| | | GGGGAAAGAAAAAATATAAATTAAAAC | |
| | | ATATAGTATGGGCAAGCAGGGAGCTAG | |
| | | AACGATTCGCAGTTAATCCTGGCCTGT | |
| | | TAGAAACATCAGAAGGCTGTAGACAAA | |
| | | TACTGGGACAGCTACAACCATCCCTTC | |
| | | AGACAGGATCAGAAGAACTTAGATCAT | |
| | | TATATAATACAGTAGCAACCCTCTATT | |
| | | GTGTGCATCAAAGGATAGAGATAAAAG | |
| | | ACACCAAGGAAGCTTTAGACAAGATAG | |
| | | AGGAAGAGCAAAACAAAAGTAAGACCA | |
| | | CCGCACAGCAAGCGGCCGCTGATCTTC | |
| | | AGACCTGGAGGAGGAGATATGAGGGAC | |
| | | AATTGGAGAAGTGAATTATATAAATAT | |
| | | AAAGTAGTAAAAATTGAACCATTAGGA | |
| | | GTAGCACCCACCAAGGCAAAGAGAAGA | |
| | | GTGGTGCAGAGAGAAAAAAGAGCAGTG | |
| | | GGAATAGGAGCTTTGTTCCTTGGGTTC | |
| | | TTGGGAGCAGCAGGAAGCACTATGGGC | |
| | | GCAGCGTCAATGACGCTGACGGTACAG | |
| | | GCCAGACAATTATTGTCTGGTATAGTG | |
| | | CAGCAGCAGAACAATTTGCTGAGGGCT | |
| | | ATTGAGGCGCAACAGCATCTGTTGCAA | |
| | | CTCACAGTCTGGGGCATCAAGCAGCTC | |
| | | CAGGCAAGAATCCTGGCTGTGGAAAGA | |
| | | TACCTAAAGGATCAACAGCTCCTGGGG | |
| | | ATTTGGGGTTGCTCTGGAAAACTCATT | |
| | | TGCACCACTGCTGTGCCTTGGAATGCT | |
| | | AGTTGGAGTAATAAATCTCTGGAACAG | |
| | | ATTTGGAATCACACGACCTGGATGGAG | |
| | | TGGGACAGAGAAATTAACAATTACACA | |
| | | AGCTTAATACACTCCTTAATTGAAGAA | |
| | | TCGCAAAACCAGCAAGAAAAGAATGAA | |
| | | CAAGAATTATTGGAATTAGATAAATGG | |
| | | GCAAGTTTGTGGAATTGGTTTAACATA | |
| | | ACAAATTGGCTGTGGTATATAAAATTA | |
| | | TTCATAATGATAGTAGGAGGCTTGGTA | |
| | | GGTTTAAGAATAGTTTTTGCTGTACTT | |
| | | TCTATAGTGAATAGAGTTAGGCAGGGA | |
| | | TATTCACCATTATCGTTTCAGACCCAC | |
| | | CTCCCAACCCCGAGGGGACCCGACAGG | |
| | | CCCGAAGGAATAGAAGAAGAAGGTGGA | |
| | | GAGAGAGACAGAGACAGATCCATTCGA | |
| | | TTAGTGAACGGATCGGCACTGCGTGCG | |
| | | CCAATTCTGCAGACAAATGGCAGTATT | |
| | | CATCCACAATTTTAAAAGAAAAGGGGG | |
| | | GATTGGGGGGTACAGTGCAGGGGAAAG | |
| | | AATAGTAGACATAATAGCAACAGACAT | |
| | | ACAAACTAAAGAATTACAAAAACAAAT | |
| | | TACAAAAATTCAAAATTTTCGGGTTTA | |
| | | TTACAGGGACAGCAGAGATCCAGTTTG | |
| | | GTTAccagTGTGATGGATATCTGCAGA | |
| | | ATTCGCCCTTGGATCCGAATTCCTGCA | |
| | | GCCCCGACTTTCACTTTTCTCTATCAC | |
| | | TGATAGGGAGTGGTAAACTCGACTTTC | |
| | | ACTTTTCTCTATCACTGATAGGGAGTG | |
| | | GTAAACTCGACTTTCACTTTTCTCTAT | |
| | | CACTGATAGGGAGTGGTAAACTCGACT | |
| | | TTCACTTTTCTCTATCACTGATAGGGA | |
| | | GTGGTAAACTCGACTTTCACTTTTCTC | |
| | | TATCACTGATAGGGAGTGGTAAACTCG | |
| | | ACTTTCACTTTTCTCTATCACTGATAG | |
| | | GGAGTGGTAAACTCGACTTTCACTTTT | |
| | | CTCTATCACTGATAGGGAGTGGTAAAC | |
| | | TCGAGGGGGATCCACTAGCATGAAGGG | |
| | | CGAATTCCAGCACActggTAAcccgtg | |
| | | tcggctccagatctGGCCTCCGCGCCG | |
| | | GGTTTTGGCGcctcccgcgggcgcccc | |
| | | cctcctcacggcgagccgcgTTGACAT | |
| | | TGATTATTGACTAGGCTTTTGCAAAAA | |
| | | GCTTTGCAAAGATGGATAAAGTTTTAA | |
| | | ACAGAGAGGAATCTTTGCAGCTAATGG | |
| | | ACCTTCTAGGTCTTGAAAGGAGTGGGA | |
| | | ATTGGCTCCGGTGCCCGTCAGTGGGCA | |
| | | GAGCGCACATCGCCCACAGTCCCCGAG | |
| | | AAGTTGGGGGGAGGGGTCGGCAATTGA | |
| | | ACCGGTGCCTAGAGAAGGTGGCGCGGG | |
| | | GTAAACTGGGAAAGTGATGTCGTGTAC | |
| | | TGGCTCCGCCTTTTTCCCGAGGGTGGG | |
| | | GGAGAACCGTATATAAGTGCAGTAGTC | |
| | | GCCGTGAACGTTCTTTTTCGCAACGGG | |
| | | TTTGCCGCCAGAACACAGGTAAGTGCC | |
| | | GTGTGTGGTTCCCGCGGGCCTGGCCTC | |
| | | TTTACGGGTTATGGCCCTTGCGTGCCT | |
| | | TGAATTACTTCCACCTGGCTGCAGTAC | |
| | | GTGATTCTTGATCCCGAGCTTCGGGTT | |
| | | GGAAGTGGGTGGGAGAGTTCGAGGCCT | |
| | | TGCGCTTAAGGAGCCCCTTCGCCTCGT | |
| | | GCTTGAGTTGAGGCCTGGCCTGGGCGC | |
| | | TGGGGCCGCCGCGTGCGAATCTGGTGG | |
| | | CACCTTCGCGCCTGTCTCGCTGCTTTC | |
| | | GATAAGTCTCTAGCCATTTAAAATTTT | |
| | | TGATGACCTGCTGCGACGCTTTTTTTC | |
| | | TGGCAAGATAGTCTTGTAAATGCGGGC | |
| | | CAAGATCTGCACACTGGTATTTCGGTT | |
| | | TTTGGGGCCGCGGGCGGCGACGGGGCC | |
| | | CGTGCGTCCCAGCGCACATGTTCGGCG | |
| | | AGGCGGGGCCTGCGAGCGCGGCCACCG | |
| | | AGAATCGGACGGGGGTAGTCTCAAGCT | |
| | | GGCCGGCCTGCTCTGGTGCCTGGCCTC | |
| | | GCGCCGCCGTGTATCGCCCCGCCCTGG | |
| | | GCGGCAAGGCTGGCCCGGTCGGCACCA | |
| | | GTTGCGTGAGCGGAAAGATGGCCGCTT | |
| | | CCCGGCCCTGCTGCAGGGAGCTCAAAA | |
| | | TGGAGGACGCGGCGCTCGGGAGAGCGG | |
| | | GCGGGTGAGTCACCCACACAAAGGAAA | |
| | | AGGGCCTTTCCGTCCTCAGCCGTCGCT | |
| | | TCATGTGACTCCACGGAGTACCGGGCG | |
| | | CCGTCCAGGCACCTCGATTAGTTCTCG | |
| | | AGCTTTTGGAGTACGTCGTCTTTAGGT | |
| | | TGGGGGGAGGGGTTTTATGCGATGGAG | |
| | | TTTCCCCACACTGAGTGGGTGGAGACT | |
| | | GAAGTTAGGCCAGCTTGGCACTTGATG | |
| | | TAATTCTCCTTGGAATTTGCCCTTTTT | |
| | | GAGTTTGGATCTTGGTTCATTCTCAAG | |
| | | CCTCAGACAGTGGTTCAAAGTTTTTTT | |
| | | CTTCCATTTCAGGTGTCGTGAGGAATT | |
| | | AGCTTGGTACTAATACGACTCACTATA | |
| | | GGGAGACCCAAGCTGGCTAGGTAAGCT | |
| | | TGGTACCGAGCTCGGATCCACTAGTCC | |
| | | AGTGTGGTGGAATTCTGCAGATATCCA | |
| | | GCACAGTGGCGGCCGCTCGAGtctaga | |
| | | ggatccccgggtaccggtcgccaccAT | |
| | | GGTGAGCAAGGGCGAGGAGCTGTTCAC | |
| | | CGGGGTGGTGCCCATCCTGGTCGAGCT | |
| | | GGACGGCGACGTAAACGGCCACAAGTT | |
| | | CAGCGTGTCCGGCGAGGGCGAGGGCGA | |
| | | TGCCACCTACGGCAAGCTGACCCTGAA | |
| | | GTTCATCTGCACCACCGGCAAGCTGCC | |
| | | CGTGCCCTGGCCCACCCTCGTGACCAC | |
| | | CCTGACCTACGGCGTGCAGTGCTTCAG | |
| | | CCGCTACCCCGACCACATGAAGCAGCA | |
| | | CGACTTCTTCAAGTCCGCCATGCCCGA | |
| | | AGGCTACGTCCAGGAGCGCACCATCTT | |
| | | CTTCAAGGACGACGGCAACTACAAGAC | |
| | | CCGCGCCGAGGTGAAGTTCGAGGGCGA | |
| | | CACCCTGGTGAACCGCATCGAGCTGAA | |
| | | GGGCATCGACTTCAAGGAGGACGGCAA | |
| | | CATCCTGGGGCACAAGCTGGAGTACAA | |
| | | CTACAACAGCCACAACGTCTATATCAT | |
| | | GGCCGACAAGCAGAAGAACGGCATCAA | |
| | | GGTGAACTTCAAGATCCGCCACAACAT | |
| | | CGAGGACGGCAGCGTGCAGCTCGCCGA | |
| | | CCACTACCAGCAGAACACCCCCATCGG | |
| | | CGACGGCCCCGTGCTGCTGCCCGACAA | |
| | | CCACTACCTGAGCACCCAGTCCGCCCT | |
| | | GAGCAAAGACCCCAACGAGAAGCGCGA | |
| | | TCACATGGTCCTGCTGGAGTTCGTGAC | |
| | | CGCCGCCGGGATCACTCTCGGCATGGA | |
| | | CGAGCtgtacactcgaggttaacgaat | |
| | | tctaccgggtaggggaggcgcttttcc | |
| | | caaggcagtctggagcatgcgctttag | |
| | | cagccccgctgggcacttggcgctaca | |
| | | caagtggcctctggcctcgcacacatt | |
| | | ccacatccaccggtaggcgccaaccgg | |
| | | ctccgttctttggtggccccttcgcgc | |
| | | caccttctactcctcccctagtcagga | |
| | | agttcccccccgccccgcagctcgcgt | |
| | | cgtgcaggacgtgacaaatggaagtag | |
| | | cacgtctcactagtctcgtgcagatgg | |
| | | acagcaccgctgagcaatggaagcggg | |
| | | taggcctttggggcagcggccaatagc | |
| | | agctttgctccttcgctttctgggctc | |
| | | agaggctgggaaggggtgggtccgggg | |
| | | ggggctcaggggcgggctcaggggggg | |
| | | gggggcccgaaggtcctccggaggccc | |
| | | ggcattctgcacgcttcaaaagcgcac | |
| | | gtctgccgcgctgttctcctcttcctc | |
| | | atctccgggcctttcgacctgcatccc | |
| | | gccaccATGAAAAAGCCTGAACTCACC | |
| | | GCGACGTCTGTCGAGAAGTTTCTGATC | |
| | | GAAAAGTTCGACAGCGTCTCCGACCTG | |
| | | ATGCAGCTCTCGGAGGGCGAAGAATCT | |
| | | CGTGCTTTCAGCTTCGATGTAGGAGGG | |
| | | CGTGGATATGTCCTGCGGGTAAATAGC | |
| | | TGCGCCGATGGTTTCTACAAAGATCGT | |
| | | TATGTTTATCGGCACTTTGCATCGGCC | |
| | | GCGCTCCCGATTCCGGAAGTGCTTGAC | |
| | | ATTGGGGAATTCAGCGAGAGCCTGACC | |
| | | TATTGCATCTCCCGCCGTGCACAGGGT | |
| | | GTCACGTTGCAAGACCTGCCTGAAACC | |
| | | GAACTGCCCGCTGTTCTGCAGCCGGTC | |
| | | GCGGAGGCCATGGATGCGATCGCTGCG | |
| | | GCCGATCTTAGCCAGACGAGCGGGTTC | |
| | | GGCCCATTCGGACCGCAAGGAATCGGT | |
| | | CAATACACTACATGGCGTGATTTCATA | |
| | | TGCGCGATTGCTGATCCCCATGTGTAT | |
| | | CACTGGCAAACTGTGATGGACGACACC | |
| | | GTCAGTGCGTCCGTCGCGCAGGCTCTC | |
| | | GATGAGCTGATGCTTTGGGCCGAGGAC | |
| | | TGCCCCGAAGTCCGGCACCTCGTGCAC | |
| | | GCGGATTTCGGCTCCAACAATGTCCTG | |
| | | ACGGACAATGGCCGCATAACAGCGGTC | |
| | | ATTGACTGGAGCGAGGCGATGTTCGGG | |
| | | GATTCCCAATACGAGGTCGCCAACATC | |
| | | TTCTTCTGGAGGCCGTGGTTGGCTTGT | |
| | | ATGGAGCAGCAGACGCGCTACTTCGAG | |
| | | CGGAGGCATCCGGAGCTTGCAGGATCG | |
| | | CCGCGGCTCCGGGGCGTATATGCTCCG | |
| | | CATTGGTCTTGACCAACTCTATCAGAG | |
| | | CTTGGTTGACGGCAATTTCGATGATGC | |
| | | AGCTTGGGCGCAGGGTCGATGCGACGC | |
| | | AATCGTCCGATCCGGAGCCGGGACTGT | |
| | | CGGGCGTACACAAATCGCCCGCAGAAG | |
| | | CGCGGCCGTCTGGACCGATGGCTGTGT | |
| | | AGAAGTACTCGCCGATAGTGGAAACCG | |
| | | ACGCCCCAGCACTCGTCCGAGGGCAAA | |
| | | GGAATAGAGTAGATGCCGACCGAACAA | |
| | | GAGCTGATTTCGAGAACGCCTCAGCCA | |
| | | GCAACTCGCGCGAGCCTAGCAAGGCAA | |
| | | ATGCGAGAGAACGGCCTTACGCTTGGT | |
| | | GGCACAGTTCTCGTCCACAGTTCGCTA | |
| | | AGCTCGCTCGGCTGGGTCGCGGGAGGG | |
| | | CCGGTCGCAGTGATTCAGGCCCTTCTG | |
| | | GATTGTGTTGGTCCCCAGGGCACGATT | |
| | | GTCATGCCCACGCACTCGGGTGATCTG | |
| | | ACTGATCCCGCAGATTGGAGATCGCCG | |
| | | CCCGTGCCTGCCGATTGGGTGCAGATC | |
| | | CGTCGAGttaacAAAAGAAAAGGGGGG | |
| | | ACTGGAAGGGCTAATTCACTCCCAACG | |
| | | AAGACAAGATatcataacttcgtatag | |
| | | catacattatacgaagttatcggctag | |
| | | ctggtccggaCTGTACTGGGTCTCTCT | |
| | | GGTTAGACCAGATCTGAGCCTGGGAGC | |
| | | TCTCTGGCTAACTAGGGAACCCACTGC | |
| | | TTAAGCCTCAATAAAGCTTGCCTTGAG | |
| | | TGCTTCAAGTAGTGTGTGCCCGTCTGT | |
| | | TGTGTGACTCTGGTAACTAGAGATCCC | |
| | | TCAGACCCTTTTAGTCAGTGTGGAAAA | |
| | | TCTCTAGCAGGGCCCGTTTAAACCCGC | |
| | | TGATCAGCCTCGACTGTGCCTTCTAGT | |
| | | TGCCAGCCATCTGTTGTTTGCCCCTCC | |
| | | CCCGTGCCTTCCTTGACCCTGGAAGGT | |
| | | GCCACTCCCACTGTCCTTTCCTAATAA | |
| | | AATGAGGAAATTGCATCGCATTGTCTG | |
| | | AGTAGGTGTCATTCTATTCTGGGGGGT | |
| | | GGGGTGGGGCAGGACAGCAAGGGGGAG | |
| | | GATTGGGAAGACAATAGCAGGCATGCT | |
| | | GGGGATGCGGTGGGCTCTATGGCTTCT | |
| | | GAGGCGGAAAGAACCAGCTGGGGCTCT | |
| | | AGGGGGTATCCCCACGCGCCCTGTAGC | |
| | | GGCGCATTAAGCGCGGCGGGTGTGGTG | |
| | | GTTACGCGCAGCGTGACCGCTACACTT | |
| | | GCCAGCGCCCTAGCGCCCGCTCCTTTC | |
| | | GCTTTCTTCCCTTCCTTTCTCGCCACG | |
| | | TTCGCCGGCTTTCCCCGTCAAGCTCTA | |
| | | AATCGGGGGCTCCCTTTAGGGTTCCGA | |
| | | TTTAGTGCTTTACGGCACCTCGACCCC | |
| | | AAAAAACTTGATTAGGGTGATGGTTCA | |
| | | CGTAGTGGGCCATCGCCCTGATAGACG | |
| | | GTTTTTCGCCCTTTGACGTTGGAGTCC | |
| | | ACGTTCTTTAATAGTGGACTCTTGTTC | |
| | | CAAACTGGAACAACACTCAACCCTATC | |
| | | TCGGTCTATTCTTTTGATTTATAAGGG | |
| | | ATTTTGCCGATTTCGGCCTATTGGTTA | |
| | | AAAAATGAGCTGATTTAACAAAAATTT | |
| | | AACGCGAATTAATTCTGTGGAATGTGT | |
| | | GTCAGTTAGGGTGTGGAAAGTCCCCAG | |
| | | GCTCCCCAGCAGGCAGAAGTATGCAAA | |
| | | GCATGCATCTCAATTAGTCAGCAACCA | |
| | | GGTGTGGAAAGTCCCCAGGCTCCCCAG | |
| | | CAGGCAGAAGTATGCAAAGCATGCATC | |
| | | TCAATTAGTCAGCAACCATAGTCCCGC | |
| | | CCCTAACTCCGCCCATCCCGCCCCTAA | |
| | | CTCCGCCCAGTTCCGCCCATTCTCCGC | |
| | | CCCATGGCTGACTAATTTTTTTTATTT | |
| | | ATGCAGAGGCCGAGGCCGCCTCTGCCT | |
| | | CTGAGCTATTCCAGAAGTAGTGAGGAG | |
| | | GCTTTTTTGGAGGCCTAGGCTTTTGCA | |
| | | AAAAGCTCCCGGGAGCTTGTATATCCA | |
| | | TTTTCGGATCTGATCAGCACGTGTTGA | |
| | | CAATTAATCATCGGCATAGTATATCGG | |
| | | CATAGTATAATACGACAAGGTGAGGAA | |
| | | CTAAACCATGGCCAAGTTGACCAGTGC | |
| | | CGTTCCGGTGCTCACCGCGCGCGACGT | |
| | | CGCCGGAGCGGTCGAGTTCTGGACCGA | |
| | | CCGGCTCGGGTTCTCCCGGGACTTCGT | |
| | | GGAGGACGACTTCGCCGGTGTGGTCCG | |
| | | GGACGACGTGACCCTGTTCATCAGCGC | |
| | | GGTCCAGGACCAGGTGGTGCCGGACAA | |
| | | CACCCTGGCCTGGGTGTGGGTGCGCGG | |
| | | CCTGGACGAGCTGTACGCCGAGTGGTC | |
| | | GGAGGTCGTGTCCACGAACTTCCGGGA | |
| | | CGCCTCCGGGCCGGCCATGACCGAGAT | |
| | | CGGCGAGCAGCCGTGGGGGCGGGAGTT | |
| | | CGCCCTGCGCGACCCGGCCGGCAACTG | |
| | | CGTGCACTTCGTGGCCGAGGAGCAGGA | |
| | | CTGACACGTGCTACGAGATTTCGATTC | |
| | | CACCGCCGCCTTCTATGAAAGGTTGGG | |
| | | CTTCGGAATCGTTTTCCGGGACGCCGG | |
| | | CTGGATGATCCTCCAGCGCGGGGATCT | |
| | | CATGCTGGAGTTCTTCGCCCACCCCAA | |
| | | CTTGTTTATTGCAGCTTATAATGGTTA | |
| | | CAAATAAAGCAATAGCATCACAAATTT | |
| | | CACAAATAAAGCATTTTTTTCACTGCA | |
| | | TTCTAGTTGTGGTTTGTCCAAACTCAT | |
| | | CAATGTATCTTATCATGTCTGTATACC | |
| | | GTCGACCTCTAGCTAGAGCTTGGCGTA | |
| | | ATCATGGTCATAGCTGTTTCCTGTGTG | |
| | | AAATTGTTATCCGCTCACAATTCCACA | |
| | | CAACATACGAGCCGGAAGCATAAAGTG | |
| | | TAAAGCCTGGGGTGCCTAATGAGTGAG | |
| | | CTAACTCACATTAATTGCGTTGCGCTC | |
| | | ACTGCCCGCTTTCCAGTCGGGAAACCT | |
| | | GTCGTGCCAGCTGCATTAATGAATCGG | |
| | | CCAACGCGCGGGGAGAGGCGGTTTGCG | |
| | | TATTGGGCGCTCTTCCGCTTCCTCGCT | |
| | | CACTGACTCGCTGCGCTCGGTCGTTCG | |
| | | GCTGCGGCGAGCGGTATCAGCTCACTC | |
| | | AAAGGCGGTAATACGGTTATCCACAGA | |
| | | ATCAGGGGATAACGCAGGAAAGAACAT | |
| | | GTGAGCAAAAGGCCAGCAAAAGGCCAG | |
| | | GAACCGTAAAAAGGCCGCGTTGCTGGC | |
| | | GTTTTTCCATAGGCTCCGCCCCCCTGA | |
| | | CGAGCATCACAAAAATCGACGCTCAAG | |
| | | TCAGAGGTGGCGAAACCCGACAGGACT | |
| | | ATAAAGATACCAGGCGTTTCCCCCTGG | |
| | | AAGCTCCCTCGTGCGCTCTCCTGTTCC | |
| | | GACCCTGCCGCTTACCGGATACCTGTC | |
| | | CGCCTTTCTCCCTTCGGGAAGCGTGGC | |
| | | GCTTTCTCATAGCTCACGCTGTAGGTA | |
| | | TCTCAGTTCGGTGTAGGTCGTTCGCTC | |
| | | CAAGCTGGGCTGTGTGCACGAACCCCC | |
| | | CGTTCAGCCCGACCGCTGCGCCTTATC | |
| | | CGGTAACTATCGTCTTGAGTCCAACCC | |
| | | GGTAAGACACGACTTATCGCCACTGGC | |
| | | AGCAGCCACTGGTAACAGGATTAGCAG | |
| | | AGCGAGGTATGTAGGCGGTGCTACAGA | |
| | | GTTCTTGAAGTGGTGGCCTAACTACGG | |
| | | CTACACTAGAAGAACAGTATTTGGTAT | |
| | | CTGCGCTCTGCTGAAGCCAGTTACCTT | |
| | | CGGAAAAAGAGTTGGTAGCTCTTGATC | |
| | | CGGCAAACAAACCACCGCTGGTAGCGG | |
| | | TGGTTTTTTTGTTTGCAAGCAGCAGAT | |
| | | TACGCGCAGAAAAAAAGGATCTCAAGA | |
| | | AGATCCTTTGATCTTTTCTACGGGGTC | |
| | | TGACGCTCAGTGGAACGAAAACTCACG | |
| | | TTAAGGGATTTTGGTCATGAGATTATC | |
| | | AAAAAGGATCTTCACCTAGATCCTTTT | |
| | | AAATTAAAAATGAAGTTTTAAATCAAT | |
| | | CTAAAGTATATATGAGTAAACTTGGTC | |
| | | TGACAGTTACCAATGCTTAATCAGTGA | |
| | | GGCACCTATCTCAGCGATCTGTCTATT | |
| | | TCGTTCATCCATAGTTGCCTGACTCCC | |
| | | CGTCGTGTAGATAACTACGATACGGGA | |
| | | GGGCTTACCATCTGGCCCCAGTGCTGC | |
| | | AATGATACCGCGAGACCCACGCTCACC | |
| | | GGCTCCAGATTTATCAGCAATAAACCA | |
| | | GCCAGCCGGAAGGGCCGAGCGCAGAAG | |
| | | TGGTCCTGCAACTTTATCCGCCTCCAT | |
| | | CCAGTCTATTAATTGTTGCCGGGAAGC | |
| | | TAGAGTAAGTAGTTCGCCAGTTAATAG | |
| | | TTTGCGCAACGTTGTTGCCATTGCTAC | |
| | | AGGCATCGTGGTGTCACGCTCGTCGTT | |
| | | TGGTATGGCTTCATTCAGCTCCGGTTC | |
| | | CCAACGATCAAGGCGAGTTACATGATC | |
| | | CCCCATGTTGTGCAAAAAAGCGGTTAG | |
| | | CTCCTTCGGTCCTCCGATCGTTGTCAG | |
| | | AAGTAAGTTGGCCGCAGTGTTATCACT | |
| | | CATGGTTATGGCAGCACTGCATAATTC | |
| | | TCTTACTGTCATGCCATCCGTAAGATG | |
| | | CTTTTCTGTGACTGGTGAGTACTCAAC | |
| | | CAAGTCATTCTGAGAATAGTGTATGCG | |
| | | GCGACCGAGTTGCTCTTGCCCGGCGTC | |
| | | AATACGGGATAATACCGCGCCACATAG | |
| | | CAGAACTTTAAAAGTGCTCATCATTGG | |
| | | AAAACGTTCTTCGGGGCGAAAACTCTC | |
| | | AAGGATCTTACCGCTGTTGAGATCCAG | |
| | | TTCGATGTAACCCACTCGTGCACCCAA | |
| | | CTGATCTTCAGCATCTTTTACTTTCAC | |
| | | CAGCGTTTCTGGGTGAGCAAAAACAGG | |
| | | AAGGCAAAATGCCGCAAAAAAGGGAAT | |
| | | AAGGGCGACACGGAAATGTTGAATACT | |
| | | CATACTCTTCCTTTTTCAATATTATTG | |
| | | AAGCATTTATCAGGGTTATTGTCTCAT | |
| | | GAGCGGATACATATTTGAATGTATTTA | |
| | | GAAAAATAAACAAATAGGGGTTCCGCG | |
| | | CACATTTCCCCGAAAAGTGCCACCTGA | |
| | | C | |
| 59 | TLCV2 | ATGGACAAGAAGTACAGCATCGGCCTG | CAS9 fragment |
| | | GACATCGGCACCAACTCTGTGGGCTGG | source to clone in |
| | | GCCGTGATCACCGACGAGTACAAGGTG | GEMS: |
| | | CCCAGCAAGAAATTCAAGGTGCTGGGC | Addgene #87360, |
| | | AACACCGACCGGCACAGCATCAAGAAG | the sequence of |
| | | AACCTGATCGGAGCCCTGCTGTTCGAC | which is accessible |
| | | AGCGGCGAAACAGCCGAGGCCACCCGG | at addgene.org/ |
| | | CTGAAGAGAACCGCCAGAAGAAGATAC | 87360/ and |
| | | ACCAGACGGAAGAACCGGATCTGCTAT | incorporated |
| | | CTGCAAGAGATCTTCAGCAACGAGATG | by reference |
| | | GCCAAGGTGGACGACAGCTTCTTCCAC | |
| | | AGACTGGAAGAGTCCTTCCTGGTGGAA | |
| | | GAGGATAAGAAGCACGAGCGGCACCCC | |
| | | ATCTTCGGCAACATCGTGGACGAGGTG | |
| | | GCCTACCACGAGAAGTACCCCACCATC | |
| | | TACCACCTGAGAAAGAAACTGGTGGAC | |
| | | AGCACCGACAAGGCCGACCTGCGGCTG | |
| | | ATCTATCTGGCCCTGGCCCACATGATC | |
| | | AAGTTCCGGGGCCACTTCCTGATCGAG | |
| | | GGCGACCTGAACCCCGACAACAGCGAC | |
| | | GTGGACAAGCTGTTCATCCAGCTGGTG | |
| | | CAGACCTACAACCAGCTGTTCGAGGAA | |
| | | AACCCCATCAACGCCAGCGGCGTGGAC | |
| | | GCCAAGGCCATCCTGTCTGCCAGACTG | |
| | | AGCAAGAGCAGACGGCTGGAAAATCTG | |
| | | ATCGCCCAGCTGCCCGGCGAGAAGAAG | |
| | | AATGGCCTGTTCGGAAACCTGATTGCC | |
| | | CTGAGCCTGGGCCTGACCCCCAACTTC | |
| | | AAGAGCAACTTCGACCTGGCCGAGGAT | |
| | | GCCAAACTGCAGCTGAGCAAGGACACC | |
| | | TACGACGACGACCTGGACAACCTGCTG | |
| | | GCCCAGATCGGCGACCAGTACGCCGAC | |
| | | CTGTTTCTGGCCGCCAAGAACCTGTCC | |
| | | GACGCCATCCTGCTGAGCGACATCCTG | |
| | | AGAGTGAACACCGAGATCACCAAGGCC | |
| | | CCCCTGAGCGCCTCTATGATCAAGAGA | |
| | | TACGACGAGCACCACCAGGACCTGACC | |
| | | CTGCTGAAAGCTCTCGTGCGGCAGCAG | |
| | | CTGCCTGAGAAGTACAAAGAGATTTTC | |
| | | TTCGACCAGAGCAAGAACGGCTACGCC | |
| | | GGCTACATTGACGGCGGAGCCAGCCAG | |
| | | GAAGAGTTCTACAAGTTCATCAAGCCC | |
| | | ATCCTGGAAAAGATGGACGGCACCGAG | |
| | | GAACTGCTCGTGAAGCTGAACAGAGAG | |
| | | GACCTGCTGCGGAAGCAGCGGACCTTC | |
| | | GACAACGGCAGCATCCCCCACCAGATC | |
| | | CACCTGGGAGAGCTGCACGCCATTCTG | |
| | | CGGCGGCAGGAAGATTTTTACCCATTC | |
| | | CTGAAGGACAACCGGGAAAAGATCGAG | |
| | | AAGATCCTGACCTTCCGCATCCCCTAC | |
| | | TACGTGGGCCCTCTGGCCAGGGGAAAC | |
| | | AGCAGATTCGCCTGGATGACCAGAAAG | |
| | | AGCGAGGAAACCATCACCCCCTGGAAC | |
| | | TTCGAGGAAGTGGTGGACAAGGGCGCT | |
| | | TCCGCCCAGAGCTTCATCGAGCGGATG | |
| | | ACCAACTTCGATAAGAACCTGCCCAAC | |
| | | GAGAAGGTGCTGCCCAAGCACAGCCTG | |
| | | CTGTACGAGTACTTCACCGTGTATAAC | |
| | | GAGCTGACCAAAGTGAAATACGTGACC | |
| | | GAGGGAATGAGAAAGCCCGCCTTCCTG | |
| | | AGCGGCGAGCAGAAAAAGGCCATCGTG | |
| | | GACCTGCTGTTCAAGACCAACCGGAAA | |
| | | GTGACCGTGAAGCAGCTGAAAGAGGAC | |
| | | TACTTCAAGAAAATCGAGTGCTTCGAC | |
| | | TCCGTGGAAATCTCCGGCGTGGAAGAT | |
| | | CGGTTCAACGCCTCCCTGGGCACATAC | |
| | | CACGATCTGCTGAAAATTATCAAGGAC | |
| | | AAGGACTTCCTGGACAATGAGGAAAAC | |
| | | GAGGACATTCTGGAAGATATCGTGCTG | |
| | | ACCCTGACACTGTTTGAGGACAGAGAG | |
| | | ATGATCGAGGAACGGCTGAAAACCTAT | |
| | | GCCCACCTGTTCGACGACAAAGTGATG | |
| | | AAGCAGCTGAAGCGGCGGAGATACACC | |
| | | GGCTGGGGCAGGCTGAGCCGGAAGCTG | |
| | | ATCAACGGCATCCGGGACAAGCAGTCC | |
| | | GGCAAGACAATCCTGGATTTCCTGAAG | |
| | | TCCGACGGCTTCGCCAACAGAAACTTC | |
| | | ATGCAGCTGATCCACGACGACAGCCTG | |
| | | ACCTTTAAAGAGGACATCCAGAAAGCC | |
| | | CAGGTGTCCGGCCAGGGCGATAGCCTG | |
| | | CACGAGCACATTGCCAATCTGGCCGGC | |
| | | AGCCCCGCCATTAAGAAGGGCATCCTG | |
| | | CAGACAGTGAAGGTGGTGGACGAGCTC | |
| | | GTGAAAGTGATGGGCCGGCACAAGCCC | |
| | | GAGAACATCGTGATCGAAATGGCCAGA | |
| | | GAGAACCAGACCACCCAGAAGGGACAG | |
| | | AAGAACAGCCGCGAGAGAATGAAGCGG | |
| | | ATCGAAGAGGGCATCAAAGAGCTGGGC | |
| | | AGCCAGATCCTGAAAGAACACCCCGTG | |
| | | GAAAACACCCAGCTGCAGAACGAGAAG | |
| | | CTGTACCTGTACTACCTGCAGAATGGG | |
| | | CGGGATATGTACGTGGACCAGGAACTG | |
| | | GACATCAACCGGCTGTCCGACTACGAT | |
| | | GTGGACCATATCGTGCCTCAGAGCTTT | |
| | | CTGAAGGACGACTCCATCGACAACAAG | |
| | | GTGCTGACCAGAAGCGACAAGAACCGG | |
| | | GGCAAGAGCGACAACGTGCCCTCCGAA | |
| | | GAGGTCGTGAAGAAGATGAAGAACTAC | |
| | | TGGCGGCAGCTGCTGAACGCCAAGCTG | |
| | | ATTACCCAGAGAAAGTTCGACAATCTG | |
| | | ACCAAGGCCGAGAGAGGCGGCCTGAGC | |
| | | GAACTGGATAAGGCCGGCTTCATCAAG | |
| | | AGACAGCTGGTGGAAACCCGGCAGATC | |
| | | ACAAAGCACGTGGCACAGATCCTGGAC | |
| | | TCCCGGATGAACACTAAGTACGACGAG | |
| | | AATGACAAGCTGATCCGGGAAGTGAAA | |
| | | GTGATCACCCTGAAGTCCAAGCTGGTG | |
| | | TCCGATTTCCGGAAGGATTTCCAGTTT | |
| | | TACAAAGTGCGCGAGATCAACAACTAC | |
| | | CACCACGCCCACGACGCCTACCTGAAC | |
| | | GCCGTCGTGGGAACCGCCCTGATCAAA | |
| | | AAGTACCCTAAGCTGGAAAGCGAGTTC | |
| | | GTGTACGGCGACTACAAGGTGTACGAC | |
| | | GTGCGGAAGATGATCGCCAAGAGCGAG | |
| | | CAGGAAATCGGCAAGGCTACCGCCAAG | |
| | | TACTTCTTCTACAGCAACATCATGAAC | |
| | | TTTTTCAAGACCGAGATTACCCTGGCC | |
| | | AACGGCGAGATCCGGAAGCGGCCTCTG | |
| | | ATCGAGACAAACGGCGAAACCGGGGAG | |
| | | ATCGTGTGGGATAAGGGCCGGGATTTT | |
| | | GCCACCGTGCGGAAAGTGCTGAGCATG | |
| | | CCCCAAGTGAATATCGTGAAAAAGACC | |
| | | GAGGTGCAGACAGGCGGCTTCAGCAAA | |
| | | GAGTCTATCCTGCCCAAGAGGAACAGC | |
| | | GATAAGCTGATCGCCAGAAAGAAGGAC | |
| | | TGGGACCCTAAGAAGTACGGCGGCTTC | |
| | | GACAGCCCCACCGTGGCCTATTCTGTG | |
| | | CTGGTGGTGGCCAAAGTGGAAAAGGGC | |
| | | AAGTCCAAGAAACTGAAGAGTGTGAAA | |
| | | GAGCTGCTGGGGATCACCATCATGGAA | |
| | | AGAAGCAGCTTCGAGAAGAATCCCATC | |
| | | GACTTTCTGGAAGCCAAGGGCTACAAA | |
| | | GAAGTGAAAAAGGACCTGATCATCAAG | |
| | | CTGCCTAAGTACTCCCTGTTCGAGCTG | |
| | | GAAAACGGCCGGAAGAGAATGCTGGCC | |
| | | TCTGCCGGCGAACTGCAGAAGGGAAAC | |
| | | GAACTGGCCCTGCCCTCCAAATATGTG | |
| | | AACTTCCTGTACCTGGCCAGCCACTAT | |
| | | GAGAAGCTGAAGGGCTCCCCCGAGGAT | |
| | | AATGAGCAGAAACAGCTGTTTGTGGAA | |
| | | CAGCACAAGCACTACCTGGACGAGATC | |
| | | ATCGAGCAGATCAGCGAGTTCTCCAAG | |
| | | AGAGTGATCCTGGCCGACGCTAATCTG | |
| | | GACAAAGTGCTGTCCGCCTACAACAAG | |
| | | CACCGGGATAAGCCCATCAGAGAGCAG | |
| | | GCCGAGAATATCATCCACCTGTTTACC | |
| | | CTGACCAATCTGGGAGCCCCTGCCGCC | |
| | | TTCAAGTACTTTGACACCACCATCGAC | |
| | | CGGAAGAGGTACACCAGCACCAAAGAG | |
| | | GTGCTGGACGCCACCCTGATCCACCAG | |
| | | AGCATCACCGGCCTGTACGAGACACGG | |
| | | ATCGACCTGTCTCAGCTGGGAGGCGAC | |
| 60 | pCMV-dCas13-M3nls | GTGATGCGGTTTTGGCAGTACATCAAT | Targeted m6A |
| | | GGGCGTGGATAGCGGTTTGACTCACGG | RNA methylation |
| | | GGATTTCCAAGTCTCCACCCCATTGAC | in mammalian cells |
| | | GTCAATGGGAGTTTGTTTTGGCACCAA | Addgene #155366, |
| | | AATCAACGGGACTTTCCAAAATGTCGT | the sequence of |
| | | AACAACTCCGCCCCATTGACGCAAATG | which is accessible |
| | | GGCGGTAGGCGTGTACGGTGGGAGGTC | at addgene.org/ |
| | | TATATAAGCAGAGCTGGTTTAGTGAAC | 155366/ and |
| | | CGTCAGATCCGCTAGAGATCCGCGGCC | incorporated |
| | | GCTAATACGACTCACTATAGGGAGAGC | by reference |
| | | CGCCACCATGAAACGGACAGCCGACGG | |
| | | AAGCGAGTTCGAGTCACCAAAGAAGAA | |
| | | GCGGAAAGTCAACATCCCCGCTCTGGT | |
| | | GGAAAACCAGAAGAAGTACTTTGGCAC | |
| | | CTACAGCGTGATGGCCATGCTGAACGC | |
| | | TCAGACCGTGCTGGACCACATCCAGAA | |
| | | GGTGGCCGATATTGAGGGCGAGCAGAA | |
| | | CGAGAACAACGAGAATCTGTGGTTTCA | |
| | | CCCCGTGATGAGCCACCTGTACAACGC | |
| | | CAAGAACGGCTACGACAAGCAGCCCGA | |
| | | GAAAACCATGTTCATCATCGAGCGGCT | |
| | | GCAGAGCTACTTCCCATTCCTGAAGAT | |
| | | CATGGCCGAGAACCAGAGAGAGTACAG | |
| | | CAACGGCAAGTACAAGCAGAACCGCGT | |
| | | GGAAGTGAACAGCAACGACATCTTCGA | |
| | | GGTGCTGAAGCGCGCCTTCGGCGTGCT | |
| | | GAAGATGTACAGGGACCTGACCAACGC | |
| | | ATACAAGACCTACGAGGAAAAGCTGAA | |
| | | CGACGGCTGCGAGTTCCTGACCAGCAC | |
| | | AGAGCAACCTCTGAGCGGCATGATCAA | |
| | | CAACTACTACACAGTGGCCCTGCGGAA | |
| | | CATGAACGAGAGATACGGCTACAAGAC | |
| | | AGAGGACCTGGCCTTCATCCAGGACAA | |
| | | GCGGTTCAAGTTCGTGAAGGACGCCTA | |
| | | CGGCAAGAAAAAGTCCCAAGTGAATAC | |
| | | CGGATTCTTCCTGAGCCTGCAGGACTA | |
| | | CAACGGCGACACACAGAAGAAGCTGCA | |
| | | CCTGAGCGGAGTGGGAATCGCCCTGCT | |
| | | GATCTGCCTGTTCCTGGACAAGCAGTA | |
| | | CATCAACATCTTTCTGAGCAGGCTGCC | |
| | | CATCTTCTCCAGCTACAATGCCCAGAG | |
| | | CGAGGAACGGCGGATCATCATCAGATC | |
| | | CTTCGGCATCAACAGCATCAAGCTGCC | |
| | | CAAGGACCGGATCCACAGCGAGAAGTC | |
| | | CAACAAGAGCGTGGCCATGGATATGCT | |
| | | CAACGAAGTGAAGCGGTGCCCCGACGA | |
| | | GCTGTTCACAACACTGTCTGCCGAGAA | |
| | | GCAGTCCCGGTTCAGAATCATCAGCGA | |
| | | CGACCACAATGAAGTGCTGATGAAGCG | |
| | | GAGCAGCGACAGATTCGTGCCTCTGCT | |
| | | GCTGCAGTATATCGATTACGGCAAGCT | |
| | | GTTCGACCACATCAGGTTCCACGTGAA | |
| | | CATGGGCAAGCTGAGATACCTGCTGAA | |
| | | GGCCGACAAGACCTGCATCGACGGCCA | |
| | | GACCAGAGTCAGAGTGATCGAGCAGCC | |
| | | CCTGAACGGCTTCGGCAGACTGGAAGA | |
| | | GGCCGAGACAATGCGGAAGCAAGAGAA | |
| | | CGGCACCTTCGGCAACAGCGGCATCCG | |
| | | GATCAGAGACTTCGAGAACATGAAGCG | |
| | | GGACGACGCCAATCCTGCCAACTATCC | |
| | | CTACATCGTGGACACCTACACACACTA | |
| | | CATCCTGGAAAACAACAAGGTCGAGAT | |
| | | GTTTATCAACGACAAAGAGGACAGCGC | |
| | | CCCACTGCTGCCCGTGATCGAGGATGA | |
| | | TAGATACGTGGTCAAGACAATCCCCAG | |
| | | CTGCCGGATGAGCACCCTGGAAATTCC | |
| | | AGCCATGGCCTTCCACATGTTTCTGTT | |
| | | CGGCAGCAAGAAAACCGAGAAGCTGAT | |
| | | CGTGGACGTGCACAACCGGTACAAGAG | |
| | | ACTGTTCCAGGCCATGCAGAAAGAAGA | |
| | | AGTGACCGCCGAGAATATCGCCAGCTT | |
| | | CGGAATCGCCGAGAGCGACCTGCCTCA | |
| | | GAAGATCCTGGATCTGATCAGCGGCAA | |
| | | TGCCCACGGCAAGGATGTGGACGCCTT | |
| | | CATCAGACTGACCGTGGACGACATGCT | |
| | | GACCGACACCGAGCGGAGAATCAAGAG | |
| | | ATTCAAGGACGACCGGAAGTCCATTCG | |
| | | GAGCGCCGACAACAAGATGGGAAAGAG | |
| | | AGGCTTCAAGCAGATCTCCACAGGCAA | |
| | | GCTGGCCGACTTCCTGGCCAAGGACAT | |
| | | CGTGCTGTTTCAGCCCAGCGTGAACGA | |
| | | TGGCGAGAACAAGATCACCGGCCTGAA | |
| | | CTACCGGATCATGCAGAGCGCCATTGC | |
| | | CGTGTACGATAGCGGCGACGATTACGA | |
| | | GGCCAAGCAGCAGTTCAAGCTGATGTT | |
| | | CGAGAAGGCCCGGCTGATCGGCAAGGG | |
| | | CACAACAGAGCCTCATCCATTTCTGTA | |
| | | CAAGGTGTTCGCCCGCAGCATCCCCGC | |
| | | CAATGCCGTCGAGTTCTACGAGCGCTA | |
| | | CCTGATCGAGCGGAAGTTCTACCTGAC | |
| | | CGGCCTGTCCAACGAGATCAAGAAAGG | |
| | | CAACAGAGTGGATGTGCCCTTCATCCG | |
| | | GCGGGACCAGAACAAGTGGAAAACACC | |
| | | CGCCATGAAGACCCTGGGCAGAATCTA | |
| | | CAGCGAGGATCTGCCCGTGGAACTGCC | |
| | | CAGACAGATGTTCGACAATGAGATCAA | |
| | | GTCCCACCTGAAGTCCCTGCCACAGAT | |
| | | GGAAGGCATCGACTTCAACAATGCCAA | |
| | | CGTGACCTATCTGATCGCCGAGTACAT | |
| | | GAAGAGAGTGCTGGACGACGACTTCCA | |
| | | GACCTTCTACCAGTGGAACCGCAACTA | |
| | | CCGGTACATGGACATGCTTAAGGGCGA | |
| | | GTACGACAGAAAGGGCTCCCTGCAGCA | |
| | | CTGCTTCACCAGCGTGGAAGAGAGAGA | |
| | | AGGCCTCTGGAAAGAGCGGGCCTCCAG | |
| | | AACAGAGCGGTACAGAAAGCAGGCCAG | |
| | | CAACAAGATCCGCAGCAACCGGCAGAT | |
| | | GAGAAACGCCAGCAGCGAAGAGATCGA | |
| | | GACAATCCTGGATAAGCGGCTGAGCAA | |
| | | CAGCCGGAACGAGTACCAGAAAAGCGA | |
| | | GAAAGTGATCCGGCGCTACAGAGTGCA | |
| | | GGATGCCCTGCTGTTTCTGCTGGCCAA | |
| | | AAAGACCCTGACCGAACTGGCCGATTT | |
| | | CGACGGCGAGAGGTTCAAACTGAAAGA | |
| | | AATCATGCCCGACGCCGAGAAGGGAAT | |
| | | CCTGAGCGAGATCATGCCCATGAGCTT | |
| | | CACCTTCGAGAAAGGCGGCAAGAAGTA | |
| | | CACCATCACCAGCGAGGGCATGAAGCT | |
| | | GAAGAACTACGGCGACTTCTTTGTGCT | |
| | | GGCTAGCGACAAGAGGATCGGCAACCT | |
| | | GCTGGAACTCGTGGGCAGCGACATCGT | |
| | | GTCCAAAGAGGATGGATCCAAAAGAAC | |
| | | CGCCGACGGCAGCGAATTCGAGCCCAA | |
| | | GAAGAAGAGGAAAGTCTCTGGCAGCGA | |
| | | GACACCAGGAACAAGCGAGTCAGCAAC | |
| | | ACCAGAGAGCCAAGAGTTCTGTGACTA | |
| | | TGGCACCAAAGAGGAGTGCATGAAGGC | |
| | | TAGCGACGCTGATCGTCCATGCCGTAA | |
| | | GCTGCACTTCCGTCGCATCATTAACAA | |
| | | ACACACCGACGAGAGCCTGGGCGACTG | |
| | | CAGCTTCCTGAACACCTGCTTTCACAT | |
| | | GGACACCTGCAAGTACGTGCACTATGA | |
| | | GATCGACGCGTGCATGGATAGCGAAGC | |
| | | TCCGGGCAGCAAAGATCACACCCCGAG | |
| | | CCAGGAACTGGCCCTGACCCAGAGCGT | |
| | | GGGTGGTGACAGCAGCGCGGATCGTCT | |
| | | GTTCCCACCACAGTGGATCTGCTGCGA | |
| | | CATTCGTTACCTGGATGTGAGCATCCT | |
| | | GGGCAAGTTTGCTGTTGTGATGGCCGA | |
| | | CCCGCCGTGGGATATTCACATGGAGCT | |
| | | GCCGTATGGTACCCTGACCGACGATGA | |
| | | AATGCGTCGCCTGAACATCCCGGTGCT | |
| | | GCAGGACGATGGCTTCCTGTTTCTGTG | |
| | | GGTGACGGGTCGTGCTATGGAGCTGGG | |
| | | TCGTGAATGCCTGAACCTGTGGGGTTA | |
| | | CGAGCGTGTGGACGAAATCATTTGGGT | |
| | | GAAAACCAACCAGCTGCAGCGTATCAT | |
| | | TCGCACCGGCCGTACCGGTCACTGGCT | |
| | | GAACCACGGCAAGGAGCACTGCCTGGT | |
| | | GGGCGTGAAAGGCAACCCGCAGGGCTT | |
| | | TAACCAGGGTCTGGACTGCGATGTGAT | |
| | | CGTGGCTGAAGTGCGCAGCACCAGCCA | |
| | | CAAGCCGGACGAGATCTACGGCATGAT | |
| | | TGAACGCCTGAGCCCGGGTACCCGTAA | |
| | | AATTGAGCTGTTCGGCCGTCCGCACAA | |
| | | CGTGCAGCCGAACTGGATCACCCTGGG | |
| | | CAACCAGCTGGACGGTATTCACCTGCT | |
| | | GGACCCAGATGTGGTGGCTCGCTTTAA | |
| | | ACAGCGTTATCCGGATGGCATCATTAG | |
| | | CAAACCGAAGAATCTGTAACCGGTCAT | |
| | | CATCACCATCACCATTGAGTTTAAACC | |
| | | CGCTGATCAGCCTCGACTGTGCCTTCT | |
| | | AGTTGCCAGCCATCTGTTGTTTGCCCC | |
| | | TCCCCCGTGCCTTCCTTGACCCTGGAA | |
| | | GGTGCCACTCCCACTGTCCTTTCCTAA | |
| | | TAAAATGAGGAAATTGCATCGCATTGT | |
| | | CTGAGTAGGTGTCATTCTATTCTGGGG | |
| | | GGTGGGGTGGGGCAGGACAGCAAGGGG | |
| | | GAGGATTGGGAAGACAATAGCAGGCAT | |
| | | GCTGGGGATGCGGTGGGCTCTATGGCT | |
| | | TCTGAGGCGGAAAGAACCAGCTGGGGC | |
| | | TCGATACCGTCGACCTCTAGCTAGAGC | |
| | | TTGGCGTAATCATGGTCATAGCTGTTT | |
| | | CCTGTGTGAAATTGTTATCCGCTCACA | |
| | | ATTCCACACAACATACGAGCCGGAAGC | |
| | | ATAAAGTGTAAAGCCTAGGGTGCCTAA | |
| | | TGAGTGAGCTAACTCACATTAATTGCG | |
| | | TTGCGCTCACTGCCCGCTTTCCAGTCG | |
| | | GGAAACCTGTCGTGCCAGCTGCATTAA | |
| | | TGAATCGGCCAACGCGCGGGGAGAGGC | |
| | | GGTTTGCGTATTGGGCGCTCTTCCGCT | |
| | | TCCTCGCTCACTGACTCGCTGCGCTCG | |
| | | GTCGTTCGGCTGCGGCGAGCGGTATCA | |
| | | GCTCACTCAAAGGCGGTAATACGGTTA | |
| | | TCCACAGAATCAGGGGATAACGCAGGA | |
| | | AAGAACATGTGAGCAAAAGGCCAGCAA | |
| | | AAGGCCAGGAACCGTAAAAAGGCCGCG | |
| | | TTGCTGGCGTTTTTCCATAGGCTCCGC | |
| | | CCCCCTGACGAGCATCACAAAAATCGA | |
| | | CGCTCAAGTCAGAGGTGGCGAAACCCG | |
| | | ACAGGACTATAAAGATACCAGGCGTTT | |
| | | CCCCCTGGAAGCTCCCTCGTGCGCTCT | |
| | | CCTGTTCCGACCCTGCCGCTTACCGGA | |
| | | TACCTGTCCGCCTTTCTCCCTTCGGGA | |
| | | AGCGTGGCGCTTTCTCATAGCTCACGC | |
| | | TGTAGGTATCTCAGTTCGGTGTAGGTC | |
| | | GTTCGCTCCAAGCTGGGCTGTGTGCAC | |
| | | GAACCCCCCGTTCAGCCCGACCGCTGC | |
| | | GCCTTATCCGGTAACTATCGTCTTGAG | |
| | | TCCAACCCGGTAAGACACGACTTATCG | |
| | | CCACTGGCAGCAGCCACTGGTAACAGG | |
| | | ATTAGCAGAGCGAGGTATGTAGGCGGT | |
| | | GCTACAGAGTTCTTGAAGTGGTGGCCT | |
| | | AACTACGGCTACACTAGAAGAACAGTA | |
| | | TTTGGTATCTGCGCTCTGCTGAAGCCA | |
| | | GTTACCTTCGGAAAAAGAGTTGGTAGC | |
| | | TCTTGATCCGGCAAACAAACCACCGCT | |
| | | GGTAGCGGTGGTTTTTTTGTTTGCAAG | |
| | | CAGCAGATTACGCGCAGAAAAAAAGGA | |
| | | TCTCAAGAAGATCCTTTGATCTTTTCT | |
| | | ACGGGGTCTGACACTCAGTGGAACGAA | |
| | | AACTCACGTTAAGGGATTTTGGTCATG | |
| | | AGATTATCAAAAAGGATCTTCACCTAG | |
| | | ATCCTTTTAAATTAAAAATGAAGTTTT | |
| | | AAATCAATCTAAAGTATATATGAGTAA | |
| | | ACTTGGTCTGACAGTTACCAATGCTTA | |
| | | ATCAGTGAGGCACCTATCTCAGCGATC | |
| | | TGTCTATTTCGTTCATCCATAGTTGCC | |
| | | TGACTCCCCGTCGTGTAGATAACTACG | |
| | | ATACGGGAGGGCTTACCATCTGGCCCC | |
| | | AGTGCTGCAATGATACCGCGAGACCCA | |
| | | CGCTCACCGGCTCCAGATTTATCAGCA | |
| | | ATAAACCAGCCAGCCGGAAGGGCCGAG | |
| | | CGCAGAAGTGGTCCTGCAACTTTATCC | |
| | | GCCTCCATCCAGTCTATTAATTGTTGC | |
| | | CGGGAAGCTAGAGTAAGTAGTTCGCCA | |
| | | GTTAATAGTTTGCGCAACGTTGTTGCC | |
| | | ATTGCTACAGGCATCGTGGTGTCACGC | |
| | | TCGTCGTTTGGTATGGCTTCATTCAGC | |
| | | TCCGGTTCCCAACGATCAAGGCGAGTT | |
| | | ACATGATCCCCCATGTTGTGCAAAAAA | |
| | | GCGGTTAGCTCCTTCGGTCCTCCGATC | |
| | | GTTGTCAGAAGTAAGTTGGCCGCAGTG | |
| | | TTATCACTCATGGTTATGGCAGCACTG | |
| | | CATAATTCTCTTACTGTCATGCCATCC | |
| | | GTAAGATGCTTTTCTGTGACTGGTGAG | |
| | | TACTCAACCAAGTCATTCTGAGAATAG | |
| | | TGTATGCGGCGACCGAGTTGCTCTTGC | |
| | | CCGGCGTCAATACGGGATAATACCGCG | |
| | | CCACATAGCAGAACTTTAAAAGTGCTC | |
| | | ATCATTGGAAAACGTTCTTCGGGGCGA | |
| | | AAACTCTCAAGGATCTTACCGCTGTTG | |
| | | AGATCCAGTTCGATGTAACCCACTCGT | |
| | | GCACCCAACTGATCTTCAGCATCTTTT | |
| | | ACTTTCACCAGCGTTTCTGGGTGAGCA | |
| | | AAAACAGGAAGGCAAAATGCCGCAAAA | |
| | | AAGGGAATAAGGGCGACACGGAAATGT | |
| | | TGAATACTCATACTCTTCCTTTTTCAA | |
| | | TATTATTGAAGCATTTATCAGGGTTAT | |
| | | TGTCTCATGAGCGGATACATATTTGAA | |
| | | TGTATTTAGAAAAATAAACAAATAGGG | |
| | | GTTCCGCGCACATTTCCCCGAAAAGTG | |
| | | CCACCTGACGTCGACGGATCGGGAGAT | |
| | | CGATCTCCCGATCCCCTAGGGTCGACT | |
| | | CTCAGTACAATCTGCTCTGATGCCGCA | |
| | | TAGTTAAGCCAGTATCTGCTCCCTGCT | |
| | | TGTGTGTTGGAGGTCGCTGAGTAGTGC | |
| | | GCGAGCAAAATTTAAGCTACAACAAGG | |
| | | CAAGGCTTGACCGACAATTGCATGAAG | |
| | | AATCTGCTTAGGGTTAGGCGTTTTGCG | |
| | | CTGCTTCGCGATGTACGGGCCAGATAT | |
| | | ACGCGTTGACATTGATTATTGACTAGT | |
| | | TATTAATAGTAATCAATTACGGGGTCA | |
| | | TTAGTTCATAGCCCATATATGGAGTTC | |
| | | CGCGTTACATAACTTACGGTAAATGGC | |
| | | CCGCCTGGCTGACCGCCCAACGACCCC | |
| | | CGCCCATTGACGTCAATAATGACGTAT | |
| | | GTTCCCATAGTAACGCCAATAGGGACT | |
| | | TTCCATTGACGTCAATGGGTGGAGTAT | |
| | | TTACGGTAAACTGCCCACTTGGCAGTA | |
| | | CATCAAGTGTATCATATGCCAAGTACG | |
| | | CCCCCTATTGACGTCAATGACGGTAAA | |
| | | TGGCCCGCCTGGCATTATGCCCAGTAC | |
| | | ATGACCTTATGGGACTTTCCTACTTGG | |
| | | CAGTACATCTACGTATTAGTCATCGCT | |
| | | ATTACCATG | |
| 61 | pC016 | ATTGATTTAAAACTTCATTTTTAATTT | Backbone for |
| | | AAAAGGATCTAGGTGAAGATCCTTTTT | cloning LwCas13a |
| | | GATAATCTCATGACCAAAATCCCTTAA | guides under U6 |
| | | CGTGAGTTTTCGTTCCACTGAGCGTCA | promoter |
| | | GACCCCGTAGAAAAGATCAAAGGATCT | Addgene #91906, |
| | | TCTTGAGATCCTTTTTTTCTGCGCGTA | the sequence of |
| | | ATCTGCTGCTTGCAAACAAAAAAACCA | which is accessible |
| | | CCGCTACCAGCGGTGGTTTGTTTGCCG | at addgene.org/ |
| | | GATCAAGAGCTACCAACTCTTTTTCCG | 91906/ and |
| | | AAGGTAACTGGCTTCAGCAGAGCGCAG | incorporated |
| | | ATACCAAATACTGTTCTTCTAGTGTAG | by reference |
| | | CCGTAGTTAGGCCACCACTTCAAGAAC | |
| | | TCTGTAGCACCGCCTACATACCTCGCT | |
| | | CTGCTAATCCTGTTACCAGTGGCTGCT | |
| | | GCCAGTGGCGATAAGTCGTGTCTTACC | |
| | | GGGTTGGACTCAAGACGATAGTTACCG | |
| | | GATAAGGCGCAGCGGTCGGGCTGAACG | |
| | | GGGGGTTCGTGCACACAGCCCAGCTTG | |
| | | GAGCGAACGACCTACACCGAACTGAGA | |
| | | TACCTACAGCGTGAGCTATGAGAAAGC | |
| | | GCCACGCTTCCCGAAGGGAGAAAGGCG | |
| | | GACAGGTATCCGGTAAGCGGCAGGGTC | |
| | | GGAACAGGAGAGCGCACGAGGGAGCTT | |
| | | CCAGGGGGAAACGCCTGGTATCTTTAT | |
| | | AGTCCTGTCGGGTTTCGCCACCTCTGA | |
| | | CTTGAGCGTCGATTTTTGTGATGCTCG | |
| | | TCAGGGGGGCGGAGCCTATGGAAAAAC | |
| | | GCCAGCAACGCGGCCTTTTTACGGTTC | |
| | | CTGGCCTTTTGCTGGCCTTTTGCTCAG | |
| | | CTAGCGAGGGCCTATTTCCCATGATTC | |
| | | CTTCATATTTGCATATACGATACAAGG | |
| | | CTGTTAGAGAGATAATTGGAATTAATT | |
| | | TGACTGTAAACACAAAGATATTAGTAC | |
| | | AAAATACGTGACGTAGAAAGTAATAAT | |
| | | TTCTTGGGTAGTTTGCAGTTTTAAAAT | |
| | | TATGTTTTAAAATGGACTATCATATGC | |
| | | TTACCGTAACTTGAAAGTATTTCGATT | |
| | | TCTTGGCTTTATATATCTTGTGGAAAG | |
| | | GACGAAACACCGATTTAGACTACCCCA | |
| | | AAAACGAAGGGGACTAAAACGGGTCTT | |
| | | CGAGAAGACCTTTTTTTTGAATTCTGA | |
| | | TGCGGTATTTTCTCCTTACGCATCTGT | |
| | | GCGGTATTTCACACCGCATACGTCAAA | |
| | | GCAACCATAGTACGCGCCCTGTAGCGG | |
| | | CGCATTAAGCGCGGCGGGTGTGGTGGT | |
| | | TACGCGCAGCGTGACCGCTACACTTGC | |
| | | CAGCGCCTTAGCGCCCGCTCCTTTCGC | |
| | | TTTCTTCCCTTCCTTTCTCGCCACGTT | |
| | | CGCCGGCTTTCCCCGTCAAGCTCTAAA | |
| | | TCGGGGGCTCCCTTTAGGGTTCCGATT | |
| | | TAGTGCTTTACGGCACCTCGACCCCAA | |
| | | AAAACTTGATTTGGGTGATGGTTCACG | |
| | | TAGTGGGCCATCGCCCTGATAGACGGT | |
| | | TTTTCGCCCTTTGACGTTGGAGTCCAC | |
| | | GTTCTTTAATAGTGGACTCTTGTTCCA | |
| | | AACTGGAACAACACTCAACTCTATCTC | |
| | | GGGCTATTCTTTTGATTTATAAGGGAT | |
| | | TTTGCCGATTTCGGTCTATTGGTTAAA | |
| | | AAATGAGCTGATTTAACAAAAATTTAA | |
| | | CGCGAATTTTAACAAAATATTAACGTT | |
| | | TACAATTTTATGGTGCACTCTCAGTAC | |
| | | AATCTGCTCTGATGCCGCATAGTTAAG | |
| | | CCAGCCCCGACACCCGCCAACACCCGC | |
| | | TGACGCGCCCTGACGGGCTTGTCTGCT | |
| | | CCCGGCATCCGCTTACAGACAAGCTGT | |
| | | GACCGTCTCCGGGAGCTGCATGTGTCA | |
| | | GAGGTTTTCACCGTCATCACCGAAACG | |
| | | CGCGAGACGAAAGGGCCTCGTGATACG | |
| | | CCTATTTTTATAGGTTAATGTCATGAT | |
| | | AATAATGGTTTCTTAGACGTCAGGTGG | |
| | | CACTTTTCGGGGAAATGTGCGCGGAAC | |
| | | CCCTATTTGTTTATTTTTCTAAATACA | |
| | | TTCAAATATGTATCCGCTCATGAGACA | |
| | | ATAACCCTGATAAATGCTTCAATAATA | |
| | | TTGAAAAAGGAAGAGTATGAGTATTCA | |
| | | ACATTTCCGTGTCGCCCTTATTCCCTT | |
| | | TTTTGCGGCATTTTGCCTTCCTGTTTT | |
| | | TGCTCACCCAGAAACGCTGGTGAAAGT | |
| | | AAAAGATGCTGAAGATCAGTTGGGTGC | |
| | | ACGAGTGGGTTACATCGAACTGGATCT | |
| | | CAACAGCGGTAAGATCCTTGAGAGTTT | |
| | | TCGCCCCGAAGAACGTTTTCCAATGAT | |
| | | GAGCACTTTTAAAGTTCTGCTATGTGG | |
| | | CGCGGTATTATCCCGTATTGACGCCGG | |
| | | GCAAGAGCAACTCGGTCGCCGCATACA | |
| | | CTATTCTCAGAATGACTTGGTTGAGTA | |
| | | CTCACCAGTCACAGAAAAGCATCTTAC | |
| | | GGATGGCATGACAGTAAGAGAATTATG | |
| | | CAGTGCTGCCATAACCATGAGTGATAA | |
| | | CACTGCGGCCAACTTACTTCTGACAAC | |
| | | GATCGGAGGACCGAAGGAGCTAACCGC | |
| | | TTTTTTGCACAACATGGGGGATCATGT | |
| | | AACTCGCCTTGATCGTTGGGAACCGGA | |
| | | GCTGAATGAAGCCATACCAAACGACGA | |
| | | GCGTGACACCACGATGCCTGTAGCAAT | |
| | | GGCAACAACGTTGCGCAAACTATTAAC | |
| | | TGGCGAACTACTTACTCTAGCTTCCCG | |
| | | GCAACAATTAATAGACTGGATGGAGGC | |
| | | GGATAAAGTTGCAGGACCACTTCTGCG | |
| | | CTCGGCCCTTCCGGCTGGCTGGTTTAT | |
| | | TGCTGATAAATCTGGAGCCGGTGAGCG | |
| | | TGGAAGCCGCGGTATCATTGCAGCACT | |
| | | GGGGCCAGATGGTAAGCCCTCCCGTAT | |
| | | CGTAGTTATCTACACGACGGGGAGTCA | |
| | | GGCAACTATGGATGAACGAAATAGACA | |
| | | GATCGCTGAGATAGGTGCCTCACTGAT | |
| | | TAAGCATTGGTAACTGTCAGACCAAGT | |
| | | TTACTCATATATACTTTAG | |
| 62 | LVDP-CArG-RE-GPR | atggatagcactgagaacgtcatcaag | dsRED-Express2 |
| | | cccttcatgcgcttcaaggtgcacatg | fragment source to |
| | | gagggctccgtgaacggccacgagttc | clone in GEMS |
| | | gagatcgagggcgagggcgagggcaag | Addgene #89762, |
| | | ccctacgagggcacccagaccgccaag | the sequence of |
| | | ctgcaggtgaccaagggcggccccctg | which is accessible |
| | | cccttcgcctgggacatcctgtccccc | at addgene.org/ |
| | | cagttccagtacggctccaaggtgtac | 89762/ and |
| | | gtgaagcaccccgccgacatccccgac | incorporated |
| | | tacaagaagctgtccttccccgagggc | by reference |
| | | ttcaagtgggagcgcgtgatgaacttc | |
| | | gaggacggcggcgtggtgaccgtgacc | |
| | | caggactcctccctgcaggacggcacc | |
| | | ttcatctaccacgtgaagttcatcggc | |
| | | gtgaacttcccctccgacggccccgta | |
| | | atgcagaagaagactctgggctgggag | |
| | | ccctccaccgagcgcctgtacccccgc | |
| | | gacggcgtgctgaagggcgagatccac | |
| | | aaggcgctgaagctgaagggcggcggc | |
| | | cactacctggtggagttcaagtcaatc | |
| | | tacatggccaagaagcccgtgaagctg | |
| | | cccggctactactacgtggactccaag | |
| | | ctggacatcacctcccacaacgaggac | |
| | | tacaccgtggtggagcagtacgagcgc | |
| | | gccgaggcccgccaccacctgttccag | |
| | | tag | |
| 63 | EGFP sequence | ATGGTGAGCAAGGGCGAGGAGCTGTTC | |
| | | ACCGGGGTGGTGCCCATCCTGGTCGAG | |
| | | CTGGATGGCGATGTAAATGGCCACAAG | |
| | | TTCAGCGTGTCCGGCGAGGGCGAGGGC | |
| | | GATGCCACCTACGGCAAGCTCACCCTG | |
| | | AAGTTCATCTGCACCACCGGCAAGCTG | |
| | | CCCGTGCCCTGGCCCACCCTCGTCACC | |
| | | ACCCTCACCTACGGCGTGCAGTGCTTC | |
| | | AGCCGCTACCCCGATCACATGAAGCAG | |
| | | CACGATTTCTTCAAGTCCGCCATGCCC | |
| | | GAAGGCTACGTCCAGGAGCGCACCATC | |
| | | TTCTTCAAGGATGATGGCAATTACCGT | |
| | | ACCCGCGCCGAGGTGAAGTTCGAGGGC | |
| | | GATACCCTGGTGAATCGCATCGAGCTG | |
| | | AAGGGCATCGATTTCAAGGAGGATGGC | |
| | | AATATCCTGGGGCACAAGCTGGAGTAC | |
| | | AATTACAATAGCCACAATGTCTATATC | |
| | | ATGGCCGATAAGCAGAAGAATGGCATC | |
| | | AAGGTGAATTTCAAGATCCGCCACAAT | |
| | | ATCGAGGATGGCAGCGTGCAGCTCGCC | |
| | | GATCACTACCAGCAGAATACCCCCATC | |
| | | GGCGATGGCCCCGTGCTGCTGCCCGAT | |
| | | AATCACTACCTGAGCACCCAGTCCGCC | |
| | | CTGAGCAAAGATCCCAATGAGAAGCGC | |
| | | GATCACATGGTCCTGCTGGAGTTCGTC | |
| | | ACCGCCGCCGGGATCACTCTCGGCATG | |
| | | GATGAGCTGTACAAG | |
| 64 | m⁶A sensor linker | GCGGACTTACGACAGTTGCGTTACACC | |
| | sequence | CTTTCTCGACAAAACCTAACTTGCGCA | |
| | | GAAAACATGCCAATCTCATCTTGGCTT | |
| 65 | PEST domain | TTGCTTAGCCATGGCTTCCCGCCGGAG | |
| | sequence | GTGGAGGAGCAGGATGATGGCACGCTG | |
| | | CCCATGTCTTGTGCCCAGGAGAGCGGG | |
| | | ATGGACCGTCACCCTGCAGCCTGTGCT | |
| | | TCTGCTAGGATCAATGTGTTAGATGCG | |
| 66 | m⁶A sensor sequence | GACTTACGACAG | |
| 67 | amino acid sequence | PHPVLEKLRSINNYNPKDFDWNLKHGR | |
| | of YTHDF2-YTH | VFIIKSYSEDDIHRSIKYNIWCSTEHG | |
| | | NKRLDAAYRSMNGKGPVYLLFSVNGSG | |
| | | HFCGVAEMKSAVDYNTCAGVWSQDKWK | |
| | | GRFDVRWIFVKDVPNSQLRHIRLENNE | |
| | | NKPVTNSRDTQEVPLEKAKQVLKIIAS | |
| | | YKHTTSIFDDFSHYEKRQEEEESVKKE | |
| | | RQGRGK | |
| 68 | amino acid sequence | PHPVLEKLRSINNYNPKDFDWNLKHGR | |
| | of YTHDF2- | VFIIKSYSEDDIHRSIKYNIACSTEHG | |
| | YTH_W432A_W486A | NKRLDAAYRSMNGKGPVYLLFSVNGSG | |
| | | HFCGVAEMKSAVDYNTCAGVASQDKWK | |
| | | GRFDVRWIFVKDVPNSQLRHIRLENNE | |
| | | NKPVTNSRDTQEVPLEKAKQVLKIIAS | |
| | | YKHTTSIFDDFSHYEKRQEEEESVKKE | |
| | | RQGRGK | |
| 69 | amino acid sequence | GRVFIIKSYSEDDIHRSIKYNIWCSTE | |
| | of YTHDF2-YTHmut | HGNKRLDAAYRSMNGKGPVYLLFSVNG | |
| | | SGHFCGVAEMKSAVDYNTCAGVWSQDK | |
| | | WKGRFDVRWIFVKDVPNSQLRHIRLEN | |
| | | NENKPVTNSRDTQEVPLEKAKQVLKII | |
| | | ASYKHTTSIFDDFSHYEKRQEEEESVK | |
| | | KERQGRGK | |
| 70 | amino acid sequence | GRVFIIKSYSEDDIHRSIKYNIACSTE | |
| | of YTHDF2-YTHmut2 | HGNKRLDAAYRSMNGKGPVYLLFSVNG | |
| | | SGHFCGVAEMKSAVDYNTCAGVASQDK | |
| | | WKGRFDVRWIFVKDVPNSQLRHIRLEN | |
| | | NENKPVTNSRDTQEVPLEKAKQVLKII | |
| | | ASYKHTTSIFDDFSHYEKRQEEEESVK | |
| | | KERQGRGK | |
| 71 | amino acid sequence | PHPVLEKLRSINNYNPKDFDWNLKHGR | |
| | of YTHDF2-YTH | VFIIKSYSENDIHRSIKYNIWCSTEHG | |
| | D422N | NKRLDAAYRSMNGKGPVYLLFSVNGSG | |
| | | HFCGVAEMKSAVDYNTCAGVWSQDKWK | |
| | | GRFDVRWIFVKDVPNSQLRHIRLENNE | |
| | | NKPVTNSRDTQEVPLEKAKQVLKIIAS | |
| | | YKHTTSIFDDFSHYEKRQEEEESVKKE | |
| | | RQGRGK | |
| 72 | amino acid sequence | HPVLEKLKAAHSYNPKEFEWNLKSGRV | |
| | of YTHDF1 | FIIKSYSEDDIHRSIKYSIWCSTEHGN | |
| | | KRLDSAFRCMSSKGPVYLLFSVNGSGH | |
| | | FCGVAEMKSPVDYGTSAGVWSQDKWKG | |
| | | KFDVQWIFVKDVPNNQLRHIRLENNDN | |
| | | KPVTNSRDTQEVPLEKAKQVLKIISSY | |
| | | KHTTSIFDDFAHYEKRQEEEEVVRKER | |
| | | QSRNKQ | |
| 73 | amino acid sequence | GRVFIIKSYSEDDIHRSIKYSIWCSTE | |
| | of YTHDF1mut | HGNKRLDSAFRCMSSKGPVYLLFSVNG | |
| | | SGHFCGVAEMKSPVDYGTSAGVWSQDK | |
| | | WKGKFDVQWIFVKDVPNNQLRHIRLEN | |
| | | NDNKPVTNSRDTQEVPLEKAKQVLKII | |
| | | SSYKHTTSIFDDFAHYEKRQEEEEVVR | |
| | | KERQSRNKQ | |
| 74 | amino acid sequence | HPVLEKLKAAHSYNPKEFEWNLKSGRV | |
| | of YTHDF1 D401N | FIIKSYSEDNIHRSIKYSIWCSTEHGN | |
| | | KRLDSAFRCMSSKGPVYLLFSVNGSGH | |
| | | FCGVAEMKSPVDYGTSAGVWSQDKWKG | |
| | | KFDVQWIFVKDVPNNQLRHIRLENNDN | |
| | | KPVTNSRDTQEVPLEKAKQVLKIISSY | |
| | | KHTTSIFDDFAHYEKRQEEEEVVRKER | |
| | | QSRNKQ | |
| 75 | amino acid sequence | VHPVLEKLKAINNYNPKDFDWNLKNGR | |
| | of YTHDF3 | VFIIKSYSEDDIHRSIKYSIWCSTEHG | |
| | | NKRLDAAYRSLNGKGPLYLLFSVNGSG | |
| | | HFCGVAEMKSVVDYNAYAGVWSQDKWK | |
| | | GKFEVKWIFVKDVPNNQLRHIRLENND | |
| | | NKPVTNSRDTQEVPLEKAKQVLKIIAT | |
| | | FKHTTSIFDDFAHYEKRQEEEEAMRRE | |
| | | RNRNKQ | |
| 76 | amino acid sequence | SKLKYVLQDARFFLIKSNNHENVSLAK | |
| | of YTHDC1 | AKGVWSTLPVNEKKLNLAFRSARSVIL | |
| | | IFSVRESGKFQGFARLSSESHHGGSPI | |
| | | HWVLPAGMSAKMLGGVFKIDWICRREL | |
| | | PFTKSAHLTNPWNEHKPVKIGRDGQEI | |
| | | ELECGTQLCLLFPPDESIDLYQVIHKM | |
| | | RHK | |
| 77 | amino acid sequence | PVRYFIMKSSNLRNLEISQQKGIWSTT | |
| | of YTHDC2 | PSNERKLNRAFWESSIVYLVFSVQGSG | |
| | | HFQGFSRMSSEIGREKSQDWGSAGLGG | |
| | | VFKVEWIRKESLPFQFAHHLLNPWNDN | |
| | | KKVQISRDGQELEPLVGEQLLQLWERL | |
| | | PLGEKNTTD | |
| 78 | amino acid sequence | MSSETGPVAVDPTLRRRIEPHEFEVFF | |
| | of rAPOBEC1 | DPRELRKETCLLYEINWGGRHSIWRHT | |
| | | SQNTNKHVEVNFIEKFTTERYFCPNTR | |
| | | CSITWFLSWSPCGECSRAITEFLSRYP | |
| | | HVTLFIYIARLYHHADPRNRQGLRDLI | |
| | | SSGVTIQIMTEQESGYCWRNFVNYSPS | |
| | | NEAHWPRYPHLWVRLYVLELYCIILGL | |
| | | PPCLNILRRKQPQLTFFTIALQSCHYQ | |
| | | RLPPHILWATGLK | |
| 79 | amino acid sequence | MDSLLMNRRKFLYQFKNVRWAKGRRET | |
| | of hAICDA | YLCYVVKRRDSATSFSLDFGYLRNKNG | |
| | | CHVELLFLRYISDWDLDPGRCYRVTWF | |
| | | TSWSPCYDCARHVADFLRGNPNLSLRI | |
| | | FTARLYFCEDRKAEPEGLRRLHRAGVQ | |
| | | IAIMTFKDYFYCWNTFVENHERTFKAW | |
| | | EGLHENSVRLSRQLRRILLPLYEVDDL | |
| | | RDAFRTLGL | |
| 80 | amino acid sequence | MEASPASGPRHLMDPHIFTSNFNNGIG | |
| | of hAPOBEC3A | RHKTYLCYEVERLDNGTSVKMDQHRGF | |
| | | LHNQAKNLLCGFYGRHAELRFLDLVPS | |
| | | LQLDPAQIYRVTWFISWSPCFSWGCAG | |
| | | EVRAFLQENTHVRLRIFAARIYDYDPL | |
| | | YKEALQMLRDAGAQVSIMTYDEFKHCW | |
| | | DTFVDHQGCPFQPWDGLDEHSQALSGR | |
| | | LRAILQNQGN | |
| 81 | amino acid sequence | QLHLPQVLADAVSRLVLGKFGDLTDNF | |
| | of catalytic domain | SSPHARRKVLAGVVMTTGTDVKDAKVI | |
| | of ADAR2 | SVSTGTKCINGEYMSDRGLALNDCHAE | |
| | | IISRRSLLRFLYTQLELYLNNKDDQKR | |
| | | SIFQKSERGGFRLKENVQFHLYISTSP | |
| | | CGDARIFSPHEPILEEPADRHPNRKAR | |
| | | GQLRTKIESGQGTIPVRSNASIQTWDG | |
| | | VLQGERLLTMSCSDKIARWNVVGIQGS | |
| | | LLSIFVEPIYFSSIILGSLYHGDHLSR | |
| | | AMYQRISNIEDLPPLYTLNKPLLSGIS | |
| | | NAEARQPGKAPNFSVNWTVGDSAIEVI | |
| | | NATTGKDELGRASRLCKHALYCRWMRV | |
| | | HGKVPSHLLRSKITKPNVYHESKLAAK | |
| | | EYQAAKARLFTAFIKAGLGAWVEKPTE | |
| | | QDQFSLT | |
| 82 | | SGSETPGTSESATPE | |
| 83 | | SGSETPGTSESATPES | |
| 84 | | (GGGGS)3 | |
| 85 | | ((GGGGS)10) | |
| 86 | | (A(EAAAK)3A) | |
| 87 | E. coli codon | ATGAGCAGCGAAACCGGTCCGGTGGCG | |
| | optimized APOBEC1- | GTTGACCCGACCCTGCGTCGTCGTATT | |
| | YTH for protein | GAGCCGCACGAGTTCGAAGTGTTCTTT | |
| | purification | GATCCGCGTGAGCTGCGTAAGGAAACC | |
| | | TGCCTGCTGTACGAAATTAACTGGGGT | |
| | | GGCCGTCACAGCATCTGGCGTCACACC | |
| | | AGCCAGAACACCAACAAGCACGTTGAG | |
| | | GTGAACTTCATCGAAAAATTTACCACC | |
| | | GAGCGTTACTTCTGCCCGAACACCCGT | |
| | | TGCAGCATTACCTGGTTTCTGAGCTGG | |
| | | AGCCCGTGCGGTGAATGCAGCCGTGCG | |
| | | ATCACCGAGTTCCTGAGCCGTTATCCG | |
| | | CACGTTACCCTGTTTATCTACATTGCG | |
| | | CGTCTGTATCACCACGCGGACCCGCGT | |
| | | AACCGTCAAGGTCTGCGTGATCTGATC | |
| | | AGCAGCGGCGTGACCATCCAGATTATG | |
| | | ACCGAGCAAGAAAGCGGTTACTGCTGG | |
| | | CGTAACTTCGTTAACTATAGCCCGAGC | |
| | | AACGAAGCGCATTGGCCGCGTTACCCG | |
| | | CACCTGTGGGTGCGTCTGTACGTTCTG | |
| | | GAGCTGTATTGCATCATTCTGGGCCTG | |
| | | CCGCCGTGCCTGAACATTCTGCGTCGT | |
| | | AAGCAGCCGCAACTGACCTTCTTTACC | |
| | | ATCGCGCTGCAGAGCTGCCACTACCAA | |
| | | CGTCTGCCGCCGCACATTCTGTGGGCG | |
| | | ACCGGTCTGAAGAGCGGCAGCGAAACC | |
| | | CCGGGTACCAGCGAAAGCGCGACCCCG | |
| | | GAGCCGCACCCGGTGCTGGAGAAACTG | |
| | | CGTAGCATCAACAACTATAACCCGAAG | |
| | | GACTTCGATTGGAACCTGAAACACGGT | |
| | | CGTGTTTTTATCATTAAGAGCTACAGC | |
| | | GAAGACGATATCCACCGTAGCATTAAA | |
| | | TATAACATCTGGTGCAGCACCGAGCAC | |
| | | GGCAACAAGCGTCTGGACGCGGCGTAC | |
| | | CGTAGCATGAACGGTAAAGGCCCGGTG | |
| | | TATCTGCTGTTCAGCGTTAACGGTAGC | |
| | | GGCCACTTTTGCGGTGTGGCGGAAATG | |
| | | AAAAGCGCGGTTGATTACAACACCTGC | |
| | | GCGGGTGTGTGGAGCCAGGACAAGTGG | |
| | | AAAGGCCGTTTCGATGTTCGTTGGATT | |
| | | TTTGTGAAGGACGTTCCGAACAGCCAA | |
| | | CTGCGTCACATCCGTCTGGAGAACAAC | |
| | | GAAAACAAACCGGTGACCAACAGCCGT | |
| | | GATACCCAGGAAGTGCCGCTGGAAAAG | |
| | | GCGAAACAAGTTCTGAAGATCATTGCG | |
| | | AGCTACAAACACACCACCAGCATCTTC | |
| | | GACGATTTTAGCCACTATGAGAAGCGT | |
| | | CAGGAAGAGGAAGAGAGCGTGAAGAAG | |
| | | GAGCGTCAAGGTCGTGGCAAACTGGAG | |
| | | TACCCGTATGACGTTCCGGATTATGCG | |
| | | TAAATTGGAAGTGGATAA | |
| 88 | E. coli codon | ATGAGCAGCGAAACCGGTCCGGTGGCG | |
| | optimized APOBEC1- | GTTGACCCGACCCTGCGTCGTCGTATT | |
| | YTHmut for protein | GAGCCGCACGAGTTCGAAGTGTTCTTT | |
| | purification | GATCCGCGTGAGCTGCGTAAGGAAACC | |
| | | TGCCTGCTGTACGAAATTAACTGGGGT | |
| | | GGCCGTCACAGCATCTGGCGTCACACC | |
| | | AGCCAGAACACCAACAAGCACGTTGAG | |
| | | GTGAACTTCATCGAAAAATTTACCACC | |
| | | GAGCGTTACTTCTGCCCGAACACCCGT | |
| | | TGCAGCATTACCTGGTTTCTGAGCTGG | |
| | | AGCCCGTGCGGTGAATGCAGCCGTGCG | |
| | | ATCACCGAGTTCCTGAGCCGTTATCCG | |
| | | CACGTTACCCTGTTTATCTACATTGCG | |
| | | CGTCTGTATCACCACGCGGACCCGCGT | |
| | | AACCGTCAAGGTCTGCGTGATCTGATC | |
| | | AGCAGCGGCGTGACCATCCAGATTATG | |
| | | ACCGAGCAAGAAAGCGGTTACTGCTGG | |
| | | CGTAACTTCGTTAACTATAGCCCGAGC | |
| | | AACGAAGCGCATTGGCCGCGTTACCCG | |
| | | CACCTGTGGGTGCGTCTGTACGTTCTG | |
| | | GAGCTGTATTGCATCATTCTGGGCCTG | |
| | | CCGCCGTGCCTGAACATTCTGCGTCGT | |
| | | AAGCAGCCGCAACTGACCTTCTTTACC | |
| | | ATCGCGCTGCAGAGCTGCCACTACCAA | |
| | | CGTCTGCCGCCGCACATTCTGTGGGCG | |
| | | ACCGGTCTGAAGAGCGGCAGCGAAACC | |
| | | CCGGGTACCAGCGAAAGCGCGACCCCG | |
| | | GAGGGTCGTGTTTTTATCATTAAGAGC | |
| | | TACAGCGAAGACGATATCCACCGTAGC | |
| | | ATTAAATATAACATCTGGTGCAGCACC | |
| | | GAGCACGGCAACAAGCGTCTGGACGCG | |
| | | GCGTACCGTAGCATGAACGGTAAAGGC | |
| | | CCGGTGTATCTGCTGTTCAGCGTTAAC | |
| | | GGTAGCGGCCACTTTTGCGGTGTGGCG | |
| | | GAAATGAAAAGCGCGGTTGATTACAAC | |
| | | ACCTGCGCGGGTGTGTGGAGCCAGGAC | |
| | | AAGTGGAAAGGCCGTTTCGATGTTCGT | |
| | | TGGATTTTTGTGAAGGACGTTCCGAAC | |
| | | AGCCAACTGCGTCACATCCGTCTGGAG | |
| | | AACAACGAAAACAAACCGGTGACCAAC | |
| | | AGCCGTGATACCCAGGAAGTGCCGCTG | |
| | | GAAAAGGCGAAACAAGTTCTGAAGATC | |
| | | ATTGCGAGCTACAAACACACCACCAGC | |
| | | ATCTTCGACGATTTTAGCCACTATGAG | |
| | | AAGCGTCAGGAAGAGGAAGAGAGCGTG | |
| | | AAGAAGGAGCGTCAAGGTCGTGGCAAA | |
| | | CTGGAGTACCCGTATGACGTTCCGGAT | |
| | | TATGCGTAAATTGGAAGTGGATAA | |
| 89 | | PKKKRKV | |
| 90 | | LPPLERLTL | |
| 91 | | MDPVVVLGLCLSCLLLLSLWKQSYGGG | |
| 92 | | METDTLLLWVLLLWVPGSTGD | |
| 93 | | EQKLISEEDL | |
| 94 | | GKPIPNPLLGLDST | |
| 95 | | IPNPLLGLD | |
| 96 | | DYKDDDDK | |
| 97 | | DYKDHDGDYKDHDIDYKDDDDK | |
| 98 | DHFR domain | ISLIAALAVDHVIGMETVMPWNLPADL | |
| | | AWFKRNTLNKPVIMGRHTWESIGRPLP | |
| | | GRKNIILSSQPSTDDRVTWVKSVDEAI | |
| | | AACGDVPEIMVIGGGRVYEQFLPKAQK | |
| | | LYLTHIDAEVEGDTHFPDYEPDDWESV | |
| | | FSEFHDADAQNSHSYCFEILERR | |
| 99 | | GACUUAUGACAG | |
| 100 | | GACUUACGACAG | |
| 101 | | GGACTTACGACAGTT | |
| 102 | | GGCUUACGACAG | |
| 103 | | GACUUACGAGAG | |
| 104 | | HHHHHH | |
| 105 | Construct comprising | gtcgacggatcgggagatctcccgatc | |
| | a nucleic acid | ccctatggtgcactctcagtacaatct | |
| | encoding GFP, a m6A | gctctgatgccgcatagttaagccagt | |
| | reporter sequence and | atctgctccctgcttgtgtgttggagg | |
| | DHFR; and a nucleic | tcgctgagtagtgcgcgagcaaaattt | |
| | acid encoding | aagctacaacaaggcaaggcttgaccg | |
| | APOBEC1-YTH (5′-3′) | acaattgcatgaagaatctgcttaggg | |
| | | ttaggcgttttgcgctgcttcgcgatg | |
| | | tacgggccagatatacgcgttgacatt | |
| | | gattattgactagttattaatagtaat | |
| | | caattacggggtcattagttcatagcc | |
| | | catatatggagttccgcgttacataac | |
| | | ttacggtaaatggcccgcctggctgac | |
| | | cgcccaacgacccccgcccattgacgt | |
| | | caataatgacgtatgttcccatagtaa | |
| | | cgccaatagggactttccattgacgtc | |
| | | aatgggtggagtatttacggtaaactg | |
| | | cccacttggcagtacatcaagtgtatc | |
| | | atatgccaagtacgccccctattgacg | |
| | | tcaatgacggtaaatggcccgcctggc | |
| | | attatgcccagtacatgaccttatggg | |
| | | actttcctacttggcagtacatctacg | |
| | | tattagtcatcgctattaccatggtga | |
| | | tgcggttttggcagtacatcaatgggc | |
| | | gtggatagcggtttgactcacggggat | |
| | | ttccaagtctccaccccattgacgtca | |
| | | atgggagtttgttttggcaccaaaatc | |
| | | aacgggactttccaaaatgtogtaaca | |
| | | actccgccccattgacgcaaatgggcg | |
| | | gtaggcgtgtacggtgggaggtctata | |
| | | taagcagagctggtttagtgaaccgtc | |
| | | agatccgctagagatccgcggccgcgc | |
| | | tagcgtttaaacgggccctctagagcc | |
| | | gccatggtgagcaagggcgaggagctg | |
| | | ttcaccggggtggtgcccatcctggtc | |
| | | gagctggatggcgatgtaaatggccac | |
| | | aagttcagcgtgtccggcgagggcgag | |
| | | ggcgatgccacctacggcaagctcacc | |
| | | ctgaagttcatctgcaccaccggcaag | |
| | | ctgcccgtgccctggcccaccctcgtc | |
| | | accaccctcacctacggcgtgcagtgc | |
| | | ttcagccgctaccccgatcacatgaag | |
| | | cagcacgatttcttcaagtccgccatg | |
| | | cccgaaggctacgtccaggagcgcacc | |
| | | atcttcttcaaggatgatggcaattac | |
| | | cgtacccgcgccgaggtgaagttcgag | |
| | | ggcgataccctggtgaatcgcatcgag | |
| | | ctgaagggcatcgatttcaaggaggat | |
| | | ggcaatatcctggggcacaagctggag | |
| | | tacaattacaatagccacaatgtctat | |
| | | atcatggccgataagcagaagaatggc | |
| | | atcaaggtgaatttcaagatccgccac | |
| | | aatatcgaggatggcagcgtgcagctc | |
| | | gccgatcactaccagcagaataccccc | |
| | | atcggcgatggccccgtgctgctgccc | |
| | | gataatcactacctgagcacccagtcc | |
| | | gccctgagcaaagatcccaatgagaag | |
| | | cgcgatcacatggtcctgctggagttc | |
| | | gtcaccgccgccgggatcactctcggc | |
| | | atggatgagctgtacaaggcggactta | |
| | | cgacagttgcgttacaccctttctcga | |
| | | caaaacctaacttgcgcagaaaacatg | |
| | | ccaatctcatcttggcttatcagtctg | |
| | | attgcggcgttagcggtagatcacgtt | |
| | | atcggcatggaaaccgtcatgccgtgg | |
| | | aacctgcctgccgatctcgcctggttt | |
| | | aaacgcaacaccttaaataaacccgtg | |
| | | attatgggccgccatacctgggaatca | |
| | | atcggtcgtccgttgccaggacgcaaa | |
| | | aatattatcctcagcagtcaaccgagt | |
| | | acggacgatcgcgtaacgtgggtgaag | |
| | | tcggtggatgaagccatcgcggcgtgt | |
| | | ggtgacgtaccagaaatcatggttatt | |
| | | ggcggcggtcgcgtttatgaacagttc | |
| | | ttgccaaaagcgcaaaaactgtatctg | |
| | | acgcatatcgacgcagaagtggaaggc | |
| | | gacacccatttcccggattacgagccg | |
| | | gatgactgggaatcggtattcagcgaa | |
| | | ttccacgatgctgatgcgcagaactct | |
| | | cacagctattgctttgagattctggag | |
| | | cggcgataagcctcattgtgcattctc | |
| | | tcgagtacccctacgacgtgcccgact | |
| | | acgcctgagggacccgacaggcccgaa | |
| | | ggaatagaagaagaaggtggagagaga | |
| | | gacagagacagatccattcgattagtg | |
| | | aacggatcggcactgcgtgcgccaatt | |
| | | ctgcagacaaatggcagtattcatcca | |
| | | caattttaaaagaaaaggggggattgg | |
| | | ggggtacagtgcaggggaaagaatagt | |
| | | agacataatagcaacagacatacaaac | |
| | | taaagaattacaaaaacaaattacaaa | |
| | | aattcaaaattttcgggtttattacag | |
| | | ggacagcagagatccagtttggttacc | |
| | | agtgtgatggatatctgcagaattcgc | |
| | | ccttggatccgaattcctgcagccccg | |
| | | actttcacttttctctatcactgatag | |
| | | ggagtggtaaactcgactttcactttt | |
| | | ctctatcactgatagggagtggtaaac | |
| | | tcgactttcacttttctctatcactga | |
| | | tagggagtggtaaactcgactttcact | |
| | | tttctctatcactgatagggagtggta | |
| | | aactcgactttcacttttctctatcac | |
| | | tgatagggagtggtaaactcgactttc | |
| | | acttttctctatcactgatagggagtg | |
| | | gtaaactcgactttcacttttctctat | |
| | | cactgatagggagtggtaaactcgagg | |
| | | gggatccactagcatgaagggcgaatt | |
| | | ccagcacactggtaacccgtgtcggct | |
| | | ccagatctggcctccgcgccgggtttt | |
| | | ggcgcctcccgcgggcgcccccctcct | |
| | | cacggcgagccgcgttgacattgatta | |
| | | ttgactaggcttttgcaaaaagctttg | |
| | | caaagatggataaagttttaaacagag | |
| | | aggaatctttgcagctaatggaccttc | |
| | | taggtcttgaaaggagtgggaattggc | |
| | | tccggtgcccgtcagtgggcagagcgc | |
| | | acatcgcccacagtccccgagaagttg | |
| | | gggggaggggtcggcaattgaaccggt | |
| | | gcctagagaaggtggcgcggggtaaac | |
| | | tgggaaagtgatgtcgtgtactggctc | |
| | | cgcctttttcccgagggtgggggagaa | |
| | | ccgtatataagtgcagtagtcgccgtg | |
| | | aacgttctttttcgcaacgggtttgcc | |
| | | gccagaacacaggtaagtgccgtgtgt | |
| | | ggttcccgcgggcctggcctctttacg | |
| | | ggttatggcccttgcgtgccttgaatt | |
| | | acttccacctggctgcagtacgtgatt | |
| | | cttgatcccgagcttcgggttggaagt | |
| | | gggtgggagagttcgaggccttgcgct | |
| | | taaggagccccttcgcctcgtgcttga | |
| | | gttgaggcctggcctgggcgctggggc | |
| | | cgccgcgtgcgaatctggtggcacctt | |
| | | cgcgcctgtctcgctgctttcgataag | |
| | | tctctagccatttaaaatttttgatga | |
| | | cctgctgcgacgctttttttctggcaa | |
| | | gatagtcttgtaaatgcgggccaagat | |
| | | ctgcacactggtatttcggtttttggg | |
| | | gccgcgggcggcgacggggcccgtgcg | |
| | | tcccagcgcacatgttcggcgaggcgg | |
| | | ggcctgcgagcgcggccaccgagaatc | |
| | | ggacgggggtagtctcaagctggccgg | |
| | | cctgctctggtgcctggcctcgcgccg | |
| | | ccgtgtatcgccccgccctgggcggca | |
| | | aggctggcccggtcggcaccagttgcg | |
| | | tgagcggaaagatggccgcttcccggc | |
| | | cctgctgcagggagctcaaaatggagg | |
| | | acgcggcgctcgggagagcgggcgggt | |
| | | gagtcacccacacaaaggaaaagggcc | |
| | | tttccgtcctcagccgtcgcttcatgt | |
| | | gactccacggagtaccgggcgccgtcc | |
| | | aggcacctcgattagttctcgagcttt | |
| | | tggagtacgtcgtctttaggttggggg | |
| | | gaggggttttatgcgatggagtttccc | |
| | | cacactgagtgggtggagactgaagtt | |
| | | aggccagcttggcacttgatgtaattc | |
| | | tccttggaatttgccctttttgagttt | |
| | | ggatcttggttcattctcaagcctcag | |
| | | acagtggttcaaagtttttttcttcca | |
| | | tttcaggtgtcgtgaggaattagcttg | |
| | | gtactaatacgactcactatagggaga | |
| | | cccaagctggctaggtaagcttggtac | |
| | | cgagctcggatccactagtccagtgtg | |
| | | gtggaattctgcagatatccagcacag | |
| | | tggggtttagtgaaccgtcagatccgc | |
| | | tagagatccgcggccgctaatacgact | |
| | | cactatagggagagccgccaccatgag | |
| | | ctcagagactggcccagtggctgtgga | |
| | | ccccacattgagacggcggatcgagcc | |
| | | ccatgagtttgaggtattcttcgatcc | |
| | | gagagagctccgcaaggagacctgcct | |
| | | gctttacgaaattaattgggggggccg | |
| | | gcactccatttggcgacatacatcaca | |
| | | gaacactaacaagcacgtcgaagtcaa | |
| | | cttcatcgagaagttcacgacagaaag | |
| | | atatttctgtccgaacacaaggtgcag | |
| | | cattacctggtttctcagctggagccc | |
| | | atgcggcgaatgtagtagggccatcac | |
| | | tgaattcctgtcaaggtatccccacgt | |
| | | cactctgtttatttacatcgcaaggct | |
| | | gtaccaccacgctgacccccgcaatcg | |
| | | acaaggcctgcgggatttgatctcttc | |
| | | aggtgtgactatccaaattatgactga | |
| | | gcaggagtcaggatactgctggagaaa | |
| | | ctttgtgaattatagcccgagtaatga | |
| | | agcccactggcctaggtatccccatct | |
| | | gtgggtacgactgtacgttcttgaact | |
| | | gtactgcatcatactgggcctgcctcc | |
| | | ttgtctcaacattctgagaaggaagca | |
| | | gccacagctgacattctttaccatcgc | |
| | | tcttcagtcttgtcattaccagcgact | |
| | | gcccccacacattctctgggccaccgg | |
| | | gttgaaaagcggcagcgagactcccgg | |
| | | gacctcagagtccgccacaccagaacc | |
| | | ccacccagtgttggagaagcttcggtc | |
| | | cattaataactataaccccaaagattt | |
| | | tgactggaatctgaaacatggccgggt | |
| | | tttcatcattaagagctactctgagga | |
| | | cgatattcaccgttccattaagtataa | |
| | | tatttggtgcagcacagagcatggtaa | |
| | | caagagactggatgctgcttatcgttc | |
| | | catgaacgggaaaggccccgtttactt | |
| | | acttttcagtgtcaacggcagtggaca | |
| | | cttctgtggcgtggcagaaatgaaatc | |
| | | tgctgtggactacaacacatgtgcagg | |
| | | tgtgtggtcccaggacaaatggaaggg | |
| | | tcgttttgatgtcaggtggatttttgt | |
| | | gaaggacgttcccaatagccaactgcg | |
| | | acacattcgcctagagaacaacgagaa | |
| | | taaaccagtgaccaactctagggacac | |
| | | tcaggaagtgcctctggaaaaggctaa | |
| | | gcaggtgttgaaaattatagccagcta | |
| | | caagcacaccacttccatttttgatga | |
| | | cttctcacactatgagaaacgccaaga | |
| | | ggaagaagaaagtgttaaaaaggaacg | |
| | | tcaaggtcgtgggaaactcgagtaccc | |
| | | ctacgacgtgcccgactacgcctgagt | |
| | | ttaaaatcgatggtacactcgaggtta | |
| | | acgaattctaccgggtaggggaggcgc | |
| | | ttttcccaaggcagtctggagcatgcg | |
| | | ctttagcagccccgctgggcacttggc | |
| | | gctacacaagtggcctctggcctcgca | |
| | | cacattccacatccaccggtaggcgcc | |
| | | aaccggctccgttctttggtggcccct | |
| | | tcgcgccaccttctactcctcccctag | |
| | | tcaggaagttcccccccgccccgcagc | |
| | | tcgcgtcgtgcaggacgtgacaaatgg | |
| | | aagtagcacgtctcactagtctcgtgc | |
| | | agatggacagcaccgctgagcaatgga | |
| | | agcgggtaggcctttggggcagcggcc | |
| | | aatagcagctttgctccttcgctttct | |
| | | gggctcagaggctgggaagggggggtc | |
| | | cgggggcgggctcaggggcgggctcag | |
| | | gggcggggcgggcgcccgaaggtcctc | |
| | | cggaggcccggcattctgcacgcttca | |
| | | aaagcgcacgtctgccgcgctgttctc | |
| | | ctcttcctcatctccgggcctttcgac | |
| | | ctgcatcccgccaccatgaccgagtac | |
| | | aagcccacggtgcgcctcgccacccgc | |
| | | gacgacgtccccagggccgtacgcacc | |
| | | ctcgccgccgegttcgccgactacccc | |
| | | gccacgegccacaccgtcgatccggac | |
| | | cgccacatcgaggggtcaccgagctgc | |
| | | aagaactcttcctcacgcgcgtcgggc | |
| | | tcgacatcggcaaggtgtgggtcgcgg | |
| | | acgacggcgccgggtggcggtctggac | |
| | | cacgccggagagcgtcgaagcgggggc | |
| | | ggtgttcgccgagatcggcccgcgcat | |
| | | ggccgagttgagcggttcccggctggc | |
| | | cgcgcagcaacagatggaaggcctcct | |
| | | ggcgccgcaccggcccaaggagcccgc | |
| | | gtggttcctggccaccgtcggagtctc | |
| | | gcccgaccaccagggcaagggtctggg | |
| | | cagcgccgtcgtgctccccggagtgga | |
| | | ggcggccgagcgcgccggggtgcccgc | |
| | | cttcctggagacctccgcgccccgcaa | |
| | | cctccccttctacgagcggctcggctt | |
| | | caccgtcaccgccgacgtcgaggtgcc | |
| | | cgaaggaccgcgcacctggtgcatgac | |
| | | ccgcaagcccggtgccggttccggcgc | |
| | | aacaaacttctctctgctgaaacaagc | |
| | | cggagatgtcgaagagaatcctggacc | |
| | | gatggctagattagataaaagtaaagt | |
| | | gattaacagcgcattagagctgcttaa | |
| | | tgaggtcggaatcgaaggtttaacaac | |
| | | ccgtaaactegcccagaagctaggtgt | |
| | | agagcagcctacattgtattggcatgt | |
| | | aaaaaataagcgggctttgctcgacgc | |
| | | cttagccattgagatgttagataggca | |
| | | ccatactcacttttgccctttagaagg | |
| | | ggaaagctggcaagattttttacgtaa | |
| | | taacgctaaaagttttagatgtgcttt | |
| | | actaagtcatcgcgatggagcaaaagt | |
| | | acatttaggtacacggcctacagaaaa | |
| | | acagtatgaaactctcgaaaatcaatt | |
| | | agcctttttatgccaacaaggtttttc | |
| | | actagagaatgcattatatgcactcag | |
| | | cgctgtggggcattttactttaggttg | |
| | | cgtattggaagatcaagagcatcaagt | |
| | | cgctaaagaagaaagggaaacacctac | |
| | | tactgatagtatgccgccattattacg | |
| | | acaagctatcgaattatttgatcacca | |
| | | aggtgcagagccagccttcttattcgg | |
| | | ccttgaattgatcatatgcggattaga | |
| | | aaaacaacttaaatgtgaaagtgggtc | |
| | | gccaaaaaagaagagaaaggtcgacgg | |
| | | cggtggtgctttgtctcctcagcactc | |
| | | tgctgtcactcaaggaagtatcatcaa | |
| | | gaacaaggagggcatggatgctaagtc | |
| | | actaactgcctggtcccggacactggt | |
| | | gaccttcaaggatgtatttgtggactt | |
| | | caccagggaggagtggaagctgctgga | |
| | | cactgctcagcagatcgtgtacagaaa | |
| | | tgtgatgctggagaactataagaacct | |
| | | ggtttccttgggttatcagcttactaa | |
| | | gccagatgtgatcctccggttggagaa | |
| | | gggagaagagccctggctggtgtaaag | |
| | | tagatgccgaccgaacaagagctgatt | |
| | | tcgagaacgcctcagccagcaactcgc | |
| | | gcgagcctagcaaggcaaatgcgagag | |
| | | aacggccttacgcttggtggcacagtt | |
| | | ctcgtccacagttcgctaagctcgctc | |
| | | ggctgggtcgcgggagggccggtcgca | |
| | | gtgattcaggcccttctggattgtgtt | |
| | | ggtccccagggcacgattgtcatgccc | |
| | | acgcactcgggtgatctgactgatccc | |
| | | gcagattggagatcgccgcccgtgcct | |
| | | gccgattgggtgcagatccgtcgagtt | |
| | | aacaaaagaaaaggggggactggaagg | |
| | | gctaattcactcccaacgaagacaaga | |
| | | tatcataacttcgtatagcatacatta | |
| | | tacgaagttatcggctagctggtccgg | |
| | | actgtactgggtctctctggttagacc | |
| | | agatctgagcctgggagctctctggct | |
| | | aactagggaacccactgcttaagcctc | |
| | | aataaagcttgccttgagtgcttcaag | |
| | | tagtgtgtgcccgtctgttgtgtgact | |
| | | ctggtaactagagatccctcagaccct | |
| | | tttagtcagtgtggaaaatctctagca | |
| | | gggcccgtttaaacccgctgatcagcc | |
| | | tcgactgtgccttctagttgccagcca | |
| | | tctgttgtttgcccctcccccgtgcct | |
| | | tccttgaccctggaaggtgccactccc | |
| | | actgtcctttcctaataaaatgaggaa | |
| | | attgcatcgcattgtctgagtaggtgt | |
| | | cattctattctggggggtggggtgggg | |
| | | caggacagcaagggggaggattgggaa | |
| | | gacaatagcaggcatgctggggatgcg | |
| | | gtgggctctatggcttctgaggcggaa | |
| | | agaaccagctggggctctagggggtat | |
| | | ccccacgcgccctgtagcggcgcatta | |
| | | agcgcggcgggtgtggtggttacgcgc | |
| | | agcgtgaccgctacacttgccagcgcc | |
| | | ctagcgcccgctcctttcgctttcttc | |
| | | ccttcctttctcgccacgttcgccggc | |
| | | tttccccgtcaagctctaaatcggggg | |
| | | ctccctttagggttccgatttagtgct | |
| | | ttacggcacctcgaccccaaaaaactt | |
| | | gattagggtgatggttcacgtagtggg | |
| | | ccatcgccctgatagacggtttttcgc | |
| | | cctttgacgttggagtccacgttcttt | |
| | | aatagtggactcttgttccaaactgga | |
| | | acaacactcaaccctatctcggtctat | |
| | | tcttttgatttataagggattttgccg | |
| | | atttcggcctattggttaaaaaatgag | |
| | | ctgatttaacaaaaatttaacgcgaat | |
| | | taattctgtggaatgtgtgtcagttag | |
| | | ggtgtggaaagtccccaggctccccag | |
| | | caggcagaagtatgcaaagcatgcatc | |
| | | tcaattagtcagcaaccaggtgtggaa | |
| | | agtccccaggctccccagcaggcagaa | |
| | | gtatgcaaagcatgcatctcaattagt | |
| | | cagcaaccatagtcccgcccctaactc | |
| | | cgcccatcccgcccctaactccgccca | |
| | | gttccgcccattetccgccccatggct | |
| | | gactaattttttttatttatgcagagg | |
| | | ccgaggccgcctctgcctctgagctat | |
| | | tccagaagtagtgaggaggcttttttg | |
| | | gaggcctaggcttttgcaaaaagctcc | |
| | | cgggagcttgtatatccattttcggat | |
| | | ctgatcagcacgtgttgacaattaatc | |
| | | atcggcatagtatatcggcatagtata | |
| | | atacgacaaggtgaggaactaaaccat | |
| | | ggccaagttgaccagtgccgttccggt | |
| | | gctcaccgcgcgcgacgtcgccggagc | |
| | | ggtcgagttctggaccgaccggctcgg | |
| | | gttctcccgggacttcgtggaggacga | |
| | | cttcgccggtgtggtccgggacgacgt | |
| | | gaccctgttcatcagcgcggtccagga | |
| | | ccaggtggtgccggacaacaccctggc | |
| | | ctgggtgtgggtgcgcggcctggacga | |
| | | gctgtacgccgagtggtcggaggtcgt | |
| | | gtccacgaacttccgggacgcctccgg | |
| | | gccggccatgaccgagatcggcgagca | |
| | | gccgtggggggggagttcgccctgcgc | |
| | | gacccggccggcaactgcgtgcacttc | |
| | | gtggccgaggagcaggactgacacgtg | |
| | | ctacgagatttcgattccaccgccgcc | |
| | | ttctatgaaaggttgggcttcggaatc | |
| | | gttttccgggacgccggctggatgatc | |
| | | ctccagcgcggggatctcatgctggag | |
| | | ttcttcgcccaccccaacttgtttatt | |
| | | gcagcttataatggttacaaataaagc | |
| | | aatagcatcacaaatttcacaaataaa | |
| | | gcatttttttcactgcattctagttgt | |
| | | ggtttgtccaaactcatcaatgtatot | |
| | | tatcatgtctgtataccgtcgacctct | |
| | | agctagagcttggcgtaatcatggtca | |
| | | tagctgtttcctgtgtgaaattgttat | |
| | | ccgctcacaattccacacaacatacga | |
| | | gccggaagcataaagtgtaaagcctgg | |
| | | ggtgcctaatgagtgagctaactcaca | |
| | | ttaattgcgttgcgctcactgcccgct | |
| | | ttccagtcgggaaacctgtcgtgccag | |
| | | ctgcattaatgaatcggccaacgcgcg | |
| | | gggagaggcggtttgcgtattgggcgc | |
| | | tcttccgcttcctcgctcactgactcg | |
| | | ctgcgctcggtcgttcggctgcggcga | |
| | | gcggtatcagctcactcaaaggcggta | |
| | | atacggttatccacagaatcaggggat | |
| | | aacgcaggaaagaacatgtgagcaaaa | |
| | | ggccagcaaaaggccaggaaccgtaaa | |
| | | aaggccgcgttgctggcgtttttccat | |
| | | aggctccgcccccctgacgagcatcac | |
| | | aaaaatcgacgctcaagtcagaggtgg | |
| | | cgaaacccgacaggactataaagatac | |
| | | caggcgtttccccctggaagctccctc | |
| | | gtgcgctctcctgttccgaccctgccg | |
| | | cttaccggatacctgtccgcctttctc | |
| | | ccttcgggaagcgtggcgctttctcat | |
| | | agctcacgctgtaggtatctcagttcg | |
| | | gtgtaggtcgttcgctccaagctgggc | |
| | | tgtgtgcacgaaccccccgttcagccc | |
| | | gaccgctgcgccttatccggtaactat | |
| | | cgtcttgagtccaacccggtaagacac | |
| | | gacttatcgccactggcagcagccact | |
| | | ggtaacaggattagcagagcgaggtat | |
| | | gtaggcggtgctacagagttcttgaag | |
| | | tggtggcctaactacggctacactaga | |
| | | agaacagtatttggtatctgcgctctg | |
| | | ctgaagccagttaccttcggaaaaaga | |
| | | gttggtagctcttgatccggcaaacaa | |
| | | accaccgctggtagcggtggttttttt | |
| | | gtttgcaagcagcagattacgcgcaga | |
| | | aaaaaaggatctcaagaagatcctttg | |
| | | atcttttctacggggtctgacgctcag | |
| | | tggaacgaaaactcacgttaagggatt | |
| | | ttggtcatgagattatcaaaaaggatc | |
| | | ttcacctagatccttttaaattaaaaa | |
| | | tgaagttttaaatcaatctaaagtata | |
| | | tatgagtaaacttggtctgacagttac | |
| | | caatgcttaatcagtgaggcacctatc | |
| | | tcagcgatctgtctatttcgttcatcc | |
| | | atagttgcctgactccccgtcgtgtag | |
| | | ataactacgatacgggagggcttacca | |
| | | tctggccccagtgctgcaatgataccg | |
| | | cgagacccacgctcaccggctccagat | |
| | | ttatcagcaataaaccagccagccgga | |
| | | agggccgagcgcagaagtggtcctgca | |
| | | actttatccgcctccatccagtctatt | |
| | | aattgttgccgggaagctagagtaagt | |
| | | agttcgccagttaatagtttgcgcaac | |
| | | gttgttgccattgctacaggcatcgtg | |
| | | gtgtcacgctcgtcgtttggtatggct | |
| | | tcattcagctccggttcccaacgatca | |
| | | aggcgagttacatgatcccccatgttg | |
| | | tgcaaaaaagcggttagctccttcggt | |
| | | cctccgatcgttgtcagaagtaagttg | |
| | | gccgcagtgttatcactcatggttatg | |
| | | gcagcactgcataattctcttactgtc | |
| | | atgccatccgtaagatgcttttctgtg | |
| | | actggtgagtactcaaccaagtcattc | |
| | | tgagaatagtgtatgcggcgaccgagt | |
| | | tgctcttgcccggcgtcaatacgggat | |
| | | aataccgcgccacatagcagaacttta | |
| | | aaagtgctcatcattggaaaacgttct | |
| | | tcggggcgaaaactctcaaggatctta | |
| | | ccgctgttgagatccagttcgatgtaa | |
| | | cccactcgtgcacccaactgatcttca | |
| | | gcatcttttactttcaccagcgtttct | |
| | | gggtgagcaaaaacaggaaggcaaaat | |
| | | gccgcaaaaaagggaataagggcgaca | |
| | | cggaaatgttgaatactcatactcttc | |
| | | ctttttcaatattattgaagcatttat | |
| | | cagggttattgtctcatgagcggatac | |
| | | atatttgaatgtatttagaaaaataaa | |
| | | caaataggggttccgcgcacatttccc | |
| | | cgaaaagtgccacctgac | |
| 106 | Construct comprising | agggagtggtaaactcgactttcactt | |
| | a nucleic acid | ttctctatcactgatagggagtggtaa | |
| | sequence encoding | actcgactttcacttttctctatcact | |
| | GFP, a m6A reporter | gatagggagtggtaaactcgactttca | |
| | sequence, and DHFR; | cttttctctatcactgatagggagtgg | |
| | a nucleic acid | taaactcgactttcacttttctctatc | |
| | sequence encoding | actgatagggagtggtaaactcgaggg | |
| | APOBEC1-YTH (5′-3′); | ggatccactagcatgaagggcgaattc | |
| | and a nucleic acid | cagcacactggtaacccgtgtcggctc | |
| | sequence encoding | cagatctggcctccgcgccgggttttg | |
| | dsRed | gcgcctcccgcgggcgcccccctcctc | |
| | | acggcgagccgcgttgacattgattat | |
| | | tgactaggcttttgcaaaaagctttgc | |
| | | aaagatggataaagttttaaacagaga | |
| | | ggaatctttgcagctaatggaccttct | |
| | | aggtcttgaaaggagtgggaattggct | |
| | | ccggtgcccgtcagtgggcagagcgca | |
| | | catcgcccacagtccccgagaagttgg | |
| | | ggggaggggtcggcaattgaaccggtg | |
| | | cctagagaaggtggcgcggggtaaact | |
| | | gggaaagtgatgtcgtgtactggctcc | |
| | | gcctttttcccgagggtgggggagaac | |
| | | cgtatataagtgcagtagtcgccgtga | |
| | | acgttctttttcgcaacgggtttgccg | |
| | | ccagaacacaggtaagtgccgtgtgtg | |
| | | gttcccgcgggcctggcctctttacgg | |
| | | gttatggcccttgcgtgccttgaatta | |
| | | cttccacctggctgcagtacgtgattc | |
| | | ttgatcccgagcttcgggttggaagtg | |
| | | gggggagagttcgaggccttgcgctta | |
| | | aggagccccttcgcctcgtgcttgagt | |
| | | tgaggcctggcctgggcgctggggccg | |
| | | ccgcgtgcgaatctggtggcaccttcg | |
| | | cgcctgtctcgctgctttcgataagtc | |
| | | tctagccatttaaaatttttgatgacc | |
| | | tgctgcgacgctttttttctggcaaga | |
| | | tagtcttgtaaatgcgggccaagatct | |
| | | gcacactggtatttcggtttttggggc | |
| | | cgcgggcggcgacggggcccgtgcgtc | |
| | | ccagcgcacatgttcggcgaggcgggg | |
| | | cctgcgagcgcggccaccgagaatcgg | |
| | | acgggggtagtctcaagctggccggcc | |
| | | tgctctggtgcctggcctcgcgccgcc | |
| | | gtgtatcgccccgccctgggcggcaag | |
| | | gctggcccggtcggcaccagttgcgtg | |
| | | agcggaaagatggccgcttcccggccc | |
| | | tgctgcagggagctcaaaatggaggac | |
| | | gcggcgctcgggagagcggggggtgag | |
| | | tcacccacacaaaggaaaagggccttt | |
| | | ccgtcctcagccgtcgcttcatgtgac | |
| | | tccacggagtaccgggcgccgtccagg | |
| | | cacctcgattagttctcgagcttttgg | |
| | | agtacgtcgtctttaggttggggggag | |
| | | gggttttatgcgatggagtttccccac | |
| | | actgagtgggtggagactgaagttagg | |
| | | ccagcttggcacttgatgtaattctcc | |
| | | ttggaatttgccctttttgagtttgga | |
| | | tcttggttcattctcaagcctcagaca | |
| | | gtggttcaaagtttttttcttccattt | |
| | | caggtgtcgtgaggaattagcttggta | |
| | | ctaatacgactcactatagggagaccc | |
| | | aagctggctaggtaagcttggtaccga | |
| | | gctcggatccactagtccagtgtggtg | |
| | | gaattctgcagatatccagcacagtgg | |
| | | ggtttagtgaaccgtcagatccgctag | |
| | | agatccgcggccgctaatacgactcac | |
| | | tatagggagagccgccaccatgagctc | |
| | | agagactggcccagtggctgtggaccc | |
| | | cacattgagacggcggatcgagcccca | |
| | | tgagtttgaggtattcttcgatccgag | |
| | | agagctccgcaaggagacctgcctgct | |
| | | ttacgaaattaattgggggggccggca | |
| | | ctccatttggcgacatacatcacagaa | |
| | | cactaacaagcacgtcgaagtcaactt | |
| | | catcgagaagttcacgacagaaagata | |
| | | tttctgtccgaacacaaggtgcagcat | |
| | | tacctggtttctcagctggagcccatg | |
| | | cggcgaatgtagtagggccatcactga | |
| | | attcctgtcaaggtatccccacgtcac | |
| | | tctgtttatttacatcgcaaggctgta | |
| | | ccaccacgctgacccccgcaatcgaca | |
| | | aggcctgcgggatttgatctcttcagg | |
| | | tgtgactatccaaattatgactgagca | |
| | | ggagtcaggatactgctggagaaactt | |
| | | tgtgaattatagcccgagtaatgaagc | |
| | | ccactggcctaggtatccccatctgtg | |
| | | ggtacgactgtacgttcttgaactgta | |
| | | ctgcatcatactgggcctgcctccttg | |
| | | tctcaacattctgagaaggaagcagcc | |
| | | acagctgacattctttaccatcgctct | |
| | | tcagtcttgtcattaccagegactgcc | |
| | | cccacacattctctgggccaccgggtt | |
| | | gaaaagcggcagcgagactcccgggac | |
| | | ctcagagtccgccacaccagaacccca | |
| | | cccagtgttggagaagcttcggtccat | |
| | | taataactataaccccaaagattttga | |
| | | ctggaatctgaaacatggccgggtttt | |
| | | catcattaagagctactctgaggacga | |
| | | tattcaccgttccattaagtataatat | |
| | | ttggtgcagcacagagcatggtaacaa | |
| | | gagactggatgctgcttatcgttccat | |
| | | gaacgggaaaggccccgtttacttact | |
| | | tttcagtgtcaacggcagtggacactt | |
| | | ctgtggcgtggcagaaatgaaatctgc | |
| | | tgtggactacaacacatgtgcaggtgt | |
| | | gtggtcccaggacaaatggaagggtcg | |
| | | ttttgatgtcaggtggatttttgtgaa | |
| | | ggacgttoccaatagccaactgcgaca | |
| | | cattcgcctagagaacaacgagaataa | |
| | | accagtgaccaactctagggacactca | |
| | | ggaagtgcctctggaaaaggctaagca | |
| | | ggtgttgaaaattatagccagctacaa | |
| | | gcacaccacttccatttttgatgactt | |
| | | ctcacactatgagaaacgccaagagga | |
| | | agaagaaagtgttaaaaaggaacgtca | |
| | | aggtcgtgggaaactcgagtaccccta | |
| | | cgacgtgcccgactacgcctgagttta | |
| | | aaatcgatggtacactcgaggttaacg | |
| | | aattctaccttacccagagtgcaggtg | |
| | | tgtggagatccctcctgccttgacatt | |
| | | gagcagccttagagggtgggggaggct | |
| | | caggggtcaggtctctgttcctgctta | |
| | | ttggggagttcctggcctggcccttct | |
| | | atgtctccccaggtaccccagtttttc | |
| | | tgggttcacccagagtgcagatgcttg | |
| | | aggaggtgggaagggactatttggggg | |
| | | tgtctggctcaggtgccatgcctcact | |
| | | ggggctggttggcacctgcatttcctg | |
| | | ggagtggggctgtctcagggtagctgg | |
| | | gcacggtgttcccttgagtgggggtgt | |
| | | agtgagtgttcctagctgccacgcctt | |
| | | tgccttcacctatgggatcgtggctgt | |
| | | cagttaattaaccttccgcgggagctc | |
| | | acggggagagccccccgccaaagcccc | |
| | | cagggatgtaattgcatccctcttccg | |
| | | ctagggggcagcagcgagccgcccggg | |
| | | gctccgctccggtccggcgctcccccc | |
| | | gcatccccgagccggagccggcagcgt | |
| | | gcggggacagcccggcacggggaaggt | |
| | | ggcacgcgatcgctttcctctgaacgc | |
| | | ttctcgctgctctttgagcctgcagac | |
| | | acctggggggatacggggaaaaagctt | |
| | | taggctgaaagagagatttagaatgac | |
| | | agaatcatagaatggcctgggttgcaa | |
| | | aggagcacagtgctcacccagctccaa | |
| | | ccccctgctatgtgcagggtcgccaac | |
| | | cagcagcccaggctgcccagagccaca | |
| | | tccagcctggccttgaatgcctgcagg | |
| | | gatggggcatccacagcctccttgggc | |
| | | aacctgttcagtgcgtcacggatccaa | |
| | | ttccacggggttggggttgcgcctttt | |
| | | ccaaggcagccctgggtttgcgcaggg | |
| | | acgcggctgctctgggcgtggttccgg | |
| | | gaaacgcagcggcgccgaccctgggtc | |
| | | tcgcacattcttcacgtccgttcgcag | |
| | | cgtcacccggatcttcgccgctaccct | |
| | | tgtgggccccccggcgacgcttcctgc | |
| | | tccgcccctaagtcgggaaggttcctt | |
| | | gcggttcgcggcgtgccggacgtgaca | |
| | | aacggaagccgcacgtctcactagtac | |
| | | cctcgcagacggacagcgccagggagc | |
| | | aatggcagcgcgccgaccgcgatgggc | |
| | | tgtggccaatagcggctgctcagcagg | |
| | | gcgcgccgagagcagcggccgggaagg | |
| | | ggcggtgcgggaggcggggtgtggggc | |
| | | ggtagtgtgggccctgttcctgcccgc | |
| | | gcggtgttccgcattctgcaagcctcc | |
| | | ggagcgcacgtcggcagtcggctccct | |
| | | cgttgaccgaatcaccgacctctctcc | |
| | | ccagctgtagctagcacaaccatggat | |
| | | agcactgagaacgtcatcaagcccttc | |
| | | atgcgcttcaaggtgcacatggagggc | |
| | | tccgtgaacggccacgagttcgagatc | |
| | | gagggcgagggcgagggcaagccctac | |
| | | gagggcacccagaccgccaagctgcag | |
| | | gtgaccaagggcggccccctgcccttc | |
| | | gcctgggacatcctgtccccccagttc | |
| | | cagtacggctccaaggtgtacgtgaag | |
| | | caccccgccgacatccccgactacaag | |
| | | aagctgtccttccccgagggcttcaag | |
| | | tgggagcgcgtgatgaacttcgaggac | |
| | | ggcggcgtggtgaccgtgacccaggac | |
| | | tcctccctgcaggacggcaccttcatc | |
| | | taccacgtgaagttcatcggcgtgaac | |
| | | ttcccctccgacggccccgtaatgcag | |
| | | aagaagactctgggctgggagccctcc | |
| | | accgagcgcctgtacccccgcgacggc | |
| | | gtgctgaagggcgagatccacaaggcg | |
| | | ctgaagctgaagggcggcggccactac | |
| | | ctggtggagttcaagtcaatctacatg | |
| | | gccaagaagcccgtgaagctgcccggc | |
| | | tactactacgtggactccaagctggac | |
| | | atcacctcccacaacgaggactacacc | |
| | | gtggtggagcagtacgagcgcgccgag | |
| | | gcccgccaccacctgttccagtagggc | |
| | | tagctggtccggactgtactgggtctc | |
| | | tctggttagaccagatctgagcctggg | |
| | | agctctctggctaactagggaacccac | |
| | | tgcttaagcctcaataaagcttgcctt | |
| | | gagtgcttcaagtagtgtgtgcccgtc | |
| | | tgttgtgtgactctggtaactagagat | |
| | | ccctcagacccttttagtcagtgtgga | |
| | | aaatctctagcagggcccgtttaaacc | |
| | | cgctgatcagcctcgactgtgccttct | |
| | | agttgccagccatctgttgtttgcccc | |
| | | tcccccgtgccttccttgaccctggaa | |
| | | ggtgccactcccactgtcctttcctaa | |
| | | taaaatgaggaaattgcatcgcattgt | |
| | | ctgagtaggtgtcattctattctgggg | |
| | | ggtggggtggggcaggacagcaagggg | |
| | | gaggattgggaagacaatagcaggcat | |
| | | gctggggatgcggtgggctctatggct | |
| | | tctgaggcggaaagaaccagctggggc | |
| | | tctagggggtatccccacgcgccctgt | |
| | | agcggcgcattaagcgcggcgggtgtg | |
| | | gtggttacgcgcagcgtgaccgctaca | |
| | | cttgccagcgccctagegcccgctcct | |
| | | ttcgctttettcccttcctttctcgcc | |
| | | acgttcgccggctttccccgtcaagct | |
| | | ctaaatcgggggctccctttagggttc | |
| | | cgatttagtgctttacggcacctcgac | |
| | | cccaaaaaacttgattagggtgatggt | |
| | | tcacgtagtgggccatcgccctgatag | |
| | | acggtttttcgccctttgacgttggag | |
| | | tccacgttctttaatagtggactcttg | |
| | | ttccaaactggaacaacactcaaccct | |
| | | atctcggtctattcttttgatttataa | |
| | | gggattttgccgatttcggcctattgg | |
| | | ttaaaaaatgagctgatttaacaaaaa | |
| | | tttaacgcgaattaattctgtggaatg | |
| | | tgtgtcagttagggtgtggaaagtccc | |
| | | caggctccccagcaggcagaagtatgc | |
| | | aaagcatgcatctcaattagtcagcaa | |
| | | ccaggtgtggaaagtccccaggctccc | |
| | | cagcaggcagaagtatgcaaagcatgc | |
| | | atctcaattagtcagcaaccatagtcc | |
| | | cgcccctaactcegcccatccegcccc | |
| | | taactccgcccagttccgcccattctc | |
| | | cgccccatggctgactaatttttttta | |
| | | tttatgcagaggccgaggccgcctctg | |
| | | cctctgagctattccagaagtagtgag | |
| | | gaggcttttttggaggcctaggctttt | |
| | | gcaaaaagctcccgggagcttgtatat | |
| | | ccattttcggatctgatcagcacgtgt | |
| | | tgacaattaatcatcggcatagtatat | |
| | | cggcatagtataatacgacaaggtgag | |
| | | gaactaaaccatggccaagttgaccag | |
| | | tgccgttccggtgctcaccgcgcgcga | |
| | | cgtcgccggagcggtcgagttctggac | |
| | | cgaccggctcgggttctcccgggactt | |
| | | cgtggaggacgacttcgccggtgtggt | |
| | | ccgggacgacgtgaccctgttcatcag | |
| | | cgcggtccaggaccaggtggtgccgga | |
| | | caacaccctggcctgggtgtgggtgcg | |
| | | cggcctggacgagctgtacgccgagtg | |
| | | gtcggaggtcgtgtccacgaacttccg | |
| | | ggacgcctccgggccggccatgaccga | |
| | | gatcggcgagcagccgtggggggggag | |
| | | ttcgccctgcgcgacccggccggcaac | |
| | | tgcgtgcacttcgtggccgaggagcag | |
| | | gactgacacgtgctacgagatttcgat | |
| | | tccaccgccgccttctatgaaaggttg | |
| | | ggcttcggaatcgttttccgggacgcc | |
| | | ggctggatgatcctccagcgcggggat | |
| | | ctcatgctggagttcttcgcccacccc | |
| | | aacttgtttattgcagcttataatggt | |
| | | tacaaataaagcaatagcatcacaaat | |
| | | ttcacaaataaagcatttttttcactg | |
| | | cattctagttgtggtttgtccaaactc | |
| | | atcaatgtatcttatcatgtctgtata | |
| | | ccgtcgacctctagctagagcttggcg | |
| | | taatcatggtcatagctgtttcctgtg | |
| | | tgaaattgttatccgctcacaattcca | |
| | | cacaacatacgagccggaagcataaag | |
| | | tgtaaagcctggggtgcctaatgagtg | |
| | | agctaactcacattaattgcgttgcgc | |
| | | tcactgcccgctttccagtcgggaaac | |
| | | ctgtcgtgccagctgcattaatgaatc | |
| | | ggccaacgcgcggggagaggcggtttg | |
| | | cgtattgggcgctcttccgcttcctcg | |
| | | ctcactgactcgctgcgctcggtcgtt | |
| | | cggctgcggcgagcggtatcagctcac | |
| | | tcaaaggcggtaatacggttatccaca | |
| | | gaatcaggggataacgcaggaaagaac | |
| | | atgtgagcaaaaggccagcaaaaggcc | |
| | | aggaaccgtaaaaaggccgcgttgctg | |
| | | gcgtttttccataggctccgcccccct | |
| | | gacgagcatcacaaaaatcgacgctca | |
| | | agtcagaggtggcgaaacccgacagga | |
| | | ctataaagataccaggcgtttccccct | |
| | | ggaagctccctcgtgegctctcctgtt | |
| | | ccgaccctgccgcttaccggatacctg | |
| | | tccgcctttctcccttcgggaagcgtg | |
| | | gcgctttctcatagctcacgctgtagg | |
| | | tatctcagttcggtgtaggtcgttcgc | |
| | | tccaagctgggctgtgtgcacgaaccc | |
| | | cccgttcagcccgaccgctgcgcctta | |
| | | tccggtaactatcgtcttgagtccaac | |
| | | ccggtaagacacgacttatcgccactg | |
| | | gcagcagccactggtaacaggattagc | |
| | | agagcgaggtatgtaggcggtgctaca | |
| | | gagttcttgaagtggtggcctaactac | |
| | | ggctacactagaagaacagtatttggt | |
| | | atctgcgctctgctgaagccagttacc | |
| | | ttcggaaaaagagttggtagctcttga | |
| | | tccggcaaacaaaccaccgctggtagc | |
| | | ggtggtttttttgtttgcaagcagcag | |
| | | attacgcgcagaaaaaaaggatctcaa | |
| | | gaagatcctttgatcttttctacgggg | |
| | | tctgacgctcagtggaacgaaaactca | |
| | | cgttaagggattttggtcatgagatta | |
| | | tcaaaaaggatcttcacctagatcctt | |
| | | ttaaattaaaaatgaagttttaaatca | |
| | | atctaaagtatatatgagtaaacttgg | |
| | | tctgacagttaccaatgcttaatcagt | |
| | | gaggcacctatctcagcgatctgtcta | |
| | | tttcgttcatccatagttgcctgactc | |
| | | cccgtcgtgtagataactacgatacgg | |
| | | gagggcttaccatctggccccagtgct | |
| | | gcaatgataccgcgagacccacgctca | |
| | | ccggctccagatttatcagcaataaac | |
| | | cagccagccggaagggccgagcgcaga | |
| | | agtggtcctgcaactttatccgcctcc | |
| | | atccagtctattaattgttgccgggaa | |
| | | gctagagtaagtagttcgccagttaat | |
| | | agtttgcgcaacgttgttgccattgct | |
| | | acaggcatcgtggtgtcacgctcgtcg | |
| | | tttggtatggcttcattcagctccggt | |
| | | tcccaacgatcaaggcgagttacatga | |
| | | tcccccatgttgtgcaaaaaagcggtt | |
| | | agctccttcggtcctccgatcgttgtc | |
| | | agaagtaagttggccgcagtgttatca | |
| | | ctcatggttatggcagcactgcataat | |
| | | tctcttactgtcatgccatccgtaaga | |
| | | tgcttttctgtgactggtgagtactca | |
| | | accaagtcattctgagaatagtgtatg | |
| | | cggcgaccgagttgctcttgcccggcg | |
| | | tcaatacgggataataccgcgccacat | |
| | | agcagaactttaaaagtgctcatcatt | |
| | | ggaaaacgttcttcggggcgaaaactc | |
| | | tcaaggatcttaccgctgttgagatcc | |
| | | agttcgatgtaacccactcgtgcaccc | |
| | | aactgatcttcagcatcttttactttc | |
| | | accagcgtttctgggtgagcaaaaaca | |
| | | ggaaggcaaaatgccgcaaaaaaggga | |
| | | ataagggcgacacggaaatgttgaata | |
| | | ctcatactcttcctttttcaatattat | |
| | | tgaagcatttatcagggttattgtctc | |
| | | atgagcggatacatatttgaatgtatt | |
| | | tagaaaaataaacaaataggggttccg | |
| | | cgcacatttccccgaaaagtgccacct | |
| | | gac | |
| 107 | Construct comprising | agggagtggtaaactcgactttcactt | |
| | a nucleic acid | ttctctatcactgatagggagtggtaa | |
| | sequence encoding | actcgactttcacttttctctatcact | |
| | GFP-PEST, a m6A | gatagggagtggtaaactcgactttca | |
| | reporter sequence, | cttttctctatcactgatagggagtgg | |
| | and DHFR; a nucleic | taaactcgactttcacttttctctatc | |
| | acid sequenc | actgatagggagtggtaaactcgaggg | |
| | encoding APOBEC1-YTH | ggatccactagcatgaagggcgaattc | |
| | (5′-3′) | cagcacactggtaacccgtgtcggctc | |
| | | cagatctggcctccgcgccgggttttg | |
| | | gcgcctcccgcgggcgcccccctectc | |
| | | acggcgagccgcgttgacattgattat | |
| | | tgactaggcttttgcaaaaagctttgc | |
| | | aaagatggataaagttttaaacagaga | |
| | | ggaatctttgcagctaatggaccttct | |
| | | aggtcttgaaaggagtgggaattggct | |
| | | ccggtgcccgtcagtgggcagagcgca | |
| | | catcgcccacagtccccgagaagttgg | |
| | | ggggaggggtcggcaattgaaccggtg | |
| | | cctagagaaggtggcgcggggtaaact | |
| | | gggaaagtgatgtcgtgtactggctcc | |
| | | gcctttttcccgaggggggggagaacc | |
| | | gtatataagtgcagtagtcgccgtgaa | |
| | | cgttctttttcgcaacgggtttgccgc | |
| | | cagaacacaggtaagtgccgtgtgtgg | |
| | | ttcccgcgggcctggcctctttacggg | |
| | | ttatggcccttgcgtgccttgaattac | |
| | | ttccacctggctgcagtacgtgattct | |
| | | tgatcccgagcttcgggttggaagtgg | |
| | | ggggagagttcgaggccttgcgcttaa | |
| | | ggagccccttcgcctcgtgcttgagtt | |
| | | gaggcctggcctgggcgctggggccgc | |
| | | cgcgtgcgaatctggtggcaccttcgc | |
| | | gcctgtctcgctgctttcgataagtct | |
| | | ctagccatttaaaatttttgatgacct | |
| | | gctgcgacgctttttttctggcaagat | |
| | | agtcttgtaaatgcgggccaagatctg | |
| | | cacactggtatttcggtttttggggcc | |
| | | gcgggcggcgacggggcccgtgcgtcc | |
| | | cagcgcacatgttcggcgaggcggggc | |
| | | ctgcgagcgcggccaccgagaatcgga | |
| | | cgggggtagtctcaagctggccggcct | |
| | | gctctggtgcctggcctcgcgccgccg | |
| | | tgtatcgccccgccctgggcggcaagg | |
| | | ctggcccggtcggcaccagttgcgtga | |
| | | gcggaaagatggccgcttcccggccct | |
| | | gctgcagggagctcaaaatggaggacg | |
| | | cggcgctcgggagagcggggggtgagt | |
| | | cacccacacaaaggaaaagggcctttc | |
| | | cgtcctcagccgtcgcttcatgtgact | |
| | | ccacggagtaccgggcgccgtccaggc | |
| | | acctogattagttctcgagcttttgga | |
| | | gtacgtcgtctttaggttggggggagg | |
| | | ggttttatgcgatggagtttccccaca | |
| | | ctgagtgggtggagactgaagttaggc | |
| | | cagcttggcacttgatgtaattctcct | |
| | | tggaatttgccctttttgagtttggat | |
| | | cttggttcattctcaagcctcagacag | |
| | | tggttcaaagtttttttcttccatttc | |
| | | aggtgtcgtgaggaattagcttggtac | |
| | | taatacgactcactatagggagaccca | |
| | | agctggctaggtaagcttggtaccgag | |
| | | ctcggatccactagtccagtgtggtgg | |
| | | aattctgcagatatccagcacagtggg | |
| | | gtttagtgaaccgtcagatccgctaga | |
| | | gatccgcggccgctaatacgactcact | |
| | | atagggagagccgccaccatgagctca | |
| | | gagactggcccagtggctgtggacccc | |
| | | acattgagacggcggatcgagccccat | |
| | | gagtttgaggtattcttcgatccgaga | |
| | | gagctccgcaaggagacctgcctgctt | |
| | | tacgaaattaattgggggggccggcac | |
| | | tccatttggcgacatacatcacagaac | |
| | | actaacaagcacgtcgaagtcaacttc | |
| | | atcgagaagttcacgacagaaagatat | |
| | | ttctgtccgaacacaaggtgcagcatt | |
| | | acctggtttctcagctggagcccatgc | |
| | | ggcgaatgtagtagggccatcactgaa | |
| | | ttcctgtcaaggtatccccacgtcact | |
| | | ctgtttatttacatcgcaaggctgtac | |
| | | caccacgctgacccccgcaatcgacaa | |
| | | ggcctgcgggatttgatctcttcaggt | |
| | | gtgactatccaaattatgactgagcag | |
| | | gagtcaggatactgctggagaaacttt | |
| | | gtgaattatagcccgagtaatgaagcc | |
| | | cactggcctaggtatccccatctgtgg | |
| | | gtacgactgtacgttcttgaactgtac | |
| | | tgcatcatactgggcctgcctccttgt | |
| | | ctcaacattctgagaaggaagcagcca | |
| | | cagctgacattctttaccatcgctctt | |
| | | cagtcttgtcattaccagcgactgccc | |
| | | ccacacattctctgggccaccgggttg | |
| | | aaaagcggcagcgagactcccgggacc | |
| | | tcagagtccgccacaccagaaccccac | |
| | | ccagtgttggagaagcttcggtccatt | |
| | | aataactataaccccaaagattttgac | |
| | | tggaatctgaaacatggccgggttttc | |
| | | atcattaagagctactctgaggacgat | |
| | | attcaccgttccattaagtataatatt | |
| | | tggtgcagcacagagcatggtaacaag | |
| | | agactggatgctgcttatcgttccatg | |
| | | aacgggaaaggccccgtttacttactt | |
| | | ttcagtgtcaacggcagtggacacttc | |
| | | tgtggcgtggcagaaatgaaatctgct | |
| | | gtggactacaacacatgtgcaggtgtg | |
| | | tggtcccaggacaaatggaagggtcgt | |
| | | tttgatgtcaggtggatttttgtgaag | |
| | | gacgttcccaatagccaactgcgacac | |
| | | attcgcctagagaacaacgagaataaa | |
| | | ccagtgaccaactctagggacactcag | |
| | | gaagtgcctctggaaaaggctaagcag | |
| | | gtgttgaaaattatagccagctacaag | |
| | | cacaccacttccatttttgatgacttc | |
| | | tcacactatgagaaacgccaagaggaa | |
| | | gaagaaagtgttaaaaaggaacgtcaa | |
| | | ggtcgtgggaaactcgagtacccctac | |
| | | gacgtgcccgactacgcctgagtttaa | |
| | | aatcgatggtacactcgaggttaacga | |
| | | attctaccttacccagagtgcaggtgt | |
| | | gtggagatccctcctgccttgacattg | |
| | | agcagccttagaggggggggaggctca | |
| | | ggggtcaggtctctgttcctgcttatt | |
| | | ggggagttcctggcctggcccttctat | |
| | | gtctccccaggtaccccagtttttctg | |
| | | ggttcacccagagtgcagatgcttgag | |
| | | gaggtgggaagggactatttgggggtg | |
| | | tctggctcaggtgccatgcctcactgg | |
| | | ggctggttggcacctgcatttcctggg | |
| | | agtggggctgtctcagggtagctgggc | |
| | | acggtgttcccttgagtgggggtgtag | |
| | | tgagtgttcctagctgccacgcctttg | |
| | | ccttcacctatgggatcgtggctgtca | |
| | | gttaattaaccttccgcgggagctcac | |
| | | ggggagagccccccgccaaagccccca | |
| | | gggatgtaattgcatccctcttccgct | |
| | | agggggcagcagcgagccgcccggggc | |
| | | tccgctccggtccggcgctccccccgc | |
| | | atccccgagccggagccggcagcgtgc | |
| | | ggggacagcccggcacggggaaggtgg | |
| | | cacgcgatcgctttcctctgaacgctt | |
| | | ctcgctgctctttgagcctgcagacac | |
| | | ctggggggatacggggaaaaagcttta | |
| | | ggctgaaagagagatttagaatgacag | |
| | | aatcatagaatggcctgggttgcaaag | |
| | | gagcacagtgctcacccagctccaacc | |
| | | ccctgctatgtgcagggtcgccaacca | |
| | | gcagcccaggctgcccagagccacatc | |
| | | cagcctggccttgaatgcctgcaggga | |
| | | tggggcatccacagcctccttgggcaa | |
| | | cctgttcagtgcgtcacggatccaatt | |
| | | ccacggggttggggttgcgccttttcc | |
| | | aaggcagccctgggtttgcgcagggac | |
| | | gcggctgctctgggcgtggttccggga | |
| | | aacgcagcggcgccgaccctgggtctc | |
| | | gcacattcttcacgtccgttcgcagcg | |
| | | tcacccggatcttcgccgctacccttg | |
| | | tgggccccccggcgacgcttcctgctc | |
| | | cgcccctaagtcgggaaggttccttgc | |
| | | ggttcgcggcgtgccggacgtgacaaa | |
| | | cggaagccgcacgtctcactagtaccc | |
| | | tcgcagacggacagcgccagggagcaa | |
| | | tggcagcgcgccgaccgcgatgggctg | |
| | | tggccaatagcggctgctcagcagggc | |
| | | gcgccgagagcagcggccgggaagggg | |
| | | cggtgcgggaggcggggtgtggggcgg | |
| | | tagtgtgggccctgttcctgcccgcgc | |
| | | ggtgttccgcattctgcaagcctccgg | |
| | | agcgcacgtcggcagtcggctccctcg | |
| | | ttgaccgaatcaccgacctctctcccc | |
| | | agctgtagctagcacaaccatggatag | |
| | | cactgagaacgtcatcaagcccttcat | |
| | | gcgcttcaaggtgcacatggagggctc | |
| | | cgtgaacggccacgagttcgagatcga | |
| | | gggcgagggcgagggcaagccctacga | |
| | | gggcacccagaccgccaagctgcaggt | |
| | | gaccaagggggccccctgcccttcgcc | |
| | | tgggacatcctgtccccccagttccag | |
| | | tacggctccaaggtgtacgtgaagcac | |
| | | cccgccgacatccccgactacaagaag | |
| | | ctgtccttccccgagggcttcaagtgg | |
| | | gagcgcgtgatgaacttcgaggacggc | |
| | | ggcgtggtgaccgtgacccaggactcc | |
| | | tccctgcaggacggcaccttcatctac | |
| | | cacgtgaagttcatcggcgtgaacttc | |
| | | ccctccgacggccccgtaatgcagaag | |
| | | aagactctgggctgggagccctccacc | |
| | | gagcgcctgtacccccgcgacggcgtg | |
| | | ctgaagggcgagatccacaaggcgctg | |
| | | aagctgaagggcggcggccactacctg | |
| | | gtggagttcaagtcaatctacatggcc | |
| | | aagaagcccgtgaagctgcccggctac | |
| | | tactacgtggactccaagctggacatc | |
| | | acctcccacaacgaggactacaccgtg | |
| | | gtggagcagtacgagcgcgccgaggcc | |
| | | cgccaccacctgttccagtagggctag | |
| | | ctggtccggactgtactgggtctctct | |
| | | ggttagaccagatctgagcctgggagc | |
| | | tctctggctaactagggaacccactgc | |
| | | ttaagcctcaataaagcttgccttgag | |
| | | tgcttcaagtagtgtgtgcccgtctgt | |
| | | tgtgtgactctggtaactagagatccc | |
| | | tcagacccttttagtcagtgtggaaaa | |
| | | tctctagcagggcccgtttaaacccgc | |
| | | tgatcagcctcgactgtgccttctagt | |
| | | tgccagccatctgttgtttgcccctcc | |
| | | cccgtgccttccttgaccctggaaggt | |
| | | gccactcccactgtcctttcctaataa | |
| | | aatgaggaaattgcatcgcattgtctg | |
| | | agtaggtgtcattctattctggggggt | |
| | | ggggggggcaggacagcaagggggagg | |
| | | attgggaagacaatagcaggcatgctg | |
| | | gggatgcggtgggctctatggcttctg | |
| | | aggcggaaagaaccagctggggctcta | |
| | | gggggtatccccacgcgccctgtagcg | |
| | | gcgcattaagcgcggcgggtgtggtgg | |
| | | ttacgcgcagcgtgaccgctacacttg | |
| | | ccagcgccctagcgcccgctcctttcg | |
| | | ctttcttcccttcctttctcgccacgt | |
| | | tcgccggctttccccgtcaagctctaa | |
| | | atcgggggctccctttagggttccgat | |
| | | ttagtgctttacggcacctcgacccca | |
| | | aaaaacttgattagggtgatggttcac | |
| | | gtagtgggccatcgccctgatagacgg | |
| | | tttttcgccctttgacgttggagtcca | |
| | | cgttctttaatagtggactcttgttcc | |
| | | aaactggaacaacactcaaccctatct | |
| | | cggtctattcttttgatttataaggga | |
| | | ttttgccgatttcggcctattggttaa | |
| | | aaaatgagctgatttaacaaaaattta | |
| | | acgcgaattaattctgtggaatgtgtg | |
| | | tcagttagggtgtggaaagtccccagg | |
| | | ctccccagcaggcagaagtatgcaaag | |
| | | catgcatctcaattagtcagcaaccag | |
| | | gtgtggaaagtccccaggctccccagc | |
| | | aggcagaagtatgcaaagcatgcatct | |
| | | caattagtcagcaaccatagtcccgcc | |
| | | cctaactcegcccatccegcccctaac | |
| | | tccgcccagttccgcccattctccgcc | |
| | | ccatggctgactaattttttttattta | |
| | | tgcagaggccgaggccgcctctgcctc | |
| | | tgagctattccagaagtagtgaggagg | |
| | | cttttttggaggcctaggcttttgcaa | |
| | | aaagctcccgggagcttgtatatccat | |
| | | tttcggatctgatcagcacgtgttgac | |
| | | aattaatcatcggcatagtatatcggc | |
| | | atagtataatacgacaaggtgaggaac | |
| | | taaaccatggccaagttgaccagtgcc | |
| | | gttccggtgctcaccgcgcgcgacgtc | |
| | | gccggagcggtcgagttctggaccgac | |
| | | cggctcgggttctcccgggacttcgtg | |
| | | gaggacgacttcgccggtgtggtccgg | |
| | | gacgacgtgaccctgttcatcagcgcg | |
| | | gtccaggaccaggtggtgccggacaac | |
| | | accctggcctgggtgtgggtgcgcggc | |
| | | ctggacgagctgtacgccgagtggtcg | |
| | | gaggtcgtgtccacgaacttccgggac | |
| | | gcctccgggccggccatgaccgagatc | |
| | | ggcgagcagccgtggggggggagttcg | |
| | | ccctgcgcgacccggccggcaactgcg | |
| | | tgcacttcgtggccgaggagcaggact | |
| | | gacacgtgctacgagatttcgattcca | |
| | | ccgccgccttctatgaaaggttgggct | |
| | | tcggaatcgttttccgggacgccggct | |
| | | ggatgatcctccagcgcggggatctca | |
| | | tgctggagttcttcgcccaccccaact | |
| | | tgtttattgcagcttataatggttaca | |
| | | aataaagcaatagcatcacaaatttca | |
| | | caaataaagcatttttttcactgcatt | |
| | | ctagttgtggtttgtccaaactcatca | |
| | | atgtatcttatcatgtctgtataccgt | |
| | | cgacctctagctagagcttggcgtaat | |
| | | catggtcatagctgtttcctgtgtgaa | |
| | | attgttatccgctcacaattccacaca | |
| | | acatacgagccggaagcataaagtgta | |
| | | aagcctggggtgcctaatgagtgagct | |
| | | aactcacattaattgcgttgcgctcac | |
| | | tgcccgctttccagtcgggaaacctgt | |
| | | cgtgccagctgcattaatgaatcggcc | |
| | | aacgcgcggggagaggcggtttgcgta | |
| | | ttgggcgctcttccgcttcctcgctca | |
| | | ctgactcgctgcgctcggtcgttcggc | |
| | | tgcggcgagcggtatcagctcactcaa | |
| | | aggcggtaatacggttatccacagaat | |
| | | caggggataacgcaggaaagaacatgt | |
| | | gagcaaaaggccagcaaaaggccagga | |
| | | accgtaaaaaggccgcgttgctggcgt | |
| | | ttttccataggctccgcccccctgacg | |
| | | agcatcacaaaaatcgacgctcaagtc | |
| | | agaggtggcgaaacccgacaggactat | |
| | | aaagataccaggcgtttccccctggaa | |
| | | gctccctcgtgcgctctcctgttccga | |
| | | ccctgccgcttaccggatacctgtccg | |
| | | cctttctcccttcgggaagcgtggcgc | |
| | | tttctcatagctcacgctgtaggtatc | |
| | | tcagttcggtgtaggtcgttcgctcca | |
| | | agctgggctgtgtgcacgaaccccccg | |
| | | ttcagcccgaccgctgcgccttatccg | |
| | | gtaactatcgtcttgagtccaacccgg | |
| | | taagacacgacttatcgccactggcag | |
| | | cagccactggtaacaggattagcagag | |
| | | cgaggtatgtaggcggtgctacagagt | |
| | | tcttgaagtggtggcctaactacggct | |
| | | acactagaagaacagtatttggtatct | |
| | | gcgctctgctgaagccagttaccttcg | |
| | | gaaaaagagttggtagctcttgatccg | |
| | | gcaaacaaaccaccgctggtagcggtg | |
| | | gtttttttgtttgcaagcagcagatta | |
| | | cgcgcagaaaaaaaggatctcaagaag | |
| | | atcctttgatcttttctacggggtctg | |
| | | acgctcagtggaacgaaaactcacgtt | |
| | | aagggattttggtcatgagattatcaa | |
| | | aaaggatcttcacctagatccttttaa | |
| | | attaaaaatgaagttttaaatcaatct | |
| | | aaagtatatatgagtaaacttggtctg | |
| | | acagttaccaatgcttaatcagtgagg | |
| | | cacctatctcagcgatctgtctatttc | |
| | | gttcatccatagttgcctgactccccg | |
| | | tcgtgtagataactacgatacgggagg | |
| | | gcttaccatctggccccagtgctgcaa | |
| | | tgataccgcgagacccacgctcaccgg | |
| | | ctccagatttatcagcaataaaccagc | |
| | | cagccggaagggccgagcgcagaagtg | |
| | | gtcctgcaactttatccgcctccatcc | |
| | | agtctattaattgttgccgggaagcta | |
| | | gagtaagtagttcgccagttaatagtt | |
| | | tgcgcaacgttgttgccattgctacag | |
| | | gcatcgtggtgtcacgctcgtcgtttg | |
| | | gtatggcttcattcagctccggttccc | |
| | | aacgatcaaggcgagttacatgatccc | |
| | | ccatgttgtgcaaaaaagcggttagct | |
| | | ccttcggtcctccgatcgttgtcagaa | |
| | | gtaagttggccgcagtgttatcactca | |
| | | tggttatggcagcactgcataattctc | |
| | | ttactgtcatgccatccgtaagatgct | |
| | | tttctgtgactggtgagtactcaacca | |
| | | agtcattctgagaatagtgtatgcggc | |
| | | gaccgagttgctcttgcccggcgtcaa | |
| | | tacgggataataccgcgccacatagca | |
| | | gaactttaaaagtgctcatcattggaa | |
| | | aacgttcttcggggcgaaaactctcaa | |
| | | ggatcttaccgctgttgagatccagtt | |
| | | cgatgtaacccactcgtgcacccaact | |
| | | gatcttcagcatcttttactttcacca | |
| | | gcgtttctgggtgagcaaaaacaggaa | |
| | | ggcaaaatgccgcaaaaaagggaataa | |
| | | gggcgacacggaaatgttgaatactca | |
| | | tactcttcctttttcaatattattgaa | |
| | | gcatttatcagggttattgtctcatga | |
| | | gcggatacatatttgaatgtatttaga | |
| | | aaaataaacaaataggggttccgcgca | |
| | | catttccccgaaaagtgccacctgac | |
| 108 | Exemplary m6A | GCGGACTTACGACAGTTGCGTTACACC | |
| | sensor sequence-5′-3′ | CTTTCTCGACAAAACCTAACTTGCGCA | |
| | | GAAAACATGCCAATCTCATCTTGGCTT | |
| 109 | Exemplary m6A | GCGGCGTTACGACAGTTGCGTTACACC | |
| | sensor sequence-5′-3′ | CTTTCTCGACAAAACCTAACTTGCGCA | |
| | | GAAAACATGCCAATCTCATCTTGGCTT | |
| 110 | Exemplary m6A | GCGGACTTACGTCAGTTGCGTTACACC | |
| | sensor sequence-5′-3′ | CTTTCTCGACAAAACCTAACTTGCGCA | |
| | | GAAAACATGCCAATCTCATCTTGGCTT | |
| 111 | Exemplary m6A | GCGGAGTTACGACAGTTGCGTTACACC | |
| | sensor sequence-5′-3′ | CTTTCTCGTCAAAACCTAACTTGCGCA | |
| | | GAAAACATGCCAATCTCATCTTGGCTT | |
| 112 | Exemplary m6A | GCGGAGTTACGACAGTTGCGTTACACC | |
| | sensor sequence-5′-3′ | CTTTCTCGACAAAGCCTAACTTGCGCA | |
| | | GAAAACATGCCAATCTCATCTTGGCTT | |
| 113 | Exemplary m6A | GCGGAGTTACGACAGTTGCGTTACACC | |
| | sensor sequence-5′-3′ | CTTTCTCGACAAAACCTAGCTTGCGCA | |
| | | GAAAACATGCCAATCTCATCTTGGCTT | |
| 114 | Exemplary m6A | GCGGAGTTACGACAGTTGCGTTACACC | |
| | sensor sequence-5′-3′ | CTTTCTCGACAAAACCTAACTTGCGCA | |
| | | GAAAGCATGCCAATCTCATCTTGGCTT | |
| 115 | Exemplary m6A | GCGGACTTACGACAGTTGCGTCCAATC | |
| | sensor sequence-5′-3′ | TCATCTTGGCTT | |
| 116 | Exemplary m6A | GCGGCCTTACGTCAGTTGCGTTACACC | |
| | sensor sequence-5′-3′ | CTTTCTCGGCAAAGCCTAGCTTGCGCA | |
| | | GAAAGCATGCCAATCTCATCTTGGCTT | |
| 117 | | (GGGGS)20 | |
| 118 | deaminase domain of | TSNFNNGIGRHKTYLCYEVERLDNGTS | |
| | hAPOBEC3A | VKMDQHRGFLHNQAKNLLCGFYGRHAE | |
| | | LRFLDLVPSLQLDPAQIYRVTWFISWS | |
| | | PCFSWGCAGEVRAFLQENTHVRLRIFA | |
| | | ARIYDYDPLYKEALQMLRDAGAQVSIM | |
| | | TYDEFKHCWDTFVDHQGCPFQPWDGLD | |
| | | EHSQALSGRLR | |
| 119 | catalytic domain of | MDSLLMNRREFLYQFKNVRWAKGRRET | |
| | ADAR2 | YLCYVVKRRDSATSFSLDFGYLRNKNG | |
| | | CHVELLFLRYISDWDLDPGRCYRVTWF | |
| | | ISWSPCYDCARHVADFLRGNPNLSLRI | |
| | | FTARLYFCEAGRREPEGLRRLHRAGVQ | |
| | | IAIMTFKDYFYCWNTFVENHGRTFKAW | |
| | | EGLHENSVRLSRQLRRILL | |
| 120 | deaminase domain of | RRRIEPHEFEVFFDPRELRKETCLLYE | |
| | rAPOBEC1 | INWGGRHSIWRHTSQNTNKHVEVNFIE | |
| | | KFTTERYFCPNTRCSITWFLSWSPCGE | |
| | | CSRAITEFLSRYPHVTLFIYIARLYHH | |
| | | ADPRNRQGLRDLISSGVTIQIMTEQES | |
| | | GYCWRNFVNYSPSNEAHWPRYPHLWVR | |
| | | LYVLELYCIILGLPPCLNILRRKQPQL | |
| | | TFFTIALQSCHYQRLPPHILWATGLK | |
| 121 | hADAR1 | MNPRQGYSLSGYYTHPFQGYEHRQLRY | |
| | | QQPGPGSSPSSFLLKQIEFLKGQLPEA | |
| | | PVIGKQTPSLPPSLPGLRPRFPVLLAS | |
| | | STRGRQVDIRGVPRGVHLRSQGLQRGF | |
| | | QHPSPRGRSLPQRGVDCLSSHFQELSI | |
| | | YQDQEQRILKFLEELGEGKATTAHDLS | |
| | | GKLGTPKKEINRVLYSLAKKGKLQKEA | |
| | | GTPPLWKIAVSTQAWNQHSGVVRPDGH | |
| | | SQGAPNSDPSLEPEDRNSTSVSEDLLE | |
| | | PFIAVSAQAWNQHSGVVRPDSHSQGSP | |
| | | NSDPGLEPEDSNSTSALEDPLEFLDMA | |
| | | EIKEKICDYLFNVSDSSALNLAKNIGL | |
| | | TKARDINAVLIDMERQGDVYRQGTTPP | |
| | | IWHLTDKKRERMQIKRNTNSVPETAPA | |
| | | AIPETKRNAEFLTCNIPTSNASNNMVT | |
| | | TEKVENGQEPVIKLENRQEARPEPARL | |
| | | KPPVHYNGPSKAGYVDFENGQWATDDI | |
| | | PDDLNSIRAAPGEFRAIMEMPSFYSHG | |
| | | LPRCSPYKKLTECQLKNPISGLLEYAQ | |
| | | FASQTCEFNMIEQSGPPHEPRFKFQVV | |
| | | INGREFPPAEAGSKKVAKQDAAMKAMT | |
| | | ILLEEAKAKDSGKSEESSHYSTEKESE | |
| | | KTAESQTPTPSATSFFSGKSPVTTLLE | |
| | | CMHKLGNSCEFRLLSKEGPAHEPKFQY | |
| | | CVAVGAQTFPSVSAPSKKVAKQMAAEE | |
| | | AMKALHGEATNSMASDNQPEGMISESL | |
| | | DNLESMMPNKVRKIGELVRYLNTNPVG | |
| | | GLLEYARSHGFAAEFKLVDQSGPPHEP | |
| | | KFVYQAKVGGRWFPAVCAHSKKQGKQE | |
| | | AADAALRVLIGENEKAERMGFTEVTPV | |
| | | TGASLRRTMLLLSRSPEAQPKTLPLTG | |
| | | STFHDQIAMLSHRCFNTLTNSFQPSLL | |
| | | GRKILAAIIMKKDSEDMGVVVSLGTGN | |
| | | RCVKGDSLSLKGETVNDCHAEIISRRG | |
| | | FIRFLYSELMKYNSQTAKDSIFEPAKG | |
| | | GEKLQIKKTVSFHLYISTAPCGDGALF | |
| | | DKSCSDRAMESTESRHYPVFENPKQGK | |
| | | LRTKVENGEGTIPVESSDIVPTWDGIR | |
| | | LGERLRTMSCSDKILRWNVLGLQGALL | |
| | | THFLQPIYLKSVTLGYLFSQGHLTRAI | |
| | | CCRVTRDGSAFEDGLRHPFIVNHPKVG | |
| | | RVSIYDSKRQSGKTKETSVNWCLADGY | |
| | | DLEILDGTRGTVDGPRNELSRVSKKNI | |
| | | FLLFKKLCSFRYRRDLLRLSYGEAKKA | |
| | | ARDYETAKNYFKKGLKDMGYGNWISKP | |
| | | QEEKNFYLCPV | |
| 122 | hADAR1 catalytic | LPLTGSTFHDQIAMLSHRCFNTLTNSF | |
| | domain | QPSLLGRKILAAIIMKKDSEDMGVVVS | |
| | | LGTGNRCVKGDSLSLKGETVNDCHAEI | |
| | | ISRRGFIRFLYSELMKYNSQTAKDSIF | |
| | | EPAKGGEKLQIKKTVSFHLYISTAPCG | |
| | | DGALFDKSCSDRAMESTESRHYPVFEN | |
| | | PKQGKLRTKVENGEGTIPVESSDIVPT | |
| | | WDGIRLGERLRTMSCSDKILRWNVLGL | |
| | | QGALLTHFLQPIYLKSVTLGYLFSQGH | |
| | | LTRAICCRVTRDGSAFEDGLRHPFIVN | |
| | | HPKVGRVSIYDSKRQSGKTKETSVNWC | |
| | | LADGYDLEILDGTRGTVDGPRNELSRV | |
| | | SKKNIFLLFKKLCSFRYRRDLLRLSYG | |
| | | EAKKAARDYETAKNYFKKGLKDMGYGN | |
| | | WISKPQEEKNFYLCPV | |
| 123 | | A(EAAAK)₁₀A | |
| 124 | | A(EAAAK)₂₀A | |
| 125 | human TP53 amino | MEEPQSDPSVEPPLSQETFSDLWKLLP | canonical human |
| | acid sequence | ENNVLSPLPSQAMDDLMLSPDDIEQWF | p53 sequence |
| | | TEDPGPDEAPRMPEAAPPVAPAPAAPT | (accessed at |
| | | PAAPAPAPSWPLSSSVPSQKTYQGSYG | uniprot.org/uniprot |
| | | FRLGFLHSGTAKSVTCTYSPALNKMFC | kb/P04637/entry#s |
| | | QLAKTCPVQLWVDSTPPPGTRVRAMAI | equences) |
| | | YKQSQHMTEVVRRCPHHERCSDSDGLA | |
| | | PPQHLIRVEGNLRVEYLDDRNTFRHSV | |
| | | VVPYEPPEVGSDCTTIHYNYMCNSSCM | |
| | | GGMNRRPILTIITLEDSSGNLLGRNSF | |
| | | EVRVCACPGRDRRTEEENLRKKGEPHH | |
| | | ELPPGSTKRALPNNTSSSPQPKKKPLD | |
| | | GEYFTLQIRGRERFEMFRELNEALELK | |
| | | DAQAGKEPGGSRAHSSHLKSKKGQSTS | |
| | | RHKKLMFKTEGPDSD | |
| 126 | human SOCS2 amino | MTLRCLEPSGNGGEGTRSQWGTAGSAE | canonical human |
| | acid sequence | EPSPQAARLAKALRELGQTGWYWGSMT | p53 sequence |
| | | VNEAKEKLKEAPEGTFLIRDSSHSDYL | (accessed at |
| | | LTISVKTSAGPTNLRIEYQDGKFRLDS | uniprot.org/uniprot |
| | | IICVKSKLKQFDSVVHLIDYYVQMCKD | kb/O14508/entry#s |
| | | KRTGPEAPRNGTVHLYLTKPLYTSAPS | equences) |
| | | LQHLCRLTINKCTGAIWGLPLPTRLKD | |
| | | YLEEYKFQV | |

Disclosed are materials, compositions, and components that can be used for, can be used in conjunction with, can be used in preparation for, or are products of the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. An expression system comprising:
(a) a first DNA construct comprising a polynucleotide encoding a fusion protein, wherein the fusion protein comprises an N⁶-methyladenosine (m⁶A) binding domain of a YT521-B homology (YTH) domain-containing protein fused to a catalytic domain of a cytidine deaminase or a catalytic domain of an adenosine deaminase; and
(b) a second DNA construct comprising a polynucleotide encoding a heterologous polypeptide, the polynucleotide encoding a heterologous polypeptide comprising:
i. a polynucleotide encoding an effector protein;
ii. a polynucleotide encoding a m⁶A sensor sequence; and
iii. a polynucleotide encoding a dihydrofolate reductase (DHFR).

2. The expression system of claim 1, wherein the m⁶A binding domain comprises a sequence having at least 90% or greater sequence identity to SEQ ID NOs: 66 or 108-116.

3. The expression system of claim 1, wherein the m⁶A binding domain is fused to the catalytic domain via a peptide linker.

4. The expression system of claim 1, wherein the catalytic domain comprises a polypeptide having at least 95% identity to SEQ ID NO 78 or a catalytic fragment thereof, SEQ ID NO: 79 or a catalytic fragment thereof; SEQ ID NO: 80 or a catalytic fragment thereof; or SEQ ID NO: 81.

5. The expression system of claim 1, wherein a vector comprises the first DNA construct.

6. The expression system of claim 1, wherein a vector comprises the second DNA construct.

7. The expression system of claim 1, wherein a vector comprises the first DNA construct and the second DNA construct.

8. The expression system of claim 1, wherein the nucleic acid sequence encoding a fusion protein, the nucleic acid sequence encoding a heterologous polypeptide and a polypeptide encoding dihydrofolate reductase (DHFR), or both, are operably linked to a first promoter.

9. The expression system of claim 9, wherein the system further comprises a nucleic acid sequence encoding a selectable marker operably linked to a second promoter.

10. The expression system of claim 9, wherein the first promoter is a constitutive or an inducible promoter.

11. The expression system of claim 1, wherein the cytidine deaminase is APOBEC-1.

12. The expression system of claim 1, wherein the effector protein is a tumor suppressor protein or a catalytically dead RNA-guided endonuclease.

13. The expression system of claim 13, wherein the tumor suppressor protein is suppressor of cytokine signaling 2 (SOC2) or p53 or one of the proteins listed in Table 1.

14. The expression system of claim 13, wherein the catalytically dead RNA-guided endonuclease is a dCas9 or a dCas13.

15. A polynucleotide comprising a nucleic acid sequence encoding an effector protein polypeptide, a m⁶A sensor sequence, and a polypeptide encoding dihydrofolate reductase (DHFR).

16. A vector comprising the expression system of claim 1.

17. A host cell comprising the expression system of claim 1 or the vector of claim 17.

18. A non-human transgenic animal comprising the host cell of claim 18.

19. A kit comprising the expression system of claim 1.

20. A method of increasing expression of a tumor suppressor protein in one or more cells, comprising introducing the expression system of claim 1 into the one or more cells.

21. The method of claim 21, wherein the one or more cells are hepatocellular carcinoma (HCC) cells.

22. (canceled)

23. A method of reducing M6A effector regulator expression, comprising:
introducing an expression system into a subject having or suspected of having a cancer, wherein the expression system comprises:
(a) a first DNA construct comprising a polynucleotide encoding a fusion protein, wherein the fusion protein comprises an N⁶-methyladenosine (m⁶A) binding domain of a YT521-B homology (YTH) domain-containing protein fused to a catalytic domain of a cytidine deaminase or a catalytic domain of an adenosine deaminase; and
(b) a second DNA construct comprising a polynucleotide encoding a heterologous polypeptide, the polynucleotide encoding a heterologous polypeptide comprising:
iv. a polynucleotide encoding a catalytically-dead RNA-guided endonuclease;
v. a polynucleotide encoding a m⁶A sensor sequence; and
vi. a polynucleotide encoding a dihydrofolate reductase (DHFR);
(c) an sgRNA configured to bind to an m6a regulator.

24. (canceled)

25. (canceled)

26. (canceled)

27. (canceled)

28. A method of reducing M6A hypermethylation, comprising:
introducing an expression system into a subject having or suspected of having a cancer, wherein the expression system comprises:
(a) a first DNA construct comprising a polynucleotide encoding a fusion protein, wherein the fusion protein comprises an N⁶-methyladenosine (m⁶A) binding domain of a YT521-B homology (YTH) domain-containing protein fused to a catalytic domain of a cytidine deaminase or a catalytic domain of an adenosine deaminase; and
(b) a second DNA construct comprising a polynucleotide encoding a heterologous polypeptide, the polynucleotide encoding a heterologous polypeptide comprising:
vii. a polynucleotide encoding a catalytically-dead RNA-guided endonuclease;
viii. a polynucleotide encoding a m⁶A sensor sequence; and
ix. a polynucleotide encoding a dihydrofolate reductase (DHFR);
(c) an sgRNA configured to bind to an m6a regulator.

28. (canceled)

29. (canceled)

30. (canceled)

31. (canceled)

32. A method of inhibiting a cancer cell, the method comprising:
introducing the expression system of claim 1 into the cancer cell, wherein the cancer cell comprises m⁶A RNA hypermethylation, and wherein the second DNA construct comprising a polynucleotide encoding an effector protein, the effector protein comprising a tumor suppressor protein.

33. (canceled)

34. (canceled)

35. (canceled)

36. (canceled)

37. (canceled)

38. (canceled)

39. (canceled)

40. (canceled)

41. (canceled)

42. (canceled)

43. A method of treating a subject having a cancer characterized by m⁶A RNA hypermethylation, the method comprising inhibiting a cancer cell according to the method of claim 32.

44. The method of claim 43, wherein the cancer comprises at least one of acute myeloid leukemia (AML), glioblastoma (GBM), lung cancer, endometrial cancer, cervical cancer, ovarian cancer, breast cancer, colorectal cancer (CRC), a hepatocellular carcinoma (HCC), pancreatic cancer, gastric cancer, prostate cancer, or renal cell carcinoma.

45. (canceled)

46. (canceled)

47. (canceled)
